(19) United States
(12) Patent Application Publication
(10) Pub. No.: US 2026/0265737 A1
(43) Pub. Date: Sep. 10, 2026

(54) CYCLIC-DISULFIDE MODIFIED PHOSPHATE BASED OLIGONUCLEOTIDE PRODRUGS

(71) Applicant: ALNYLAM PHARMACEUTICALS, INC., Cambridge, MA (US)

(72) Inventors: Alexander V. KEL'IN, Cambridge, MA (US); Justin M. PIERSON, Cambridge, MA (US); Jayaprakash K. NAIR, Cambridge, MA (US); Cheng TANG, Cambridge, MA (US); Patrick MILLER, Cambridge, MA (US); Punit SETH, Cambridge, MA (US)

(21) Appl. No.: 18/877,507

(22) PCT Filed: Jun. 30, 2023
(86) PCT No.: PCT/US2023/069515

§ 371 Date: Dec. 20, 2024

Related U.S. Application Data
(60) Provisional application No. 63/357,050, filed on Jun. 30, 2022.

(51) Int. Cl. C12N 15/113; C07F 9/6553

(52) U.S. Cl. CPC C12N 15/113; C07F 9/655354; C12N 2310/14; C12N 2310/322; C12N 2310/323; C12N 2310/3231; C12N 2310/3233; C12N 2310/332

(57) ABSTRACT

This invention relates to a compound comprising a structure of formula (I): cyclic disulfide moiety—phosphorus coupling group (I). The cyclic disulfide moiety has the structure of: (C-I), (C-IIa)), (C-IIb), or (C-III). This invention also relates to an oligonucleotide comprising one or more compounds that comprise the structure of formula (I), wherein at least one phosphorus coupling group contains a nucleoside or oligonucleotide. The invention also relates to a pharmaceutical composition comprising the oligonucleotide described herein and a method of reducing or inhibiting the expression of a target gene by administering to the subject a therapeutically effective amount of the oligonucleotide described herein.

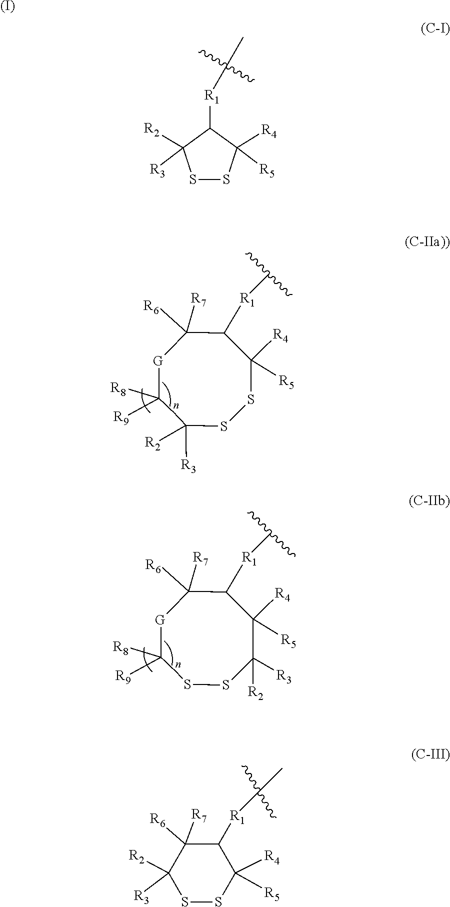

### CROSS-REFERENCE TO RELATED APPLICATIONS

This application claims benefit of priority to U.S. Provisional Application No. 63/357,050, filed Jun. 30, 2022, which is herein incorporated by reference in its entirety.

### SEQUENCE LISTING

The instant application contains a Sequence Listing which has been submitted in XML format and is hereby incorporated by reference in its entirety. Said XML copy, created on Jun. 29, 2023, is named 29520.1510-PCT__SL.xml and is 4,495,791 bytes in size.

### FIELD OF INVENTION

This invention generally relates to the field of modified phosphate based oligonucleotide prodrugs.

### BACKGROUND

Phosphate esters are important intermediates in formation of nucleotides and their assembly into RNA and DNA. Within the cell, the phosphate group commonly serves as a tunable leaving group. Phosphate esters are charged at a physiological pH, which serve to bind the phosphate esters to the active site of an enzyme. However, in order for the phosphate esters to bind the enzymes, they must first penetrate the membrane to access the enzyme, as charged molecules can have difficulty traversing the cell membrane other than by endocytosis. This limitation may be ameliorated in the case of compounds with larger, more lipophilic substituents.

Alternatively, prodrug approaches have been researched to temporarily mask any negative charges of the phosphate esters on the oligonucleotide at a physiological pH. A prodrug is an agent that is administered in an inactive or significantly less active form, and that undergoes chemical or enzymatic transformations in vivo to yield the active parent drug under different stimuli. Prodrug approaches to mask the negative charges of phosphate groups of the oligonucleotide with cell-cleavable protecting/masking groups can offer a number of advantages over their non-protected counterparts including, e.g., enhancing cell penetration and avoiding or minimizing degradation in serum via cellular sequestration.

However, the prodrug approach still has substantial challenges, partially because it is difficult to choose the best masking group. For instance, cellular cleavage of the protecting groups can often generate products which are viewed as disadvantageous or even toxic. Moreover, the protecting groups must strike a balance between allowing absorption in the intestines and allowing cleavage in the blood or target cell.

Thus, there is a continuing need for developing new and improved modified phosphate prodrugs for masking internucleotide phosphate linkages of an oligonucleotide, in order to make effective and efficient oligonucleotide-based drugs, for efficient in vivo delivery and improved in vivo efficacy of oligonucleotides.

### SUMMARY

One aspect of the invention relates to a compound comprising a structure of formula (I), or a salt or stereoisomer thereof:

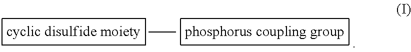

### The

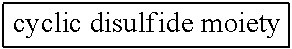

has the structure of:

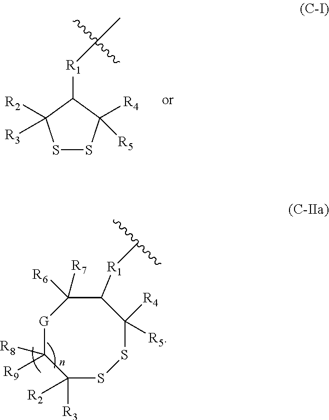

### Alternatively, the

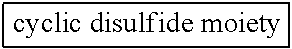

can have the structure of

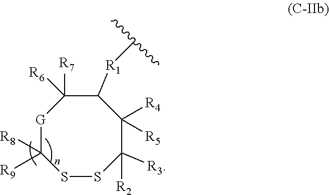

In these formulas:
R₁ is O or S, and is bonded to the P atom of the

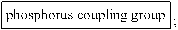

[symbol]

indicates the bond to the

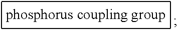

R₂, R₄, R₆, R₇, R₈, and R₉ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups;
R₃ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₃ and R₅, together with the adjacent carbon atoms and the two sulfur atoms, form a second ring;
R₂ and R₃, together with the adjacent carbon atom, can form another ring;
R₄ and R₅, together with the adjacent carbon atom, can form another ring;
R₆ and R₇, together with the adjacent carbon atom, can form another ring;
R₈ and R₉, together with the adjacent carbon atoms, can form another ring;
two or more of R₂, R₃, R₄, R₅, R₆, R₇, R₈, R₉, R¹⁴, and R¹⁵, together with the adjacent carbon atoms can form one or more rings fused with the ring containing the two sulfur atoms;
G is O, N(R′), S, or C(R¹⁴)(R¹⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″, together with the adjacent nitrogen atom, form a ring; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.

In some embodiments, in the

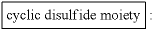

R₁ is O;
G is CH₂;
n is 0 or 1;
R₂, R₄, R₆, R₇, R₈, and R₉ are each independently H, halo, CN or C₁-C₆ alkylene-CN, C(O)OR¹³ or C₁-C₆ alkylene-C(O)OR¹³, S(O)OR¹³ or C₁-C₆ alkylene-S(O)OR¹³, C(O)N(R′)(R″) or C₁-C₆ alkylene-C(O)N(R′)(R″), OR¹³ or C₁-C₆ alkylene-OR¹³, N(R′)(R″) or C₁-C₆ alkylene-N(R′)(R″), C₁-C₆ alkyl, aryl, heteroaryl, each of which can be optionally substituted by one or more R^{sub} groups;
R₃ and R₅ are each independently H, halo, CN or C₁-C₆ alkylene-CN, C(O)OR¹³ or C₁-C₆ alkylene-C(O)OR¹³, S(O)OR¹³ or C₁-C₆ alkylene-S(O)OR¹³, C(O)N(R′)(R″) or C₁-C₆ alkylene-C(O)N(R′)(R″), OR¹³ or C₁-C₆ alkylene-OR¹³, N(R′)(R″) or C₁-C₆ alkylene-N(R′)(R″), C₁-C₆ alkyl, aryl, heteroaryl, each of which can be optionally substituted by one or more R^{sub} groups; or R₃ and R⁵, together with the adjacent carbon atoms and the two sulfur atoms, form a second ring of 6-8 atoms;
R₂ and R₃, together with the adjacent carbon atom, can form another ring of 3-7 atoms;
R₄ and R₅, together with the adjacent carbon atom, can form another ring of 3-7 atoms;
R₆ and R₇, together with the adjacent carbon atom, can form another ring of 3-7 atoms;
R₈ and R₉, together with the adjacent carbon atom, can form another ring of 3-7 atoms;
two or more of R₂, R₃, R₄, R₅, R₆, R₇, R₈, R₉, R¹⁴, and R¹⁵, together with the adjacent carbon atoms can form one or more ring of 5-7 atoms fused with the ring containing the two sulfur atoms;
R¹³ is independently for each occurrence H, C₁-C₆ alkyl, aryl, alkylcarbonyl, or arylcarbonyl; and
R′ and R″ are each independently H or C₁-C₆ alkyl.

In some embodiments, the

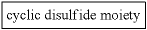

has the structure of

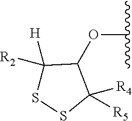

(C-Ia). R₂ may be optionally substituted aryl, for instance, optionally substituted phenyl. In some embodiments, R₂ is mono-, di-, or tri-substituted phenyl. In one embodiment, R₂ is para-substituted phenyl. In some embodiments, R₂ is optionally substituted C₁₋₆ alkyl. In one embodiment, R₂ is haloC₁₋₆ alkyl. In one embodiment, R₂ is C₁₋₆alkyl.

In some embodiments, the

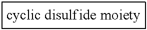

has the structure of

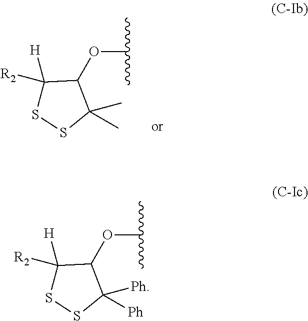

R₂ may be optionally substituted aryl, for instance, optionally substituted phenyl. In some embodiments, R₂ is mono-, di-, or tri-substituted phenyl. In one embodiment, RZ is para-substituted phenyl. In some embodiments, RZ is optionally substituted C₁₋₆ alkyl. In one embodiment, R₂ is haloC₁₋₆ alkyl. In one embodiment, R₂ is C₁₋₆ alkyl.

In some embodiments, the

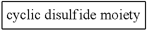

has the structure selected from one of the following Ia), Ib), and II) groups. Ia) group contains the following structures:

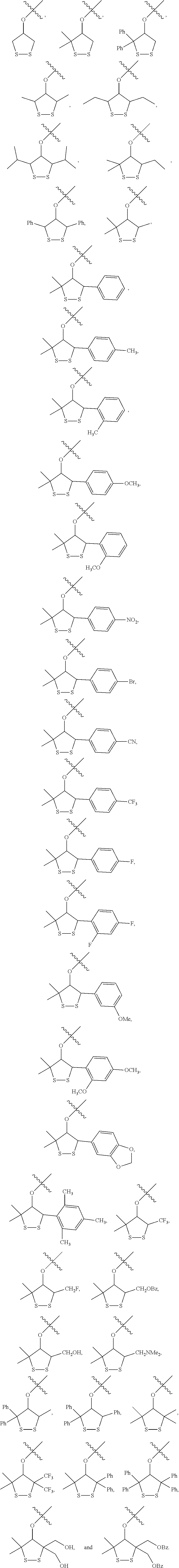

Ib) group contains the following structures:

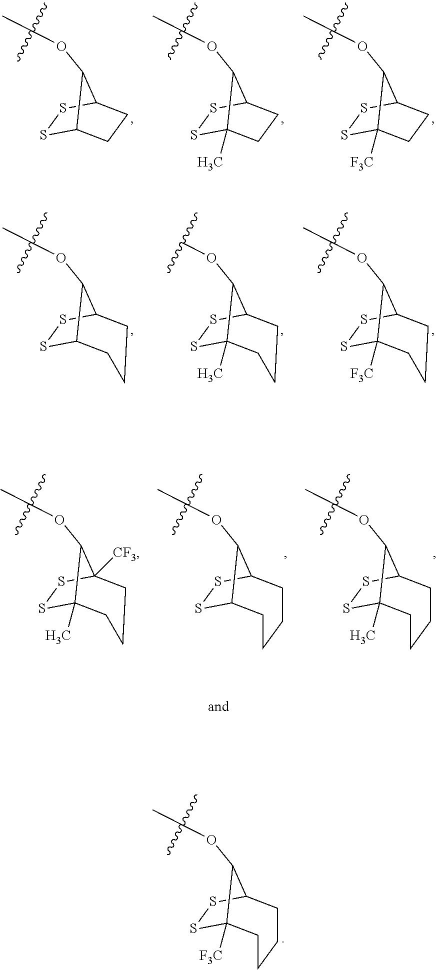

II) group contains the following structures:

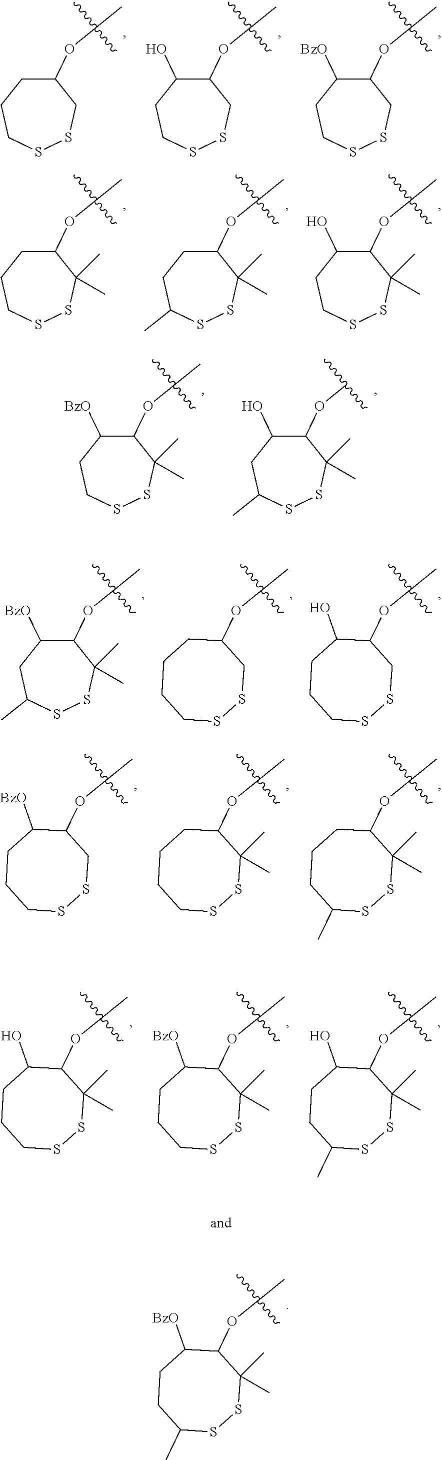

In some embodiments, the

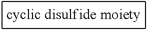

has the structure of:

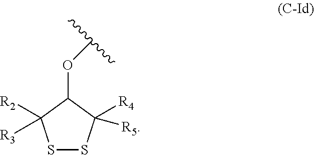

R₄ and R₅ may each be independently H, C₁₋₆ alkyl, or phenyl. In some embodiments, R₄ and R₅ are each independently C₁₋₃ alkyl. In one embodiment, R₄ and R₅ are each methyl. In some embodiments, one or both of R₄ and R₅ are phenyl. R₂ and R₃ are as defined above. In some embodiments, R₂ and R₃ are each independently H, C₁₋₆ alkyl, CN or CH₂CN, C(O)OR¹³ or CH₂C(O)OR¹³, S(O)OR¹³ or CH₂S(O)OR¹³, C(O)N(R′)(R″) or CH₂C(O)N(R′)(R″), or C(R¹⁴)(R¹⁵)(R¹⁶) or CH₂C(R¹⁴)(R¹⁵)(R¹⁶). R¹³, R¹⁴, R¹⁵, R¹⁶, R′, R″, and R^{sub} are as defined above. In some embodiments, R¹³ is independently for each occurrence H, C₁₋₆ alkyl, cycloalkyl, aryl, heteroaryl, or aralkyl. In some embodiments, R¹⁴, R¹⁵, and R¹⁶ are each independently H, halo, C₁₋₆ alkyl, alkaryl, aryl, or heteroaryl. In some embodiments, R′ and R″ are each independently H, C₁₋₆ alkyl, aryl, or heteroaryl. In some embodiments, one of R₂ and R₃ is H, and the other is an electron-withdrawing group such as CN, CF₃, CH₂CF₃, CF₂H, CF₂-phenyl, S(O)OR¹³, C(O)OR¹³, CH₂S(O)OR¹³, CH₂C(O)OR¹³, or CONHR¹³. In some embodiments, both R₂ and R₃ are electron-withdrawing groups such as CN, CF₃, CH₂CF₃, CF₂H, CF₂-phenyl, S(O)OR¹³, C(O)OR¹³, CH₂S(O)OR¹³, CH₂C(O)OR¹³, or CONHR¹³. In some embodiments, R¹³ is independently for each occurrence H, C₁₋₃ alkyl, phenyl.

In some embodiments, the

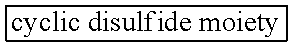

has one of the following structures:

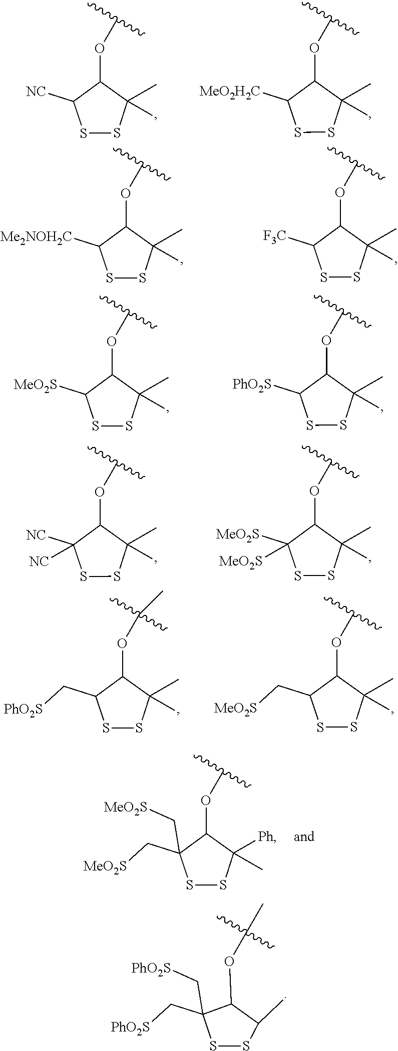

In some embodiments, the

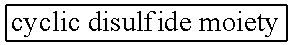

has the structure of:

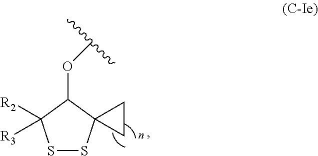

wherein n is 1 to 4. In some embodiments, n is 2, 3, or 4. R₂ and R₃ are as defined above. In some embodiments, R₂ and R₃ are each independently H, C₁₋₆ alkyl, aryl, heteroaryl, CN or CH₂CN, OR¹³ or CH₂OR¹³, C(O)OR¹³ or CH₂C(O)OR¹³, S(O)OR¹³ or CH₂S(O)OR¹³, C(O)N(R′)(R″) or CH₂C(O)N(R′)(R″), or C(R¹⁴)(R¹⁵)(R¹⁶) or CH₂C(R¹⁴)(R¹⁵)(R¹⁶), each of which can be optionally substituted by one or more R^{sub} groups. R¹³, R¹⁴, R¹⁵, R¹⁶, R′, R″, and R^{sub} are as defined above. In some embodiments, R¹³ is independently for each occurrence H, C₁₋₆ alkyl, cycloalkyl, aryl, heteroaryl, or aralkyl. In some embodiments, R¹⁴, R¹⁵, and R¹⁶ are each independently H, halo, C₁₋₆ alkyl, alkaryl, aryl, or heteroaryl. In some embodiments, R′ and R″ are each independently H, C₁₋₆ alkyl, aryl, or heteroaryl. In some embodiments, one of R₂ and R₃ is H, and the other is alkyl, aryl, CF₃, CH₂CF₃, CF₂H, CF₂-phenyl, S(O)OR¹³, C(O)OR¹³, CH₂S(O)OR¹³, CH₂C(O)OR¹³, or CONHR¹³. In some embodiments, both R₂ and R₃ are H, alkyl, aryl, CF₃, CH₂CF₃, CF₂H, CF₂-phenyl, S(O)OR¹³, C(O)OR¹³, CH₂S(O)OR¹³, CH₂C(O)OR¹³, or CONHR¹³. In some embodiments, R¹³ is independently for each occurrence H, C₁₋₃ alkyl, phenyl.

In some embodiments, the

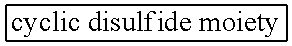

has one of the following structures:

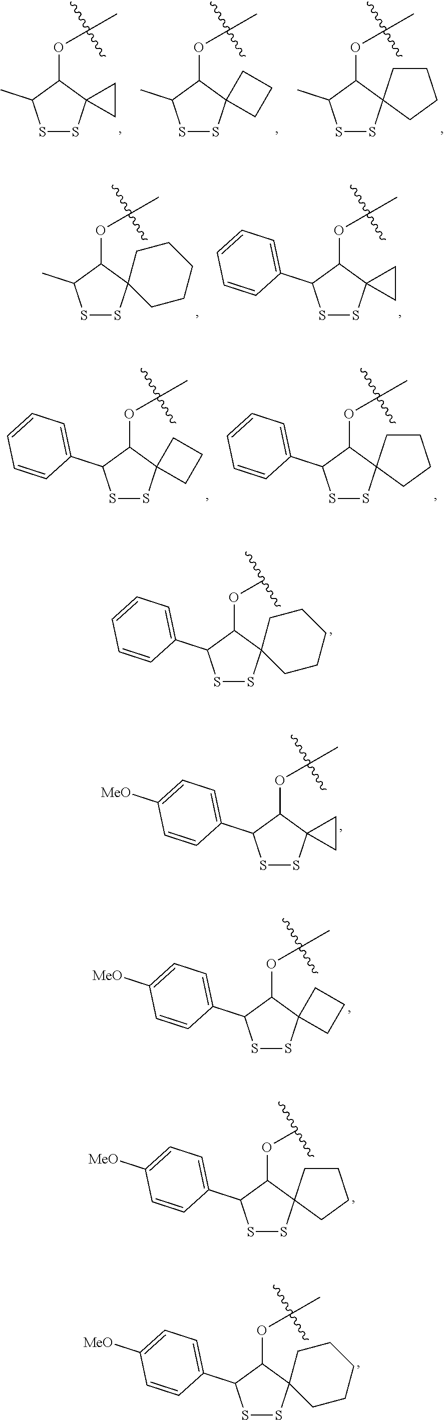

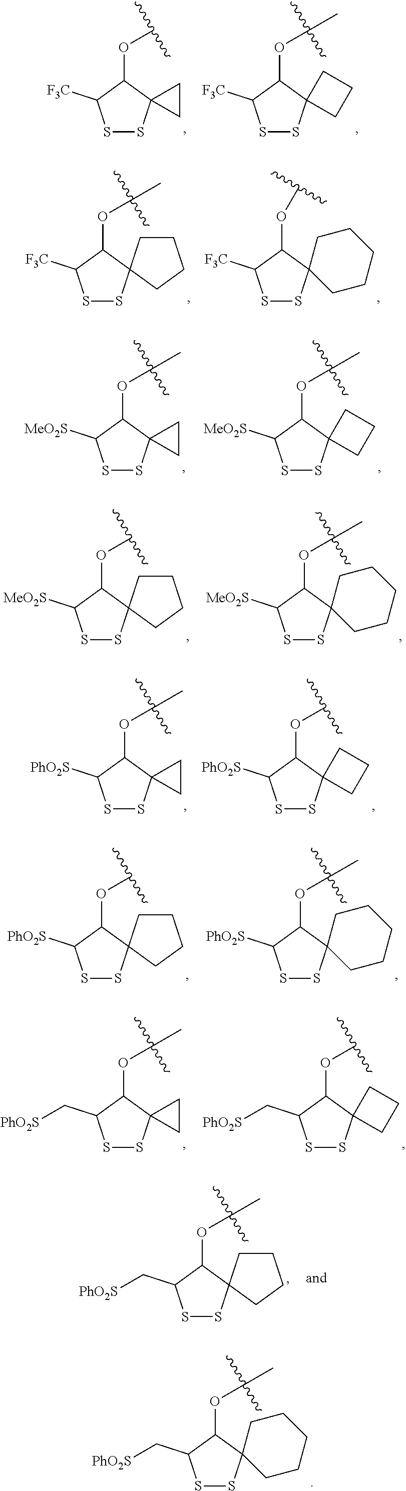

In some embodiments, the

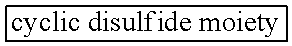

contains a disulfide-containing bridged, bicyclic structure, i.e., in formula (C-I), (C-IIa) or (C-IIb), R₃ and R₅, together with the adjacent carbon atoms and the two sulfur atoms, form a second ring.

In some embodiments, the

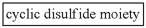

has the structure of:

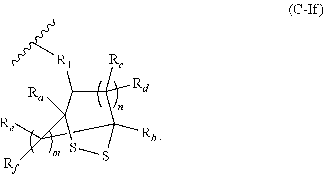

R¹ is as defined above. In one embodiment, R¹ is O. n is 0, 1, or 2. In one embodiment, n is 0. M is 1, 2, or 3. Rₐ, R_{b}, R_{c}, R_{d}, Rₑ, and R_{f} are each independently H, halo, alkyl, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), C(R¹⁴)(R¹⁵)(R¹⁶) or alkylene-C(R¹⁴)(R¹⁵)(R¹⁶), alkenyl, alkynyl, cycloalkyl, heterocyclyl, aryl, or heteroaryl. R¹³, R¹⁴, R¹⁵, R¹⁶, R′, R″, and R^{sub} are as defined above. In some embodiments, Rₑ and R_{f} are each independently H or C₁₋₆ alkyl. In some embodiments, one of Rₐ and R_{b} is H, and the other is H, C₁₋₆ alkyl, phenyl, CN, CF₃, CH₂CF₃, CF₂H, CF₂-phenyl, S(O)OR¹³, C(O)OR¹³, CH₂S(O)OR¹³, CH₂C(O)OR¹³, or CONHR¹³. In some embodiments, both Rₐ and R_{b} are H, C₁₋₆ alkyl, phenyl, CN, CF₃, CH₂CF₃, CF₂H, CF₂-phenyl, S(O)OR¹³, C(O)OR¹³, CH₂S(O)OR¹³, CH₂C(O)OR¹³, or CONHR¹³. In some embodiments, R¹³ is independently for each occurrence H, C₁₋₃ alkyl, phenyl.

In some embodiments, the

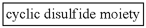

has the structure of:

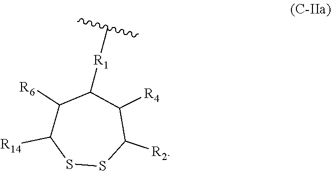

R¹ is as defined above. In one embodiment, R¹ is 0. In some embodiments, R₂ and R₄, together with the adjacent carbon atoms, form a ring (e.g., 3-7 membered ring) fused with the ring containing the two sulfur atoms; and/or R₆ and R₁₄, together with the adjacent carbon atoms, form a ring (e.g., 3-7 membered ring) fused with the ring containing the two sulfur atoms.

In some embodiments, the

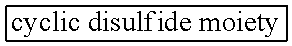

has one of the following structures:

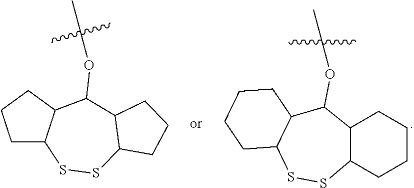 [SMILES: *OC1C2CCCC2SSC2CCCC21 ; *OC1C2CCCCC2SSC2CCCCC21]

In some embodiments, the

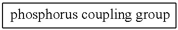

has the structure of:

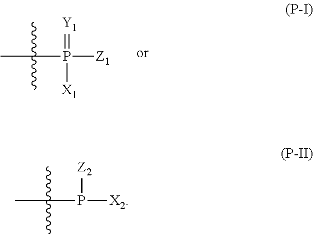

In these formulas:
[symbol] indicates the bond to the

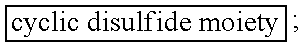

X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), NSO₂R′, N═CN(R′)(R″), B(R¹³)₃, BH₃⁻, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide,
Y₁ is S, O, or N(R′);
M is an organic or inorganic cation; and
R¹⁸ is H or alkyl, optionally substituted with one or more R^{sub} groups.

In some embodiments, the

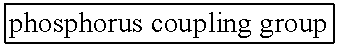

has the structure of

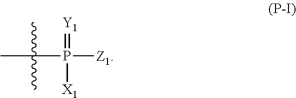

In this formula: X₁ and Z₁ are each independently OH, OM, OR¹³, SH, SM, SR¹³, C(O)H, S(O)H, C₁-C₆ alkyl optionally substituted with one or more hydroxy or halo groups, NSO₂R′, N(R′)(R″), N═CN(R′)(R″), or D-Q; D is independently for each occurrence absent, O, S, NH, C₁-C₆ alkylene optionally substituted with one or more halo groups; and Y₁ is S or O. In one embodiment, X₁ is OH or SH; and Z₁ is D-Q.

In some embodiments, the

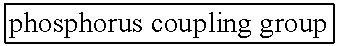

has one of the following structures:

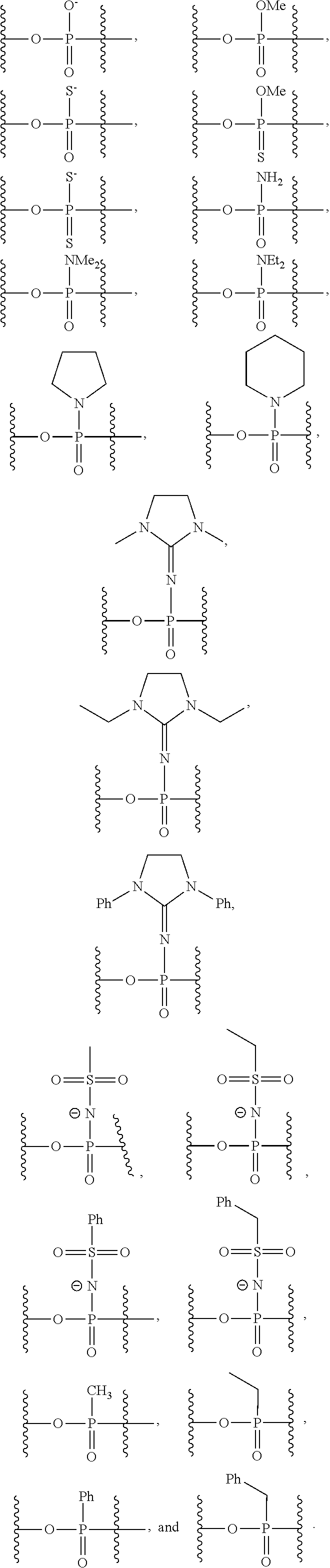

One [symbol] indicates the bond to the

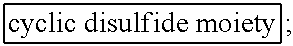

the other [symbol] indicates the bond to a nucleoside or oligonucleotide.

In some embodiments, the

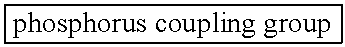

has the structure of

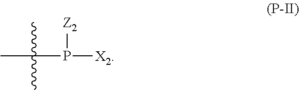

In this formula, X₂ is N(R′)(R″); Z₂ is X₂, OR¹⁸, or D-Q; R¹⁸ is H or C₁-C₆ alkyl substituted with cyano; and R′ and R″ are each independent C₁-C₆ alkyl.

In one embodiment, the

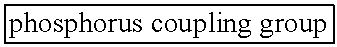

has a structure selected from the group consisting of

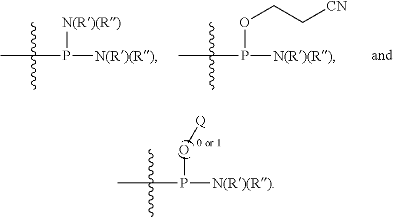

The variables R′, R″, and Q are defined as above in formulas P-I and P-II.

In one embodiment, the compound has one of the following structures:

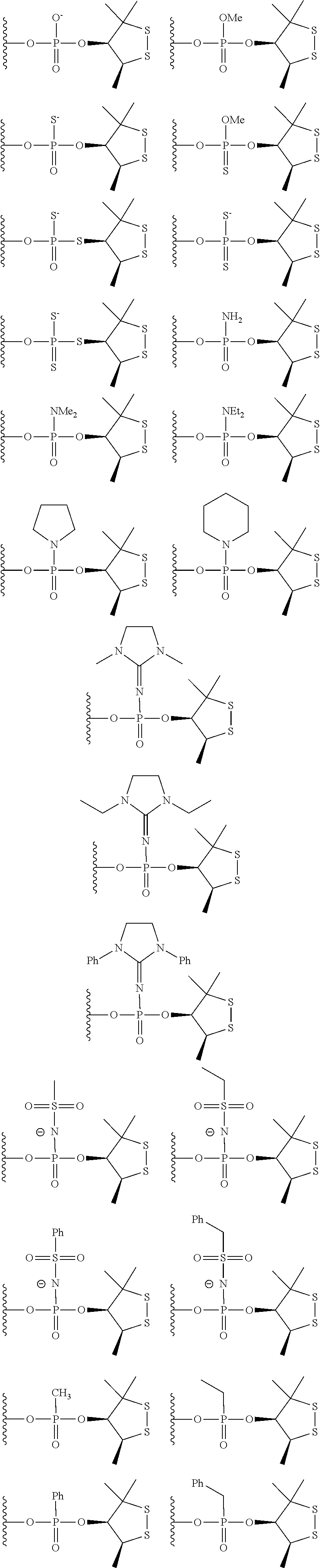

In one embodiment, the compound has one of the following structures:

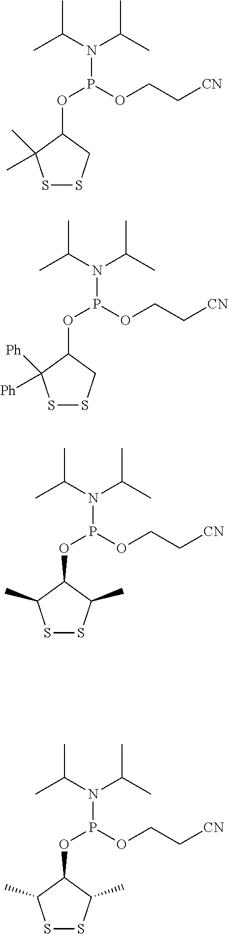

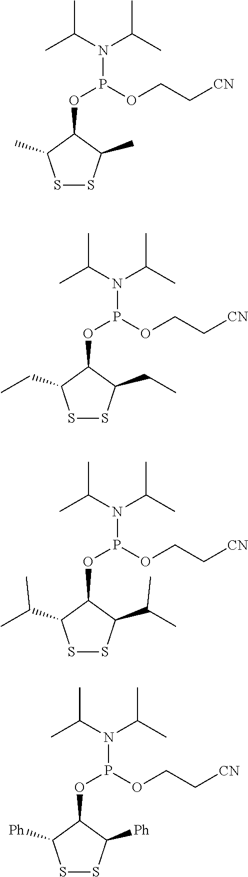

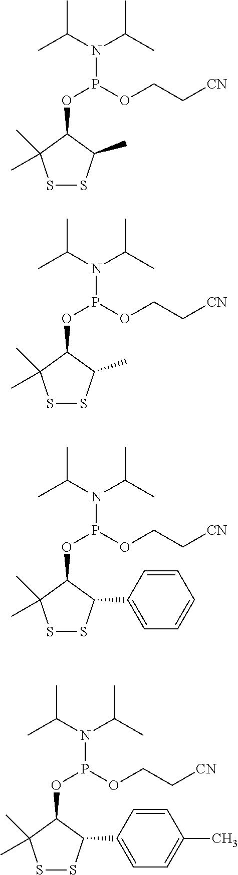

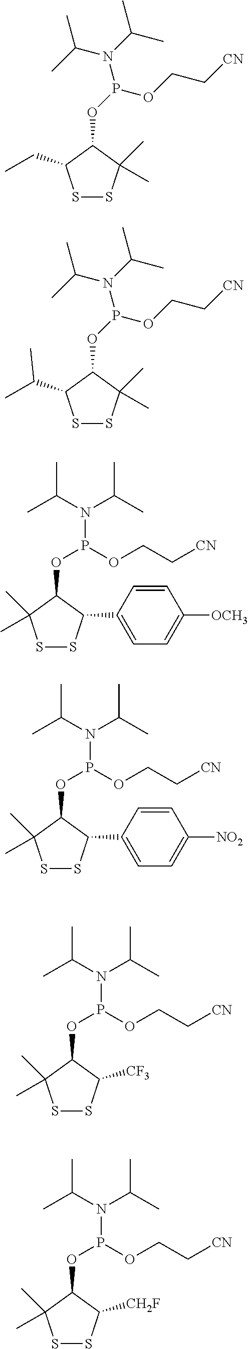

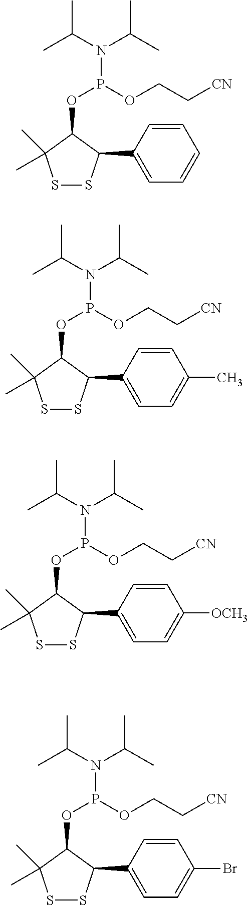

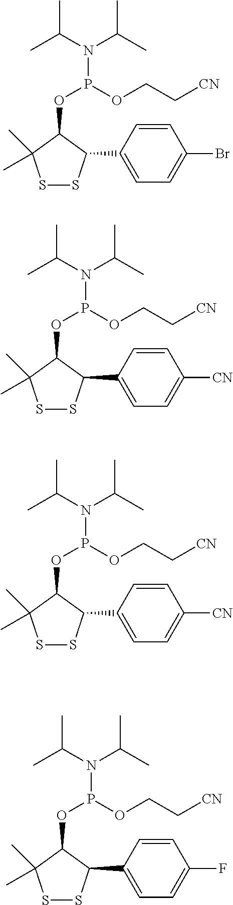

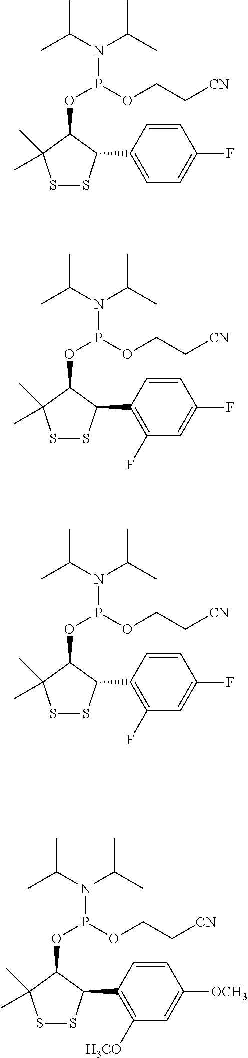 [SMILES: CC(C)N(C(C)C)P(OCCC#N)O[C@H]1[C@H](c2ccc(F)cc2)SSC1(C)C ; CC(C)N(C(C)C)P(OCCC#N)O[C@H]1[C@@H](c2ccc(F)cc2F)SSC1(C)C ; CC(C)N(C(C)C)P(OCCC#N)O[C@H]1[C@H](c2ccc(F)cc2F)SSC1(C)C ; COc1ccc([C@H]2SSC(C)(C)[C@H]2OP(OCCC#N)N(C(C)C)C(C)C)c(OC)c1]

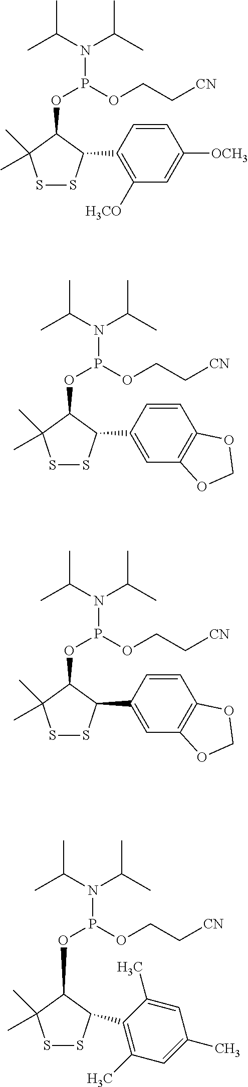

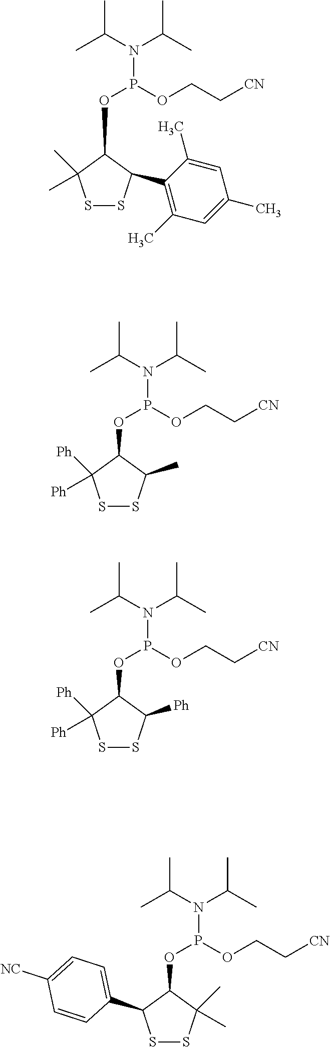

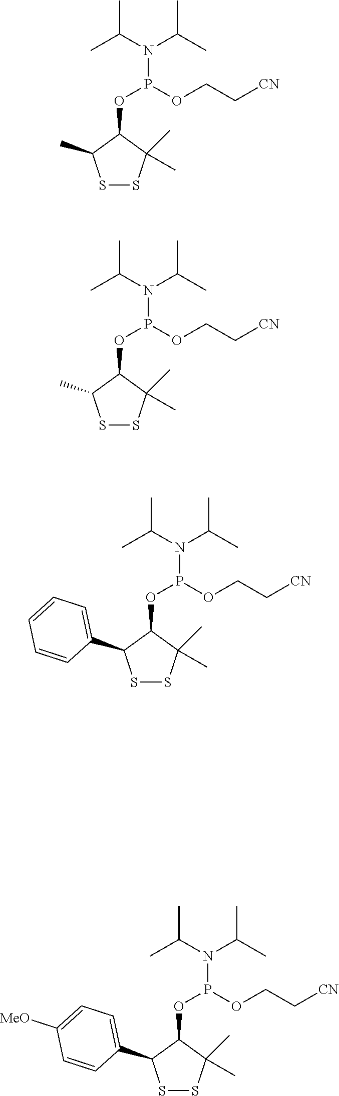 [SMILES: CC(C)N(C(C)C)P(OCCC#N)O[C@@H]1[C@H](C)SSC1(C)C ; CC(C)N(C(C)C)P(OCCC#N)O[C@@H]1[C@@H](C)SSC1(C)C ; CC(C)N(C(C)C)P(OCCC#N)O[C@@H]1[C@H](c2ccccc2)SSC1(C)C ; COc1ccc([C@@H]2SSC(C)(C)[C@@H]2OP(OCCC#N)N(C(C)C)C(C)C)cc1]

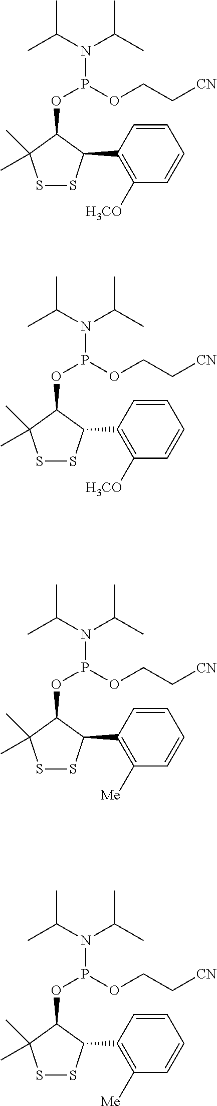 [SMILES: COc1ccccc1[C@H]1SSC(C)(C)[C@H]1OP(OCCC#N)N(C(C)C)C(C)C ; COc1ccccc1[C@@H]1SSC(C)(C)[C@H]1OP(OCCC#N)N(C(C)C)C(C)C ; Cc1ccccc1[C@H]1SSC(C)(C)[C@H]1OP(OCCC#N)N(C(C)C)C(C)C ; Cc1ccccc1[C@@H]1SSC(C)(C)[C@H]1OP(OCCC#N)N(C(C)C)C(C)C]

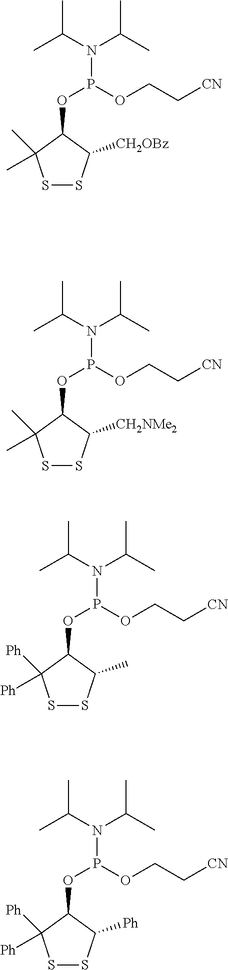

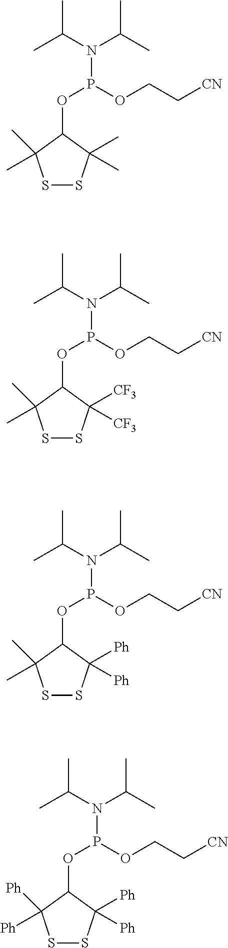 [SMILES: CC(C)N(C(C)C)P(OCCC#N)OC1C(C)(C)SSC1(C)C ; CC(C)N(C(C)C)P(OCCC#N)OC1C(C)(C)SSC1(C(F)(F)F)C(F)(F)F ; CC(C)N(C(C)C)P(OCCC#N)OC1C(C)(C)SSC1(c1ccccc1)c1ccccc1 ; CC(C)N(C(C)C)P(OCCC#N)OC1C(c2ccccc2)(c2ccccc2)SSC1(c1ccccc1)c1ccccc1]

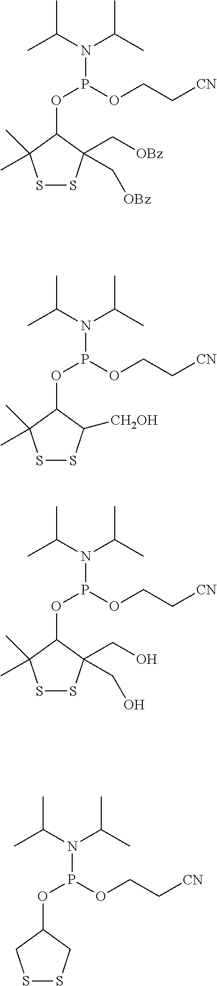

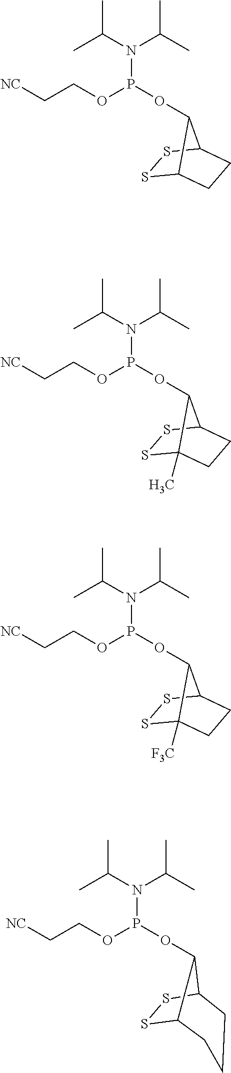 [SMILES: CC(C)N(C(C)C)P(OCCC#N)OC1C2CCC1SS2 ; CC(C)N(C(C)C)P(OCCC#N)OC1C2CCC1(C)SS2 ; CC(C)N(C(C)C)P(OCCC#N)OC1C2CCC1(C(F)(F)F)SS2 ; CC(C)N(C(C)C)P(OCCC#N)OC1C2CCCC1SS2]

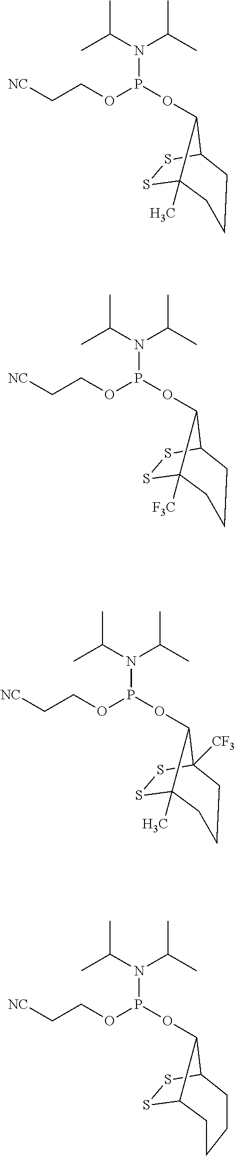 [SMILES: CC(C)N(C(C)C)P(OCCC#N)OC1C2CCCC1(C)SS2 ; CC(C)N(C(C)C)P(OCCC#N)OC1C2CCCC1(C(F)(F)F)SS2 ; CC(C)N(C(C)C)P(OCCC#N)OC1C2(C)CCCC1(C(F)(F)F)SS2 ; CC(C)N(C(C)C)P(OCCC#N)OC1C2CCCCC1SS2]

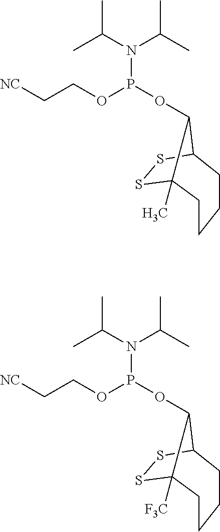 [SMILES: CC(C)N(C(C)C)P(OCCC#N)OC1C2CCCCC1(C)SS2 ; CC(C)N(C(C)C)P(OCCC#N)OC1C2CCCCC1(C(F)(F)F)SS2]

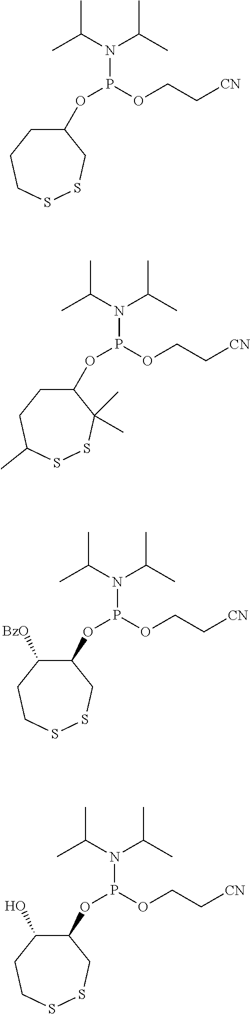

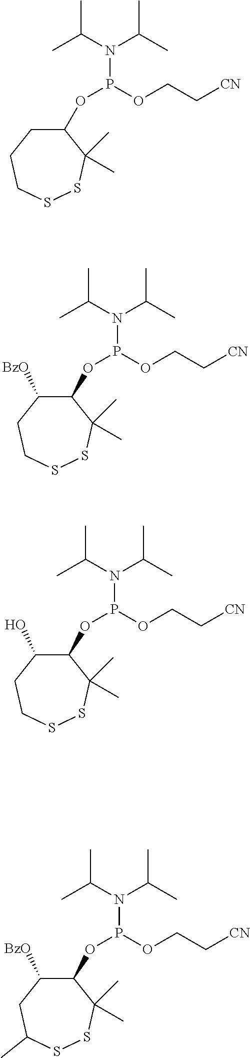

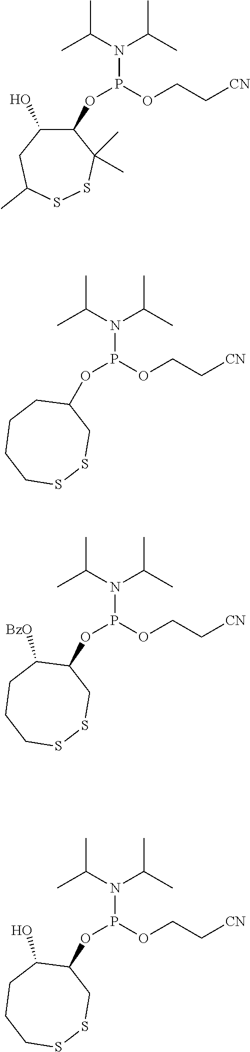

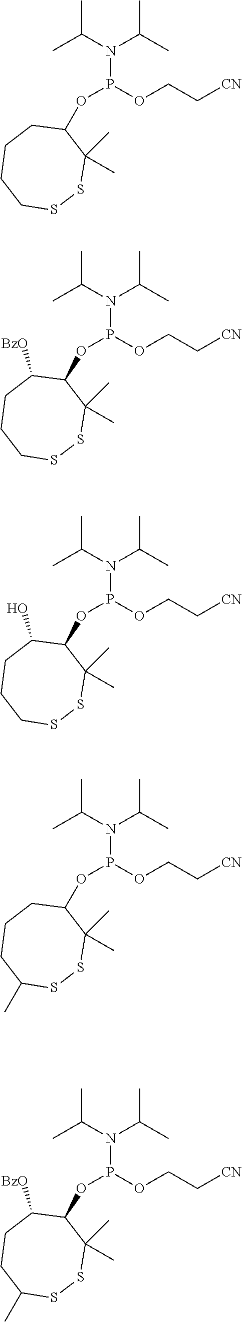

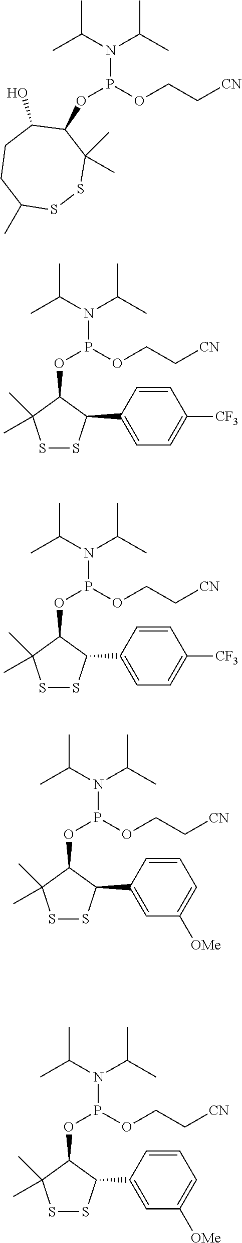 [SMILES: CC1CC[C@H](O)[C@@H](OP(OCCC#N)N(C(C)C)C(C)C)C(C)(C)SS1 ; CC(C)N(C(C)C)P(OCCC#N)O[C@H]1[C@@H](c2ccc(C(F)(F)F)cc2)SSC1(C)C ; CC(C)N(C(C)C)P(OCCC#N)O[C@H]1[C@H](c2ccc(C(F)(F)F)cc2)SSC1(C)C ; COc1cccc([C@H]2SSC(C)(C)[C@H]2OP(OCCC#N)N(C(C)C)C(C)C)c1 ; COc1cccc([C@@H]2SSC(C)(C)[C@H]2OP(OCCC#N)N(C(C)C)C(C)C)c1]

In one embodiment, the compound has one of the following structures:

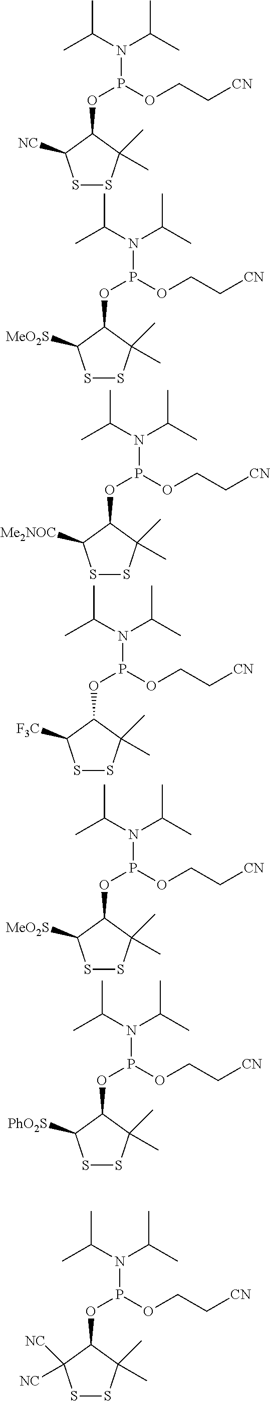

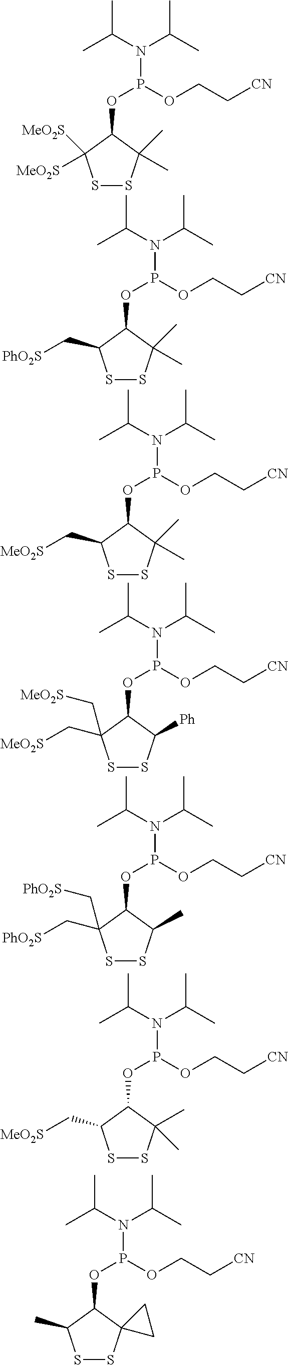

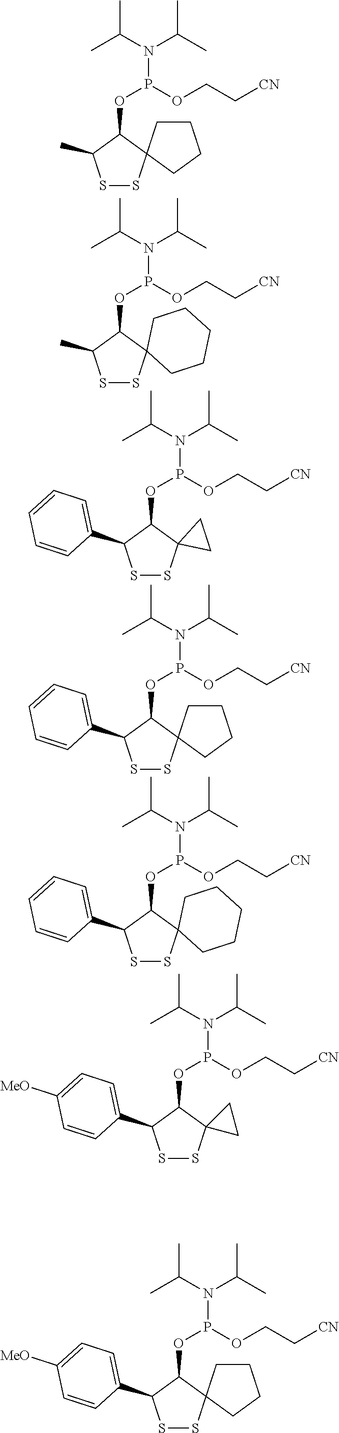

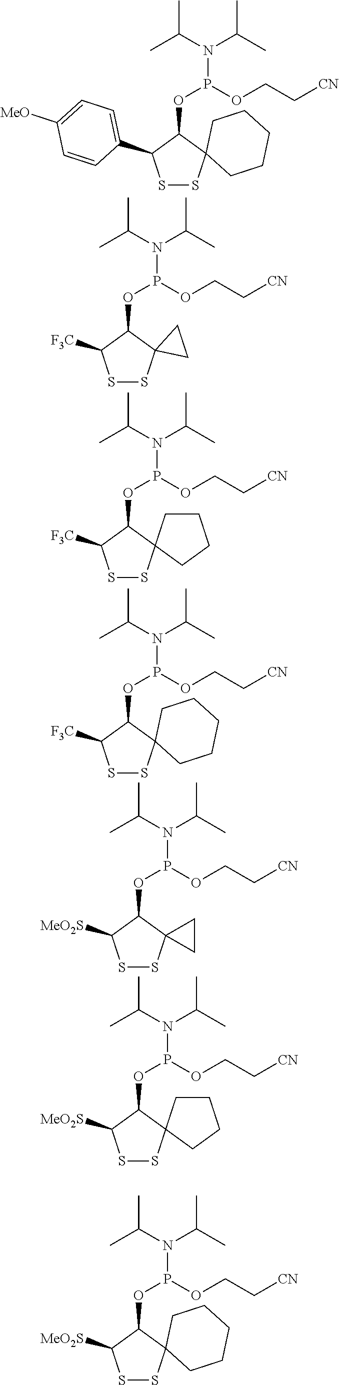

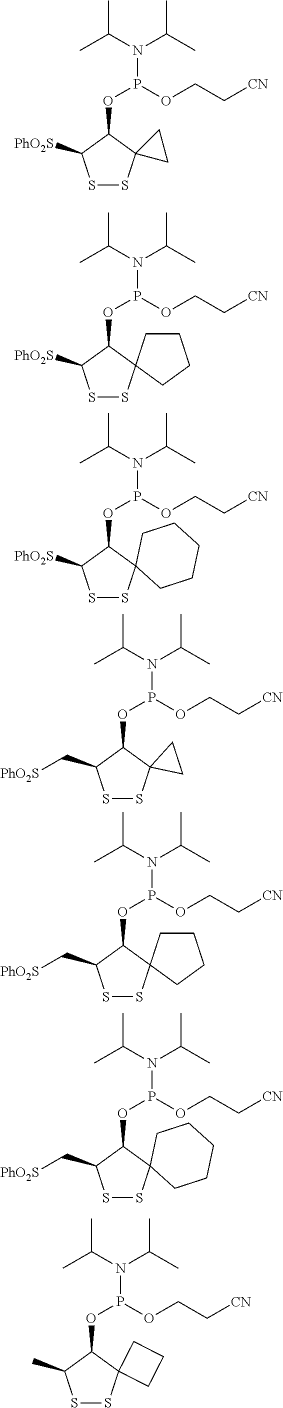

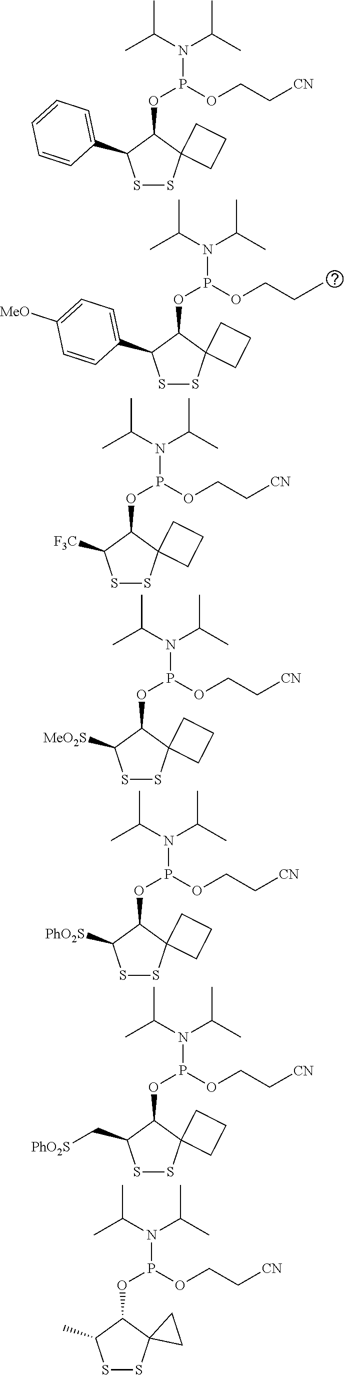

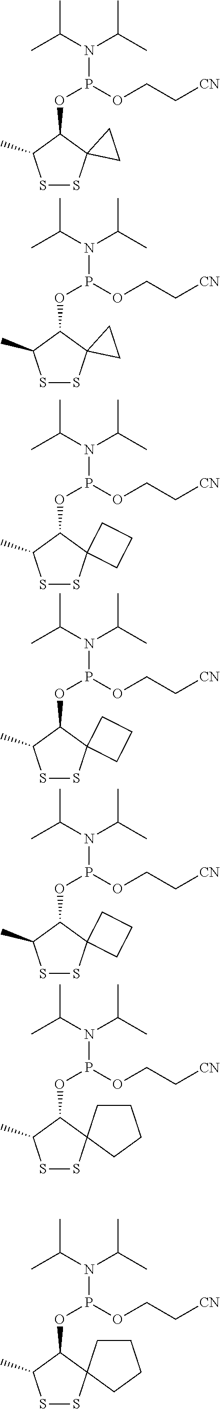

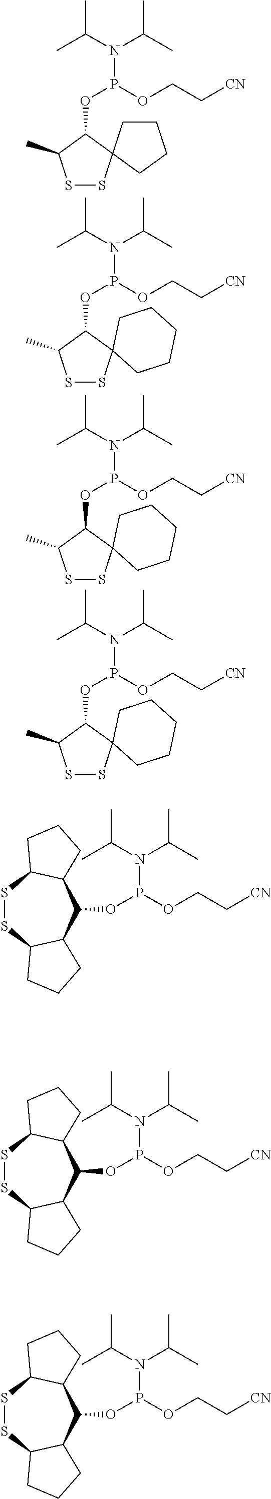

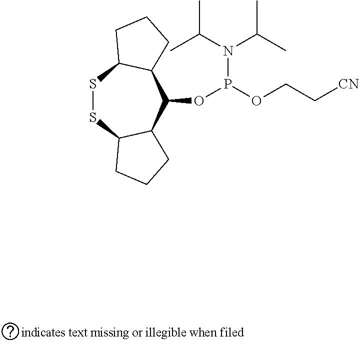

In some embodiments, the compound contains a stereoisomer of the formula (I) having a chiral purity of at least 70%.

In some embodiments, the compound contains one of the following stereoisomers, having a chiral purity of at least 70%:

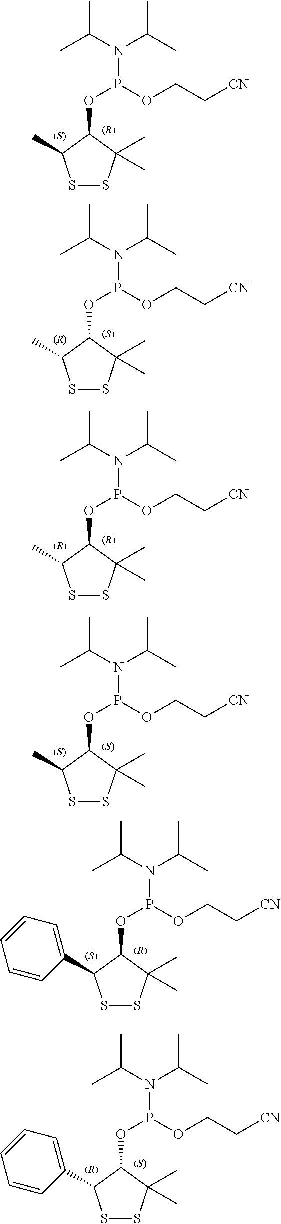

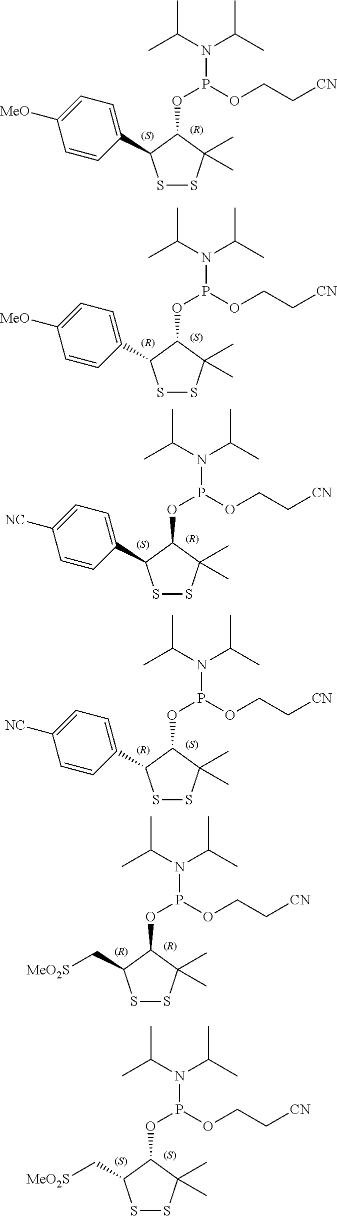

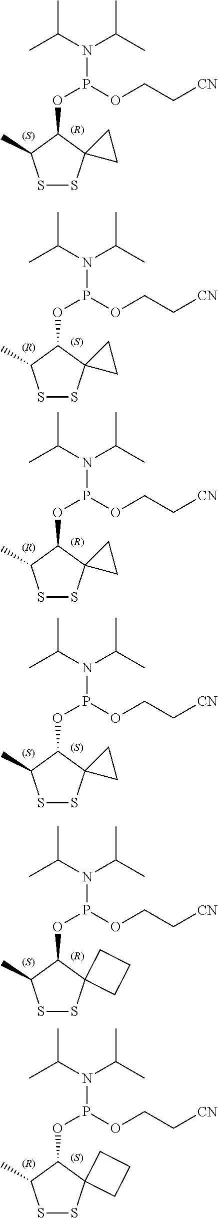

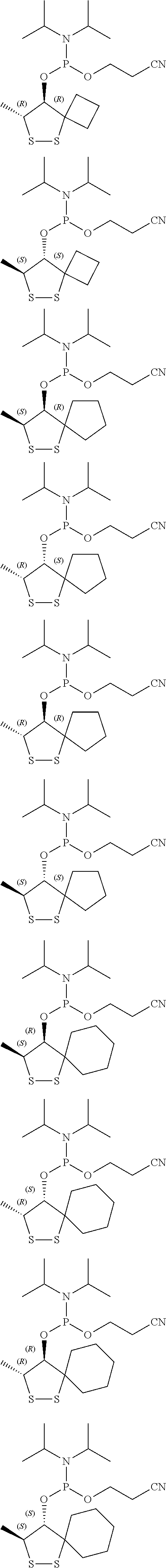

In some embodiments, the compound contains the following stereoisomers, having a chiral purity of at least 70%:

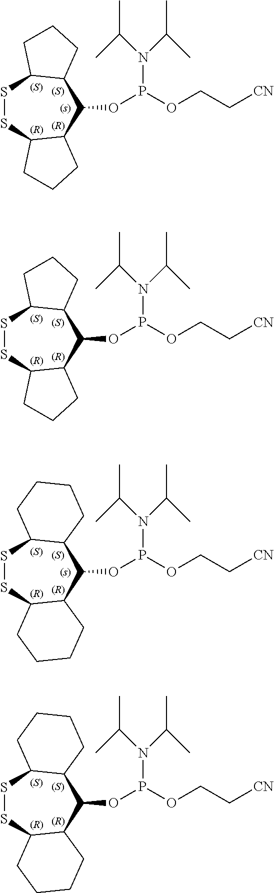

In one embodiment, the

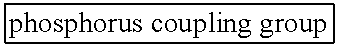

has the structure of (P-I), and the

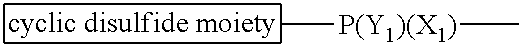

has a structure selected from the group consisting of:

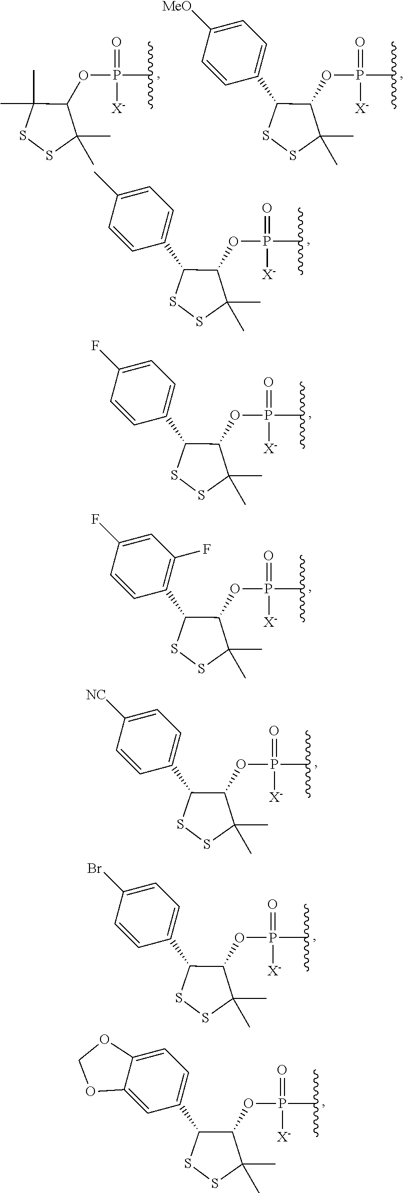

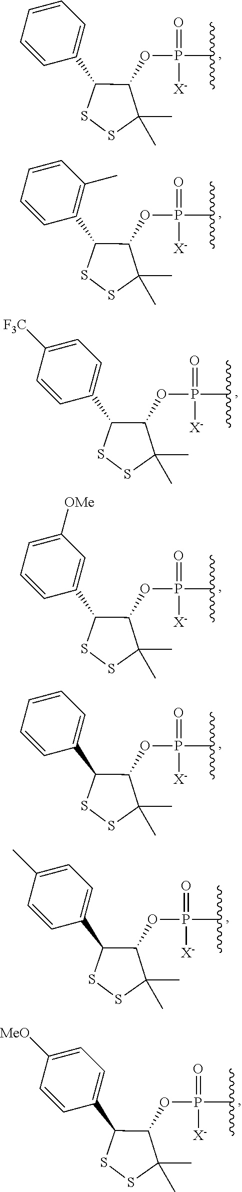

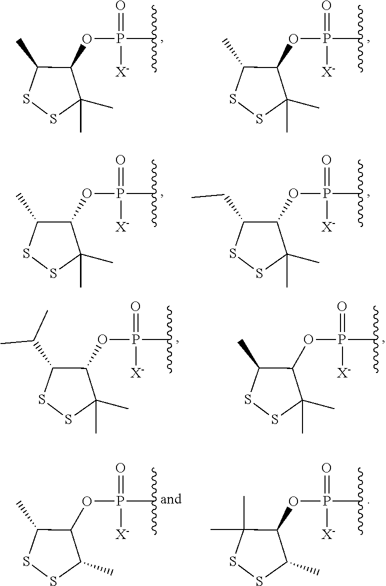

### X is or S.

In some embodiments, the compound contains one or more ligands connected to any one of R₂, R₃, R₄, R₅, R₆, R₇, R₈, and R₉ of the

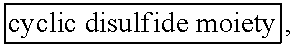

optionally via one or more linkers.

In some embodiments, the ligand is selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety (e.g., a lipophilic moiety that enhances plasma protein binding), a cholesterol, a steroid, bile acid, vitamin B12, biotin, a fluorophore, and a peptide.

In certain embodiments, at least one ligand is a carbohydrate-based ligand targeting a liver tissue. In one embodiment, the carbohydrate-based ligand is selected from the group consisting of galactose, multivalent galactose, N-acetyl-galactosamine (GalNAc), multivalent GalNAc, mannose, multivalent mannose, lactose, multivalent lactose, N-acetyl-glucosamine (GlcNAc), multivalent GlcNAc, glucose, multivalent glucose, fucose, and multivalent fucose.

In certain embodiments, the carbohydrate-based ligand is an ASGPR ligand. For example, the ASGPR ligand is one or more GalNAc derivatives attached through a bivalent or trivalent branched linker, such as:

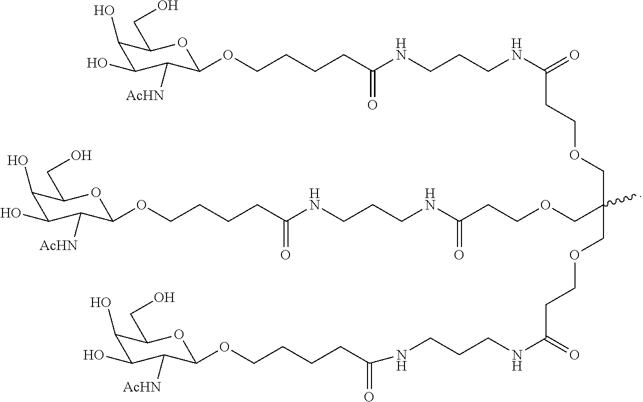 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, at least one ligand is a lipophilic moiety. In one embodiment, the lipophilicity of the lipophilic moiety, measured by log K_{ow}, exceeds 0, or the hydrophobicity of the compound, measured by the unbound fraction in the plasma protein binding assay of the compound, exceeds 0.2.

In one embodiment, the lipophilic moiety contains a saturated or unsaturated C₄-C₃₀ hydrocarbon chain, and an optional functional group selected from the group consisting of hydroxyl, amine, carboxylic acid, sulfonate, phosphate, thiol, azide, and alkyne. For instance, the lipophilic moiety contains a saturated or unsaturated C₆-C₁₈ hydrocarbon chain.

In certain embodiments, at least one ligand targets a receptor which mediates delivery to a CNS tissue. In one embodiment, the ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

In certain embodiments, at least one ligand targets a receptor which mediates delivery to an ocular tissue. In one embodiment, the ligand is selected from the group consisting of trans-retinol, RGD peptide, LDL receptor ligand, and carbohydrate based ligands.

Another aspect of the invention relates to an oligonucleotide (e.g. a single-stranded iRNA a gent or a double-stranded iRNA agent) comprising one or more structures of formula (I):

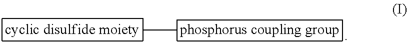

Another aspect of the invention relates to an oligonucleotide (e.g. a single-stranded iRNA agent or a double-stranded iRNA agent) comprising one or more structures of formula (II):

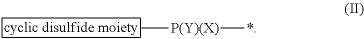

In both formulas (I) and (II), the

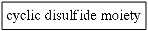

has the structure of:

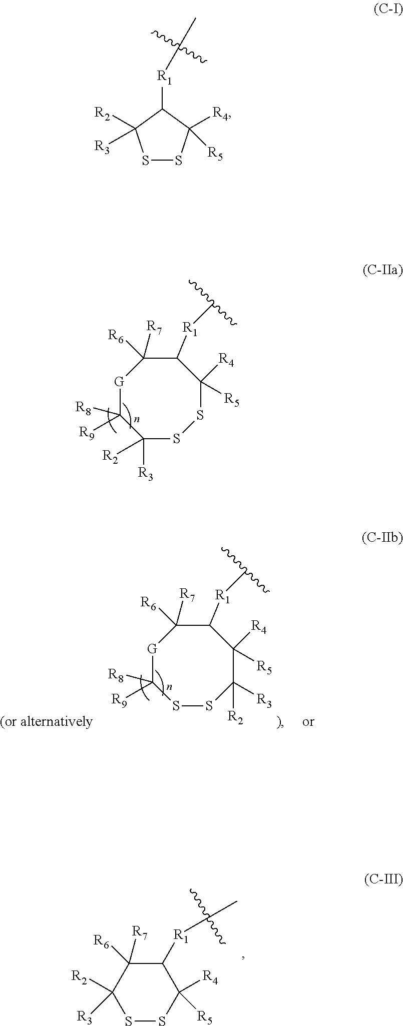

or a salt or stereoisomer thereof. In these formulas:
R₁ is O or S, and is bonded to the P atom of the

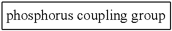

of formula (I), or the P atom of formula (II); [symbol] indicates the bond to the

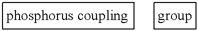

of formula (I), or the P atom of formula (II);
R₂, R₄, R₆, R₇, R₈, and R₉ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R″ groups;
R₃ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₃ and R₅, together with the adjacent carbon atoms and the two sulfur atoms, form a second ring;
R₂ and R₃, together with the adjacent carbon atom, can form another ring;
R₄ and R₅, together with the adjacent carbon atom, can form another ring;
R₆ and R₇, together with the adjacent carbon atom, can form another ring;
R₈ and R₉, together with the adjacent carbon atoms, can form another ring;
two or more of R₂, R₃, R₄, R₅, R₆, R₇, R₈, R₉, R¹⁴, and R¹⁵, together with the adjacent carbon atoms can form one or more rings fused with the ring containing the two sulfur atoms;
G is O, N(R′), S, or C(R¹⁴)(R¹⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl,
heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″, together with the adjacent nitrogen atom, form a ring; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido;
wherein, when the

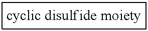

has the structure of formula (C-III), at least one

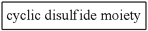

is connected at the 5′ end of the nucleoside or oligonucleotide.

In Formula (I), at least one

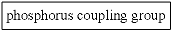

contains a nucleoside or oligonucleotide.

In Formula (II), * represents the bond to the oligonucleotide, Y is absent, N(R′), ═O, or ═S; X is OH, SH, C(O)H, S(O)H, alkyl optionally substituted with one or more R^{sub} groups, N(R′)(R″), NSO₂R′, N═CN(R′)(R″), B(R¹³)₃, BH₃, or X′, wherein X′ is N(R′)(R″), OR¹³ or SR¹³.

All the above embodiments relating to formula (C-I) and formula (C-IIa) (or C-IIb) of the

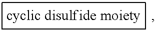

all the formulas of the

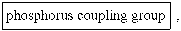

all the variables defined in these formulas, all the ligands, and all the subgenus and species structures relating to the compound, the

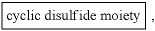

and the

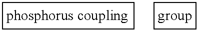

in the first aspect of the invention relating to the compound are suitable in these aspects of the invention relating to the oligonucleotide.

In some embodiments, the

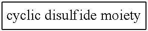

has the structure selected from one of the following Ia), Ib), and II) groups. Ia) group contains the following structures:

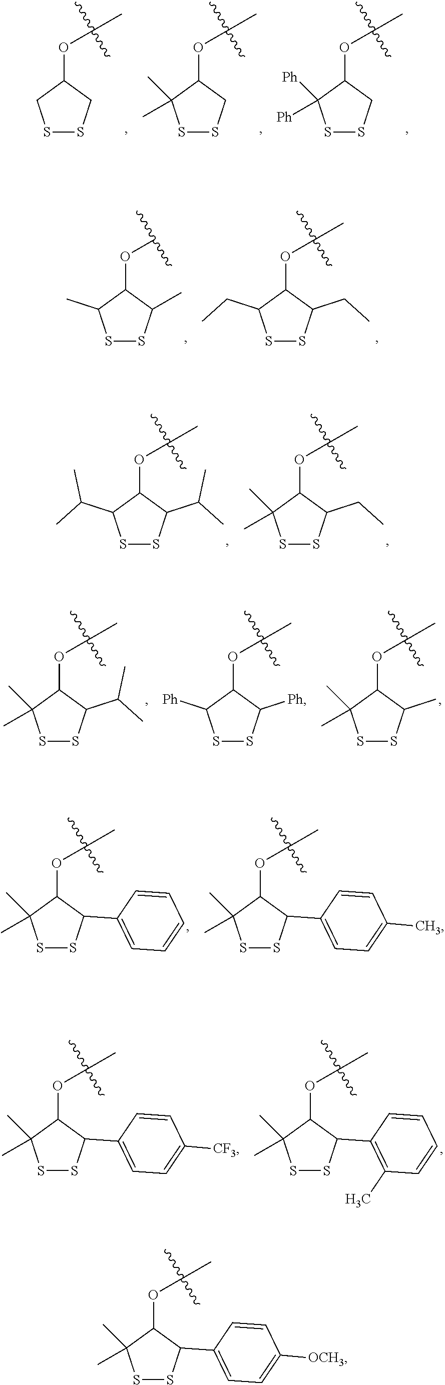

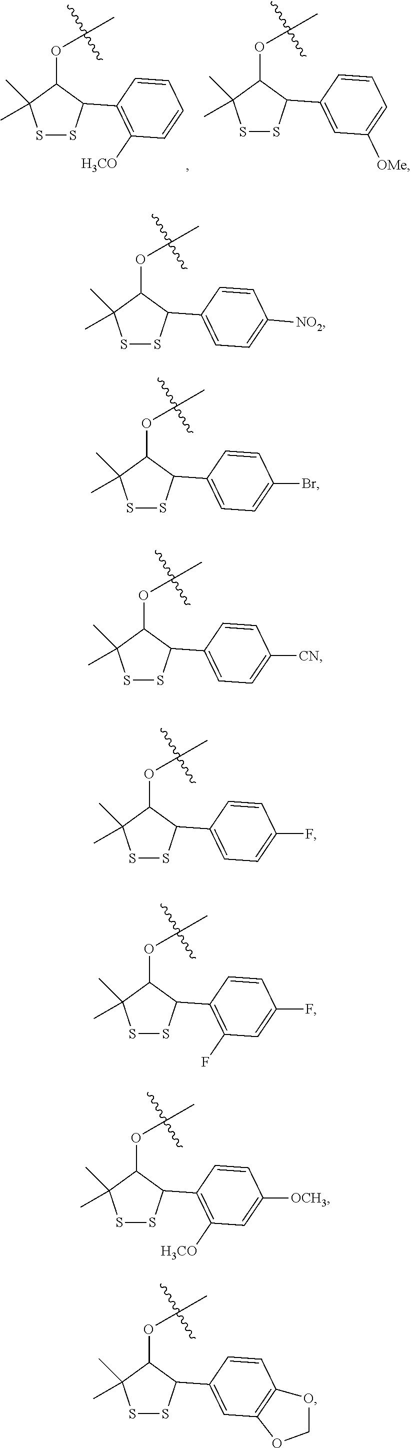

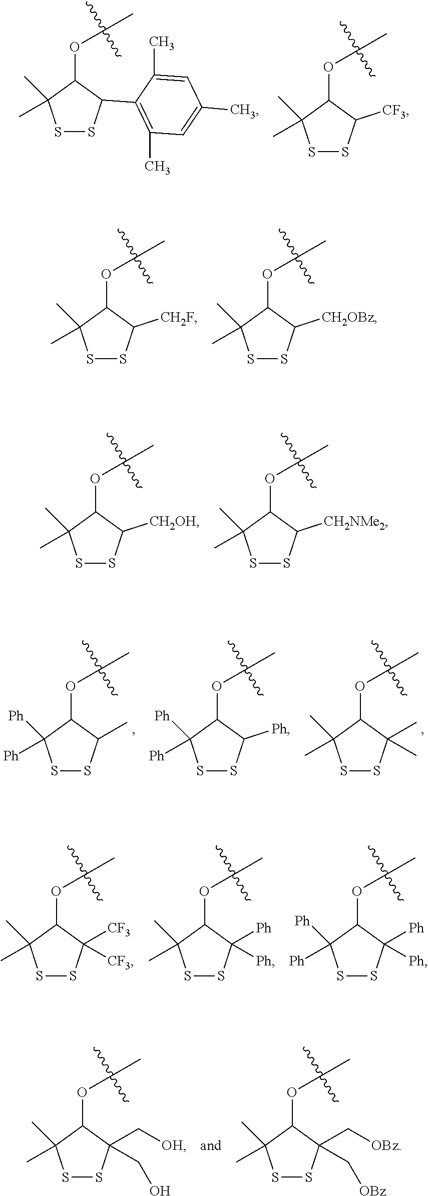

Ib) group contains the following structures:

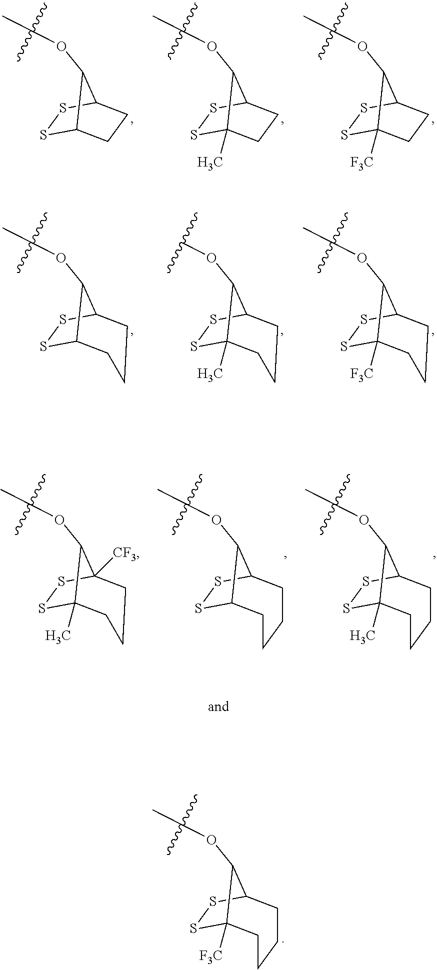

II) group contains the following structures:

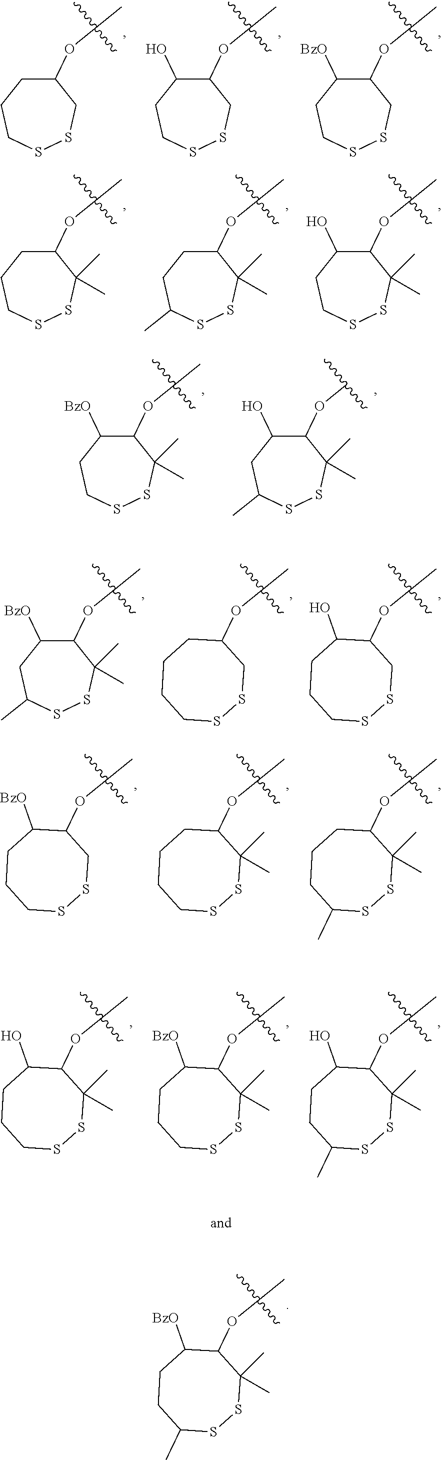

In some embodiments, the

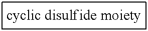

has the structure selected from one of the structures from III) group. III) group contains the following structures:

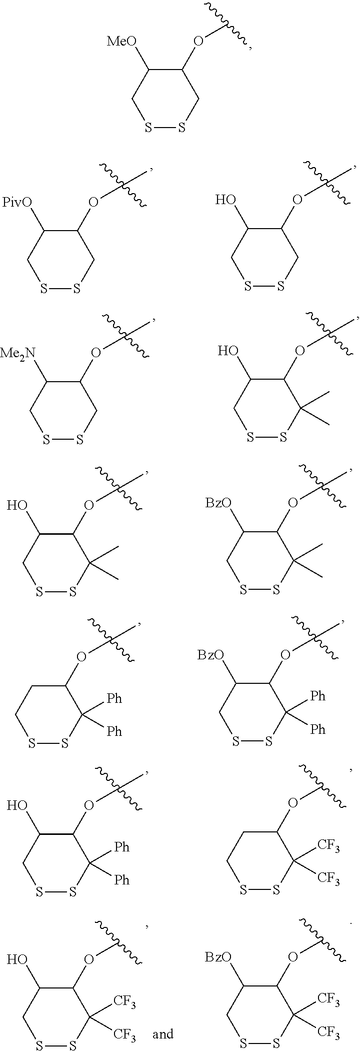

In some embodiments, the oligonucleotide contains the structure selected from one of the following group consisting of:

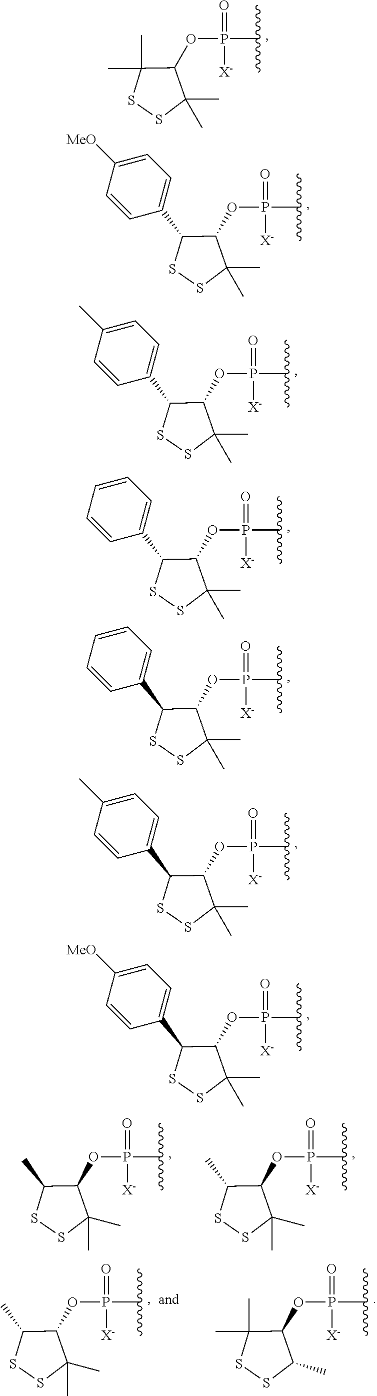

or a salt thereof, wherein X is O or S.

In some embodiments, the oligonucleotide contains a stereoisomer of formula (I) or (II) having a chiral purity of at least 70%.

In some embodiments, the oligonucleotide contains the following stereoisomers, having a chiral purity of at least 70%:

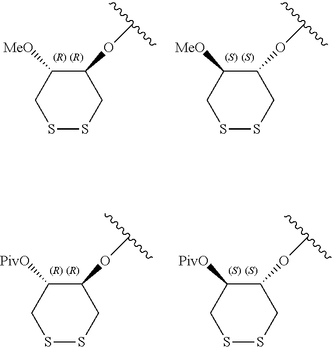

In some embodiments, the compound contains the following stereoisomers, having a chiral purity of at least 70%:

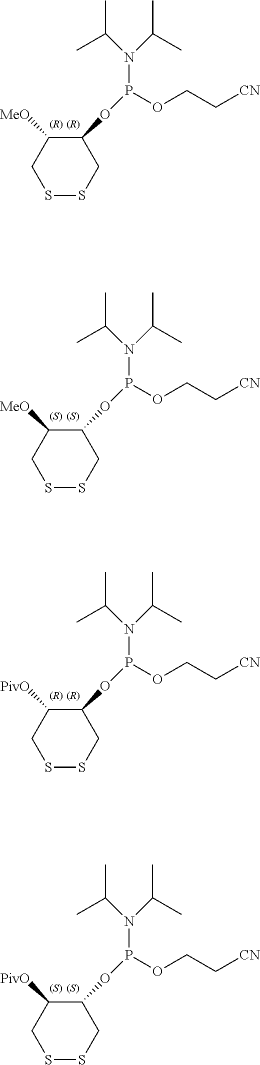

In some embodiments, the oligonucleotide contains a structure having the formula:

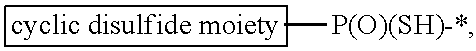

or a salt thereof.

In some embodiments, the oligonucleotide contains a structure having the formula:

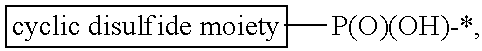

or a salt thereof.

In some embodiments, the oligonucleotide contains a structure having the formula:

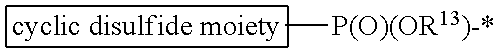

or a salt thereof. The variable R³ is as defined above.

In some embodiments, the oligonucleotide contains a structure having the formula:

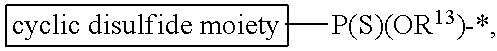

or a salt thereof. The variable R³ is as defined above.

In some embodiments, the oligonucleotide contains a structure having the formula:

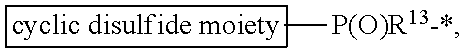

or a salt thereof. The variable R³ is as defined above.

In some embodiments, the oligonucleotide contains a structure having the formula:

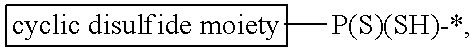

or a salt thereof.

In some embodiments, the oligonucleotide contains a structure having the formula:

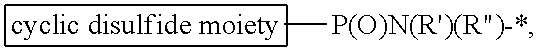

or a salt thereof. The variables R′ and R″ are as defined above.

In some embodiments, the oligonucleotide contains a structure having the formula:

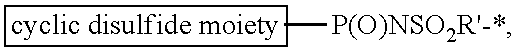

or a salt thereof. The variable R′ is as defined above.

In some embodiments, the oligonucleotide contains a structure having the formula:

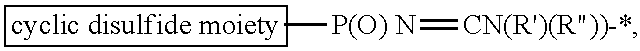

or a salt thereof. The variables R′ and R″ are as defined above.

In some embodiments, the oligonucleotide comprises a structure having one of the following formulas:

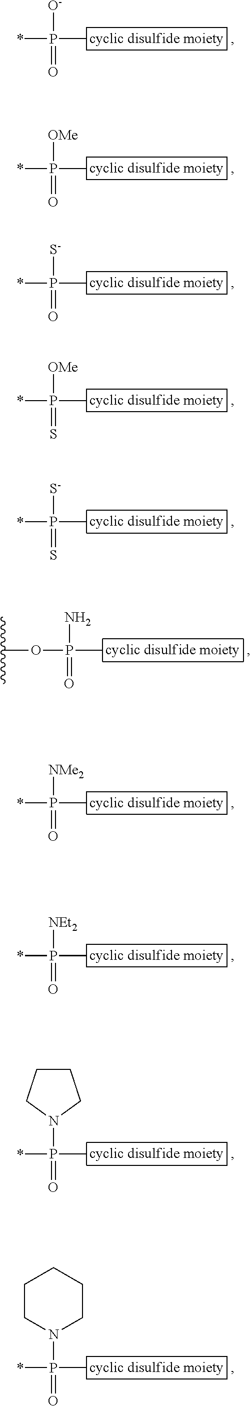

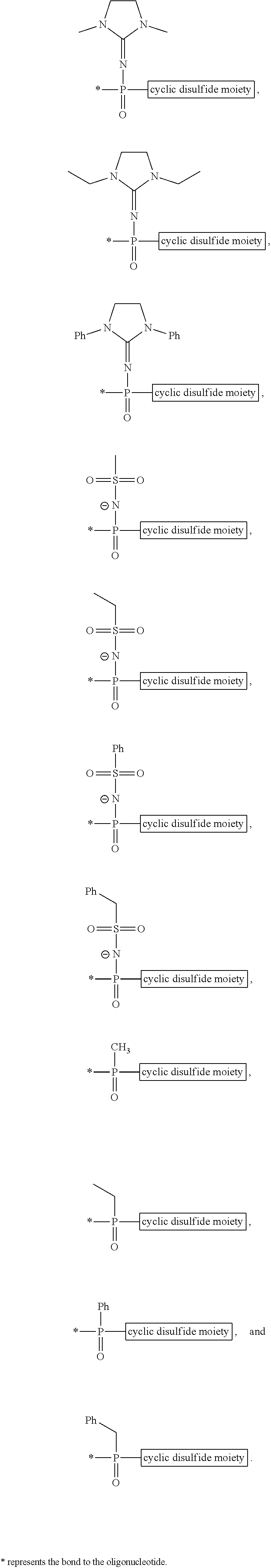

In some embodiments, the

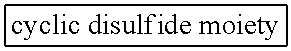

has one of the following structures:

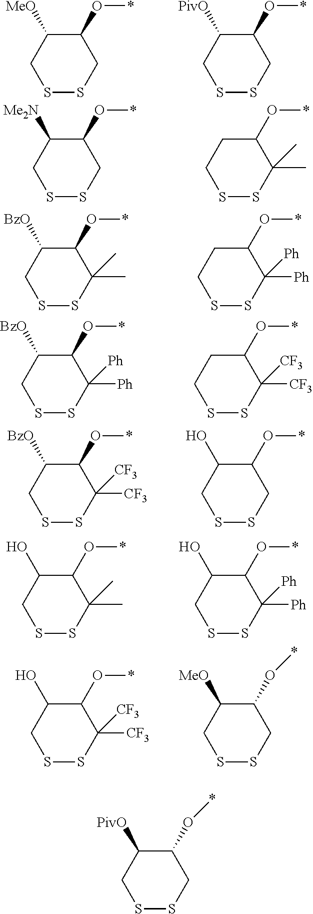

wherein * indicates the bond to the phosphorus atom of the —P(X)(Y)—* group.

In some embodiments, the compound has one of the following structures:

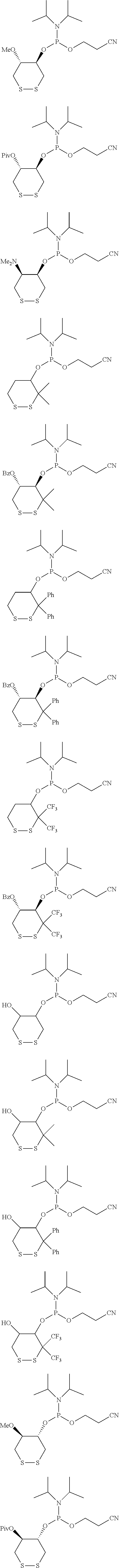

In one embodiment, the oligonucleotide contains a structure selected from the group consisting of

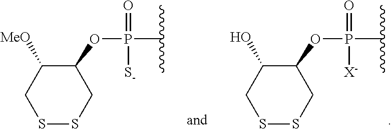

### X is O or S.

In some embodiments, the oligonucleotide contains at least one

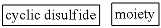

at the 5′-end of the oligonucleotide.

In some embodiments, the first nucleotide at the 5′-end of the oligonucleotide has the structure of

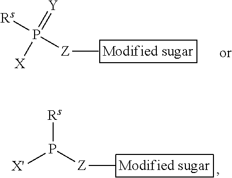

or a salt thereof, wherein:
R^{S} is the

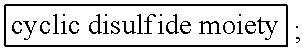

X is —OH, —SH, C(O)H, S(O)H, alkyl optionally substituted with one or more R^{sub} groups, N(R′)(R″), NSO₂R′, N═CN(R′)(R′), B(R¹³)₃, BH₃⁻, or X′, wherein X′ is N(R′)(R″), —OR¹³ or —SR¹³
Y is S, O, or N(R′);
Z is O, S, N(R′), or CH₂; and

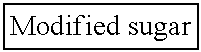

is a sugar moiety containing one or more sugar modifications selected from the group consisting of 2′-modification, LNA, isomeric modification, 5′-modification, unnatural cyclic modification, acyclic modification, and abasic modification.

In some embodiments, the sugar modification in the

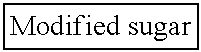

is 2′-modification, LNA, isomeric modification, 5′-modification, or abasic modification.

In some embodiments, the first nucleotide at the 5′-end of the oligonucleotide has the structure of

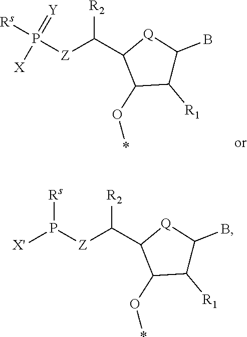

or, or a salt or stereoisomer thereof.

In these formulas:
* represents a bond to the subsequent optionally modified internucleotide linkage;
B is an optionally modified nucleobase, or H;
R^{S} is the

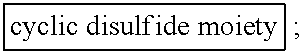

and
R₁ is H, OH, O-methoxyalkyl, O-methyl, O-allyl, CH₂-allyl, fluoro, O-N-methylacetamido (O-NMA), O-N-alkylacetamido, O-dimethoxypropyl, O-dimethylaminoethoxyethyl (O-DMAEOE), O-aminopropyl (O-AP), or ara-F; or R₁ forms a bridge with the 4′ carbon of the ribose sugar;
R₂ is H, alkyl, or aryl;
X is —OH, —SH, C(O)H, S(O)H, alkyl optionally substituted with one or more R^{sub} groups, N(R′)(R″), NSO₂R′, N═CN(R′)(R″), B(R¹³)₃, BH₃, or X′, wherein X′ is N(R′)(R″), —OR¹³ or —SR¹³;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido;
Y is S, O, or N(R′);
Z is O, S, N(R′), or CH₂; and
Q is O, S, CH₂, or N(R′).

In some embodiments, the sugar modification in the

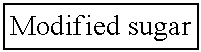

is a 2′-modification. In some embodiment, the first nucleotide at the 5′-end of the oligonucleotide has one of the following structures:

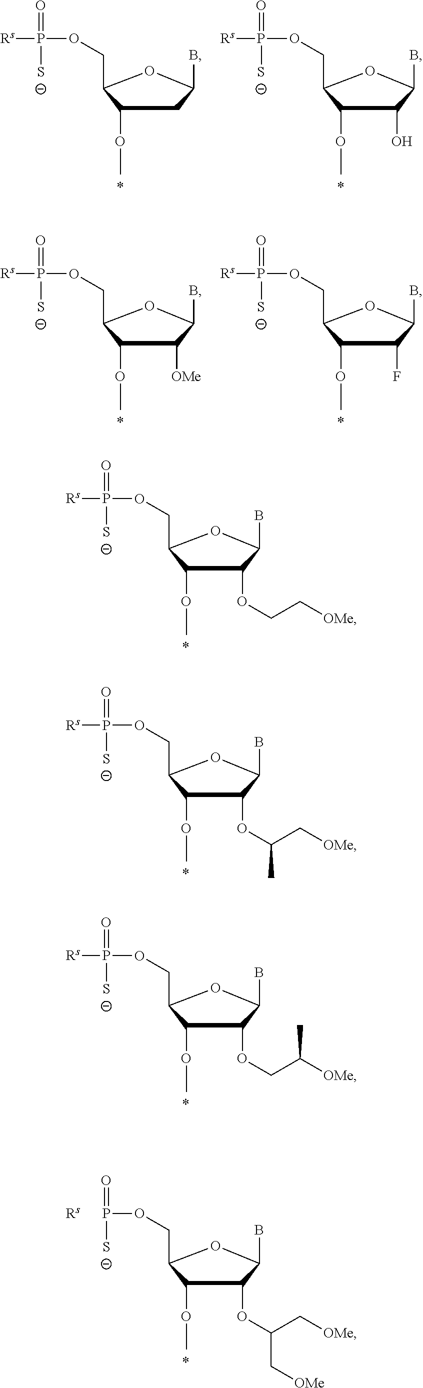

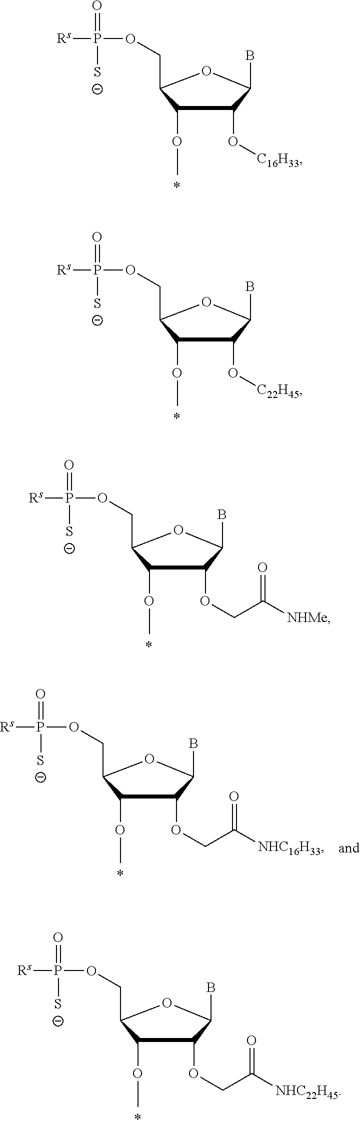

*, R^{S}, and B are as defined above.

In some embodiments, the sugar modification in the

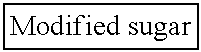

is a “locked” nucleic acid (LNA). In some embodiments, the first nucleotide at the 5′-end of the oligonucleotide has the structure of:

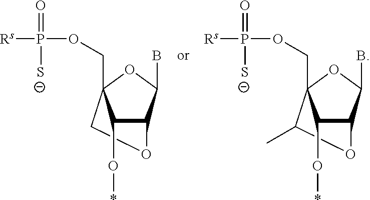

*, R^{S}, and B are as defined above.

In some embodiments, the sugar modification in the

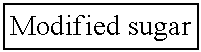

is an isomeric modification to the ribose sugar. In some embodiments, the first nucleotide at the 5′-end of the oligonucleotide has one of the following structures:

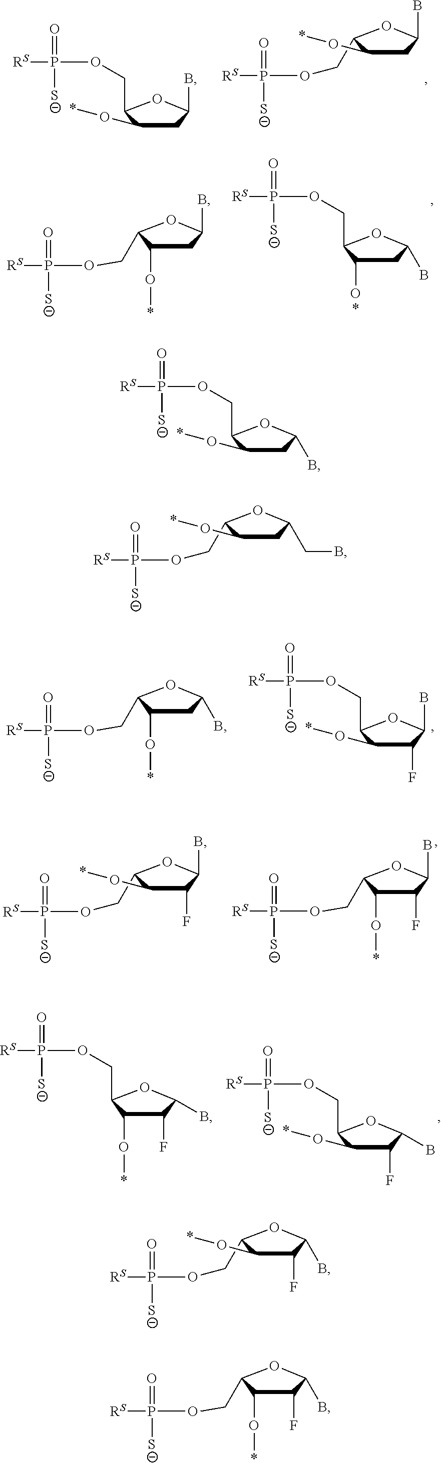

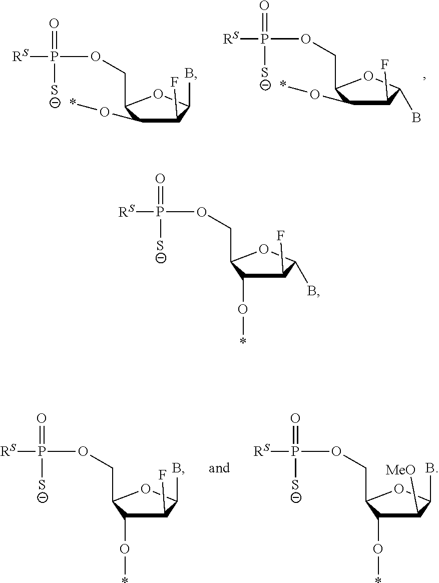

*, R^{S}, and B are as defined above.

In some embodiments, the sugar modification in the

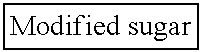

is a 5′-modification. having a substituent group at the 5′ positon of the ribose. In some embodiments, the first nucleotide at the 5′-end of the oligonucleotide has one of the following structures:

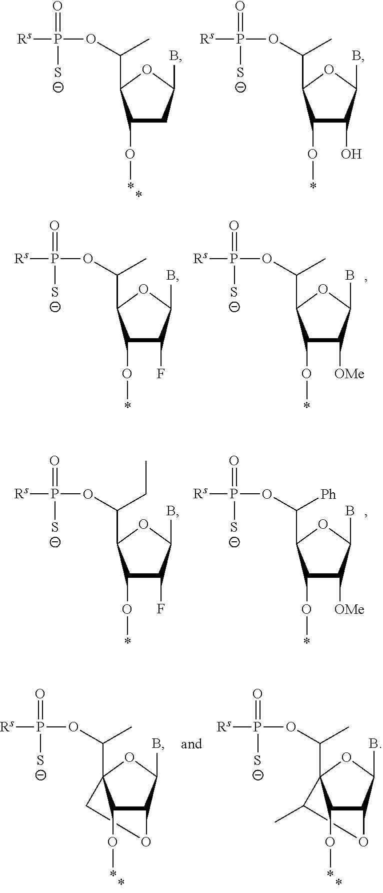

B are as defined above.

In some embodiments, the sugar modification in the

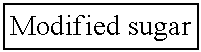

is an abasic modification. In some embodiments, the first nucleotide at the 5′-end of the oligonucleotide has one of the following structures:

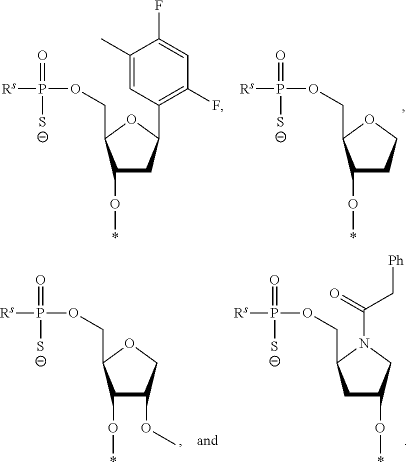

and R^{S} are as defined above.

In some embodiments, the

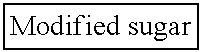

contains unnatural cyclic modification having one of the following structures:

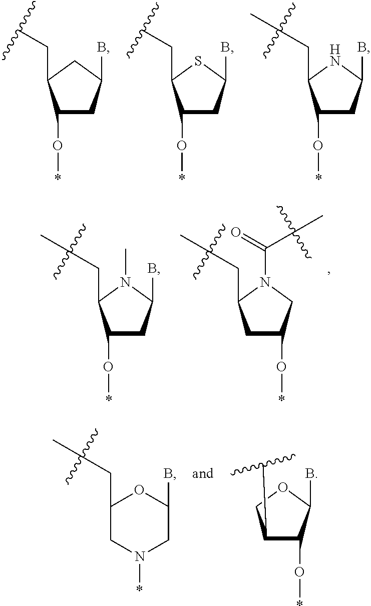

* and B are as defined above.

In some embodiments, the first nucleotide at the 5′-end of the oligonucleotide has one of the following structures:

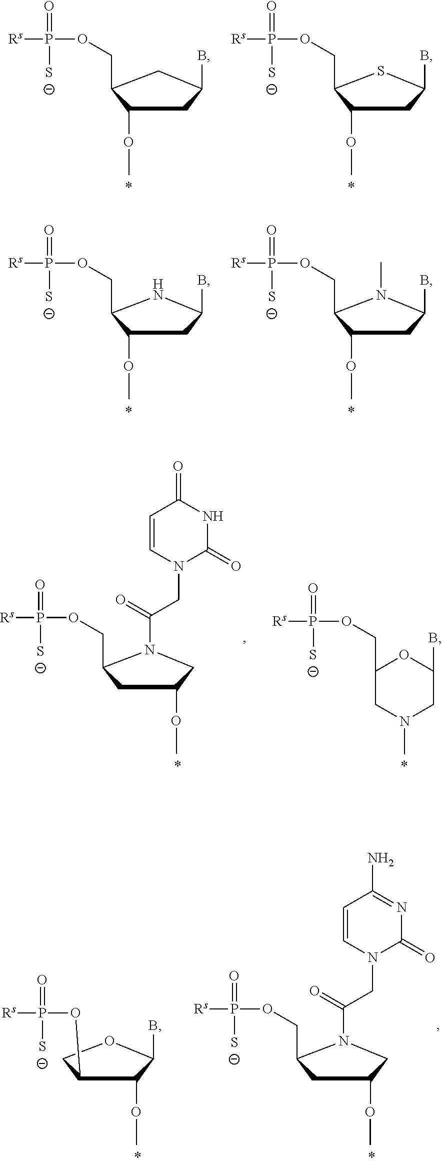

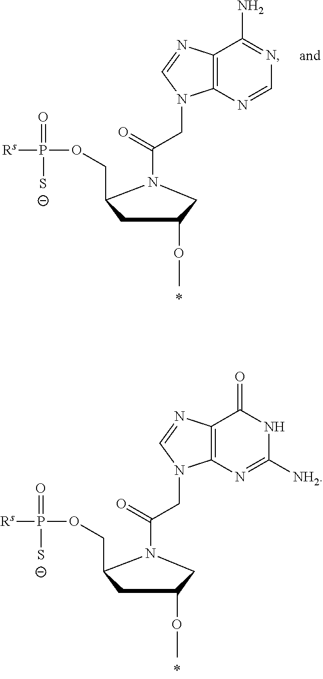

*, R^{S}, and B are as defined above.

In some embodiments, the

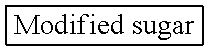

contains an acyclic modification having one of the following structures:

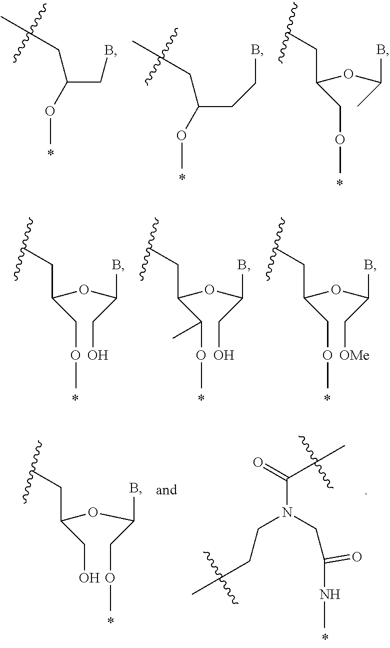

* and B are as defined above.

In some embodiments, the first nucleotide at the 5′-end of the oligonucleotide has one of the following structures:

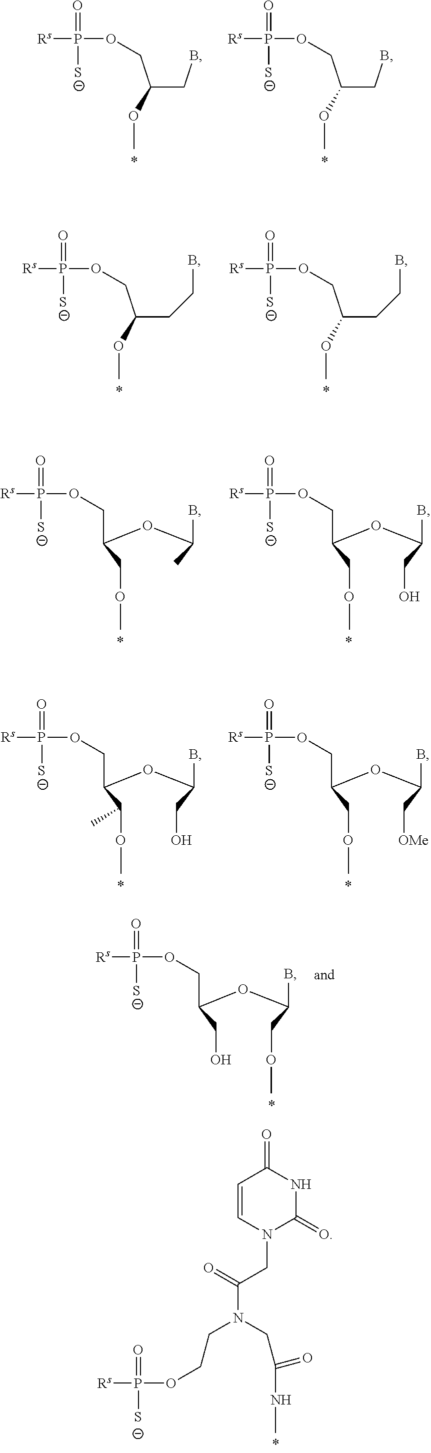

*, R^{S}, and B are as defined above.

In some embodiments, the first nucleotide at the 5′-end of the oligonucleotide has one of the following structures ( [symbol] represents a bond to the subsequent optionally modified internucleotide linkage):

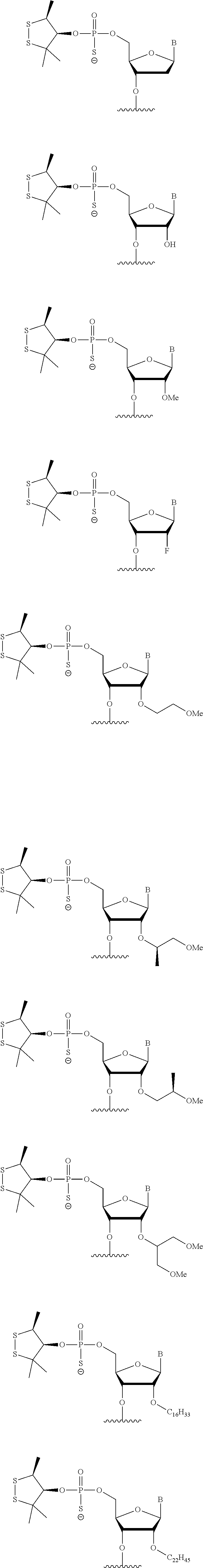

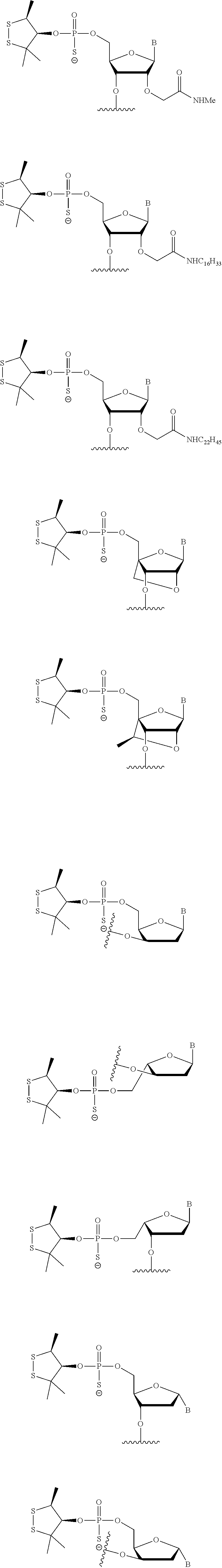

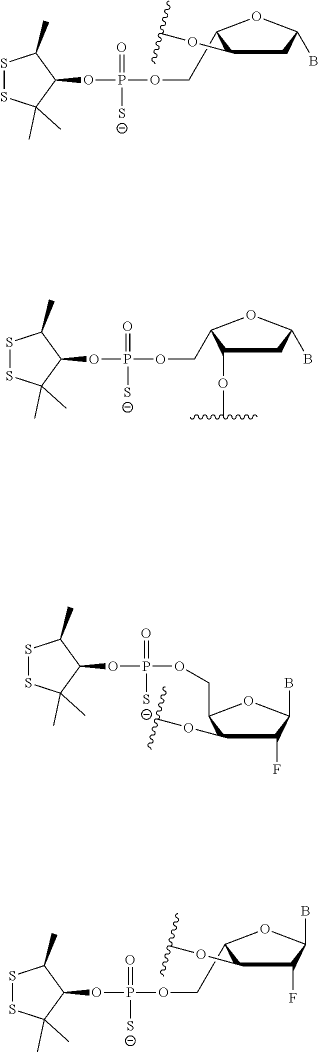

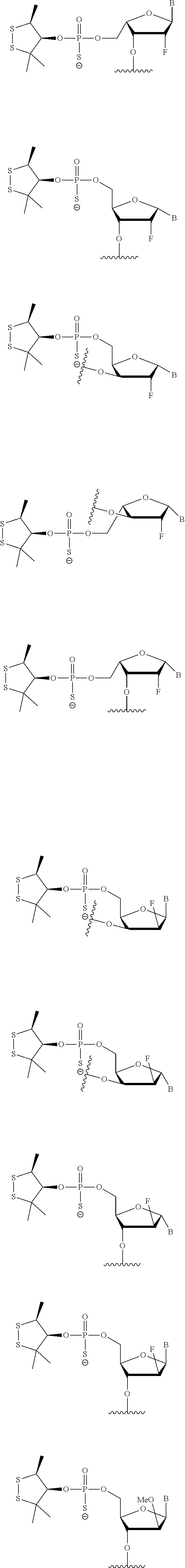

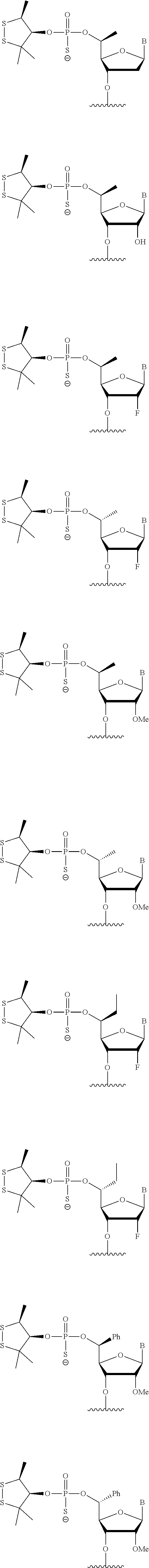

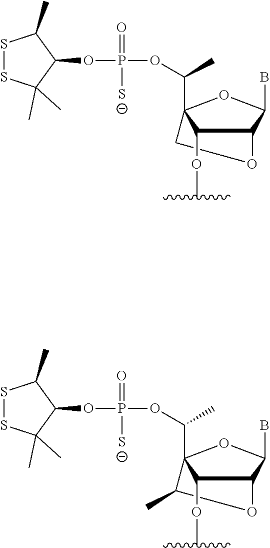

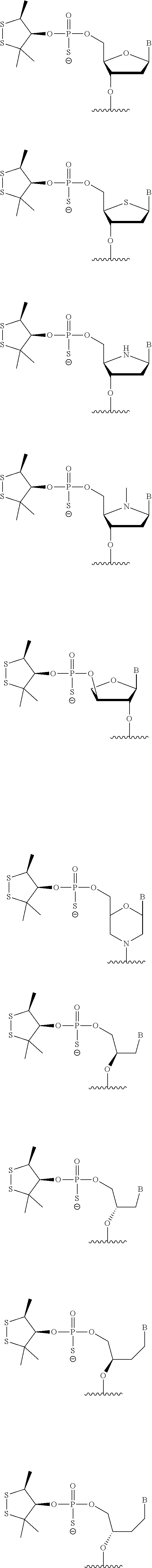

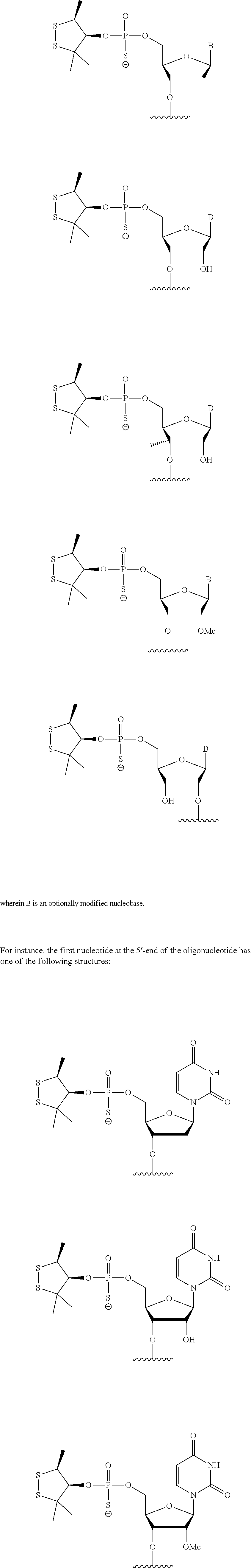

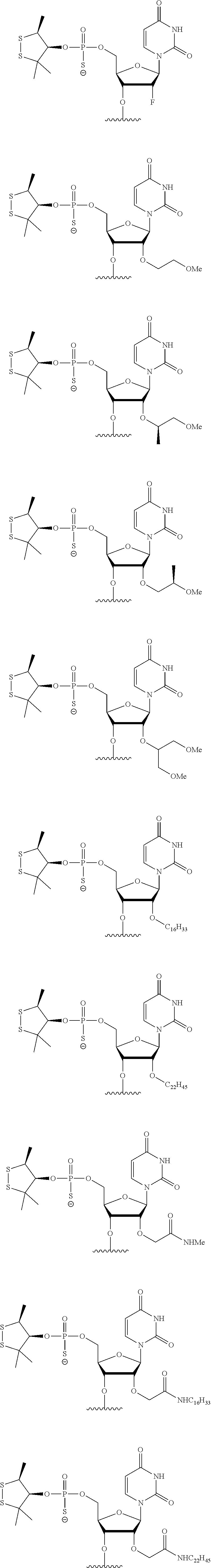

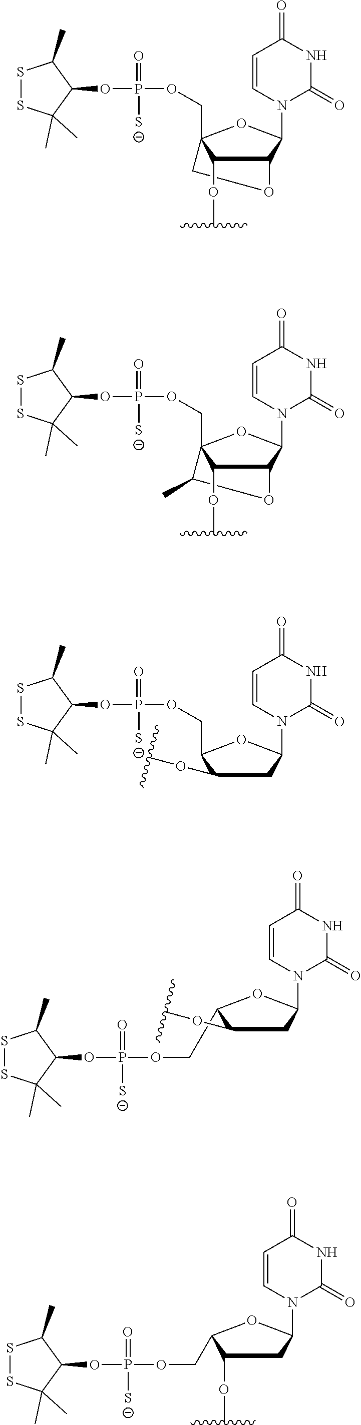

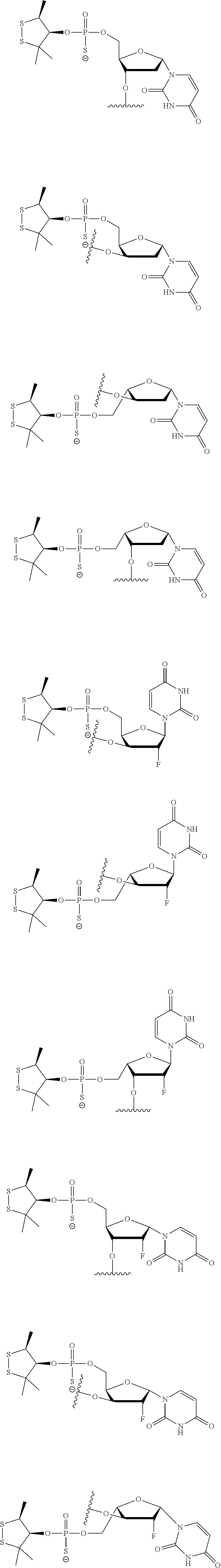

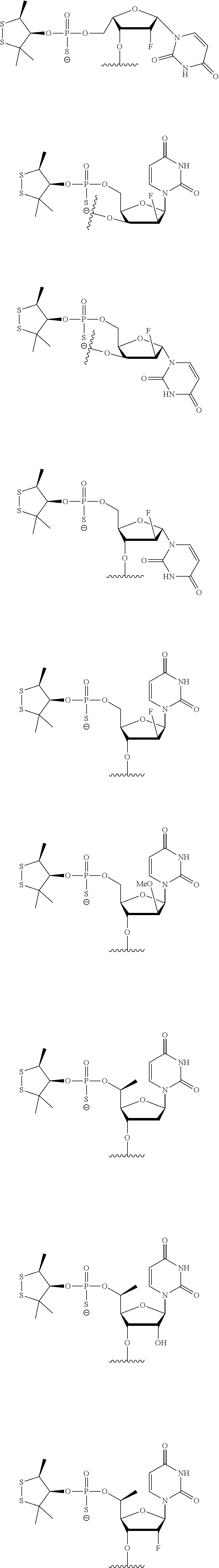

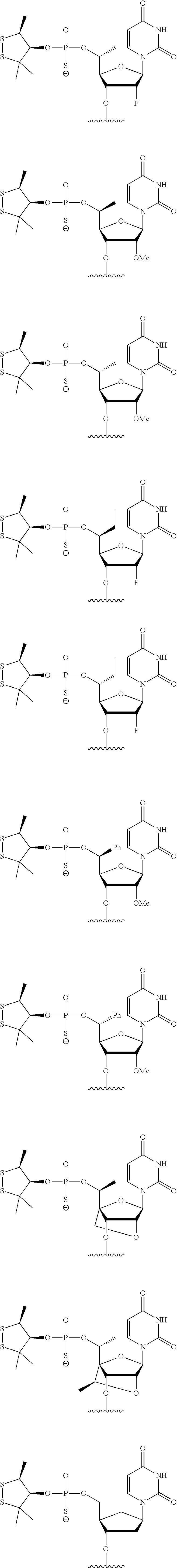

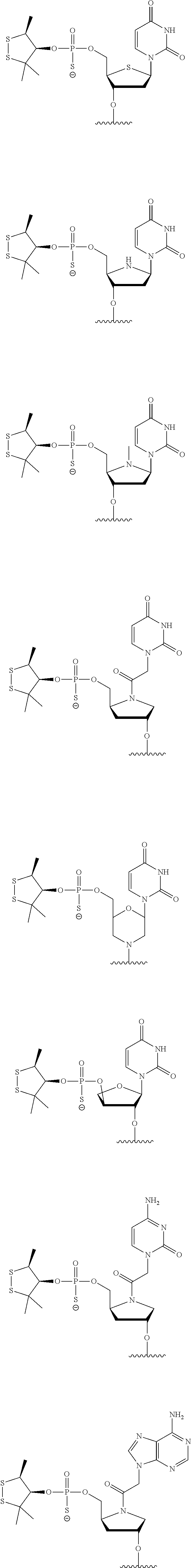

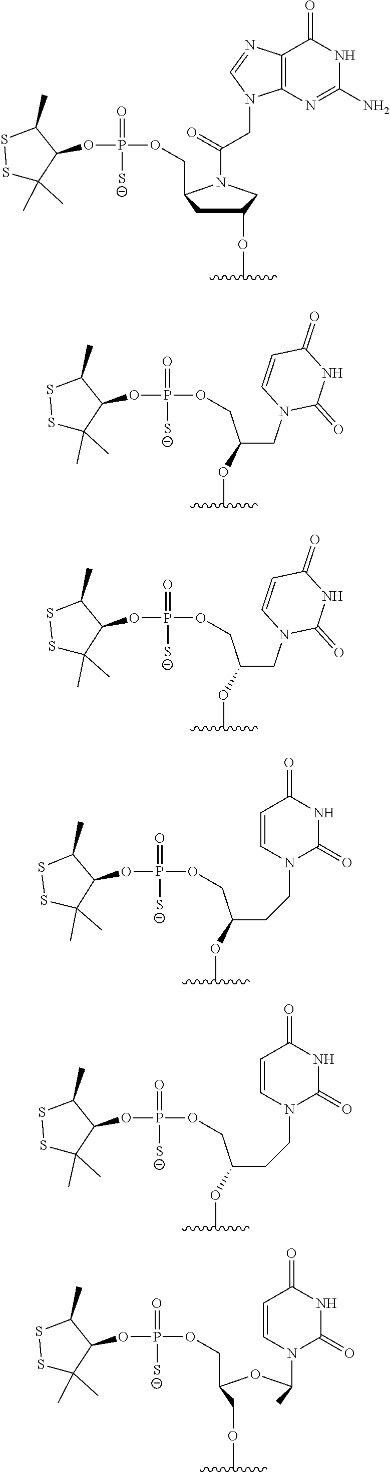

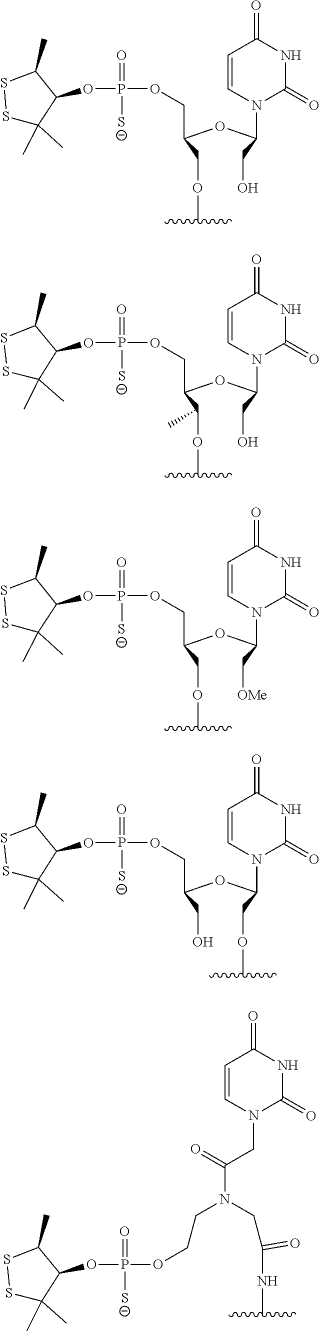 [SMILES: *OC[C@@H](COP(=O)([S-])O[C@@H]1[C@H](C)SSC1(C)C)O[C@H](CO)n1ccc(=O)[nH]c1=O ; *O[C@@H](C)[C@@H](COP(=O)([S-])O[C@@H]1[C@H](C)SSC1(C)C)O[C@H](CO)n1ccc(=O)[nH]c1=O ; *OC[C@@H](COP(=O)([S-])O[C@@H]1[C@H](C)SSC1(C)C)O[C@H](COC)n1ccc(=O)[nH]c1=O ; *OC[C@@H](O[C@@H](CO)COP(=O)([S-])O[C@@H]1[C@H](C)SSC1(C)C)n1ccc(=O)[nH]c1=O ; *NC(=O)CN(CCOP(=O)([S-])O[C@@H]1[C@H](C)SSC1(C)C)C(=O)Cn1ccc(=O)[nH]c1=O]

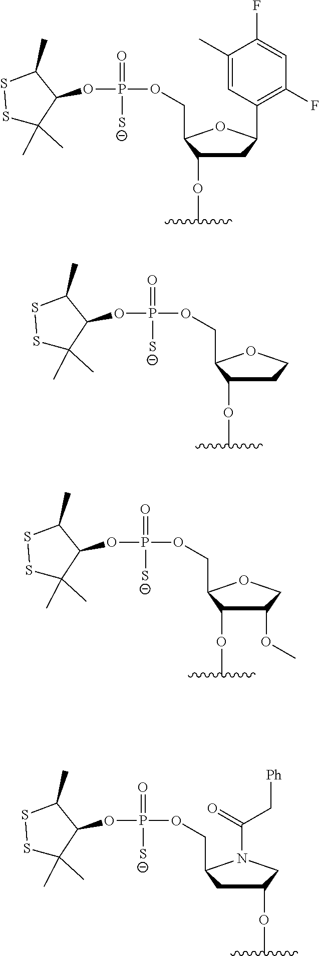

In some embodiments, the first nucleotide at the 5′-end of the oligonucleotide has the structure:

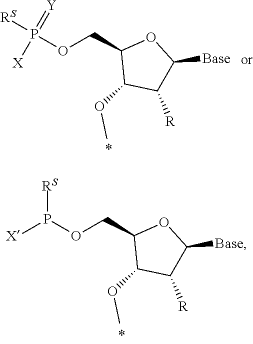

or a salt or stereoisomer thereof. In these structures:
* represents a bond to the subsequent optionally modified internucleotide linkage;
Base is an optionally modified nucleobase;
R^{S} is the

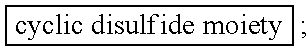

and
R is H, OH, O-methoxyalkyl, O-methyl, O-allyl, CH₂-allyl, fluoro, O-N-methylacetamido (O-NMA), O-dimethylaminoethoxyethyl (O-DMAEOE), O-aminopropyl (O-AP), or ara-F. The variables X, X′, and Y, are defined as above in formula (II).

In some embodiments, the first nucleotide at the 5′-end of the oligonucleotide has the structure:

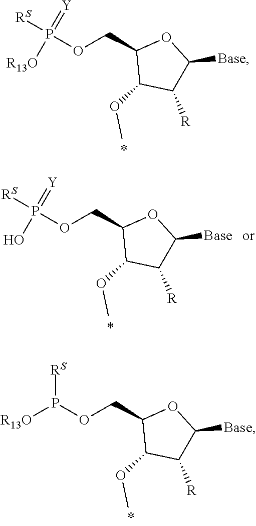

or a salt or a stereoisomer thereof. The variables Base, R^{S}, R¹³, R, and Y are as defined above.

In some embodiments, the first nucleotide at the 5′-end of the oligonucleotide has the structure:

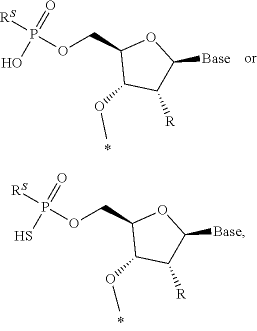

or a salt or a stereoisomer thereof. The variables Base, R^{S}, and R are as defined above.

In some embodiments, B or Base in all these sugar or modified sugar structures above is uridine. In some embodiments, R or R₁ in all these sugar or modified sugar structures above is hydroxy or methoxy. In some embodiments, R or R₁ in all these sugar or modified sugar structures above is hydrogen.

In some embodiments, the oligonucleotide contains at least one

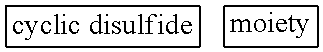

at the 3′-end of the oligonucleotide.

In some embodiments, the oligonucleotide contains at least one

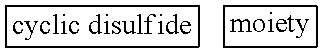

at the 5′-end of the oligonucleotide.

In some embodiments, the oligonucleotide contains at least one

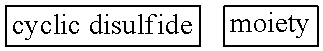

at the 5′-end of the oligonucleotide, and at least one

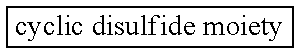

at the 3′-end of the oligonucleotide.

In some embodiments, the oligonucleotide contains at least one

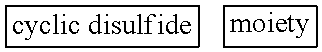

at an internal position of the oligonucleotide.

In some embodiments, the oligonucleotide is a single-stranded oligonucleotide.

In some embodiments, the oligonucleotide is a double-stranded oligonucleotide comprising a sense strand and an antisense strand.

In some embodiments, the sense and antisense strands are each 15 to 30 nucleotides in length. In one embodiment, the sense and antisense strands are each 19 to 25 nucleotides in length. In one embodiment, the sense and antisense strands are each 21 to 23 nucleotides in length.

In some embodiments, the oligonucleotide comprises a single-stranded overhang on at least one of the termini, e.g., 3′ and/or 5′ overhang(s) of 1-10 nucleotides in length, for instance, an overhang having 1, 2, 3, 4, 5, or 6 nucleotides in length. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length, optionally on at least one of the termini.

In some embodiments, the oligonucleotide may also have a blunt end, located at the 5′-end of the antisense strand (or the 3′-end of the sense strand), or vice versa. In one embodiment, the oligonucleotide comprises a 3′ overhang at the 3′-end of the antisense strand, and optionally a blunt end at the 5′-end of the antisense strand. In one embodiment, the oligonucleotide has a 5′ overhang at the 5′-end of the sense strand, and optionally a blunt end at the 5′-end of the antisense strand. In one embodiment, the oligonucleotide has two blunt ends at both ends of a double-stranded iRNA duplex.

In one embodiment, the sense strand of the oligonucleotide is 21-nucleotide in length, and the antisense strand is 23-nucleotide in length, wherein the strands form a double-stranded region of 21 consecutive base pairs having a 2-nucleotide long single-stranded overhangs at the 3′-end.

In one embodiment, the sense strand contains at least one

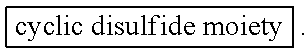

In one embodiment, the antisense strand contains at least one

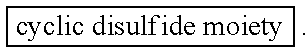

In one embodiment, both the sense strand and the antisense strand each contain at least one

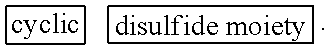

In one embodiment, the oligonucleotide contains at least one

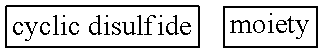

at the 5′-end of the antisense strand and at least one targeting ligand at the 3′-end of the sense strand.

In some embodiments, the sense strand further comprises at least one phosphorothioate linkage at the 3′-end. In some embodiments, the sense strand comprises at least two phosphorothioate linkages at the 3′-end.

In some embodiments, the sense strand further comprises at least one phosphorothioate linkage at the 5′-end. In some embodiments, the sense strand comprises at least two phosphorothioate linkages at the 5′-end.

In some embodiments, the antisense strand further comprises at least one phosphorothioate linkage at the 3′-end. In some embodiments, the antisense strand comprises at least two phosphorothioate linkages at the 3′-end.

In some embodiments, the oligonucleotide further comprises a phosphate or phosphate mimic at the 5′-end of the antisense strand. In one embodiment, the phosphate mimic is a 5′-vinyl phosphonate (VP).

In some embodiments, the 5′-end of the antisense strand does not contain a 5′-vinyl phosphonate (VP).

In some embodiments, the oligonucleotide further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the oligonucleotide further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the oligonucleotide further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the oligonucleotide further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the oligonucleotide further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the oligonucleotide further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the oligonucleotide has at least two phosphorothioate internucleotide linkages at the first five nucleotides on the antisense strand (counting from the 5′ end).

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, the oligonucleotide contains one or more targeting ligands connected to any one of R₂, R₃, R₄, R₅, R₆, R₇, R₈, and R₉ of the

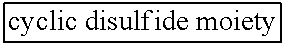

of the compound, optionally via one or more linkers.

In some embodiments, the targeting ligand is selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety that enhances plasma protein binding, a cholesterol, a steroid, bile acid, vitamin B12, biotin, a fluorophore, and a peptide.

In some embodiments, at least one targeting ligand is a lipophilic moiety. In one embodiment, the lipophilicity of the lipophilic moiety, measured by log K_{ow}, exceeds 0, or the hydrophobicity of the compound, measured by the unbound fraction in the plasma protein binding assay of the compound, exceeds 0.2. In one embodiment, the lipophilic moiety contains a saturated or unsaturated C₄-C₃₀ hydrocarbon chain, and an optional functional group selected from the group consisting of hydroxyl, amine, carboxylic acid, sulfonate, phosphate, thiol, azide, and alkyne. For instance, the lipophilic moiety contains a saturated or unsaturated C₆-C₁₈ hydrocarbon chain.

In some embodiments, at least one targeting ligand targets a receptor which mediates delivery to a specific CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

In some embodiments, at least one targeting ligand targets a receptor which mediates delivery to an ocular tissue. In one embodiment, the targeting ligand is selected from the group consisting of trans-retinol, RGD peptide, LDL receptor ligand, and carbohydrate-based ligands. In one embodiment, the targeting ligand is a RGD peptide, such as H-Gly-Arg-Gly-Asp-Ser-Pro-Lys-Cys-OH (SEQ ID. NO: 328) or Cyclo(-Arg-Gly-Asp-D-Phe-Cys) (SEQ ID. NO: 329).

In some embodiments, at least one targeting ligand targets a liver tissue. In some embodiments, the targeting ligand is a carbohydrate-based ligand. In one embodiment, the carbohydrate-based ligand is selected from the group consisting of galactose, multivalent galactose, N-acetyl-galactosamine (GalNAc), multivalent GalNAc, mannose, multivalent mannose, lactose, multivalent lactose, N-acetyl-glucosamine (GlcNAc), multivalent GlcNAc, glucose, multivalent glucose, fucose, and multivalent fucose. In one embodiment, the targeting ligand is a GalNAc conjugate. For instance, the GalNAc conjugate is one or more GalNAc derivatives attached through a bivalent or trivalent branched linker, such as:

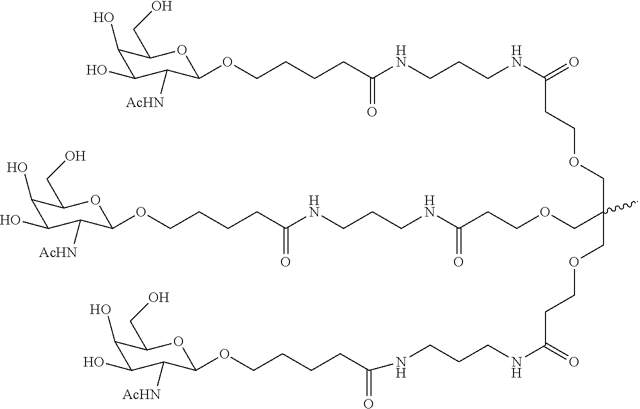 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, 100%, 95%, 90%, 85%, 80%, 75%, 70%, 65%, 60%, 55%, 50%, 45%, 40%, 35% or 30% of the antisense and sense strand of the oligonucleotide is modified. For example, when 50% of the oligonucleotide is modified, 50% of all nucleotides present in the oligonucleotide contain a modification as described herein.

In some embodiments, the antisense and sense strands of the oligonucleotide comprise at least 30%, 35%, 40%, 45%, 50%, 55%, 60%, 65%, 70%, 75%, 80%, 85%, 90%, 95%, or virtually 100% 2′-O-methyl modified nucleotides.

In one embodiment, the oligonucleotide is a double-stranded dsRNA agent, and at least 50% of the nucleotides of the double-stranded dsRNA agent is independently modified with 2′-O-methyl, 2′-O-allyl, 2′-deoxy, or 2′-fluoro.

In one embodiment, the oligonucleotide is an antisense, and at least 50% of the nucleotides of the antisense is independently modified with LNA, CeNA, 2′-methoxyethyl, or 2′-deoxy.

In some embodiments, the sense and antisense strands comprise 12 or less, 10 or less, 8 or less, 6 or less, 4 or less, 2 or less, or no 2′-F modified nucleotides. In some embodiments, the oligonucleotide has 12 or less, 10 or less, 8 or less, 6 or less, 4 or less, 2 or less, or no 2′-F modifications on the sense strand. In some embodiments, the oligonucleotide has 12 or less, 10 or less, 8 or less, 6 or less, 4 or less, 2 or less, or no 2′-F modifications on the antisense strand. In one embodiment, the sense and the antisense strands comprise no more than ten 2′-fluoro modified nucleotides.

In some embodiments, the oligonucleotide contains one or more 2′-0 modifications selected from the group consisting of 2′-deoxy, 2′-O-methoxyalkyl, 2′-O-methyl, 2′-O-allyl, 2′-C-allyl, 2′-fluoro, 2′-O-N-methylacetamido (2′-O-NMA), 2′-O-dimethylaminoethoxyethyl (2′-O-DMAEOE), 2′-O-aminopropyl (2′-O-AP), and 2′-ara-F.

In some embodiments, the oligonucleotide contains one or more 2′-F modifications on any position of the sense strand or antisense strand.

In some embodiments, the oligonucleotide has less than 20%, less than 15%, less than 10%, less than 5% non-natural nucleotide, or substantially no non-natural nucleotide. Examples of non-natural nucleotide include acyclic nucleotides, LNA, HNA, CeNA, 2′-O-methoxyalkyl (e.g., 2′-O-methoxymethyl, 2′-O-methoxyethyl, or 2′-O-2-methoxypropanyl), 2′-O-allyl, 2′-C-allyl, 2′-fluoro, 2′-O-N-methylacetamido (2′-O-NMA), a 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl.

In some embodiments, the oligonucleotide has greater than 80%, greater than 85%, greater than 90%, greater than 95%, or virtually 100% natural nucleotides. For the purpose of these embodiments, natural nucleotides can include those having 2′-OH, 2′-deoxy, and 2′-OMe.

In some embodiments, the antisense strand contains at least one unlocked nucleic acids (UNA) or glycerol nucleic acid (GNA) modification, e.g., at the seed region of the antisense strand. In one embodiment, the seed region is at positions 2-8 (or positions 5-7) of the 5′-end of the antisense strand.

In one embodiment, the oligonucleotide comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the oligonucleotide has less than 20%, less than 15%, less than 10%, less than 5% non-natural nucleotide, or substantially no non-natural nucleotide.

In one embodiment, the oligonucleotide comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the oligonucleotide has greater than 80%, greater than 85%, greater than 95%, or virtually 100% natural nucleotides, such as those having 2′-OH, 2′-deoxy, or 2′-OMe.

One aspect of the invention provides an oligonucleotide comprising a sense strand and an antisense strand, each strand independently having a length of 15 to 35 nucleotides; at least two phosphorothioate internucleotide linkages between the first five nucleotides counting from the 5′ end of the antisense strand; at least three, four, five, or six 2′-deoxy modifications on the sense and/or antisense strands; wherein the oligonucleotide has a double stranded (duplex) region of between 19 to 25 base pairs; wherein the oligonucleotide comprises a ligand.

In one embodiment, the sense strand does not comprise a glycol nucleic acid (GNA).

It is understood that the antisense strand has sufficient complementarity to a target sequence to mediate RNA interference. In other words, the oligonucleotide is capable of inhibiting the expression of a target gene.

In one embodiment, the oligonucleotide comprises at least three 2′-deoxy modifications. The 2′-deoxy modifications are at positions 2 and 14 of the antisense strand, counting from 5′-end of the antisense strand, and at position 11 of the sense strand, counting from 5′-end of the sense strand.

In one embodiment, the oligonucleotide comprises at least five 2′-deoxy modifications. The 2′-deoxy modifications are at positions 2, 12 and 14 of the antisense strand, counting from 5′-end of the antisense strand, and at positions 9 and 11 of the sense strand, counting from 5′-end of the sense strand.

In one embodiment, the oligonucleotide comprises at least seven 2′-deoxy modifications. The 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In one embodiment, the antisense strand comprises at least five 2′-deoxy modifications at positions 2, 5, 7, 12 and 14, counting from 5′-end of the antisense strand. The antisense strand has a length of 18-25 nucleotides, or a length of 18-23 nucleotides.

In one embodiment, the oligonucleotide comprises less than 20%, e.g., less than 15%, less than 10%, or less than 5% non-natural nucleotides, or comprises no non-natural nucleotides.

In one embodiment, the sense strand does not comprise a glycol nucleic acid (GNA); and wherein the oligonucleotide comprises less than 20%, e.g., less than 15%, less than 10%, or less than 5% non-natural nucleotides or comprises all natural nucleotides.

In one embodiment, at least one the sense and antisense strands comprises at least one, e.g., at least two, at least three, at least four, at least five, at least six, or at least seven or more, 2′-deoxy modifications in a central region of the sense or antisense strand.

In one embodiment, the sense strand and/or the antisense strand comprises at least one, e.g., at least two, at least three, at least four, at least five, at least six, or at least seven or more, 2′-deoxy modifications in a central region of the sense strand and/or the antisense strand.

In some embodiment, the sense strand has a length of 18 to 30 nucleotides and comprises at least two 2′-deoxy modifications in the central region of the sense strand. For example, the sense strand has a length of 18 to 30 nucleotides and comprises at least two 2′-deoxy modifications within positions 7, 8, 9, 10, 11, 12, and 13, counting from 5′-end of the sense strand.

In one embodiment, the antisense strand has a length of 18 to 30 nucleotides and comprises at least two 2′-deoxy modifications in the central region of the antisense strand. For example, the antisense strand has length of 18 to 30 nucleotides and comprises at least two 2′-deoxy modifications within positions 10, 11, 12, 13, 14, 15 and 16, counting from 5′-end of the antisense strand.

In one embodiment, the oligonucleotide comprises a sense strand and an antisense strand; wherein the sense strand has a length of 17-30 nucleotide and comprises at least one 2′-deoxy modification in the central region of the sense strand; and wherein the antisense strand independently has a length of 17-30 nucleotides and comprises at least two 2′-deoxy modifications in the central region of the antisense strand.

In one embodiment, the oligonucleotide comprises a sense strand and an antisense strand; wherein the sense strand has a length of 17-30 nucleotide and comprises at least two 2′-deoxy modifications in the central region of the sense strand; and wherein the antisense strand independently has a length of 17-30 nucleotides and comprises at least one 2′-deoxy modification in the central region of the antisense strand.

In one embodiment, the sense strand comprises at least one, e.g., at least two, at least three, at least four, at least five, at least six, at least seven or more, 2′-deoxy modifications in a central region of the sense strand.

In one embodiment, the antisense strand comprises at least one, e.g., at least two, at least three, at least four, at least five, at least six, at least seven or more, 2′-deoxy modifications in a central region of the antisense strand.

In one embodiment, the oligonucleotide comprises less than 20%, e.g., less than 15%, less than 10%, or less than 5% non-natural nucleotides or the oligonucleotide comprises all natural nucleotides; and wherein the sense strand and/or the antisense strand comprises at least one, e.g., at least two, at least three, at least four, at least five, at least six, at least seven or more, 2′-deoxy modifications in a central region of the sense strand and/or the antisense strand.

In one embodiment, the oligonucleotide comprises less than 20%, e.g., less than 15%, less than 10%, or less than 5% non-natural nucleotides or the oligonucleotide comprises all natural nucleotides; and wherein the sense strand comprises at least one, e.g., at least two, at least three, at least four, at least five, at least six, at least seven or more, 2′-deoxy modifications in a central region of the sense strand.

In one embodiment, the oligonucleotide comprises less than 20%, e.g., less than 15%, less than 10%, or less than 5% non-natural nucleotides or the oligonucleotide comprises all natural nucleotides; and wherein the antisense strand comprises at least one, e.g., at least two, at least three, at least four, at least five, at least six, at least seven or more, 2′-deoxy modifications in a central region of the antisense strand.

In one embodiment, when the oligonucleotide comprises less than 8 non-2′OMe nucleotides, the antisense stand comprises at least one DNA. For example, in any one of the embodiments of the invention when the oligonucleotide comprises less than 8 non-2′OMe nucleotides, the antisense stand comprises at least one DNA.

In one embodiment, when the antisense comprises two deoxy nucleotides and said nucleotides are at positions 2 and 14, counting from the 5′-end of the antisense strand, the oligonucleotide comprises 8 or less (e.g., 8, 7, 6, 5, 4, 3, 2, 1 or 0) non-2′OMe nucleotides. For example, in any one of the embodiments of the invention when the antisense comprises two deoxy nucleotides and said nucleotides are at positions 2 and 14, counting from the 5′-end of the antisense strand, the oligonucleotide comprises 0, 1, 2, 3, 4, 5, 6, 7 or 8 non 2′-OMe nucleotides.

Another aspect of the invention relates to a pharmaceutical composition comprising the oligonucleotide described herein, and a pharmaceutically acceptable excipient.

All the above embodiments relating to the oligonucleotide in the above aspect of the invention relating to the oligonucleotide are suitable in this aspect of the invention relating to the pharmaceutical composition.

In another aspect, the invention further provides a method for delivering the oligonucleotide of the invention to a specific target in a subject by subcutaneous or intravenous administration. The invention further provides the oligonucleotide of the invention for use in a method for delivering said agents to a specific target in a subject by subcutaneous or intravenous administration.

Another aspect of the invention relates to a method of reducing or inhibiting the expression of a target gene in a subject, comprising administering to the subject the oligonucleotide described herein above in an amount sufficient to inhibit expression of the target gene.

All the above embodiments relating to the oligonucleotide in the above aspect of the invention relating to the oligonucleotide are suitable in this aspect of the invention relating to a method of reducing the expression of a target gene in a subject.

Another aspect of the invention relates to a method for modifying an oligonucleotide comprising contacting the oligonucleotide with the compound described herein above under conditions suitable for reacting the compound with the oligonucleotide, wherein the oligonucleotide comprises a free hydroxyl group.

In some embodiments, the free hydroxyl group is part of the 5′-terminal nucleotide. In some embodiments, the free hydroxyl group is part of the 3′-terminal nucleotide.

In some embodiments, the oligonucleotide comprises a 5′-OH group. In some embodiments, the oligonucleotide comprises a 3′-OH group.

In some embodiments, the conditions suitable for reacting the compound with the oligonucleotide comprise an acidic catalyst. For instance, the acid catalyst may be a substituted tetrazole. Suitable acidic catalysts include, but not limited to, 1H-tetrazole, 5-ethylthio-1H-tetrazole, 2-benzylthiotetrazole, 4,5-dicyanoimidazole, 5-nitrophenyl-1H-tetrazole, 5-(bis-3,5-trifluoromethylphenyl)-1H-tetrazole, 5-benzylthio-1H-tetrazole, 5-mthylthio-1H-tetrazole, 1-hydroxyl benzotriazole, 1-hydroxy-6-trifluoromethyl benzotriazole, 4-nitro-1-hydroxy-6-trifluoromethyl benzotriazole, pyridinium chloride, pyridinium bromide, pyridinium 4-methylbenzenesulfonate, 2,6-di(tert-butyl)pyridinium chloride, pyridinium trifluoroacetate, N-(phenyl)imidazolium triflate (N-PhIMT), N-(phenyl)-imidazolium perchlorate (N-PhIMP), N-(methyl)benzimidazolium triflate (NMeBIT), N-(p-acetylphenyl)imidazolium triflate (N-AcPhIMT), N-(phenyl)imidazolium tetrafluoroborate (N-PhIMTFB), imidazolium perchlorate (IMP), 4-(phenyl)-imidazolium triflate (4-PhIMT), benzimidazolium tetrafluoroborate (BITFB), imidazolium tetrafluoroborate (IMTFB), imidazolium triflate (IMT), benzimidazolium triflate (BIT), 2-(phenyl)imidazolium triflate (2-PhIMT), N-(methyl)imidazolium triflate (N-MeIMT), 4-(methyl)imidazolium triflate (4-MeIMT), saccharin-1-methylimidazole, N-(cyanomethyl)pyrrolidinium triflate, trichloroacetic acid (TCA), trifluoroacetic acid (TFA), dichloroacetic acid (DCA), and 2,4-dinitrobenzoic acids (2,4-DNBA), iron chloride (FeCl3), aluminum chloride (AlCl3), trifluoroboron etherate (BF3-OEt2), zirconium(IV) chloride (ZrCl4), and bismuth(III) chloride (BiCl3), trimethylchlorosilane, 2,4-dinitrophenol, 1-methyl-5-mercapto-tetrazole, and 1-phenyl-5-mercaptotetrazole.

All the above embodiments relating to the compound and the oligonucleotide in the above aspects of the invention are suitable in this aspect of the invention relating to a method for modifying an oligonucleotide.

Another aspect of the invention relates to a method for preparing a modified oligonucleotide, comprising: oxidizing a first oligonucleotide comprising a group of formula (A):

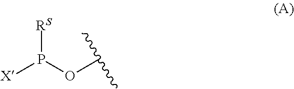

or a salt or a stereoisomer thereof, wherein:
R^{S} is a

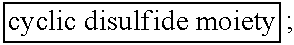

X′ is —OR¹³ or —SR¹³, wherein R¹³ is alkyl, alkenyl, alkynyl, cycloalkyl, aryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
   under conditions suitable for forming a modified oligonucleotide comprising a group of formula (B):

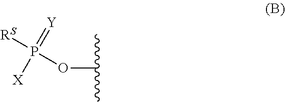

or a salt or a stereoisomer thereof, wherein Y is O or S; and X is —OH, —SH or X′. The variables Base, R^{S}, X, X′, and Y are as defined above.

In some embodiments, the first nucleotide at the 5′-end of the first oligonucleotide comprises the group of formula (A) and the first nucleotide at the 5′-end of the modified oligonucleotide comprises the group of formula (B). In some embodiments, the last nucleotide at the 3′-end of the first oligonucleotide comprises the group of formula (A) and the last nucleotide at the 3′-end of the modified oligonucleotide comprises the group of formula (B).

In some embodiments, the first nucleotide at the 5′-end of the first oligonucleotide is according to formula (C):

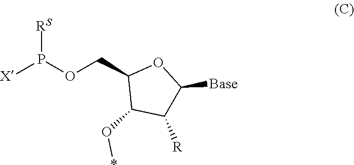

or a salt or a stereoisomer thereof, wherein:
* represents a bond to the subsequent optionally modified internucleotide linkage;
Base is an optionally modified nucleobase; and
R is H, OH, O-methoxyalkyl, O-methyl, O-allyl, CH₂-allyl, fluoro, O-N-methylacetamido (O-NMA), O-dimethylaminoethoxyethyl (O-DMAEOE), O-aminopropyl (O-AP), or ara-F. The variables R^{S} and X′ are as defined above.

In some embodiments, the first nucleotide at the 5′-end of the modified oligonucleotide has the structure of formula (D):

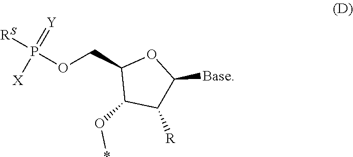

The variables Base, R, R^{S}, X, and Y are as defined above.

In some embodiments, the first nucleotide at the 5′-end of the modified oligonucleotide has the structure of formula (E) or (F):

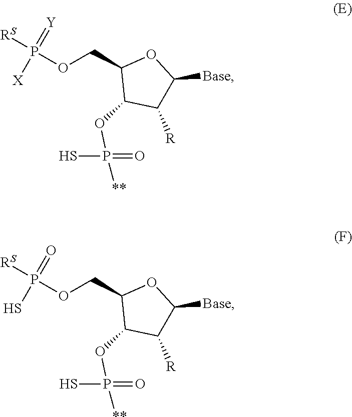

or a salt or a stereoisomer thereof, wherein ** represents the bond to the subsequent nucleotide. The variables Base, R, R^{S}, X, and Y are as defined above.

In some embodiments, the conditions suitable for forming a modified oligonucleotide comprise using an oxidizing agent selected from the group consisting of iodine; sulfur; a peroxide; a peracid; phenylacetyl disulfide; 3H-1,2-benzodithiol-3-one 1,1-dioxide; dixanthogen; 5-ethoxy-3H-1,2,4-dithiazol-3-one; 3-[(dimethylaminomethylene)amino]-3H-1,2,4-dithiazole-5-thione (DDTT); dimethyl sulfoxide; and N-bromosuccinimide. For instance, the oxidizing agents may be a peracid (e.g., m-chloroperbenzoic acid), or a peroxide (e.g., tert-butyl hydroperoxide or trimethylsilyl peroxide).

All the above embodiments relating to the compound and the oligonucleotide in the above aspects of the invention are suitable in this aspect of the invention relating to a method for preparing a modified oligonucleotide.

Another aspect of the invention relates to a precursor compound comprising a structure of formula (I), or a salt or stereoisomer thereof:

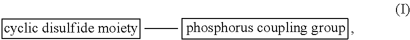

wherein the

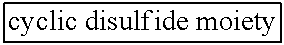

has the structure of:

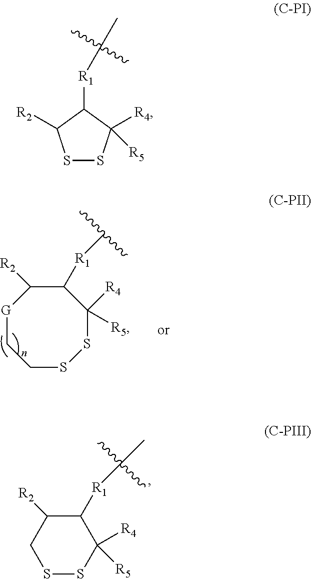

wherein:
R₁ is O or S, and is bonded to the P atom of the

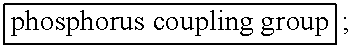

R₂ is (CH₂)ₛ—W, (CH₂)ₛ—O—(CH₂)ₛ—W, (CH₂)ₛ—(CH₂CH₂O)ₜ—CH₂)ₛ—W, or (CH₂)ₛO(CH₂CH₂O)ₜ—(CH₂)ₛ—W;
s is an integer of 0-22,
t is an integer of 1-20;
W is a reactive group;
R₄ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₄ and R₅ form a second ring;
G is O, N(R′), S, or C(R⁴)(R⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″, together with the adjacent nitrogen atom, form a ring; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.

In some embodiments, the

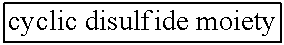

has the structure of:

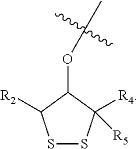

R₂ is as defined above. R₄ and R₅ are each independently H, C₁₋₆ alkyl, or phenyl. Alternatively, R₄ and R₅, together with the adjacent carbon atom, form a second ring of 3-7 atoms.

In some embodiments, the

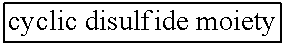

has the structure of:

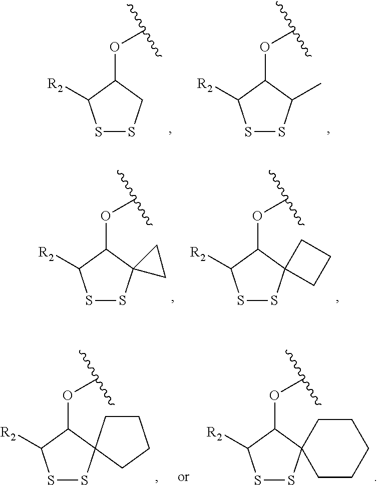

In some embodiments, the

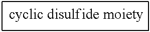

has the structure of:

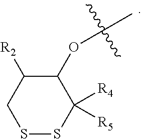

R₂ is as defined above. R₄ and R₅ are each independently H, C₁₋₆ alkyl, or phenyl. Alternatively, R₄ and R₅, together with the adjacent carbon atom, form a second ring of 3-7 atoms.

In one embodiment, the

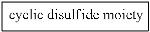

has the structure of:

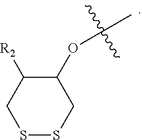

In some embodiments, W in R₂ is NHTFA (CF₃C(O)N(H)-), N₃, C≡CH, C(O)OR¹³, or OC(O)R¹³, wherein R¹³ is C₁-C₃ alkyl.

In some embodiments, the

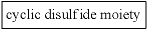

has one of the following structures:

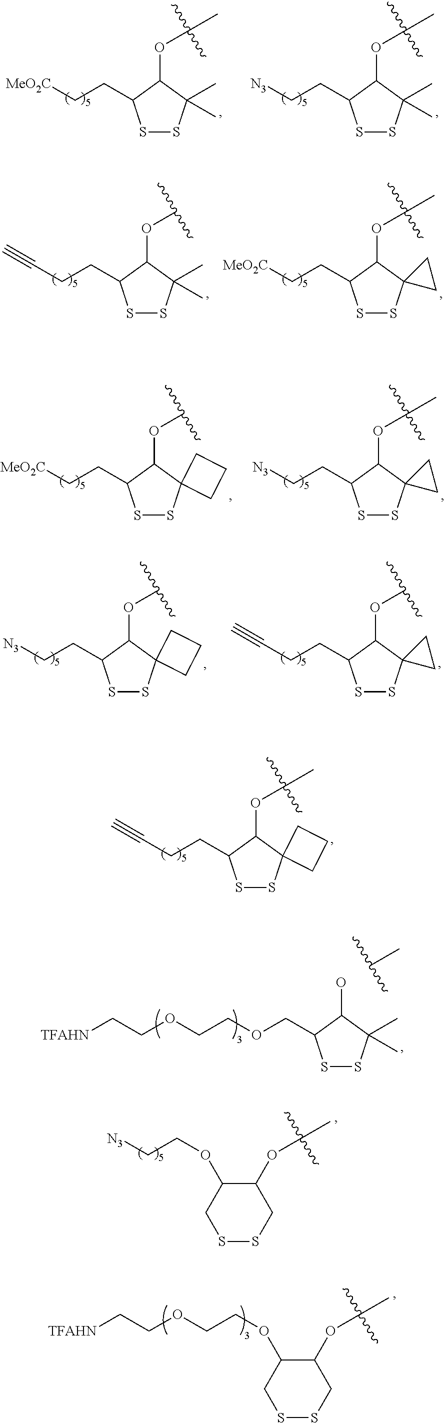

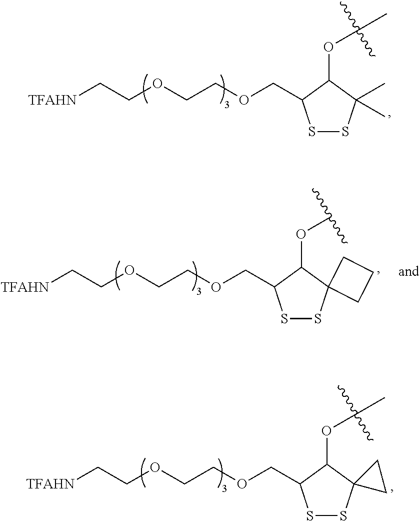

wherein TFAHN— is CF₃C(O)N(H)—.

In some embodiments, the

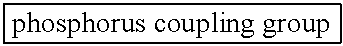

has the structure of:

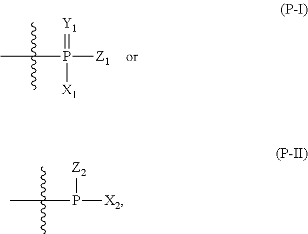

wherein:
X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), NSO₂R′, B(R¹³)₃, BH₃⁻, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R′), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide,
Y₁ is S, O, or N(R′);
M is an organic or inorganic cation; and
R¹⁸ is H or alkyl, optionally substituted with one or more R^{sub} groups.

In some embodiments, the

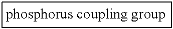

has the structure of

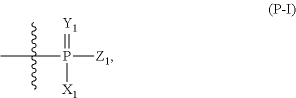

wherein:
X₁ and Z₁ are each independently OH, OM, SH, SM, C(O)H, S(O)H, C₁-C₆ alkyl optionally substituted with one or more hydroxy or halo groups, or D-Q;
D is independently for each occurrence absent, O, S, NH, C₁-C₆ alkylene optionally substituted with one or more halo groups; and
Y₁ is S or O.

In some embodiments, the

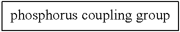

has the structure of

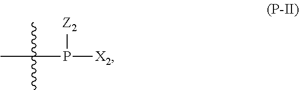

wherein:
X₂ is N(R′)(R″);
Z₂ is X₂, OR¹⁸, or D-Q;
R¹⁸ is H or C₁-C₆ alkyl substituted with cyano; and
R′ and R″ are each independent C₁-C₆ alkyl.

In some embodiments, the

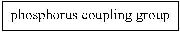

has a structure selected from the group consisting of

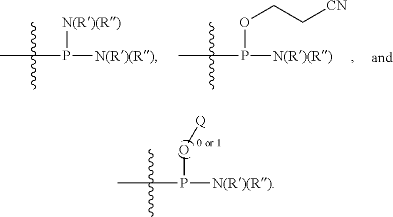

In some embodiments, the compound has one of the following structures:

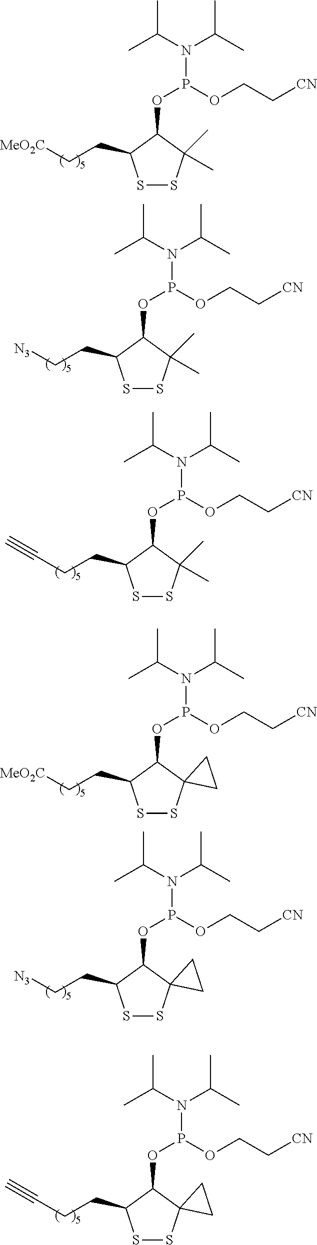

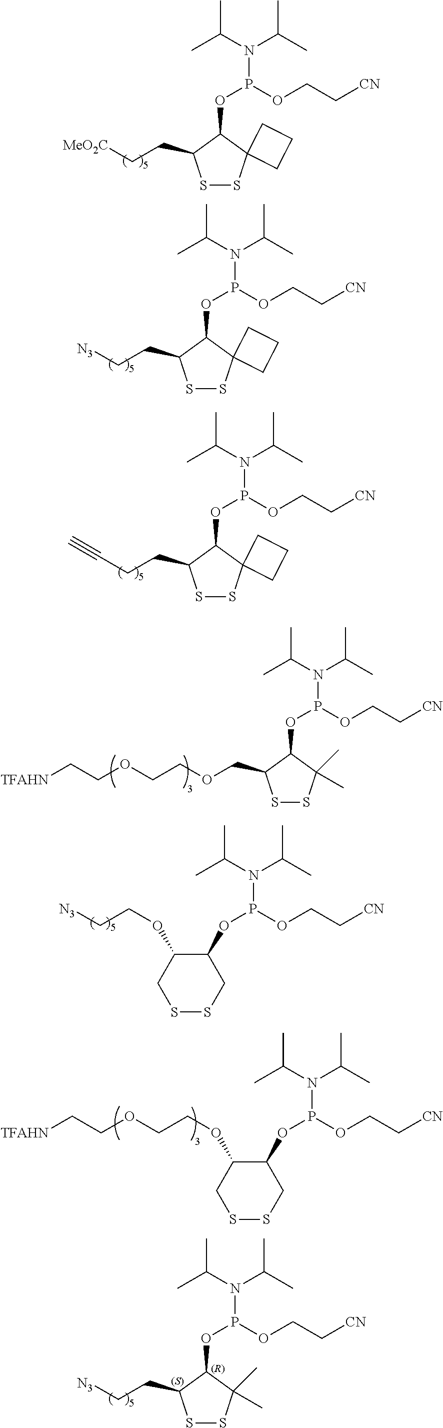

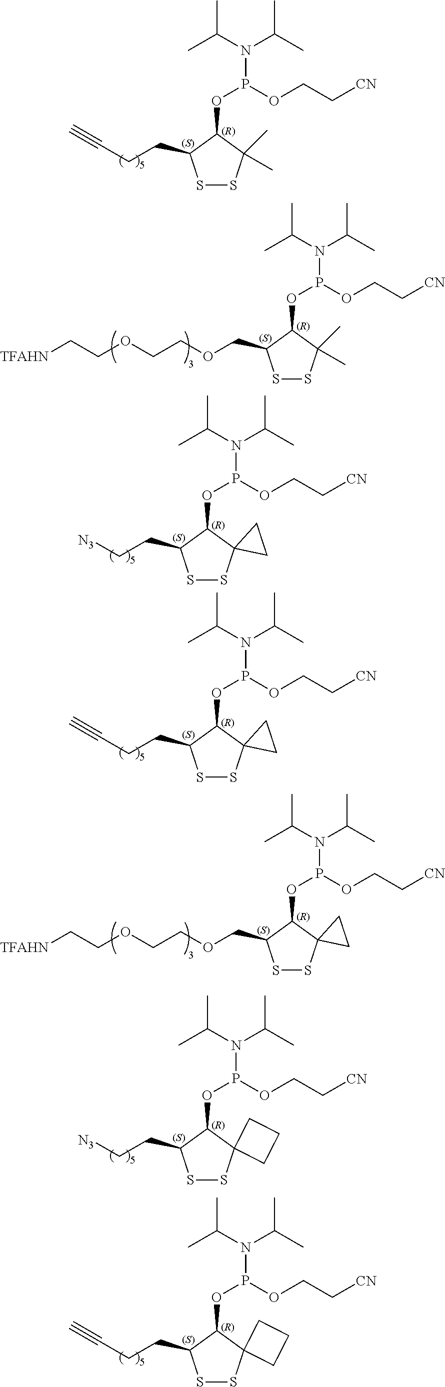

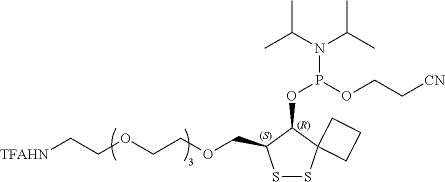

wherein TFAHN— is CF₃C(O)N(H)—.

Another aspect of the invention relates to comprising one or more structures of formula (II):

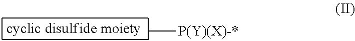

wherein the

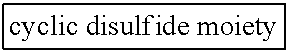

has the structure of:

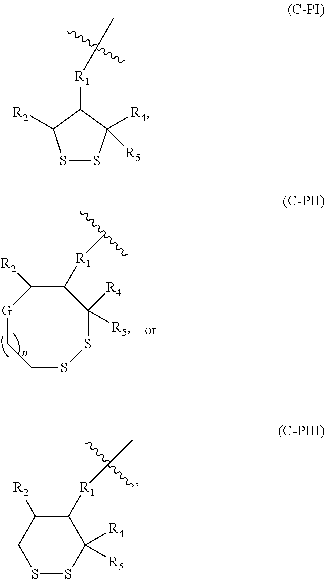

or a salt or stereoisomer thereof,
wherein:
* represents the bond to the oligonucleotide,
Y is absent, N(R′), ═O, or ═S, X is —OH, —SH, C(O)H, S(O)H, alkyl optionally substituted with one or more R^{sub} groups, N(R′)(R″), NSO₂R′, N═CN(R′)(R″), B(R¹³)₃, BH₃⁻, or X′, wherein X′ is N(R′)(R″), —OR¹³ or —SR¹³;
R₁ is O or S, and is bonded to the P atom of the —P(Y)(X)—* group;
R₂ is (CH₂)ₛ—W, (CH₂)ₛ—O—(CH₂)ₛ—W, (CH₂)ₛ—(CH₂CH₂O)ₜ—(CH₂)ₛ—W, or (CH₂)ₛO(CH₂CH₂O)ₜ—(CH₂)ₛ—W;
s is an integer of 0-22,
t is an integer of 1-20;
W is a reactive group;
R₄ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₄ and R₅ form a second ring;
G is O, N(R′), S, or C(R′)(R¹⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″ together with the adjacent nitrogen atom form a ring, and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido;
wherein at least one

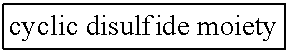

is connected at the 5′ end of the nucleoside or oligonucleotide.

In some embodiments, the

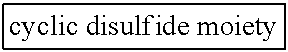

has the structure of:

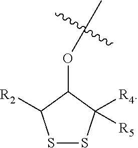

R₂ is as defined above. R₄ and R₅ are each independently H, C₁₋₆ alkyl, or phenyl. Alternatively, R₄ and R₅, together with the adjacent carbon atom, form a second ring of 3-7 atoms.

In some embodiments, the

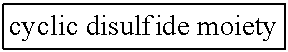

has the structure of:

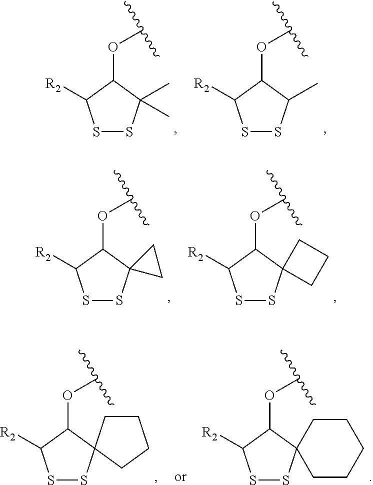

The

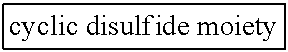

has the structure of:

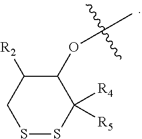

R₂ is as defined above. R₄ and R₅ are each independently H, C₁₋₆ alkyl, or phenyl. Alternatively, R₄ and R₅, together with the adjacent carbon atom, form a second ring of 3-7 atoms.

In one embodiment, the

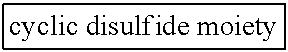

has the structure of:

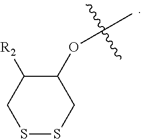

In some embodiments, W in R₂ is NHTFA, N₃, C≡CH, C(O)OR¹³, OC(O)R¹³, wherein R¹³ is C₁-C₃ alkyl.

In some embodiments, the

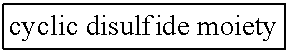

has one of the following structures:

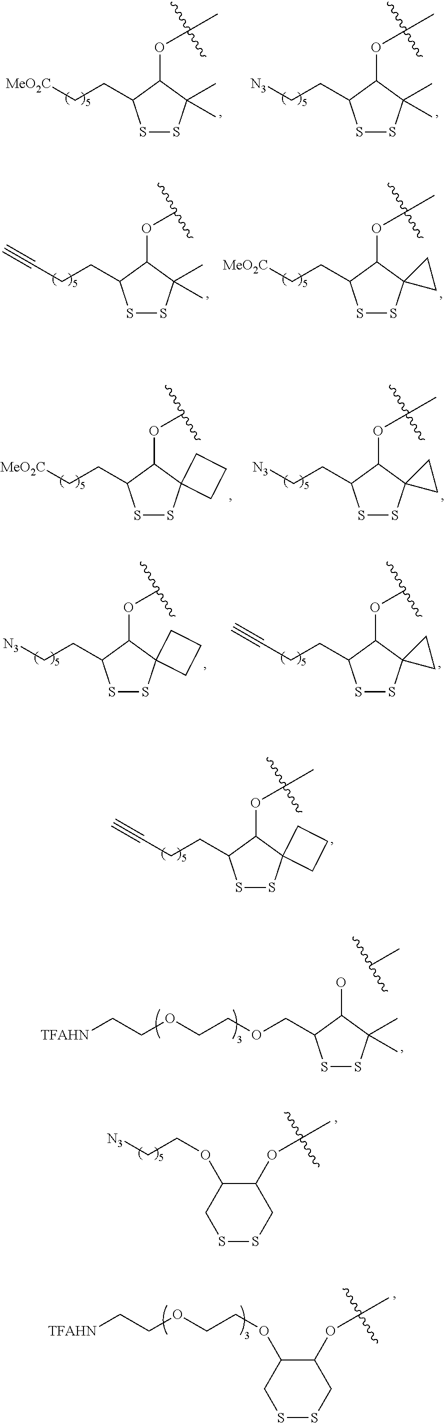

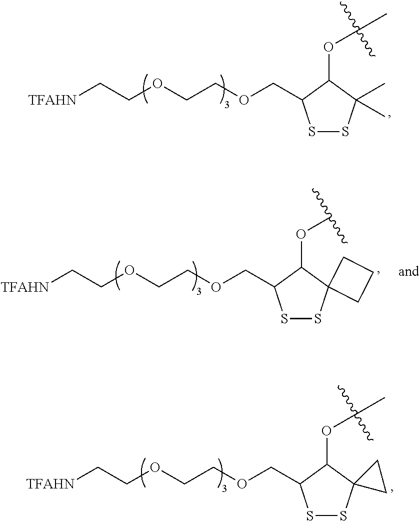

wherein TFAHN— is CF₃C(O)N(H)—.

In some embodiments, the oligonucleotide comprises a structure having the formula:

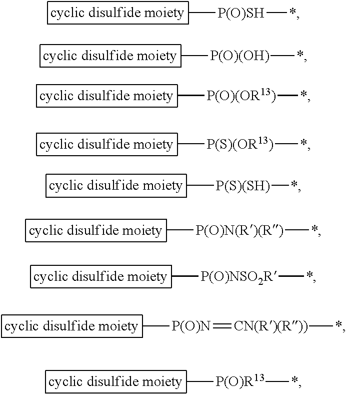

or salt thereof. R¹³, R′, R″, and * are as defined above.

The precursor compound and the oligonucleotide containing the precursor compound provided herein contains a reactive group to facilitate 5′-end conjugation. For instance, the

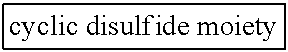

containing the reactive group W can be reacted with a ligand containing a functional group reactive to the W reactive group (NHTFA, N₃, C≡CK, C(O)O—, OC(O), etc.), for further 5′ end conjugation. In one embodiment, the

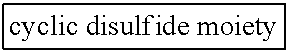

contains N₃ or C≡CH can be reacted with corresponding ligands containing C≡CH or N₃, respectively, via click chemistry.

### BRIEF DESCRIPTION OF THE DRAWINGS

FIG. 1 is a graph depicting in vitro activity of F12 siRNAs containing the modified phosphate prodrugs at the 5′ end in primary mouse hepatocytes, after transfection with RNAiMAX at 0.1, 1, 10, and 100 nm concentrations and analyzed 24 hours post-transfection. Percentage of F12 message remaining was determined by qPCR and were plotted against the control.

FIG. 2 is a graph depicting in vitro activity of F12 siRNA duplexes containing the modified phosphate prodrugs at the 5′ end in primary mouse hepatocytes after incubating at 0.1, 1, 10, and 100 nm concentrations and analyzed 48 hours post-incubation. Percentage of F12 message remaining was determined by qPCR and were plotted against the control.

FIG. 3 is a graph depicting in vitro activity of F12 siRNA duplexes containing the modified phosphate prodrugs at the 5′ end in primary mouse hepatocytes after transfection with RNAiMAX at 0.1, 1, and 10 nm concentrations and analyzed 24 hours post-transfection. Percentage of F12 message remaining was determined by qPCR and were plotted against the control.

FIGS. 4A-J show the representative LCMS spectra of oligonucleotides tested in the DTT reduction assay.

FIG. 5 is a graph depicting the relative mF12 protein in circulation by ELISA in mice following subcutaneous administration of F12 siRNA duplexes containing the modified phosphate prodrugs at the 5′ end at single dose 0.3 mg/kg, compared to PBS control.

FIG. 6 is a graph depicting the relative mF12 protein in circulation by ELISA in mice following subcutaneous administration of F12 siRNA duplexes containing the modified phosphate prodrugs at the 5′ end at single dose 0.1 mg/kg or 0.3 mg/kg, compared to PBS control.

FIG. 7 shows the possible in vivo cytosolic unmasking mechanism of the 5′ cyclic disulfide modified phosphate prodrugs to reveal 5′-phosphate.

FIG. 8 is a graph depicting the relative SOD1 mRNA remaining in thoracic spinal cord, hippocampus, frontal cortex, striatum, and heart of rats, determined by qPCR, after 14 days following intrathecal (IT) administration of SOD1 siRNA duplexes containing the modified phosphate prodrugs at the 5′ end at a single dose of 0.1 mg.

FIG. 9 is a graph depicting the relative SOD1 mRNA remaining in thoracic spinal cord, cerebellum, frontal cortex, striatum, and heart of rats, determined by qPCR, after 84 days following intrathecal (IT) administration of SOD1 siRNA duplexes containing the modified phosphate prodrugs at the 5′ end at a single dose of 0.3 mg.

FIG. 10 is a graph depicting the relative SOD1 mRNA remaining in thoracic spinal cord, hippocampus, frontal cortex, striatum, and heart of rats, determined by qPCR, after 14 days following intrathecal (IT) administration of SOD1 siRNA duplexes containing the modified phosphate prodrugs at the 5′ end at a single dose of 0.9 mg.

FIG. 11 is a graph depicting the relative SOD1 mRNA remaining in thoracic spinal cord, cerebellum, frontal cortex, striatum, and heart of rats, determined by qPCR, after 84 days following intrathecal (IT) administration of SOD1 siRNA duplexes containing the modified phosphate prodrugs at the 5′ end at a single dose of 0.9 mg.

FIG. 12 is a graph depicting the relative SOD1 mRNA remaining by qPCR in thoracic spinal cord, hippocampus, frontal cortex, striatum, and heart of rats after 14 days following intrathecal administration of SOD1 siRNA duplexes containing the modified phosphate prodrugs at the 5′ end at a single dose of 0.9 mg.

FIG. 13 is a graph depicting the relative SOD1 mRNA remaining by qPCR in thoracic spinal cord, hippocampus, frontal cortex, striatum, and heart of rats after 14 days following intrathecal administration of SOD1 siRNA duplexes containing the modified phosphate prodrugs at the 5′ end at a single dose of either 0.3 mg or 0.9 mg.

FIG. 14 is a graph depicting the relative SOD1 mRNA remaining by qPCR in right brain hemisphere of mice after 7 days following intracranial ventricular administration of SOD1 siRNA duplexes containing the modified phosphate prodrugs at the 5′ end at a single dose of 100 μg.

FIGS. 15A-15B show in vitro activity of F12 siRNAs containing cis- or trans-modified phosphate prodrugs at the 5′-end of the antisense strand in primary mouse hepatocytes. FIG. 15A shows the results after transfection with RNAiMAX, analyzed at 24 hours post transfection at 0.1, 1, 10, and 100 nM concentrations. FIG. 15B shows the results of free uptake after incubation with primary mouse hepatocytes at 0.1, 1, 10, and 100 nM concentrations for 48 hours. Percentage of F12 message remaining was determined by qPCR and were plotted against the control.

FIGS. 16A-16B show in vitro activity of F12 siRNAs containing modified phosphate prodrugs at the 5′-end of antisense strand in primary mouse hepatocytes. FIG. 16A shows the results after transfection with RNAiMAX, and analyzed 24 hours post transfection at 0.1, 1, and 10 nm concentrations. FIG. 16B shows the results of free uptake after incubation with primary mouse hepatocytes at 0.1, 1, 10, and 100 nM concentrations for 48 hours. Percentage of F12 message remaining was determined by qPCR and were plotted against the control.

FIGS. 17A-17B show in vitro activity of F12 siRNAs containing modified phosphate prodrugs at the 5′-end of antisense strand in primary mouse hepatocytes. FIG. 17A shows the results after transfection with RNAiMAX, and analyzed 24 hours post transfection at 1, 10, and 100 nm concentrations (from right to left for each siRNA data point). FIG. 17B shows the results of free uptake after incubation with primary mounse hepatocytes at 1, 10, and 100 nM concentrations (from right to left for each siRNA data point) for 48 hours. Percentage of F12 message remaining was determined by qPCR and were plotted against the control.

FIG. 18 is a graph depicting an example for rates of 5′-P-prodrug unmasking from the corresponding F12 single strands in DTT assay: 100 uM oligo, 0.1 M DTT, room temp., 0 and 24 h timepoints.

FIG. 19 is a graph depicting the rates of exemplary 5′-cyclic modified phosphate prodrugs from the corresponding F12 single strands in GHS assay, performed by using 100 μM single-stranded oligonucleotide with a 10 mM glutathione, 250 μg glutathione-S-transferase, 0.1 mg/mL NADPH in 100 mM Tris pH 7.2, with IEX. The half-lives where reported by monitoring glutathione-mediated cleavage kinetics every hour for 24 hours, relative to half-life of two controls containing linear disulfides.

FIGS. 20A-20B show in vivo activity of F12 siRNAs containing cis- or trans-modified phosphate prodrugs at the 5′-end of antisense strand in mice at 0.3 mg/kg dose. Percentage of F12 protein remaining was determined by blood draws and were plotted against the control.

FIG. 21 shows in vivo activity of F12 siRNAs containing chiral or chirally enriched versions of racemic trans-phosphate prodrugs (Pmmd) at the 5′-end of antisense strand in mice at 0.1 mg/kg dose. Percentage of F12 protein remaining was determined by blood draws and were plotted against the control.

FIGS. 22A-22B shows in vivo hepatic activity of F12 siRNAs containing 5′-phosphate prodrugs at the 5′-end of antisense strand in mice at 0.3 mg/kg dose. Percentage of F12 protein remaining was determined by blood draws and were plotted against the control.

FIG. 23 shows in vivo CNS activity of SOD1 siRNAs containing 5′-phosphate prodrugs at the 5′-end of antisense strand in mice via a single dose of 100 μg via ICV administration. The mRNAs from right brain hemisphere and liver were measured by qPCR at day 8 and plotted against aCSF control.

FIG. 24 shows in vivo CNS activity of SOD1 siRNAs containing 5′-phosphate prodrugs at the 5′-end of antisense strand in rat via a single dose of 1.5 mg in 50 μl via IT administration. The mRNAs from right brain hemisphere and liver were measured by qPCR at day 14 in various brain regions, liver, and heart and plotted against aCSF control.

FIGS. 25A-25B show in vivo muscle activity of SOD1 siRNAs containing 5′-phosphate prodrugs at the 5′-end of antisense strand in mice via a single dose of 2 mg/kg. The mRNAs from quadriceps (FIG. 25A) and gastrocnemius (FIG. 25B) were measured by qPCR at day 14 and plotted against PBS control.

FIG. 26 shows in vivo heart muscle activity of SOD1 siRNAs containing 5′-phosphate prodrugs at the 5′-end of antisense strand in mice via a single dose of 2 mg/kg. The mRNAs from heart muscle was measured by qPCR at day 14 and plotted against PBS control.

FIG. 27 shows in vivo adipose tissue activity of SOD1 siRNAs containing 5′-phosphate prodrugs at the 5′-end of antisense strand in mice via a single dose 2 mg/kg. The mRNAs from adipose tissue was measured by qPCR at day 14 and plotted against PBS control.

### DETAILED DESCRIPTION

The inventors have discovered novel categories of cyclic disulfide moieties that can be introduced to the phosphate group of an oligonucleotide (e.g., a single-stranded iRNA agent a double-stranded iRNA agent) to temporarily mask the phosphate group, and that can be in vivo cleaved via cellular activation. The cellular activation is via glutathione or dithiothreitol mediated reduction/bioconvention mechanism to release the active anionic form of the phosphate group from the masking group. The inventors have discovered that the cyclic disulfide moieties can be introduced at either the sense strand or the antisense strand or both the sense and antisense strands, at the 5′ end, 3′ end, and/or internal position(s) of a strand. Introduction of the cyclic disulfide moieties modified phosphate prodrug at the 5′ end of the antisense strand provides particularly good results.

### The Modified Phosphate Prodrug Compound

One aspect of the invention relates to a modified phosphate prodrug compound. The compound comprises a structure of formula (I):

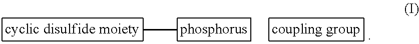

The compound can also comprise a salt or a stereoisomer of the structure of formula (I).

The

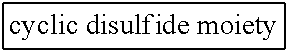

has the structure of:

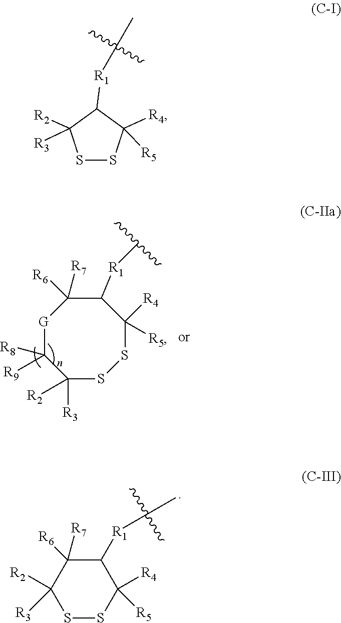

### Alternatively, the

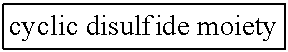

can have the structure of

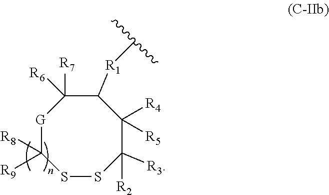

In formulas (C-I), (C-IIa), (C-IIb), and (C-III):
R₁ is O or S, and is bonded to the P atom of the

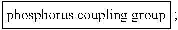

[symbol]

indicates the bond to the

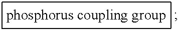

R₂, R₄, R₆, R₇, R₈, and R₉ are each independently H, halo, OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups;
R₃ and R₅ are each independently H, halo, OR¹³ or alkylene-OR¹³, N(R′)(R″) or alkylene-N(R′)(R″), alkyl, C(R¹⁴)(R⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₃ and R₅, together with the adjacent carbon atoms and the two sulfur atoms, form a second ring;
G is O, N(R′), S, or C(R¹⁴)(R¹⁵);
n is an integer of 0-6;
R¹³ is independently for each occurrence H, alkyl, alkenyl, alkynyl, cycloalkyl, aryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.
R₂, R₄, R₆, R₇, R₈, and R₉ can also each independently be CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), each of which can be optionally substituted by one or more R^{sub} groups.
R₃ and R₅ can also each independently be CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), each of which can be optionally substituted by one or more R^{sub} groups.
R₂ and R₃, together with the adjacent carbon atom, can form another ring.
R₄ and R₅, together with the adjacent carbon atom, can form another ring.
R₆ and R₇, together with the adjacent carbon atom, can form another ring.
R₈ and R₉, together with the adjacent carbon atoms, can form another ring.
two or more of R₂, R₃, R₄, R₅, R₆, R₇, R₈, R₉, R¹⁴, and R¹⁵, together with the adjacent carbon atoms can form one or more rings fused with the ring containing the two sulfur atoms.
R¹³, R′, and R″ can also each independently be heteroaryl.
R′ and R″, together with the adjacent nitrogen atom, can form a ring.

In some embodiments, in the

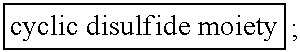

R₁ is O;
G is CH₂;
n is 0 or 1;
R₂, R₄, R₆, R₇, R₈, and R₉ are each independently H, halo, OR¹³ or C₁-C₆ alkylene-OR¹³, N(R′)(R″) or C₁-C₆ alkylene-N(R′)(R″), C₁-C₆ alkyl, aryl, heteroaryl, each of which can be optionally substituted by one or more R^{sub} groups;
R₃ and R₅ are each independently H, halo, OR¹³ or C₁-C₆ alkylene-OR¹³, N(R′)(R″) or C₁-C₆ alkylene-N(R′)(R″), C₁-C₆ alkyl, aryl, heteroaryl, each of which can be optionally substituted by one or more R^{sub} groups; or R₃ and R₅, together with the adjacent carbon atoms and the two sulfur atoms, form a second ring of 6-8 atoms;
R¹³ is independently for each occurrence H, C₁-C₆ alkyl, aryl, alkylcarbonyl, or arylcarbonyl; and
R′ and R″ are each independently H or C₁-C₆ alkyl.
R₂, R₄, R₆, R₇, R₈, and R₉ may also each independently be CN or C₁-C₆ alkylene-CN, C(O)OR¹³ or C₁-C₆ alkylene-C(O)OR¹³, S(O)OR¹³ or C₁-C₆ alkylene-S(O)OR¹³, C(O)N(R′)(R″) or C₁-C₆ alkylene-C(O)N(R′)(R″), each of which can be optionally substituted by one or more R^{sub} groups.
R₃ and R₅ may also each independently be CN or C₁-C₆ alkylene-CN, C(O)OR¹³ or C₁-C₆ alkylene-C(O)OR¹³, S(O)OR¹³ or C₁-C₆ alkylene-S(O)OR¹³, C(O)N(R′)(R″) or C₁-C₆ alkylene-C(O)N(R′)(R″), each of which can be optionally substituted by one or more R^{sub} groups.
R₂ and R₃, together with the adjacent carbon atom, may form another ring of 3-7 atoms.
R₄ and R₅, together with the adjacent carbon atom, may form another ring of 3-7 atoms.
R₆ and R₇, together with the adjacent carbon atom, may form another ring of 3-7 atoms.
R₈ and R₉, together with the adjacent carbon atom, may form another ring of 3-7 atoms.
two or more of R₂, R₃, R₄, R₅, R₆, R₇, R₈, R₉, R¹⁴, and R¹⁵, together with the adjacent carbon atoms may form one or more ring of 5-7 atoms fused with the ring containing the two sulfur atoms.

The

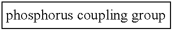

can have a structure of:

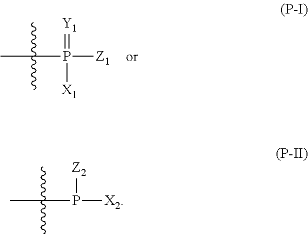

In formulas (P-I) and (P-II):
[symbol] indicates the bond to the

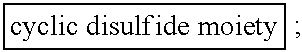

X₁ and Z₁ are each independently H, OH, OM, OR¹³, SH, SM, SR¹³, C(O)H, S(O)H, or alkyl, each of which can be optionally substituted with one or more R⁶ groups, N(R′)(R″), B(R¹³)₃, BH₃⁻, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide,
Y₁ is S, O, or N(R′);
M is an organic or inorganic cation; and
R¹⁸ is H or alkyl, optionally substituted with one or more R^{sub} groups.
X₁ and Z₁ may also each independently be NSO₂R′ or N═CN(R′)(R″).

In some embodiments, the

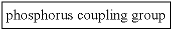

has the structure of

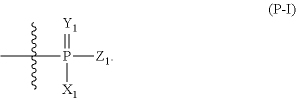

In this formula: X₁ and Z₁ are each independently OH, OM, SH, SM, C(O)H, S(O)H, C₁-C₆ alkyl optionally substituted with one or more hydroxy or halo groups, or D-Q; D is independently for each occurrence absent, O, S, NH, C₁-C₆ alkylene optionally substituted with one or more halo groups; and Y₁ is S or O. X₁ and Z₁ may also each independently be OR¹³, SR¹³, NSO₂R′, or N═CN(R′)(R″). In one embodiment, X₁ is OH or SH; and Z₁ is D-Q.

In some embodiments, the

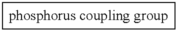

has one of the following structures:

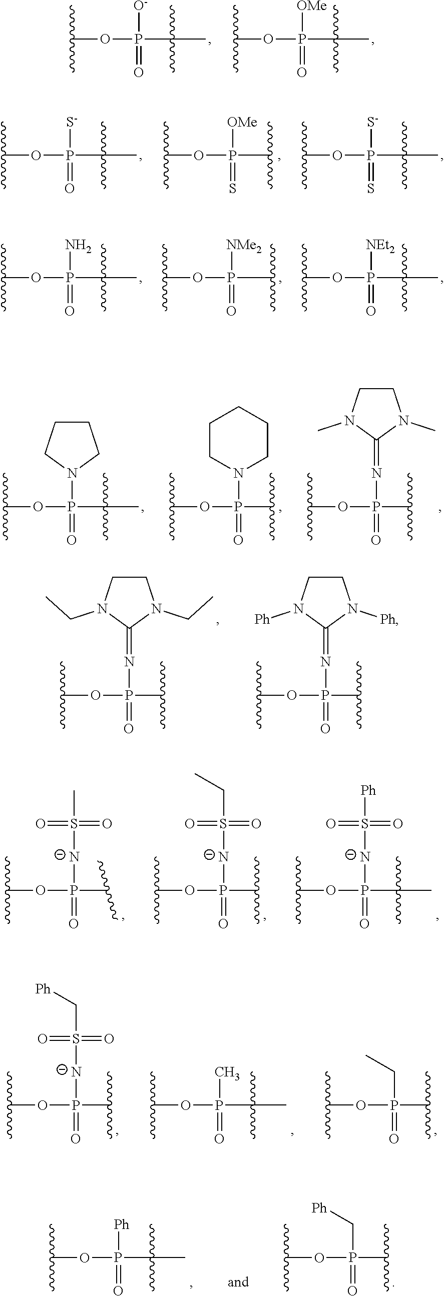

In one embodiment, the

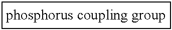

has the structure of

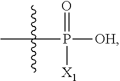

wherein X₁ is OH or SH.

In some embodiments, the

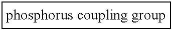

has the structure of

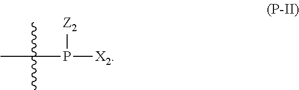

In this formula, X₂ is N(R′)(R″); Z₂ is X₂, OR¹⁸, or D-Q; R¹⁸ is H or C₁-C₆ alkyl substituted with cyano; and R′ and R″ are each independent C₁-C₆ alkyl (e.g., iso-propyl).

In one embodiment, the

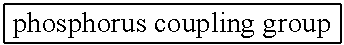

has a structure selected from the group consisting of

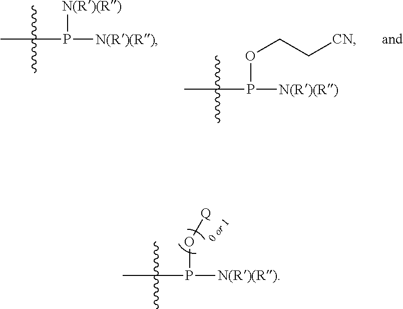

The variables R′, R″, and Q are defined as above in formulas P-I and P-II. In one embodiment, R′ and R″ are each iso-propyl.

In some embodiments, the

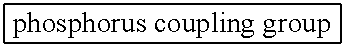

has the structure —P(Z)(X), wherein:
X is selected from the group consisting of —OCH₃, —OCH₂CH₃, —OCH₂CH₂CH₃, —OCH₂CH(CH₃)₂, —OCH₂CH₂CN, —OCH₂CH₂Si(CH₃)₃, —OCH₂CH₂Si(CH₂CH₃)₃, —OC(H)═H₂, —OCH₂C(H)═CH₂

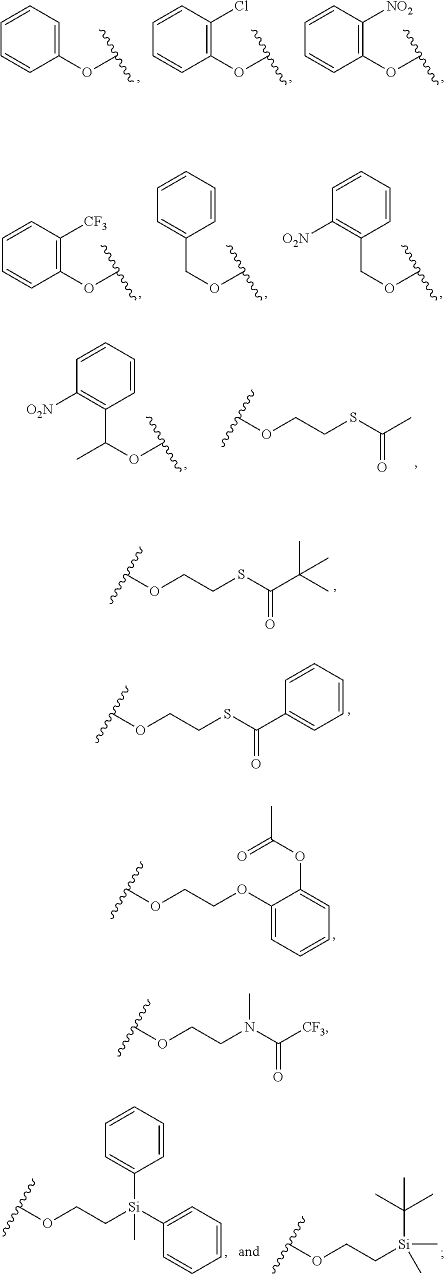

and
Z is selected from the group consisting of

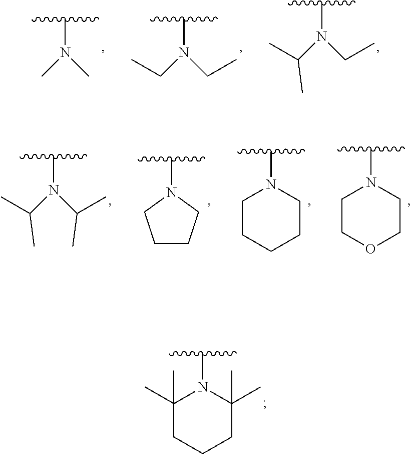

X and Z taken together with the phosphorus atom to which they are attached form a cyclic structure selected from the group consisting of

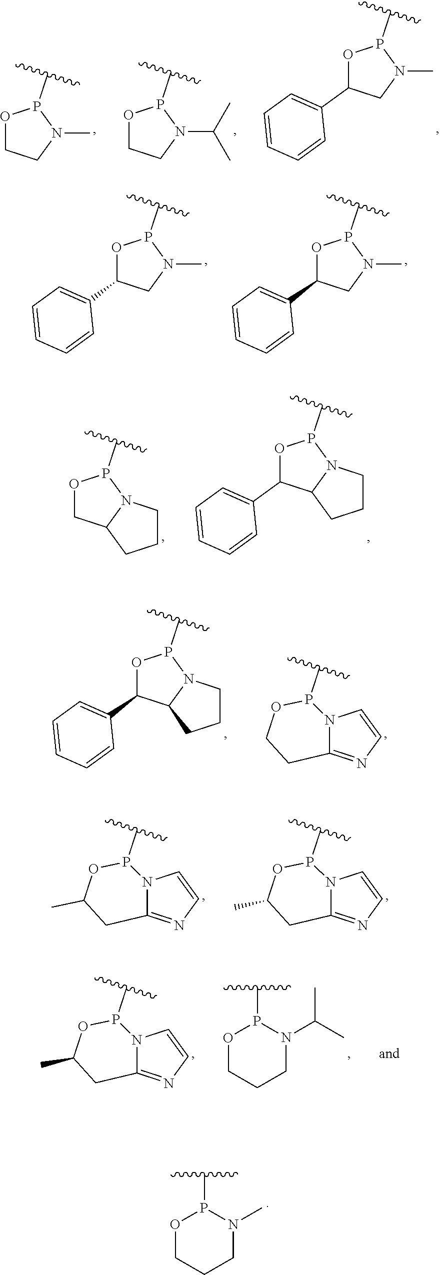

In one embodiment, the

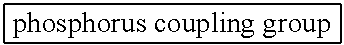

has the structure of

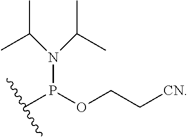 [SMILES: *P(OCCC#N)N(C(C)C)C(C)C]

In some embodiments, the

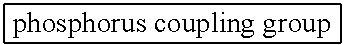

has various modifications for stabilization and has one of the following structures:

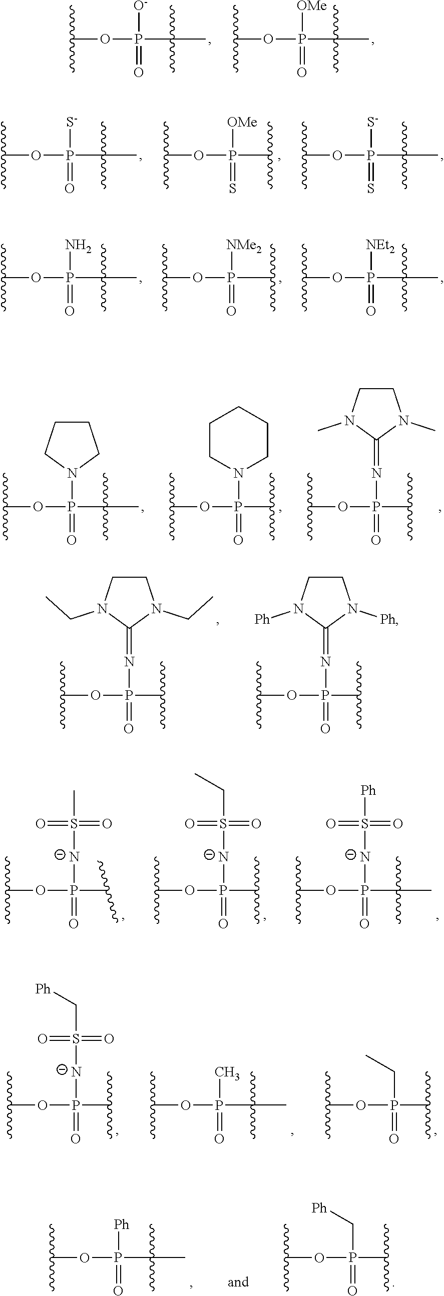

One [symbol] indicates the bond to the

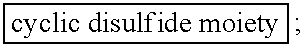

the [symbol] indicates the bond to a nucleoside or oligonucleotide.

Exemplary compounds with different stabilization at the phosphorous-containing internucleotide linkage, described above, are:

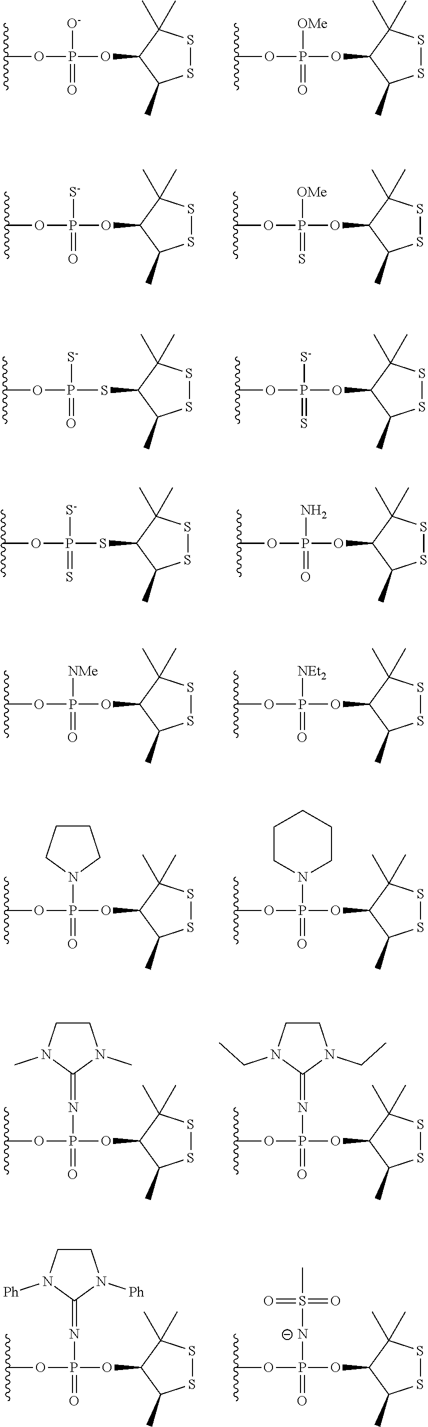

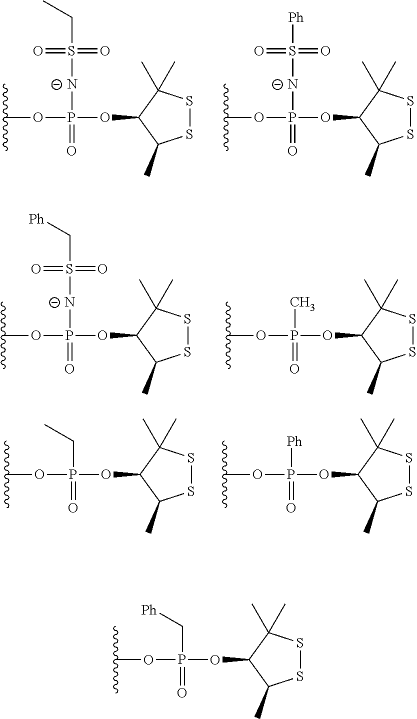

The

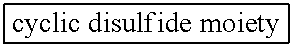

can have the structure

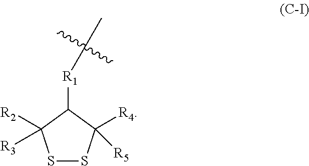

Exemplary

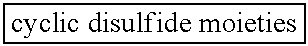

for 5-member cyclic compounds of formula (C-I) include:

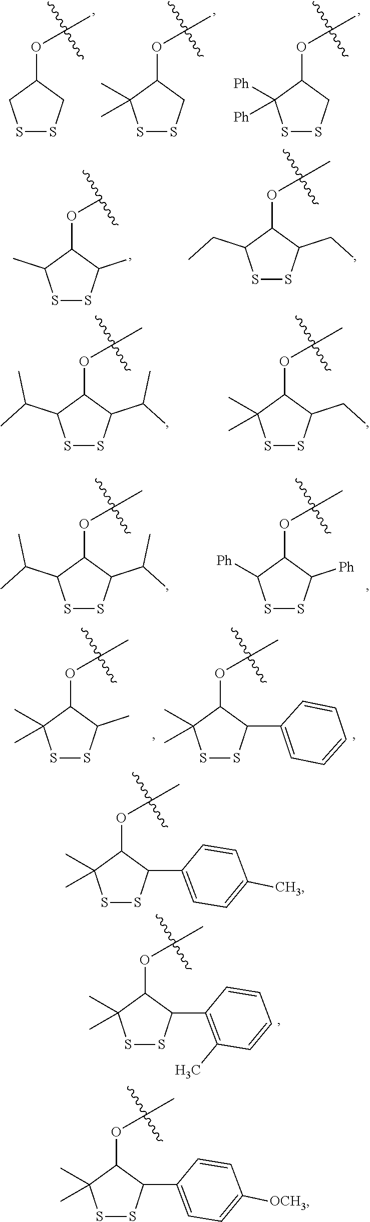

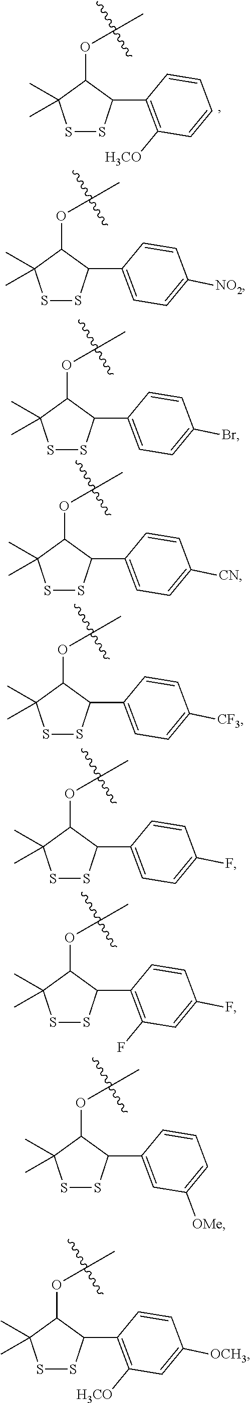

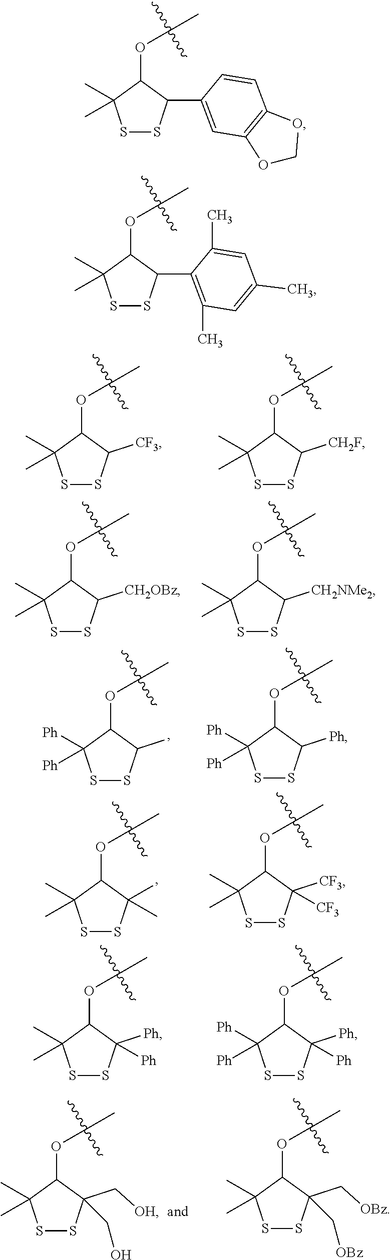

In some embodiments, the compound has the formula of

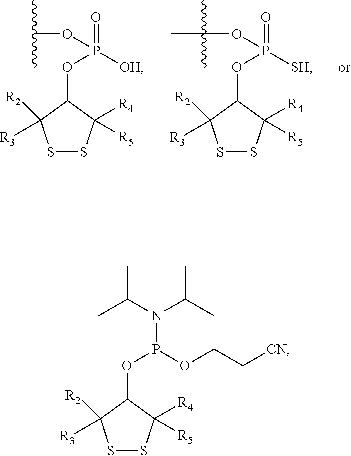

wherein: R₂, R₃, R₄, and R₅ are each independently H, alkyl (e.g., CH₃), heterocyclic, CH₂R¹⁵, aryl (e.g., phenyl), heteroaryl, CHFR¹⁵, CF₂R¹⁵, CF₃; and can be in any stereoisomeric configurations; and R¹⁵ is alkyl, heterocyclic, aryl, OH, O-alkyl, NH₂, NH(alkyl), N(alkyl)₂, CF₂ R¹⁵, or CF₃; and can be in any stereoisomeric configurations.

In some embodiments, the compound has the formula of

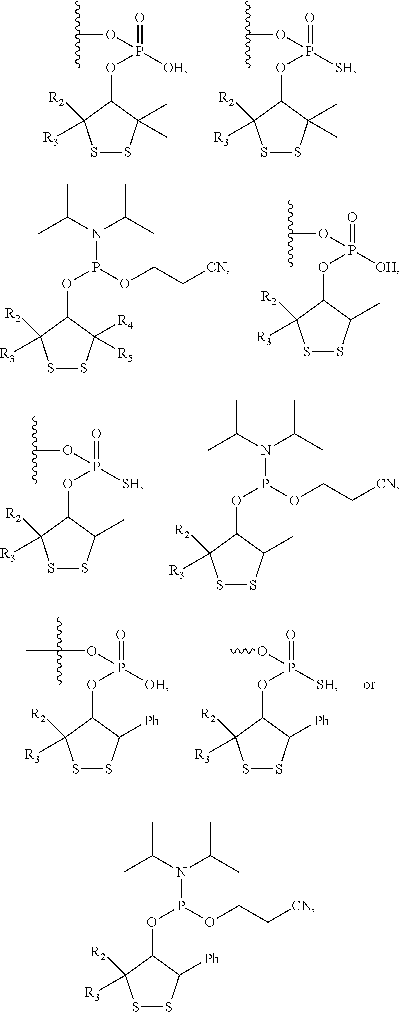

wherein: R₂ and R₃ are each independently H, alkyl (e.g., CH₃), heterocyclic, CN, CF₃, CH₂R¹⁵, heteroaryl, CHFR¹⁵, CF₂R¹⁵, C(O)NHR¹⁵, C(O)N(R¹⁵), C(O)OR¹⁵, S(O)OR¹⁵, CH₂C(O)OR¹⁵, CH₂S(O)OR¹⁵; and can be in any stereoisomeric configurations; and R¹⁵ is H, alkyl, heterocyclic, aryl (e.g., substituted or unsubstituted phenyl), heteroaryl, or CF₃; and can be in any stereoisomeric configurations.

In some embodiments, the compound has the formula of:

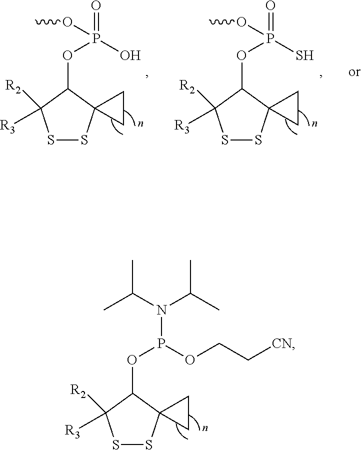

wherein n is 1, 2, 3, or 4; R₂ and R₃ are each independently H, alkyl (e.g., CH₃), heterocyclic, CN, CF₃, CH₂R¹⁵, aryl (e.g., substituted or unsubstituted phenyl), heteroaryl, CHFR¹⁵, CF₂R¹⁵, C(O)NHR¹⁵, C(O)N(R⁵)₂, C(O)OR¹⁵, S(O)OR¹⁵, CH₂C(O)OR¹⁵, CH₂S(O)OR¹⁵; and can be in any stereoisomeric configurations; and R¹⁵ is H, alkyl, heterocyclic, aryl (e.g., phenyl), heteroaryl, or CF₃; and can be in any stereoisomeric configurations.

Exemplary compounds of formula (I) with a 5-member cyclic disulfide moiety are shown in Table 1.

TABLE 1
Compounds with 5-member cyclic disulfide moiety

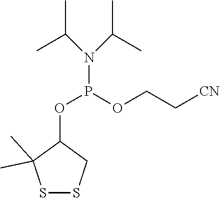 [SMILES: CC(C)N(C(C)C)P(OCCC#N)OC1CSSC1(C)C]

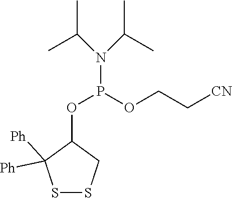 [SMILES: CC(C)N(C(C)C)P(OCCC#N)OC1CSSC1(c1ccccc1)c1ccccc1]

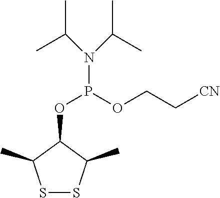 [SMILES: CC(C)N(C(C)C)P(OCCC#N)O[C@@H]1[C@H](C)SS[C@@H]1C]

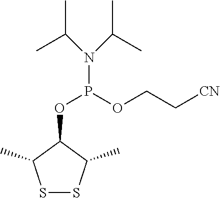 [SMILES: CC(C)N(C(C)C)P(OCCC#N)O[C@H]1[C@H](C)SS[C@@H]1C]

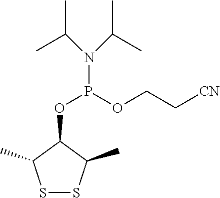

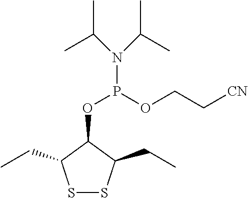

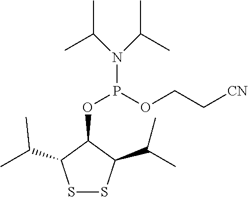

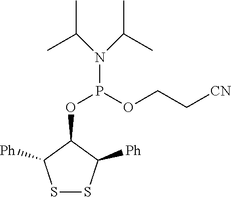

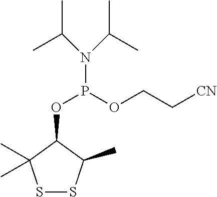 [SMILES: CC(C)N(C(C)C)P(OCCC#N)O[C@H]1[C@@H](C)SSC1(C)C]

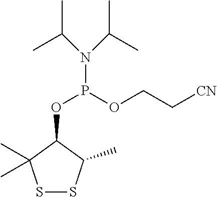 [SMILES: CC(C)N(C(C)C)P(OCCC#N)O[C@H]1[C@H](C)SSC1(C)C]

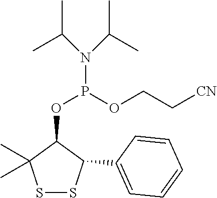 [SMILES: CC(C)N(C(C)C)P(OCCC#N)O[C@H]1[C@H](c2ccccc2)SSC1(C)C]

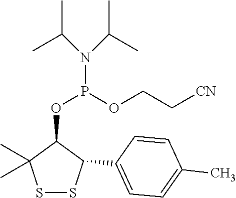

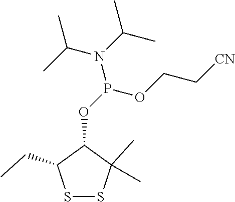 [SMILES: CC[C@H]1SSC(C)(C)[C@H]1OP(OCCC#N)N(C(C)C)C(C)C]

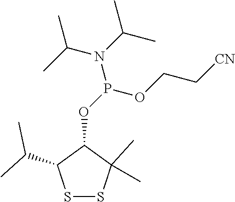 [SMILES: CC(C)[C@H]1SSC(C)(C)[C@H]1OP(OCCC#N)N(C(C)C)C(C)C]

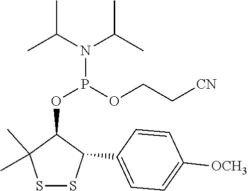 [SMILES: COc1ccc([C@@H]2SSC(C)(C)[C@H]2OP(OCCC#N)N(C(C)C)C(C)C)cc1]

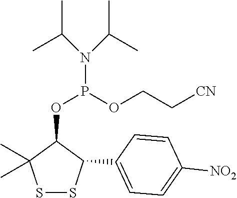 [SMILES: CC(C)N(C(C)C)P(OCCC#N)O[C@H]1[C@H](c2ccc([N+](=O)[O-])cc2)SSC1(C)C]

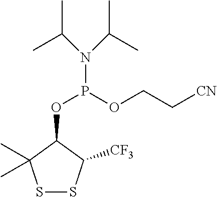 [SMILES: CC(C)N(C(C)C)P(OCCC#N)O[C@H]1[C@H](C(F)(F)F)SSC1(C)C]

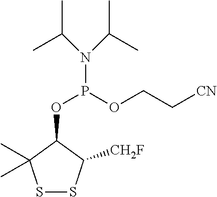

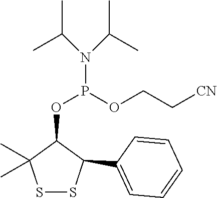 [SMILES: CC(C)N(C(C)C)P(OCCC#N)O[C@H]1[C@@H](c2ccccc2)SSC1(C)C]

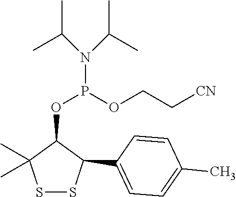

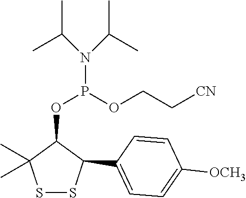 [SMILES: COc1ccc([C@H]2SSC(C)(C)[C@H]2OP(OCCC#N)N(C(C)C)C(C)C)cc1]

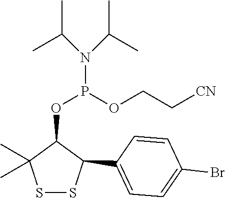 [SMILES: CC(C)N(C(C)C)P(OCCC#N)O[C@H]1[C@@H](c2ccc(Br)cc2)SSC1(C)C]

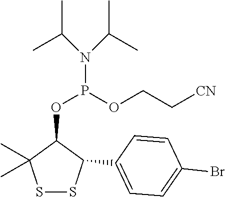 [SMILES: CC(C)N(C(C)C)P(OCCC#N)O[C@H]1[C@H](c2ccc(Br)cc2)SSC1(C)C]

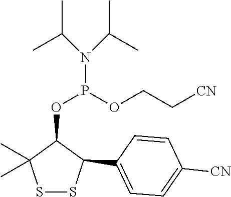 [SMILES: CC(C)N(C(C)C)P(OCCC#N)O[C@H]1[C@@H](c2ccc(C#N)cc2)SSC1(C)C]

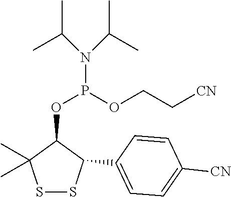 [SMILES: CC(C)N(C(C)C)P(OCCC#N)O[C@H]1[C@H](c2ccc(C#N)cc2)SSC1(C)C]

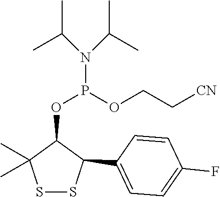 [SMILES: CC(C)N(C(C)C)P(OCCC#N)O[C@H]1[C@@H](c2ccc(F)cc2)SSC1(C)C]

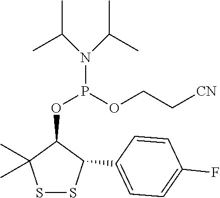 [SMILES: CC(C)N(C(C)C)P(OCCC#N)O[C@H]1[C@H](c2ccc(F)cc2)SSC1(C)C]

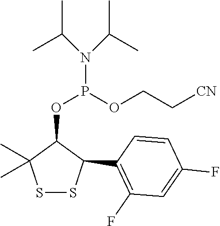 [SMILES: CC(C)N(C(C)C)P(OCCC#N)O[C@H]1[C@@H](c2ccc(F)cc2F)SSC1(C)C]

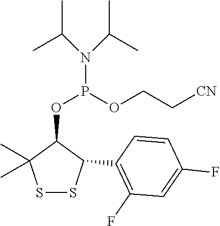 [SMILES: CC(C)N(C(C)C)P(OCCC#N)O[C@H]1[C@H](c2ccc(F)cc2F)SSC1(C)C]

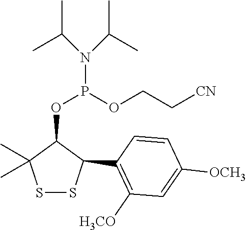 [SMILES: COc1ccc([C@H]2SSC(C)(C)[C@H]2OP(OCCC#N)N(C(C)C)C(C)C)c(OC)c1]

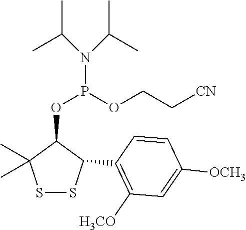 [SMILES: COc1ccc([C@@H]2SSC(C)(C)[C@H]2OP(OCCC#N)N(C(C)C)C(C)C)c(OC)c1]

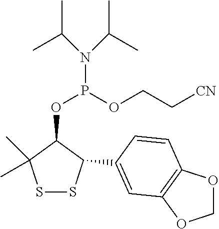 [SMILES: CC(C)N(C(C)C)P(OCCC#N)O[C@H]1[C@H](c2ccc3c(c2)OCO3)SSC1(C)C]

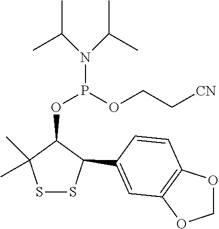 [SMILES: CC(C)N(C(C)C)P(OCCC#N)O[C@H]1[C@@H](c2ccc3c(c2)OCO3)SSC1(C)C]

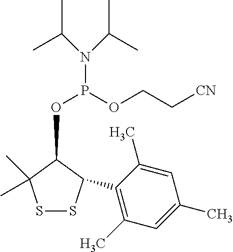

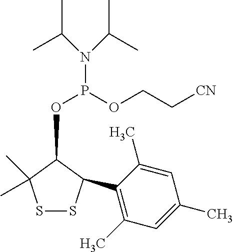

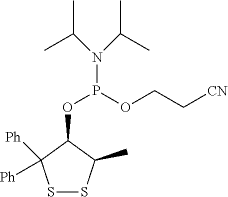 [SMILES: CC(C)N(C(C)C)P(OCCC#N)O[C@H]1[C@@H](C)SSC1(c1ccccc1)c1ccccc1]

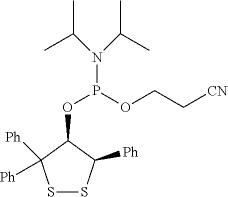 [SMILES: CC(C)N(C(C)C)P(OCCC#N)O[C@H]1[C@@H](c2ccccc2)SSC1(c1ccccc1)c1ccccc1]

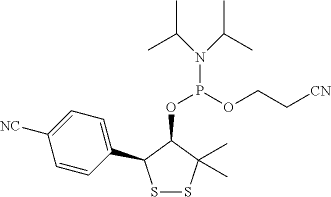 [SMILES: CC(C)N(C(C)C)P(OCCC#N)O[C@@H]1[C@H](c2ccc(C#N)cc2)SSC1(C)C]

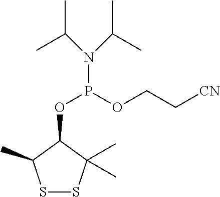 [SMILES: CC(C)N(C(C)C)P(OCCC#N)O[C@@H]1[C@H](C)SSC1(C)C]

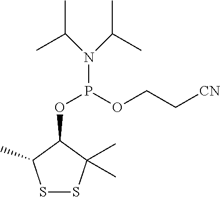 [SMILES: CC(C)N(C(C)C)P(OCCC#N)O[C@@H]1[C@@H](C)SSC1(C)C]

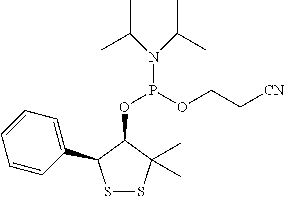 [SMILES: CC(C)N(C(C)C)P(OCCC#N)O[C@@H]1[C@H](c2ccccc2)SSC1(C)C]

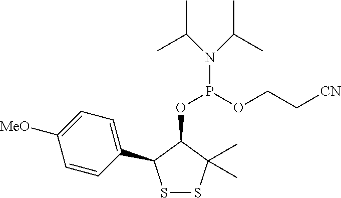 [SMILES: COc1ccc([C@@H]2SSC(C)(C)[C@@H]2OP(OCCC#N)N(C(C)C)C(C)C)cc1]

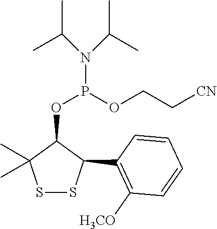 [SMILES: COc1ccccc1[C@H]1SSC(C)(C)[C@H]1OP(OCCC#N)N(C(C)C)C(C)C]

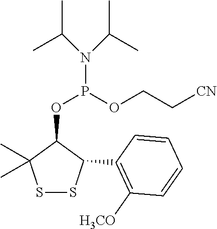 [SMILES: COc1ccccc1[C@@H]1SSC(C)(C)[C@H]1OP(OCCC#N)N(C(C)C)C(C)C]

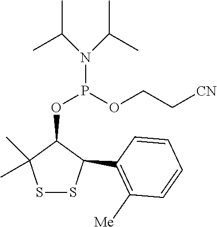 [SMILES: Cc1ccccc1[C@H]1SSC(C)(C)[C@H]1OP(OCCC#N)N(C(C)C)C(C)C]

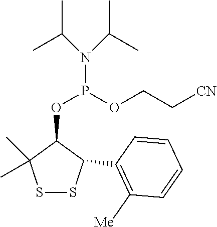 [SMILES: Cc1ccccc1[C@@H]1SSC(C)(C)[C@H]1OP(OCCC#N)N(C(C)C)C(C)C]

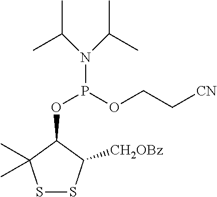

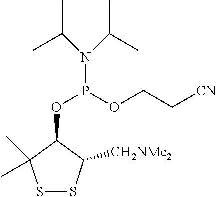

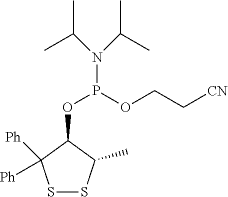 [SMILES: CC(C)N(C(C)C)P(OCCC#N)O[C@H]1[C@H](C)SSC1(c1ccccc1)c1ccccc1]

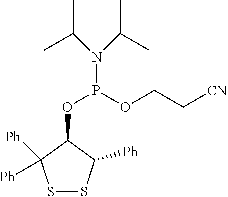 [SMILES: CC(C)N(C(C)C)P(OCCC#N)O[C@H]1[C@H](c2ccccc2)SSC1(c1ccccc1)c1ccccc1]

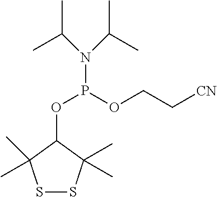 [SMILES: CC(C)N(C(C)C)P(OCCC#N)OC1C(C)(C)SSC1(C)C]

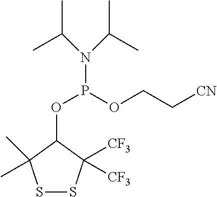 [SMILES: CC(C)N(C(C)C)P(OCCC#N)OC1C(C)(C)SSC1(C(F)(F)F)C(F)(F)F]

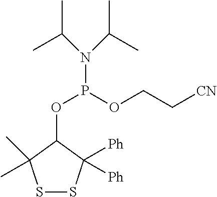 [SMILES: CC(C)N(C(C)C)P(OCCC#N)OC1C(C)(C)SSC1(c1ccccc1)c1ccccc1]

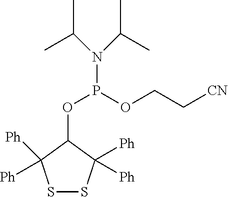 [SMILES: CC(C)N(C(C)C)P(OCCC#N)OC1C(c2ccccc2)(c2ccccc2)SSC1(c1ccccc1)c1ccccc1]

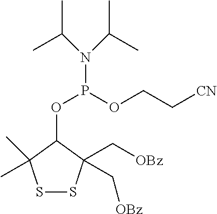

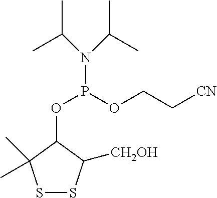 [SMILES: CC(C)N(C(C)C)P(OCCC#N)OC1C(CO)SSC1(C)C]

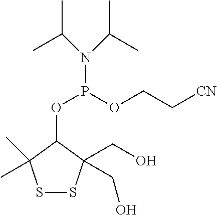 [SMILES: CC(C)N(C(C)C)P(OCCC#N)OC1C(C)(C)SSC1(CO)CO]

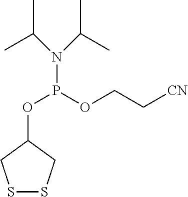 [SMILES: CC(C)N(C(C)C)P(OCCC#N)OC1CSSC1]

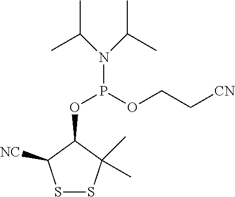 [SMILES: CC(C)N(C(C)C)P(OCCC#N)O[C@@H]1[C@H](C#N)SSC1(C)C]

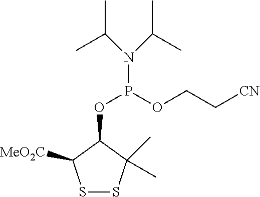 [SMILES: COC(=O)[C@@H]1SSC(C)(C)[C@@H]1OP(OCCC#N)N(C(C)C)C(C)C]

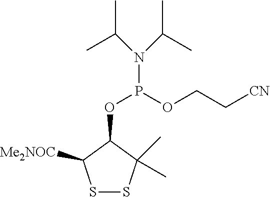

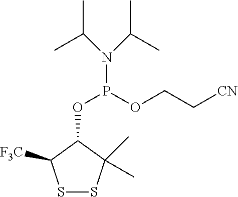 [SMILES: CC(C)N(C(C)C)P(OCCC#N)O[C@H]1[C@H](C(F)(F)F)SSC1(C)C]

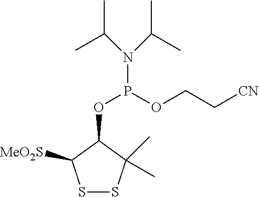 [SMILES: CC(C)N(C(C)C)P(OCCC#N)O[C@@H]1[C@H](S(C)(=O)=O)SSC1(C)C]

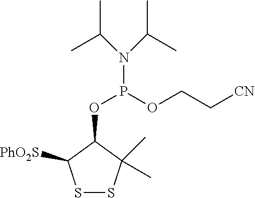

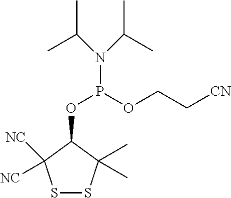 [SMILES: CC(C)N(C(C)C)P(OCCC#N)O[C@H]1C(C)(C)SSC1(C#N)C#N]

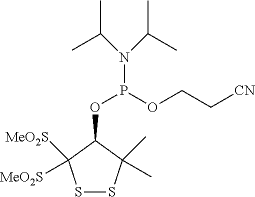 [SMILES: CC(C)N(C(C)C)P(OCCC#N)O[C@H]1C(C)(C)SSC1(S(C)(=O)=O)S(C)(=O)=O]

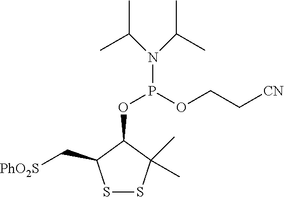

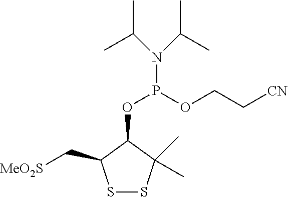 [SMILES: CC(C)N(C(C)C)P(OCCC#N)O[C@@H]1[C@H](CS(C)(=O)=O)SSC1(C)C]

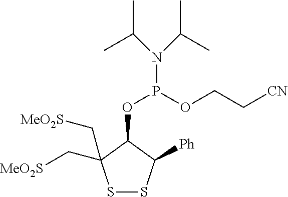 [SMILES: CC(C)N(C(C)C)P(OCCC#N)O[C@H]1[C@@H](c2ccccc2)SSC1(CS(C)(=O)=O)CS(C)(=O)=O]

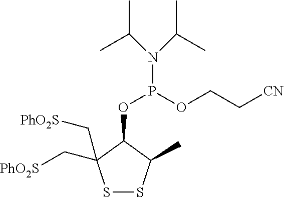

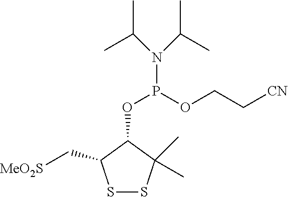 [SMILES: CC(C)N(C(C)C)P(OCCC#N)O[C@H]1[C@@H](CS(C)(=O)=O)SSC1(C)C]

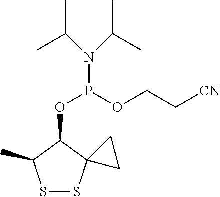 [SMILES: CC(C)N(C(C)C)P(OCCC#N)O[C@@H]1[C@H](C)SSC12CC2]

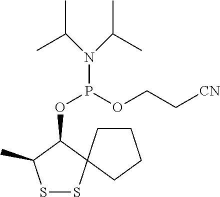 [SMILES: CC(C)N(C(C)C)P(OCCC#N)O[C@@H]1[C@H](C)SSC12CCCC2]

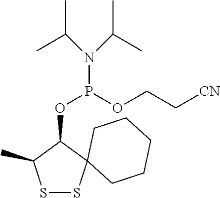 [SMILES: CC(C)N(C(C)C)P(OCCC#N)O[C@@H]1[C@H](C)SSC12CCCCC2]

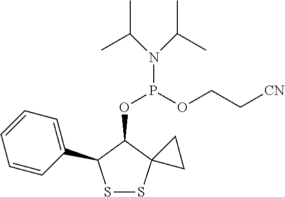 [SMILES: CC(C)N(C(C)C)P(OCCC#N)O[C@@H]1[C@H](c2ccccc2)SSC12CC2]

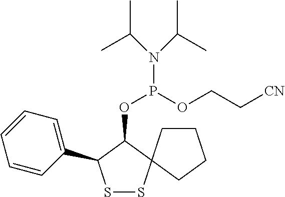 [SMILES: CC(C)N(C(C)C)P(OCCC#N)O[C@@H]1[C@H](c2ccccc2)SSC12CCCC2]

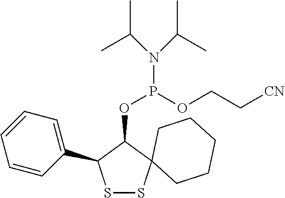 [SMILES: CC(C)N(C(C)C)P(OCCC#N)O[C@@H]1[C@H](c2ccccc2)SSC12CCCCC2]

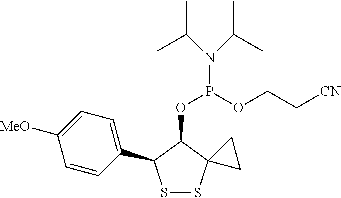 [SMILES: COc1ccc([C@@H]2SSC3(CC3)[C@@H]2OP(OCCC#N)N(C(C)C)C(C)C)cc1]

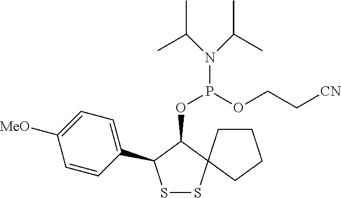 [SMILES: COc1ccc([C@@H]2SSC3(CCCC3)[C@@H]2OP(OCCC#N)N(C(C)C)C(C)C)cc1]

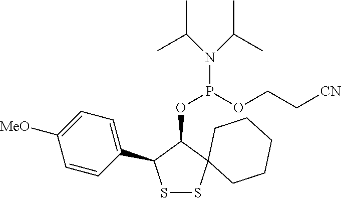 [SMILES: COc1ccc([C@@H]2SSC3(CCCCC3)[C@@H]2OP(OCCC#N)N(C(C)C)C(C)C)cc1]

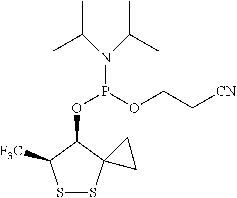 [SMILES: CC(C)N(C(C)C)P(OCCC#N)O[C@@H]1[C@H](C(F)(F)F)SSC12CC2]

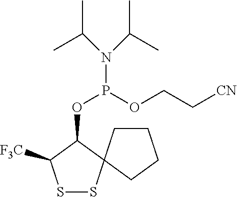 [SMILES: CC(C)N(C(C)C)P(OCCC#N)O[C@@H]1[C@H](C(F)(F)F)SSC12CCCC2]

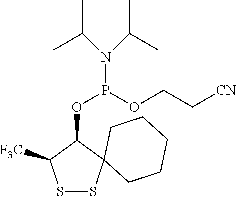 [SMILES: CC(C)N(C(C)C)P(OCCC#N)O[C@@H]1[C@H](C(F)(F)F)SSC12CCCCC2]

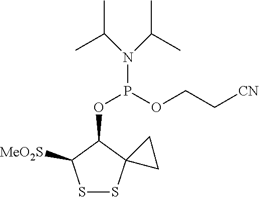 [SMILES: CC(C)N(C(C)C)P(OCCC#N)O[C@@H]1[C@H](S(C)(=O)=O)SSC12CC2]

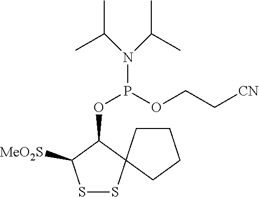 [SMILES: CC(C)N(C(C)C)P(OCCC#N)O[C@@H]1[C@H](S(C)(=O)=O)SSC12CCCC2]

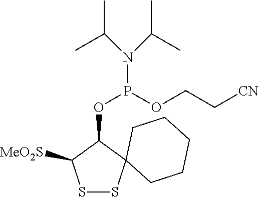 [SMILES: CC(C)N(C(C)C)P(OCCC#N)O[C@@H]1[C@H](S(C)(=O)=O)SSC12CCCCC2]

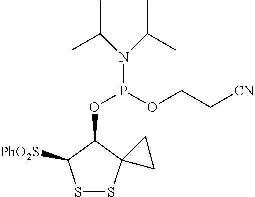

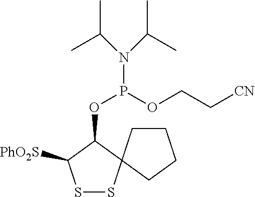

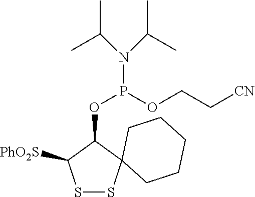

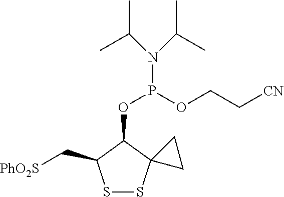

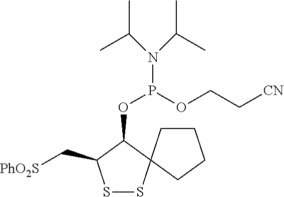

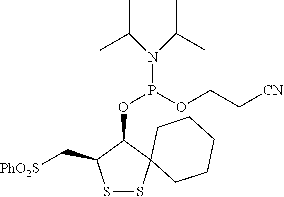

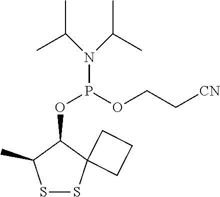 [SMILES: CC(C)N(C(C)C)P(OCCC#N)O[C@@H]1[C@H](C)SSC12CCC2]

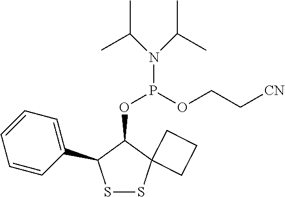 [SMILES: CC(C)N(C(C)C)P(OCCC#N)O[C@@H]1[C@H](c2ccccc2)SSC12CCC2]

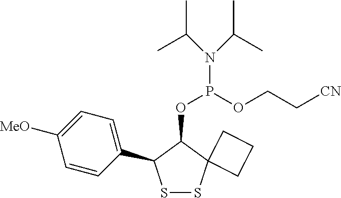 [SMILES: COc1ccc([C@@H]2SSC3(CCC3)[C@@H]2OP(OCCC#N)N(C(C)C)C(C)C)cc1]

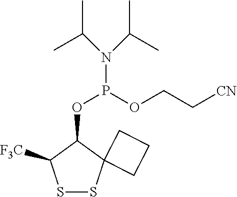 [SMILES: CC(C)N(C(C)C)P(OCCC#N)O[C@@H]1[C@H](C(F)(F)F)SSC12CCC2]

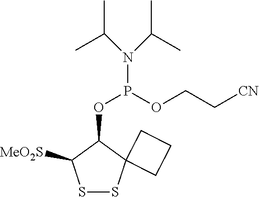 [SMILES: CC(C)N(C(C)C)P(OCCC#N)O[C@@H]1[C@H](S(C)(=O)=O)SSC12CCC2]

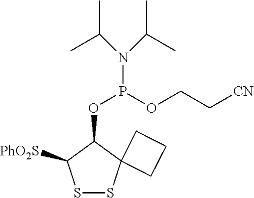

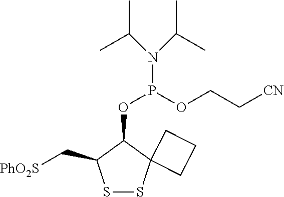

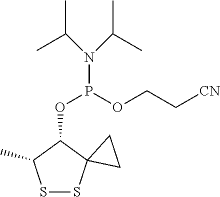 [SMILES: CC(C)N(C(C)C)P(OCCC#N)O[C@H]1[C@@H](C)SSC12CC2]

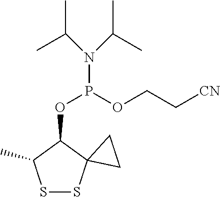 [SMILES: CC(C)N(C(C)C)P(OCCC#N)O[C@@H]1[C@@H](C)SSC12CC2]

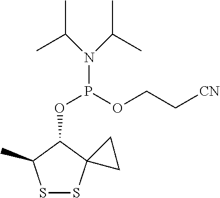 [SMILES: CC(C)N(C(C)C)P(OCCC#N)O[C@H]1[C@H](C)SSC12CC2]

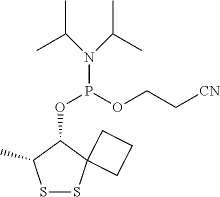 [SMILES: CC(C)N(C(C)C)P(OCCC#N)O[C@H]1[C@@H](C)SSC12CCC2]

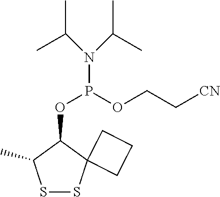 [SMILES: CC(C)N(C(C)C)P(OCCC#N)O[C@@H]1[C@@H](C)SSC12CCC2]

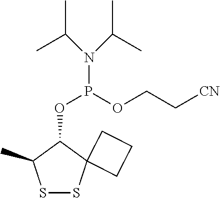 [SMILES: CC(C)N(C(C)C)P(OCCC#N)O[C@H]1[C@H](C)SSC12CCC2]

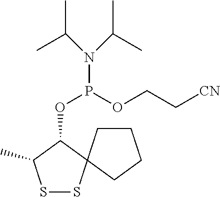 [SMILES: CC(C)N(C(C)C)P(OCCC#N)O[C@H]1[C@@H](C)SSC12CCCC2]

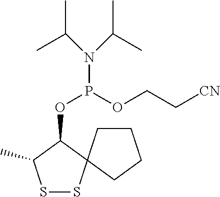 [SMILES: CC(C)N(C(C)C)P(OCCC#N)O[C@@H]1[C@@H](C)SSC12CCCC2]

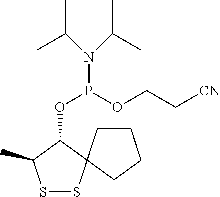 [SMILES: CC(C)N(C(C)C)P(OCCC#N)O[C@H]1[C@H](C)SSC12CCCC2]

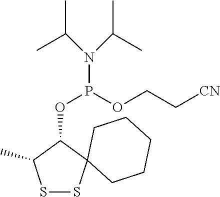 [SMILES: CC(C)N(C(C)C)P(OCCC#N)O[C@H]1[C@@H](C)SSC12CCCCC2]

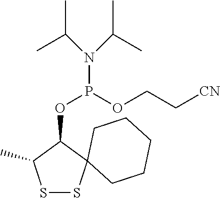 [SMILES: CC(C)N(C(C)C)P(OCCC#N)O[C@@H]1[C@@H](C)SSC12CCCCC2]

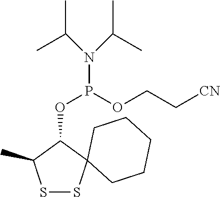 [SMILES: CC(C)N(C(C)C)P(OCCC#N)O[C@H]1[C@H](C)SSC12CCCCC2]

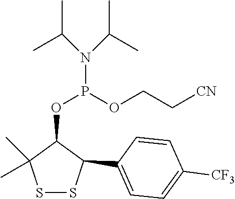 [SMILES: CC(C)N(C(C)C)P(OCCC#N)O[C@H]1[C@@H](c2ccc(C(F)(F)F)cc2)SSC1(C)C]

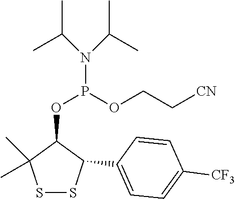 [SMILES: CC(C)N(C(C)C)P(OCCC#N)O[C@H]1[C@H](c2ccc(C(F)(F)F)cc2)SSC1(C)C]

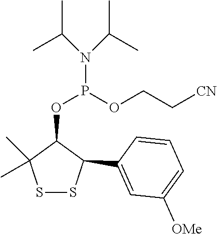 [SMILES: COc1cccc([C@H]2SSC(C)(C)[C@H]2OP(OCCC#N)N(C(C)C)C(C)C)c1]

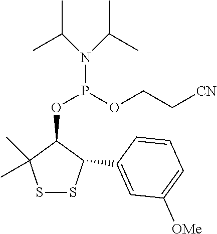 [SMILES: COc1cccc([C@@H]2SSC(C)(C)[C@H]2OP(OCCC#N)N(C(C)C)C(C)C)c1]

In some embodiments, the

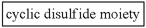

has the structure

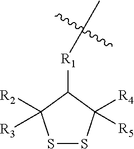

(C-I), wherein R₃ and R₅, together with the adjacent carbon atoms and the two sulfur atoms, form a second ring. In one embodiment, the second ring has 6-8 atoms.

In some embodiments, the compound has the formula of:

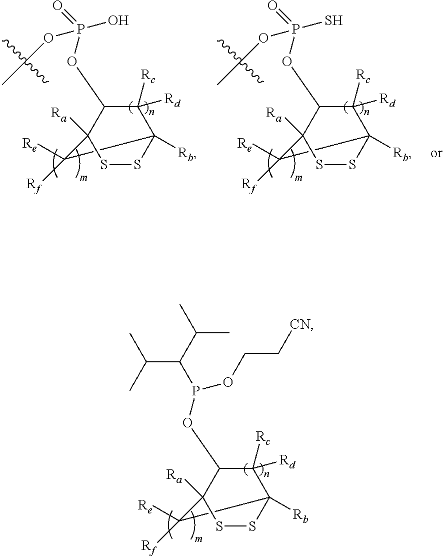

wherein n is 0, 1, or 2; m is 1, 2, or 3; Rₐ, R_{b}, R_{c}, R_{d}, Rₑ, and R_{f} are each independently H, alkyl (e.g., CH₃), heterocyclic, CF₃, CH₂R¹⁵, aryl (e.g., substituted or unsubstituted phenyl), heteroaryl, CHFR¹⁵, CF₂R¹⁵, C(O)NHR¹⁵, C(O)N(R¹⁵)₂, C(O)OR¹⁵, S(O)OR¹⁵, CH₂C(O)OR¹⁵, CH₂S(O)OR¹⁵; and can be in any stereoisomeric configurations; and R¹⁵ is H, alkyl, heterocyclic, aryl (e.g., substituted or unsubstituted phenyl), heteroaryl, hydroxyl, alkoxy, NH₂, NHalkyl, NH(alkyl)₂, or CF₃; and can be in any stereoisomeric configurations.

Exemplary

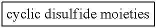

for bicyclic compounds of formula (C-I) include:

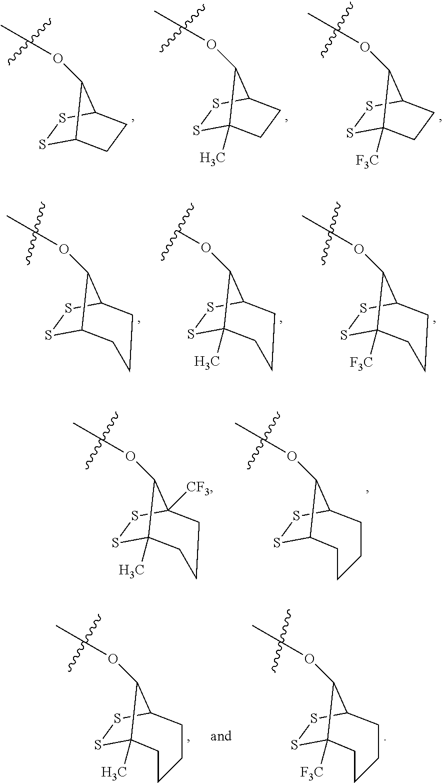

In some embodiments, the compound has the formula of

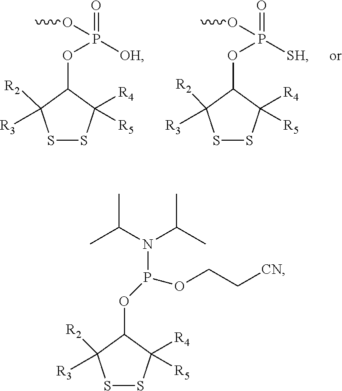

wherein R₃ and R₅, together with the adjacent carbon atoms and the two sulfur atoms, form a second ring (e.g., having 6-8 atoms).

Exemplary compounds of formula (I) with a bicyclic disulfide moiety are shown in Table 2.

TABLE 2
Compounds with bicyclic disulfide moieties

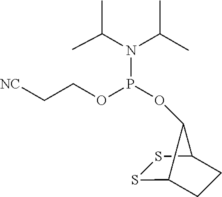 [SMILES: CC(C)N(C(C)C)P(OCCC#N)OC1C2CCC1SS2]

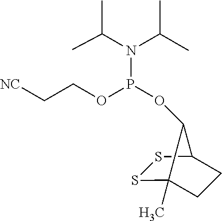 [SMILES: CC(C)N(C(C)C)P(OCCC#N)OC1C2CCC1(C)SS2]

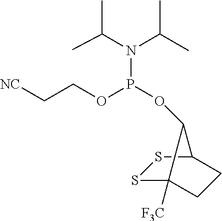 [SMILES: CC(C)N(C(C)C)P(OCCC#N)OC1C2CCC1(C(F)(F)F)SS2]

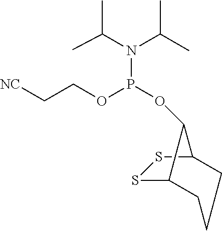 [SMILES: CC(C)N(C(C)C)P(OCCC#N)OC1C2CCCC1SS2]

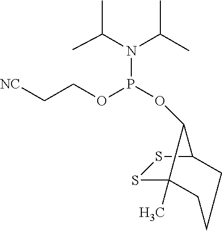 [SMILES: CC(C)N(C(C)C)P(OCCC#N)OC1C2CCCC1(C)SS2]

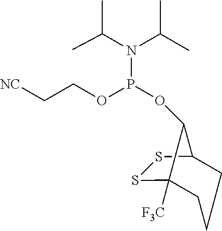 [SMILES: CC(C)N(C(C)C)P(OCCC#N)OC1C2CCCC1(C(F)(F)F)SS2]

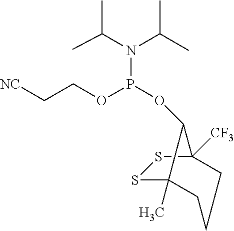 [SMILES: CC(C)N(C(C)C)P(OCCC#N)OC1C2(C)CCCC1(C(F)(F)F)SS2]

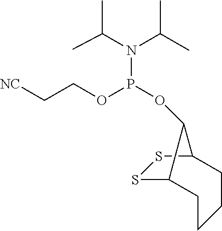 [SMILES: CC(C)N(C(C)C)P(OCCC#N)OC1C2CCCCC1SS2]

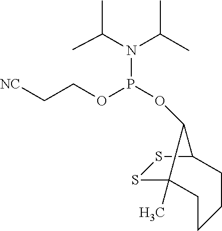 [SMILES: CC(C)N(C(C)C)P(OCCC#N)OC1C2CCCCC1(C)SS2]

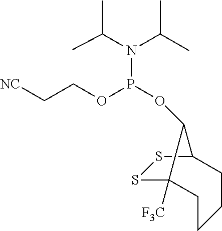 [SMILES: CC(C)N(C(C)C)P(OCCC#N)OC1C2CCCCC1(C(F)(F)F)SS2]

The

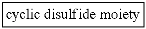

can also have the structure

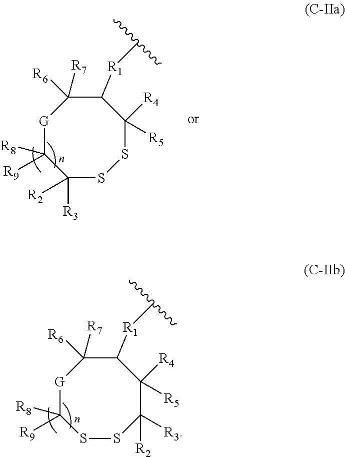

### Exemplary

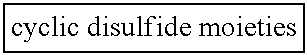

for a cyclic compounds of formula (C-IIa) or (C-IIb) include:

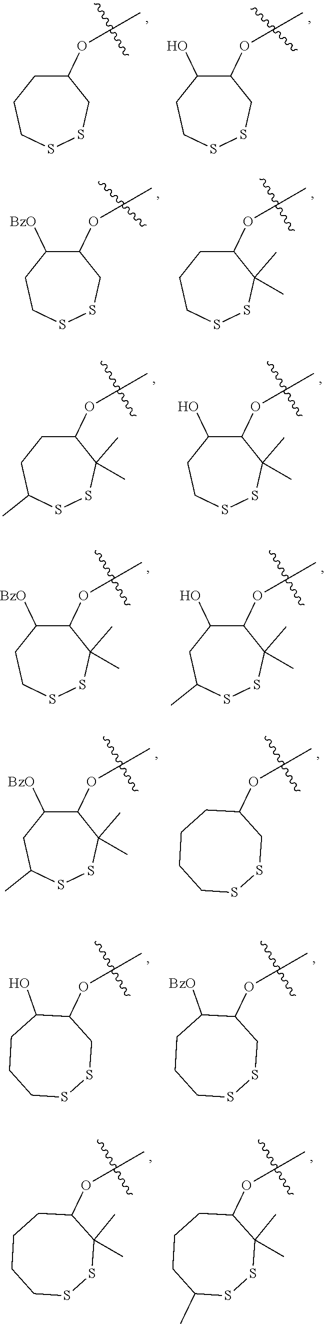

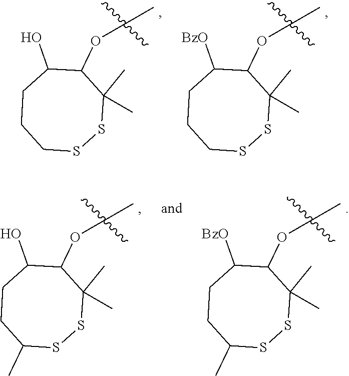

Additional exemplary

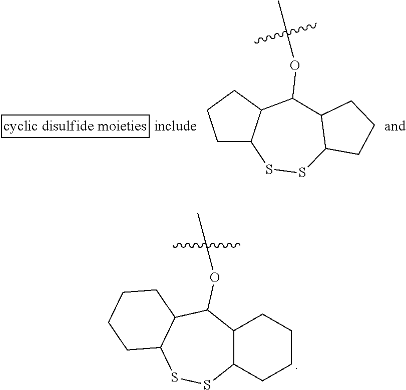 [SMILES: *OC1C2CCCC2SSC2CCCC21 ; *OC1C2CCCCC2SSC2CCCCC21]

In some embodiments, the compound has the formula of:

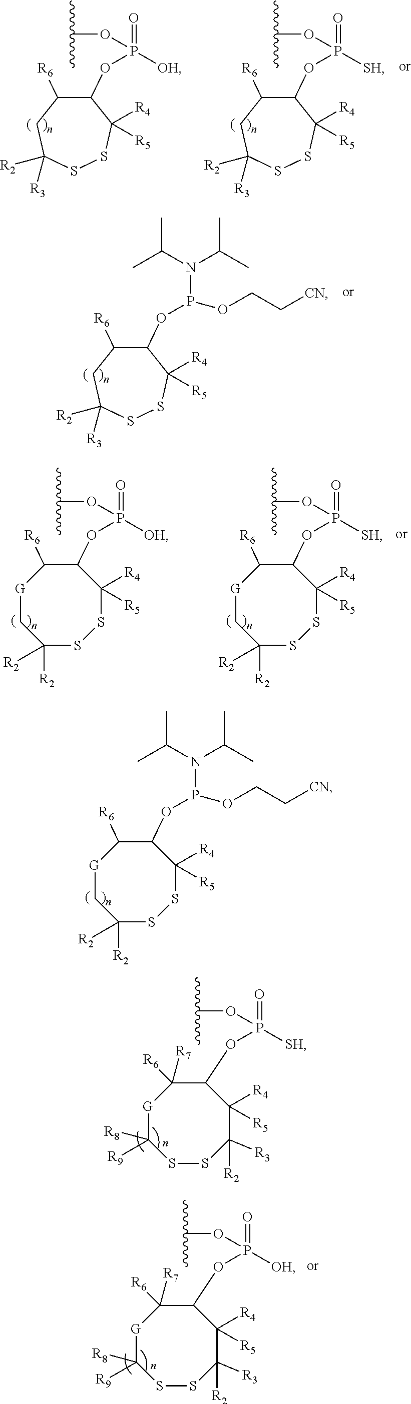

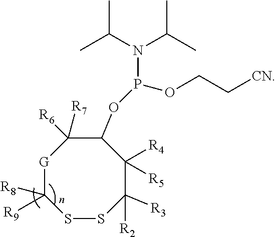

In these formulas, n is 1, 2, 3, 4, 5, or 6; G is O, NR¹⁵, S, or any other heteroatom; R₂, R₃, R₄, R₅, and R₆ are each independently H, alkyl (e.g., CH₃), heterocyclic, CH₂R¹⁵, aryl (e.g., phenyl), heteroaryl, CHFR¹⁵, CF₂R¹⁵, CF₃; and can be in any stereoisomeric configurations; and R¹⁵ is alkyl, heterocyclic, aryl, OH, O-alkyl, NH₂, NH(alkyl), N(alkyl)₂, CF₂ R¹⁵, or CF₃; and can be in any stereoisomeric configurations.

Exemplary compounds of formula (I) with a larger (7-member or larger) cyclic disulfide moiety are shown in Table 3.

TABLE 3
Compounds with 7- or 8- member cyclic disulfide moieties

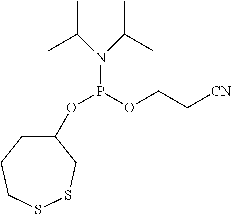 [SMILES: CC(C)N(C(C)C)P(OCCC#N)OC1CCCSSC1]

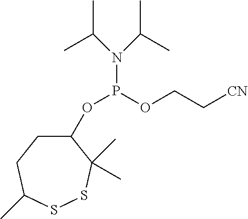 [SMILES: CC1CCC(OP(OCCC#N)N(C(C)C)C(C)C)C(C)(C)SS1]

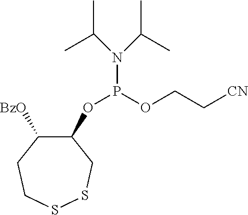

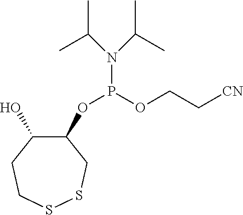 [SMILES: CC(C)N(C(C)C)P(OCCC#N)O[C@H]1CSSCC[C@@H]1O]

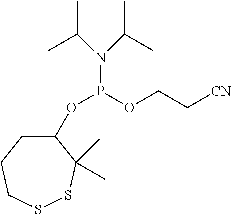 [SMILES: CC(C)N(C(C)C)P(OCCC#N)OC1CCCSSC1(C)C]

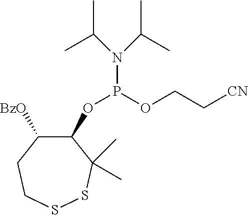

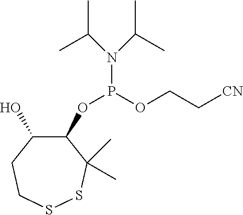 [SMILES: CC(C)N(C(C)C)P(OCCC#N)O[C@@H]1[C@@H](O)CCSSC1(C)C]

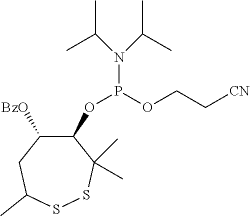

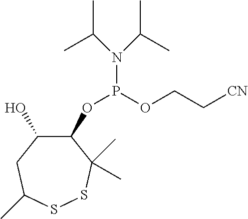 [SMILES: CC1C[C@H](O)[C@@H](OP(OCCC#N)N(C(C)C)C(C)C)C(C)(C)SS1]

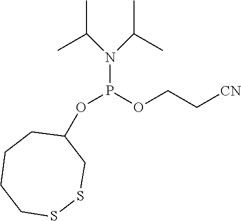 [SMILES: CC(C)N(C(C)C)P(OCCC#N)OC1CCCCSSC1]

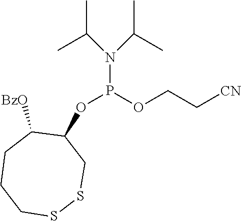

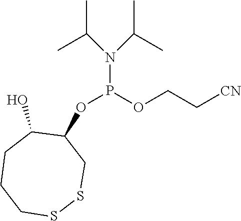 [SMILES: CC(C)N(C(C)C)P(OCCC#N)O[C@H]1CSSCCC[C@@H]1O]

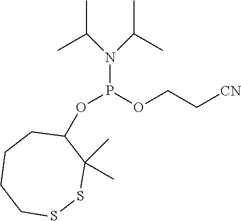 [SMILES: CC(C)N(C(C)C)P(OCCC#N)OC1CCCCSSC1(C)C]

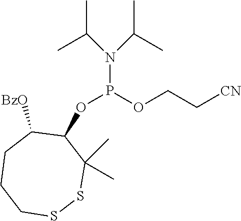

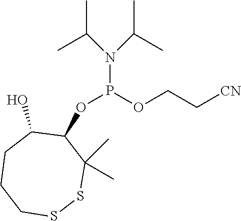 [SMILES: CC(C)N(C(C)C)P(OCCC#N)O[C@@H]1[C@@H](O)CCCSSC1(C)C]

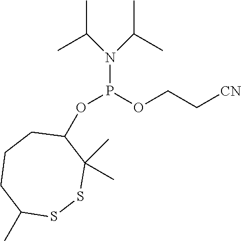 [SMILES: CC1CCCC(OP(OCCC#N)N(C(C)C)C(C)C)C(C)(C)SS1]

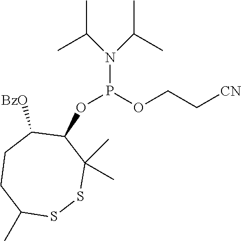

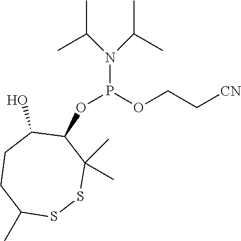 [SMILES: CC1CC[C@H](O)[C@@H](OP(OCCC#N)N(C(C)C)C(C)C)C(C)(C)SS1]

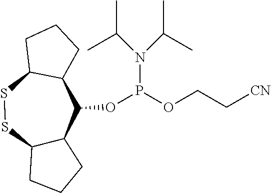 [SMILES: CC(C)N(C(C)C)P(OCCC#N)O[C@@H]1[C@H]2CCC[C@H]2SS[C@H]2CCC[C@@H]12]

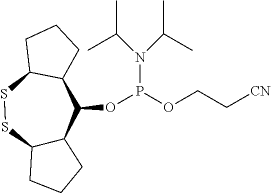 [SMILES: CC(C)N(C(C)C)P(OCCC#N)O[C@H]1[C@H]2CCC[C@H]2SS[C@H]2CCC[C@@H]12]

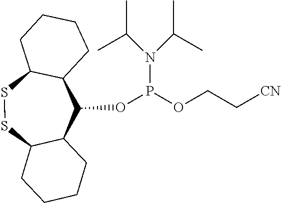 [SMILES: CC(C)N(C(C)C)P(OCCC#N)O[C@@H]1[C@H]2CCCC[C@H]2SS[C@H]2CCCC[C@@H]12]

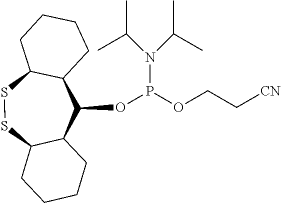 [SMILES: CC(C)N(C(C)C)P(OCCC#N)O[C@H]1[C@H]2CCCC[C@H]2SS[C@H]2CCCC[C@@H]12]

The

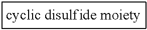

can also have the structure

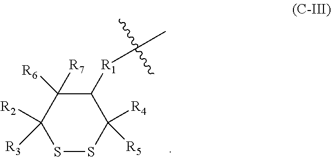

### Exemplary

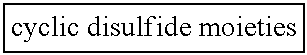

for a 6-member cyclic compounds of formula (C-III) include:

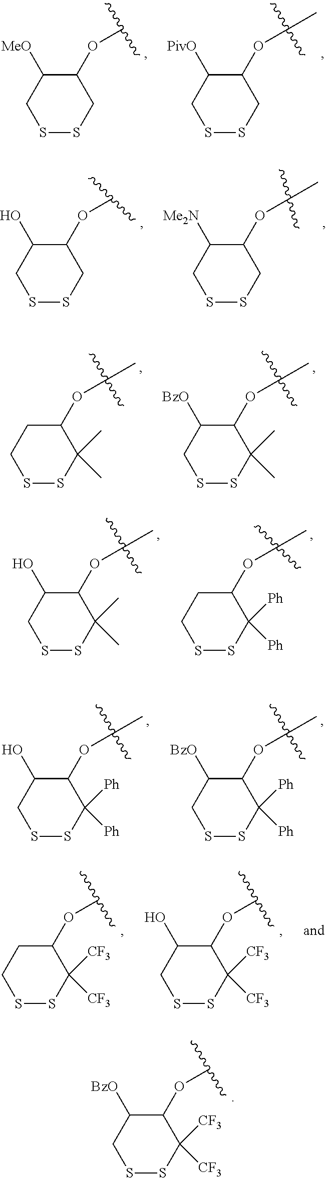

In some embodiments, the compound has the formula of

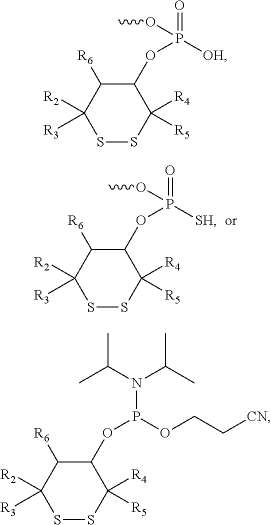

In this formula, R₂, R₃, R₄, R₅, and R₆ are each independently H, alkyl (e.g., CH₃), heterocyclic, CH₂R¹⁵, aryl (e.g., phenyl), heteroaryl, CHFR¹⁵, CF₂R¹⁵, CF₃; and can be in any stereoisomeric configurations; and R¹⁵ is alkyl, heterocyclic, aryl, OH, O-alkyl, NH₂, NH(alkyl), N(alkyl)₂, CF₂ R¹⁵, or CF₃; and can be in any stereoisomeric configurations.

Exemplary compounds of formula (I) with a 6-member cyclic disulfide moiety are shown in Table 4.

TABLE 4
Compounds with 6- member cyclic disulfide moieties

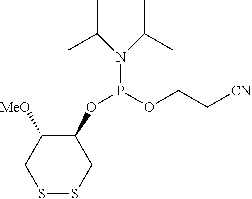 [SMILES: CO[C@H]1CSSC[C@@H]1OP(OCCC#N)N(C(C)C)C(C)C]

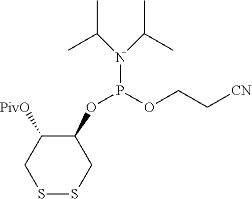

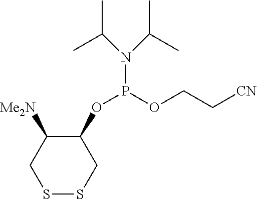 [SMILES: CC(C)N(C(C)C)P(OCCC#N)O[C@H]1CSSC[C@H]1N(C)C]

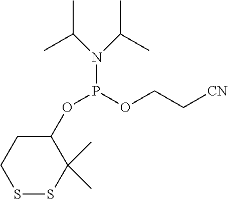 [SMILES: CC(C)N(C(C)C)P(OCCC#N)OC1CCSSC1(C)C]

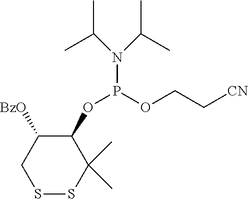

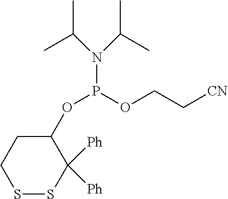 [SMILES: CC(C)N(C(C)C)P(OCCC#N)OC1CCSSC1(c1ccccc1)c1ccccc1]

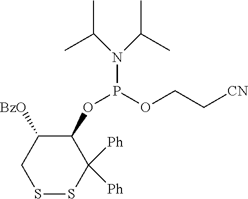

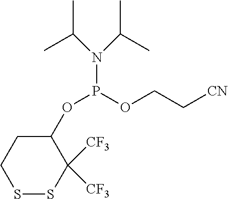 [SMILES: CC(C)N(C(C)C)P(OCCC#N)OC1CCSSC1(C(F)(F)F)C(F)(F)F]

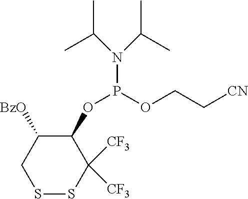

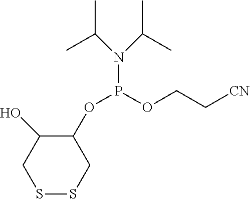 [SMILES: CC(C)N(C(C)C)P(OCCC#N)OC1CSSCC1O]

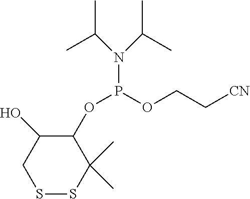 [SMILES: CC(C)N(C(C)C)P(OCCC#N)OC1C(O)CSSC1(C)C]

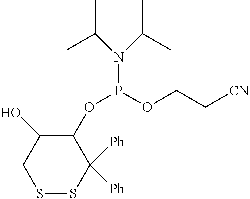 [SMILES: CC(C)N(C(C)C)P(OCCC#N)OC1C(O)CSSC1(c1ccccc1)c1ccccc1]

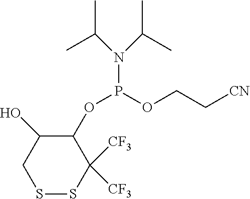 [SMILES: CC(C)N(C(C)C)P(OCCC#N)OC1C(O)CSSC1(C(F)(F)F)C(F)(F)F]

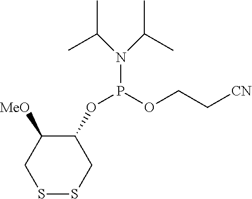 [SMILES: CO[C@@H]1CSSC[C@H]1OP(OCCC#N)N(C(C)C)C(C)C]

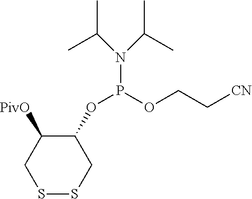

Certain terms are abbreviated within chemical structures throughout the application as would be familiar to those skilled in the art, including, e.g., methyl (Me), benzoyl (Bz), phenyl (Ph), and pivaloyl (Piv).

The term “halo” or“halogen” refers to any radical of fluorine, chlorine, bromine or iodine.

The term “aliphatic” or “aliphatic group,” as used herein, means a straight-chain or branched, substituted or unsubstituted hydrocarbon chain that is saturated or contains one or more units of unsaturation, or a monocyclic hydrocarbon or bicyclic or polycyclic hydrocarbon that is saturated or contains one or more units of unsaturation, but is not aromatic, that has a single point of attachment to the rest of the molecule. In some embodiments, aliphatic groups contain 1-50 aliphatic carbon atoms, for instance, 1-10 aliphatic carbon atoms, 1-6 aliphatic carbon atoms, 1-5 aliphatic carbon atoms, 1-4 aliphatic carbon atoms, 1-3 aliphatic carbon atoms, or 1-2 aliphatic carbon atoms. In some embodiments, “cycloaliphatic” refers to a monocyclic or bicyclic C₃-C₁₀ hydrocarbon (e.g., a monocyclic C₃-C₆ hydrocarbon) that is saturated or contains one or more units of unsaturation, but is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl, or (cycloalkyl)alkenyl.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. Unless otherwise indicated, “alkyl” generally refers to C₁-C₂₄ alkyl (e.g., C₁-C₁₂ alkyl, C₁-C₈ alkyl, or C₁-C₄ alkyl). The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Unless otherwise indicated, “alkenyl” generally refers to C₂-C₅ alkenyl (e.g., C₂-C₆ alkenyl, C₂-C₄ alkenyl, or C₂-C₃ alkenyl). Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Unless otherwise indicated, “alkynyl” generally refers to C₂-C₅ alkynyl (e.g., C₂-C₆ alkynyl, C₂-C₄ alkynyl, or C₂-C₃ alkynyl). Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The term “alkoxy” refers to an —O-alkyl radical. The term “alkylene” refers to a divalent alkyl (i.e., —R—). The term “aminoalkyl” refers to an alkyl substituted with an amino. The term “mercapto” refers to an —SH radical. The term “thioalkoxy” refers to an —S-alkyl radical.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below.

The term “alkenylene” refers to a bivalent alkenyl group. A substituted alkenylene chain is a polymethylene group containing at least one double bond in which one or more hydrogen atoms are replaced with a substituent. Suitable substituents include those described below.

The term “aryl” refers to a 6-carbon monocyclic or 10-carbon bicyclic aromatic ring system wherein 0, 1, 2, 3, or 4 atoms of each ring may be substituted by a substituent. The term “aryl” may be used interchangeably with the term “aryl ring.” Examples of aryl groups include phenyl, biphenyl, naphthyl, anthracyl, and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylalkyl” or the term “aralkyl” refers to alkyl substituted with an aryl. The term “arylalkoxy” refers to an alkoxy substituted with aryl.

The term “cycloalkyl” or “cyclyl” as employed herein includes saturated and partially unsaturated, but not aromatic, cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” or “heteroar-” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. The term also include groups in which a heteroaromatic ring is fused to one or more aryl, cycloalkyl, or heterocyclyl rings, where the radical or point of attachment is on the heteroaromatic ring. Examples of heteroaryl groups include pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl,” “heterocycle,” “heterocyclic radical,” or “heterocyclic ring” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl). Examples of heterocyclyl groups include trizolyl, tetrazolyl, piperazinyl, pyrrolidinyl, dioxanyl, dioxolanyl, diazepinyl, oxazepinyl, thiazepinyl, morpholinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, quinuclidinyl, and the like. The term “heterocyclylalkyl” refers to an alkyl group substituted by a heterocyclyl, wherein the alkyl and heterocyclyl portions independently are optionally substituted.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent can be further substituted.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

### Stereoisomer and Chirally Pure of Enriched Compounds

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the chirally pure/enriched desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the chirally pure/enriched enantiomers.

Certain embodiments of the present invention also include oligonucleotides that are substantially chirally pure or chirally enriched with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

The chiral purity with respect to the chiral linkage phosphorus atom for each terminal, chirally-modified internucleotide linkage is at least 50%, for instance, at least 60%, at least 70%, at least 80%, at least 85%, at least 90%, at least 91%, at least 92%, at least 93%, at least 94%, at least 95%, at least 96%, at least 97%, at least 98%, at least 99%, at least 99.5%, or virtually 100%.

A chirally pure (or substantially chirally pure) diastereoisomeric form of the compound or oligonucleotide may refer to a particular diastereoisomeric form of the compound or oligonucleotide having a chiral purity of at least 60%, at least 70%, at least 80%, at least 85%, at least 90%, at least 91%, at least 92%, at least 93%, at least 94%, at least 95%, at least 96%, at least 97%, at least 98%, at least 99%, at least 99.5%, or virtually 100%.

Accordingly, embodiments of the invention provide for a structure of the formula (I) or (II), formula (C-I), (C-IIa), or (C-IIb), formula (P-I) or (P-II), present in a chirally pure or enriched diastereoisomeric form, having a chiral purity of at least at least 60%, at least 70%, at least 80%, at least 85%, at least 90%, at least 91%, at least 92%, at least 93%, at least 94%, at least 95%, at least 96%, at least 97%, at least 98%, at least 99%, at least 99.5%, or virtually 100%.

Exemplary chirally pure/enriched compounds are:

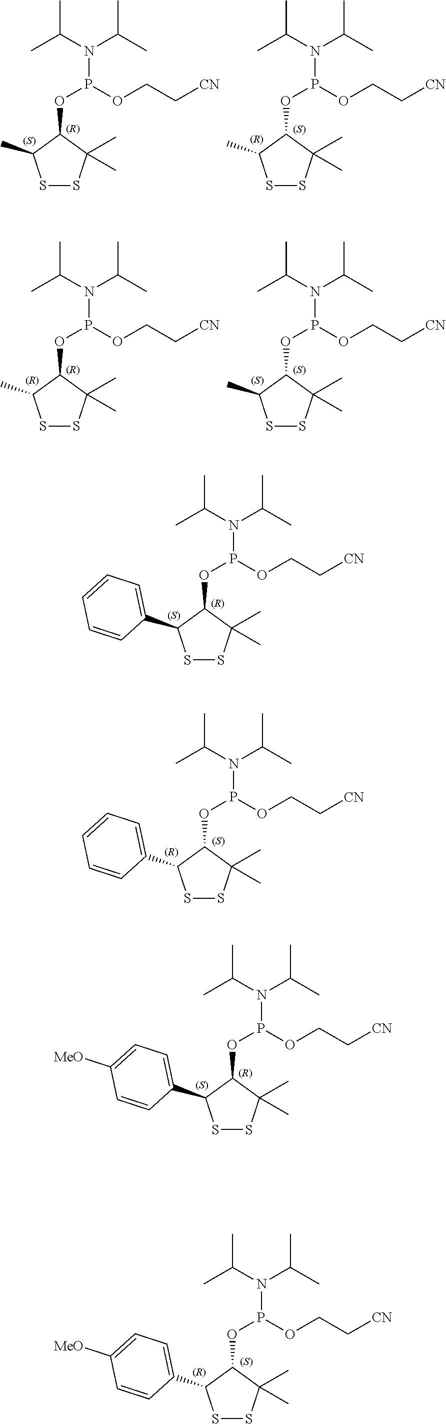

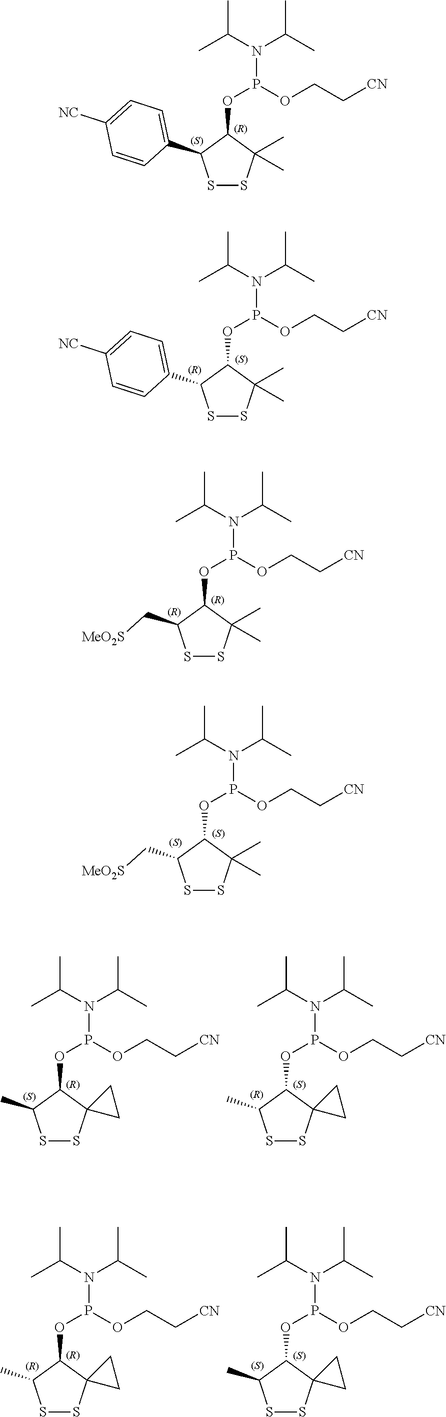

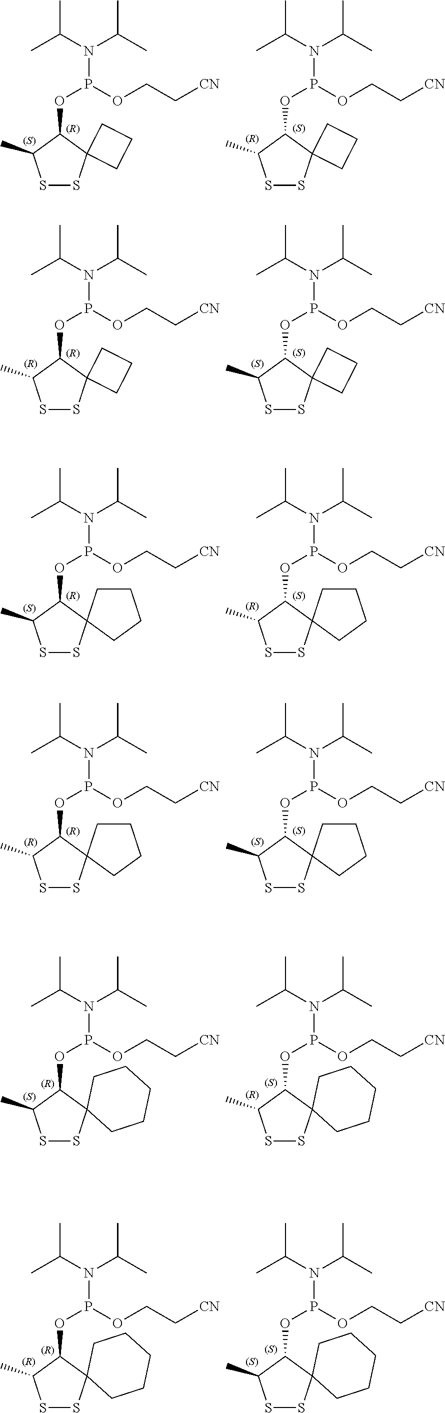

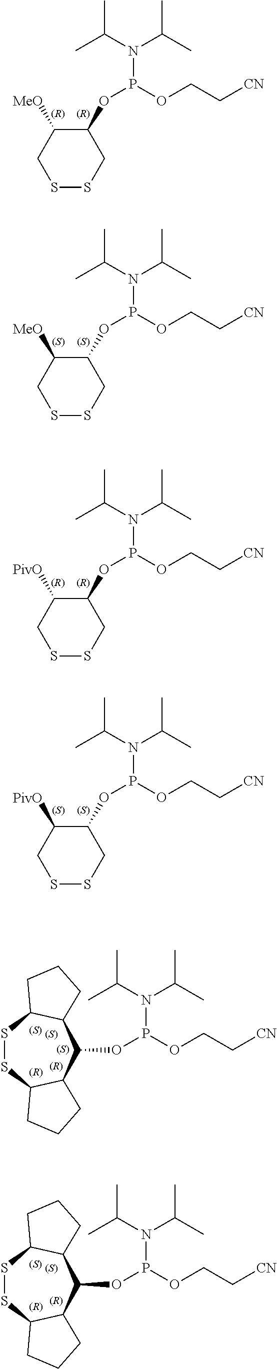

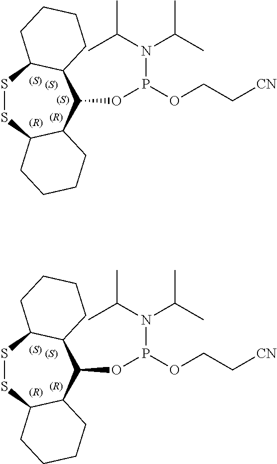

### The Oligonucleotide Prodrug

Another aspect of the invention relates to an oligonucleotide (e.g., a single-stranded iRNA agent or a double-stranded iRNA agent) comprising one or more compounds that comprise the structure of formula (I):

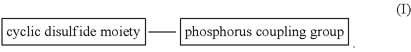

In formula (I), at least one

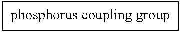

contains a nucleoside or oligonucleotide.

All the above embodiments relating to all the formulas of the

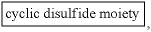

all the formulas of the

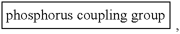

all the variables defined in these formulas, and all the subgenus and species structures relating to the compound, the

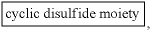

and the

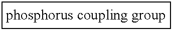

in the first aspect of the invention relating to the compound (or modified phosphate prodrug compound) are suitable in this aspect of the invention relating to the oligonucleotide.

In some embodiments, the oligonucleotide contains at least one

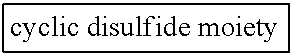

at the 5′-end of the oligonucleotide.

In some embodiments, the oligonucleotide contains at least one

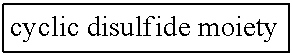

at the 3′-end of the oligonucleotide.

In some embodiments, the oligonucleotide contains at least one

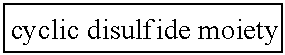

at an internal position of the oligonucleotide.

In some embodiments, when the

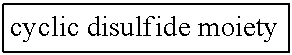

has the structure of formula (C-III), at least one

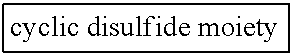

is connected at the 5′ end of the nucleoside or oligonucleotide.

Additional structures for the modified phosphate prodrug compound include those disclosed in WO 2014/088920, published on Jun. 12, 2014, the content of which is incorporated herein by reference in its entirety. In particular, these modified phosphate prodrug compounds are incorporated into the oligonucleotide at the 5′ end.

In some embodiments, the oligonucleotide is a single-stranded oligonucleotide, such as a single-stranded iRNA agent (e.g., single-stranded siRNA).

In some embodiments, the oligonucleotide is a double-stranded oligonucleotide, such as a double-stranded iRNA agent (e.g., double-stranded siRNA), comprising a sense strand and an antisense strand.

In one embodiment, the sense strand contains at least one

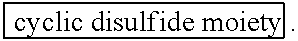

In one embodiment, the antisense strand contains at least one

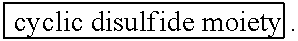

In one embodiment both the sense strand and the antisense strand each contain at least one

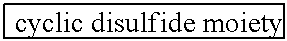

Introduction of the

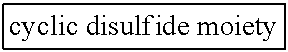

to the phosphate group as a temporary protecting group, on either the sense or antisense strand or both the sense and antisense strands, are illustrated in Schemes 10-15 in Example 9 below.

### Oligonucleotide Definitions and Designs

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18^{th} edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the overall double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA, e.g., RNAi agent. The % and/or fold difference can be calculated relative to the control or the non-control, for example,

$$\% difference = \frac{[ expression with siRNA - expression without siRNA ]}{expression without siRNA}$$

$$or$$

$$\% difference = \frac{[ expression with siRNA - expression without siRNA ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The double-stranded iRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the double-stranded iRNA comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The phrase “antisense strand” as used herein, refers to an oligonucleotide strand that is substantially or 100% complementary to a target sequence of interest. The phrase “antisense strand” includes the antisense region of both oligonucleotide strands that are formed from two separate strands, as well as unimolecular oligonucleotide strands that are capable of forming hairpin or dumbbell type structures. The terms “antisense strand” and “guide strand” are used interchangeably herein.

The phrase “sense strand” refers to an oligonucleotide strand that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, I. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand is equal to or at least 14, 15, 16, 17, 18, 19, 20, 21, 22, 23, 23, 24, 25, 26, 27, 28, 29, or 30 nucleotides in length.

In some embodiments, the sense strand is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands are each 15 to 30 nucleotides in length. In one embodiment, the sense and antisense strands are each 19 to 25 nucleotides in length. In one embodiment, the sense and antisense strands are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the oligonucleotide comprises a single-stranded overhang on at least one of the termini. In one embodiment, the single-stranded overhang is 1, 2, or 3 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the double-stranded iRNA has a ZXY structure, such as is described in PCT Publication No. 2004080406, which is hereby incorporated by reference in its entirety.

In certain embodiment, the two strands of double-stranded oligonucleotide can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligonucleotide will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligonucleotide can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligonucleotide s that can induce RNA interference are also referred to as “shRNA” herein.

In certain embodiments, two oligonucleotide strands specifically hybridize when there is a sufficient degree of complementarity to avoid non-specific binding of the antisense strand to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense strand will hybridize to its target sequence, but to a minimal number of other sequences. Stringent conditions are sequence-dependent and will be different in different circumstances, and “stringent conditions” under which antisense strand hybridize to a target sequence are determined by the nature and composition of the antisense strand and the assays in which they are being investigated.

It is understood in the art that incorporation of nucleotide affinity modifications may allow for a greater number of mismatches compared to an unmodified oligonucleotide. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

Additional dsRNA Design

In one embodiment, the oligonucleotide is an iRNA agent, and the iRNA agent is a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the oligonucleotide is an iRNA agent, and the iRNA agent is a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the oligonucleotide is an iRNA agent, and the iRNA agent is a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the oligonucleotide is an iRNA agent, and the iRNA agent comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the iRNA is blunt, while the other end is comprises a 2 nt overhang. Preferably, the 2 nt overhang is at the 3′-end of the antisense. Optionally, the iRNA agent further comprises a ligand (e.g., GalNAc₃).

In one embodiment, the oligonucleotide is an iRNA agent, and the iRNA agent comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the oligonucleotide is an iRNA agent, and the iRNA agent comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal. Optionally, the iRNA agent further comprises a ligand (e.g., GalNAc₃).

In one embodiment, the sense strand contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For an iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1′ nucleotide from the 5′-end of the antisense strand, or, the count starting from the 1′ paired nucleotide within the duplex region from the 5′-end of the antisense strand. The cleavage site in the antisense strand may also change according to the length of the duplex region of the iRNA from the 5′-end.

In some embodiments, the oligonucleotide is an iRNA agent, and the iRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the oligonucleotide is an iRNA agent, and the iRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the oligonucleotide is an iRNA agent, and the iRNA agent comprises a sense strand and antisense strand each having 14 to 30 nucleotides, wherein the sense strand contains at least one motif of three 2′-F modifications on three consecutive nucleotides at positions 9, 10, 11 from the 5′end, and wherein the antisense strand contains at least one motif of three 2′-O-methyl modifications on three consecutive nucleotides at positions 11, 12, 13 from the 5′end.

In one embodiment, the oligonucleotide is an iRNA agent, and the iRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the oligonucleotide is an iRNA agent, and the iRNA agent comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In one embodiment, 100%, 95%, 90%, 85%, 80%, 75%, 70%, 65%, 60%, 55%, 50%, 45%, 40%, 35% or 30% of the dsRNA agent is modified. For example, when 50% of the dsRNA agent is modified, 50% of all nucleotides present in the dsRNA agent contain a modification as described herein.

In some embodiments, the oligonucleotide contains one or more 2′-0 modifications selected from the group consisting of 2′-deoxy, 2′-O-methoxyalkyl, 2′-O-methyl, 2′-O-allyl, 2′-C-allyl, 2′-fluoro, 2′-O-N-methylacetamido (2′-O-NMA), 2′-O-dimethylaminoethoxyethyl (2′-O-DMAEOE), 2′-O-aminopropyl (2′-O-AP), and 2′-ara-F.

In one embodiment, each of the sense and antisense strands is independently modified with non-natural nucleotides such as acyclic nucleotides, LNA, HNA, CeNA, 2′-methoxyethyl, 2′-O-methyl, 2′-O-allyl, 2′-C-allyl, 2′-deoxy, 2′-fluoro, 2′-O-N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In some embodiments, the oligonucleotide contains one, two, three, four, five, six, seven, eight, nine, ten, eleven or twelve 2′-F modification(s). In one example, oligonucleotide contains nine or ten 2′-F modifications.

In one embodiment, the oligonucleotide does not contain any 2′-F modification.

The iRNA agent may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the oligonucleotide is an iRNA agent, and the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In one embodiment, the sense strand and/or antisense strand comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent is 100% complementary to a target RNA to hybridize thereto and inhibits its expression through RNA interference. In another embodiment, the antisense strand of the dsRNA agent is at least 95%, at least 90%, at least 85%, at least 80%, at least 75%, at least 70%, at least 65%, at least 60%, at least 55%, or at least 50% complementary to a target RNA.

In one aspect, the invention relates to an oligonucleotide (such as a dsRNA agent) as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand).

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

In one embodiment, the oligonucleotide is a dsRNA agent, and the dsRNA agent comprises:
(a) a sense strand having:
   (i) a length of 18-23 nucleotides;
   (ii) three consecutive 2′-F modifications at positions 7-15; and
(b) an antisense strand having:
   (i) a length of 18-23 nucleotides;
   (ii) at least 2′-F modifications anywhere on the strand; and
   (iii) at least two phosphorothioate internucleotide linkages at the first five nucleotides (counting from the 5′ end);
wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the oligonucleotide is a dsRNA agent, and the dsRNA agent comprises:
(a) a sense strand having:
   (i) a length of 18-23 nucleotides;
   (ii) less than four 2′-F modifications;
(b) an antisense strand having:
   (i) a length of 18-23 nucleotides;
   (ii) at less than twelve 2′-F modification; and
   (iii) at least two phosphorothioate internucleotide linkages at the first five nucleotides (counting from the 5′ end);
wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the oligonucleotide is a dsRNA agent, and the dsRNA agent comprises:
(a) a sense strand having:
   (i) a length of 19-35 nucleotides;
   (ii) less than four 2′-F modifications;
(b) an antisense strand having:
   (i) a length of 19-35 nucleotides;
   (ii) at less than twelve 2′-F modification; and
   (iii) at least two phosphorothioate internucleotide linkages at the first five nucleotides (counting from the 5′ end);
wherein the duplex region is between 19 to 25 base pairs (preferably 19, 20, 21 or 22); and wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the oligonucleotide is a dsRNA agent, and the dsRNA agent comprises a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have less than 20%, less than 15% and less than 10% non-natural nucleotide.

In one embodiment, the oligonucleotide is a dsRNA agent, and the dsRNA agent comprises a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have greater than 80%, greater than 85% and greater than 90% natural nucleotide, such as 2′-OH, 2′-deoxy and 2′-OMe are natural nucleotides.

In one embodiment, the oligonucleotide is a dsRNA agent, and the dsRNA agent comprises a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have 100% natural nucleotide, such as 2′-OH, 2′-deoxy and 2′-OMe are natural nucleotides.

In one embodiment, the oligonucleotide is a dsRNA agent, and the dsRNA agent comprise a sense strand and an antisense strand, each strand having 14 to 30 nucleotides, wherein the sense strand sequence is represented by formula (I):

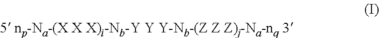

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 1, 2, 3, 4, 5, or 6 modified nucleotides;
each nₚ and n_{q} independently represent an overhang nucleotide;
wherein N_{b} and Y do not have the same modification;
wherein XXX, YYY and ZZZ each independently represent one motif of three identical modifications on three consecutive nucleotides;
wherein the dsRNA agents have one or more lipophilic monomers containing one or more lipophilic moieties conjugated to one or more positions on at least one strand; and
wherein the antisense strand of the dsRNA comprises two blocks of one, two or three phosphorothioate internucleotide linkages separated by 1, 2, 3, 4, 5, 6, 7, 8, 9, 10, 11, 12, 13, 14, 15, 16, 17, or 18 phosphate internucleotide linkages.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, the antisense strand is 100% complementary to a target RNA to hybridize thereto and inhibits its expression through RNA interference. In another embodiment, the antisense strand is at least 95%, at least 90%, at least 85%, at least 80%, at least 75%, at least 70%, at least 65%, at least 60%, at least 55%, or at least 50% complementary to a target RNA.

### Exemplary Duplex Motifs

In certain embodiments, the oligonucleotide is a dsRNA agent, and the sense strand of the dsRNA agent has one of the following modification patterns:

| | |
|---|---|
| S1 | 5′-(Lipo)snsnnnnNfnNfNfNfnnnnnnnnsnsn-3′ |
| S2 | 5′-ns(Lipo)snnnnNfnNfNfNfnnnnnnnnsnsn-3′ |
| S3 | 5′-nsns(Lipo)nnnNfnNfNfNfnnnnnnnnsnsn-3′ |
| S4 | 5′-nsnsn(Lipo)nnNfnNfNfNfnnnnnnnnsnsn-3′ |
| S5 | 5′-nsnsnn(Lipo)nNfnNfNfNfnnnnnnnnsnsn-3′ |
| S6 | 5′-nsnsnnn(Lipo)NfnNfNfNfnnnnnnnnsnsn-3′ |
| S7 | 5′-nsnsn(Lipo)nnnnNfNfNfnnnnnnnnsnsn-3′ |
| S8 | 5′-nsnsnn(Lipo)nnnNfNfNfnnnnnnnnsnsn-3′ |
| S9 | 5′-nsnsnnn(Lipo)nnNfNfNfnnnnnnnnsnsn-3′ |
| S10 | 5′-nsnsnnnn(Lipo)nNfNfNfnnnnnnnnsnsn-3′ |
| S11 | 5′-nsnsnnnnNfnNfNfNfnnn(Lipo)nnnnsnsn-3′ |
| S12 | 5′-nsnsnnnnNfnNfNfNfnnnn(Lipo)nnnsnsn-3′ |
| S13 | 5′-nsnsnnnnNfnNfNfNfnnnnn(Lipo)nnsnsn-3′ |
| S14 | 5′-nsnsnnnnNfnNfNfNfnnnnn(Lipo)nnsns(Lipo)-3′ |
| S15 | 5′-nsnsnnnnnnNfNfNfnnn(Lipo)nnnnsnsn-3′ |
| S16 | 5′-nsnsnnnnnnNfNfNfnnnn(Lipo)nnnsnsn-3′ |
| S17 | 5′-nsnsnnnnnnNfNfNfnnnnn(Lipo)nnsnsn-3′ |
| S18 | 5′-nsnsnnnnnnNfNfNfnnnnn(Lipo)nnsns(Lipo)-3′ |
| S19 | 5′-(L1)(inv)snnnnnnnnNfNfNfnnnnnnnnns(inv)(L2)-3′, |
| S20 | 5′NfsnsNfnNfnNfnNfNfNfnNfnNfnNfnNfnsNf-3′, where (Lipo) is |
| | substituted at any one of positions 4-8 or 13-18 counting from the 5′-end of |
| | the strand |
| S21 | 5′NfsnsNfnNfnNfnNfNfNfnNfnnnNfnNfsnsNf-3′, where (Lipo) is |
| | substituted at any one of positions 4-8 or 13-18 counting from the 5′-end of |
| | the strand |
| S22 | 5′-nsnsnnnnnn(dN)n(dN)nnnnnnnnsnsn-3′, where (Lipo) is substituted at |
| | any one of positions 4-8 or 13-18 counting from the 5′-end of the strand |
| S23 | 5′-nsnsnnnnNfnNfn(dN)nnnnnnnnsnsn-3′, where (Lipo) is substituted at |
| | any one of positions 4-8 or 13-18 counting from the 5′-end of the strand |

where n is a 2′-O-methyl-nucleotide;
s is a phosphorothioate internucleotide linkage;
Nf is a 2′-fluoro-modified nucleotide;
(Lipo) is n, Nf, or an optionally lipophilic-modified nucleotide (such as (Nhd)-2′-O-hexadecyl-modified nucleotide);
each (inv) is an inverted nucleotide (e.g., an inverted abasic nucleotide, such as an inverted abasic ribonucleotide); and
at least one of (L1) and (L2) is a ligand comprising a lipophilic group (e.g., comprising an C10-C30 alkyl, or a C10-C30 alkenyl group, e.g., a C16 alkyl, a C16 alkenyl, a C18 alkyl, a C18 alkenyl, a C20 alkyl, a C20 alkenyl, a C22 alkyl, a C22 alkenyl, a C24 alkyl, or a C24 alkenyl) and the other of (L1) and (L2) is absent or hydrogen.

In any of the preceding S1-S23 modification patterns, where (Lipo) is not present the 3′ end or 5′ end of the strand may be conjugated to a ligand (e.g., a targeting ligand as described herein). In one embodiment, a ligand is conjugated to the 5′-end of the strand. In another embodiment, embodiment, a ligand is conjugated to the 5′-end of the strand. Exemplary targeting ligands are described herein, such as a carbohydrate-based ligand targeting a liver tissue. In one embodiment, the carbohydrate-based ligand is selected from the group consisting of galactose, multivalent galactose, N-acetyl-galactosamine (GalNAc), multivalent GalNAc, mannose, multivalent mannose, lactose, multivalent lactose, N-acetyl-glucosamine (GlcNAc), multivalent GlcNAc, glucose, multivalent glucose, fucose, and multivalent fucose.

In some embodiments, the oligonucleotide is a dsRNA agent, and the antisense strand of the dsRNA agent has one of the following modification patterns:

| | |
|---|---|
| AS1 | 5′-Zns(dN)snn(dN)n(dN)nnnn(dN)nNfnnnnnnnsnsn-3′ |
| AS2 | 5′-Zns(dN)snn(dN)n(D)nnnn(dN)nNfnnnnnnnsnsn-3′ |
| AS3 | 5′-ZnsNfsnn(dN)n(D)nnnnnnNfnNfnnnnnsnsn-3′ |
| AS4 | 5′-ZnsNfsnnnNfnnnnnnnNfnNfnnnnnsnsn-3′ |
| AS5 | 5′-ZnsNfsnnnNfnnNfnnnnNfnNfnnnnnsnsn-3′ |
| AS6 | 5′-ZnsNfsnnnNfnNfNfnnnnNfnNfnnnnnsnsn-3′ |
| AS7 | 5′-ZnsNfsnnnNf(D)nnnnnnNfnNfnnnnnsnsn-3′ |
| AS8 | 5′-ZnsNfsnnnNf(D)nNfnnnnNfnNfnnnnnsnsn-3′ |
| AS9 | 5′-ZnsNfsnnnNf(D)NfNfnnnnNfnNfnnnnnsnsn-3′ |
| AS10 | 5′-ZnsNfsnNfnNfnNfnNfnNfnNfnNfnNfnsNfsn-3′ |
| AS11 | 5′-ZnsNfsnNfnNfnNfnNfnNfnNfnNfnNfnsnsn-3′ |
| AS12 | 5′-ZnsNfsnNfnNfnnnnnNfnNfnNfnNfnsNfsn-3′ |
| AS13 | 5′-ZnsNfsnsNfnNfnNfnNfnNfnNfnNfnNfnNfsn-3′ |
| AS14 | 5′-ZnsNfsnsNfnNfnnnnnNfnNfnNfnNfnNfsn-3′, |
| AS15 | 5′-ZnsNfsnsNfnNfnNfnNfnNfnNfnNfnNfnnsn-3′ |
| AS16 | 5′-ZnNfnNfnNfNfNfnNfnnnNfnNfnNfnNfnNfsn-3′ |
| AS17 | 5′-ZnsNfsnNfnNfnNfnNfnnnNfnNfnNfnNfnsnsn-3′ |
| AS18 | 5′-Zns(dN)snn(dN)n(dN)nnnn(dN)n(dN)n(dN)nnnnnsnsn-3′ |

n is a 2′-O-methyl-modified nucleotide; s is a phosphorothioate internucleotide linkage; (dN) is a 2′-deoxy-nucleotide; Nf is a 2′-fluoro-modified nucleotide;
(D) is a thermally destabilizing modification, such as (Ngn)—a glycol nucleic acid, 5-isomer; or (N2p)—a 2′-phosphate nucleotide (i.e., a 3′-RNA); or (MM) a nuclobase mismatch to the sense strand; or (Nul) an unlocked nucleic acid; and
Z is a compound having a structure of formula (I) as described herein, connected to the 5′-oxygen of the terminal nucleotide.

In further embodiments, Z comprises a

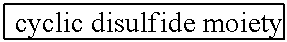

of the structure of formula (C-I) or (C-IIa) or (C-IIb), as defined above, connected at 5′end of the oligonucleotide through a

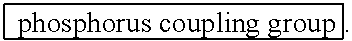

In further embodiments, Z comprises a

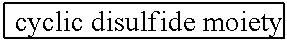

of the structure of formula (C-Ia) connected at 5′end of the oligonucleotide through a

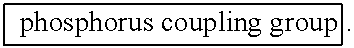

In further embodiments, Z comprises a

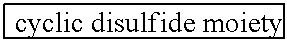

of the structure of formula (C-Ib) connected at 5′end of the oligonucleotide through a

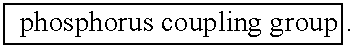

In further embodiments, Z comprises a

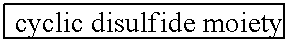

of the structure of formula (C-Ic) connected at the 5′end of the oligonucleotide through a

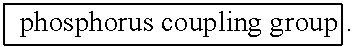

In further embodiments, Z comprises

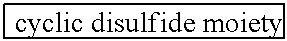

of the structure of formula (C-Id) connected at the 5′end of the oligonucleotide through a

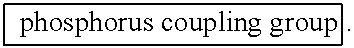

In further embodiments, Z comprises a

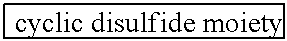

of the structure of formula (C-Ie) connected at the 5′end of the oligonucleotide through a

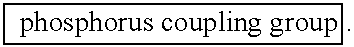

In further embodiments, Z comprises a

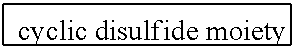

of the structure of formula (C-If) connected at the 5′end of the oligonucleotide through a

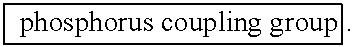

In further embodiments, Z comprises a

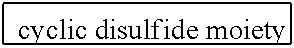

of the structure of formula (C-IIa) connected at the 5′end of the oligonucleotide through

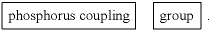

In further embodiments, Z comprises

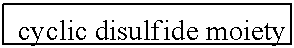

of the structure of formula (C-IIa) connected at the 5′end of the oligonucleotide through a

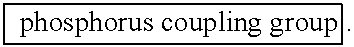

In further embodiments, Z comprises a

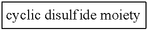

having a structure selected from the group consisting of:

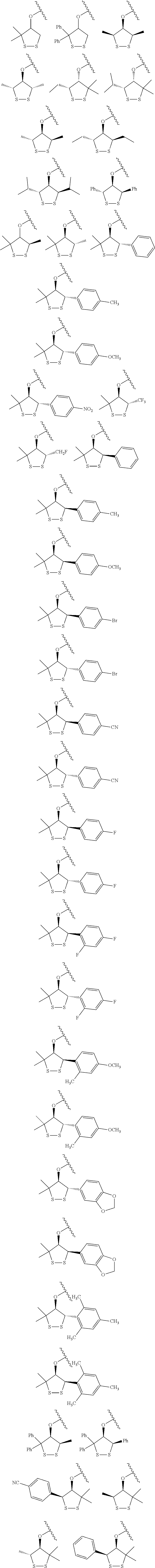

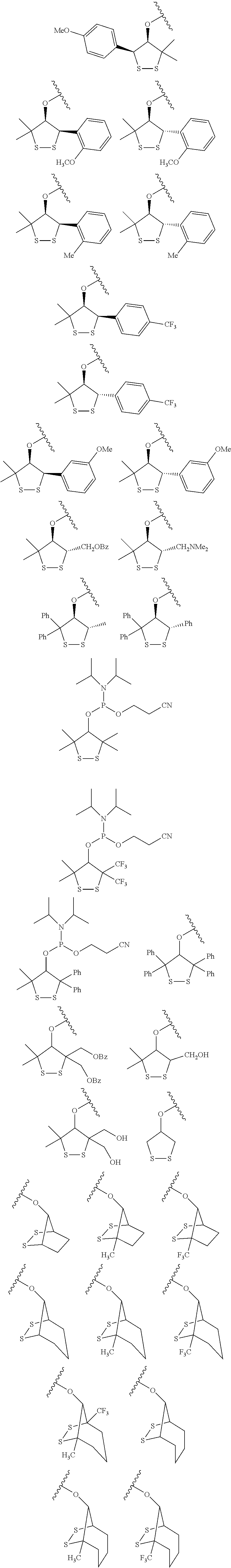

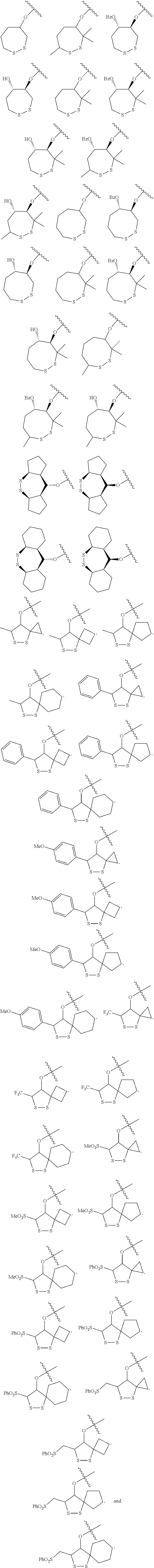

wherein the

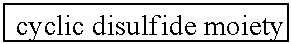

connected to the 5′-carbon at the 5′end of the oligonucleotide through a

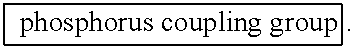

In each of the preceding structures, the

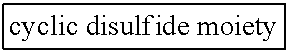

can have 2 chiral centers, and the structure can include the racemic form and individual diasteromer.

In each of the preceding embodiments, the

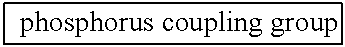

is as defined in any embodiment above; for example, the

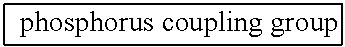

can be connected to the 5′-carbon of the 5′-terminal nucleotide and is selected from the group consisting of:

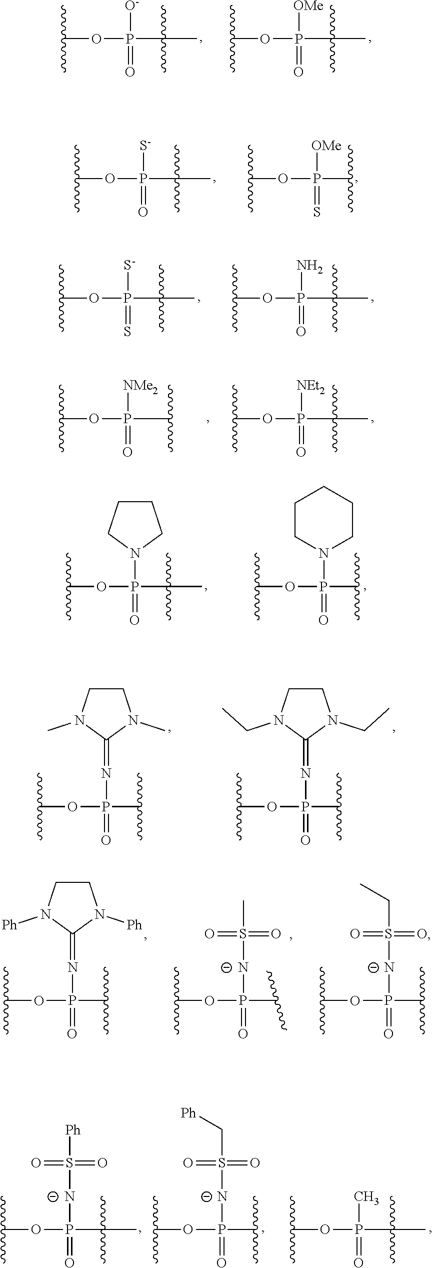

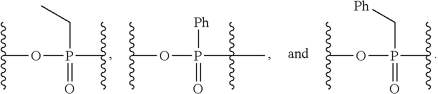

In one embodiment, the

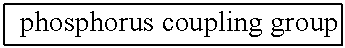

is selected from the group consisting of:

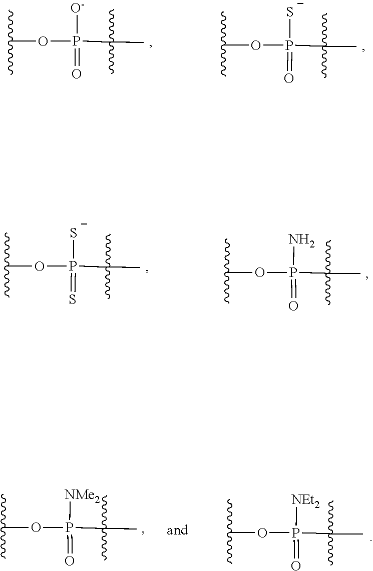

In another embodiment, the

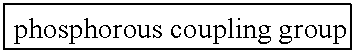

is selected from the group consisting of:

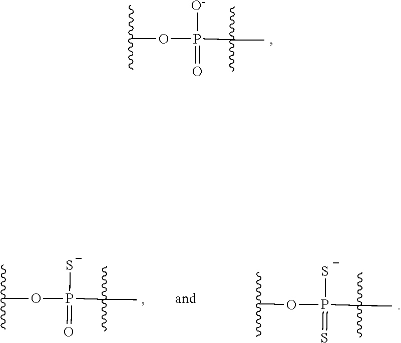 [SMILES: *OP(*)(=O)[O-] ; *OP(*)(=O)[S-] ; *OP(*)(=S)[S-]]

In another embodiment, the

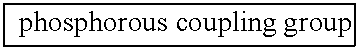

is

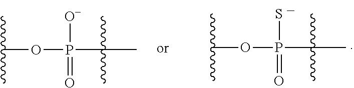 [SMILES: *OP(*)(=O)[O-] ; *OP(*)(=O)S]

In another embodiment, the

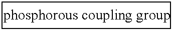

is

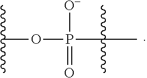 [SMILES: *OP(*)(=O)[O-]]

In another embodiment, the

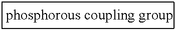

is

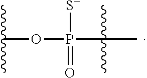 [SMILES: *OP(*)(=O)[S-]]

In a further embodiment of each of the preceding exemplary sense and antisense strands, each of sense strand S1 through S23 may be duplexed with any one of antisense strands AS1 through AS17.

### Nucleic Acid Modifications

In some embodiments, the oligonucleotide comprises at least one nucleic acid modification described herein. For example, at least one modification selected from the group consisting of modified internucleoside linkage, modified nucleobase, modified sugar, and any combinations thereof. Without limitations, such a modification can be present anywhere in the oligonucleotide. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the oligonucleotides described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligonucleotide described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic includes more complicated structures, such as for example a tricyclic phenoxazine nucleobase mimetic. Methods for preparation of the above noted modified nucleobases are well known to those skilled in the art.

### Nucleic Acid Modifications (Sugar)

The oligonucleotide provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligonucleotides comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from—[C(R1)(R2)]ₙ-, —[C(R1)(R2)]ₙ—O—, —[C(R1)(R2)]ₙ-N(R1)—, —[C(R1)(R2)]ₙ-N(R1)-O—, —[C(R1R2)]ₙ-O—N(R1)—, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)— and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C₅-C₇ alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′- wherein each R1 is, independently, H, a protecting group or C₁-C₁₂ alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′—(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE═NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE═NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligonucleotide can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The oligonucleotide disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. The oligonucleotide can also contain one or more sugars that are the L isomer, e.g. L-nucleosides. Modification to the sugar group can also include replacement of the 4′-0 with a sulfur, optionally substituted nitrogen or CH₂ group. In some embodiments, linkage between C1′ and nucleobase is in a configuration.

Sugar modifications can also include a “acyclic nucleotide,” which refers to any nucleotide having an acyclic ribose sugar, e.g., wherein a C-C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

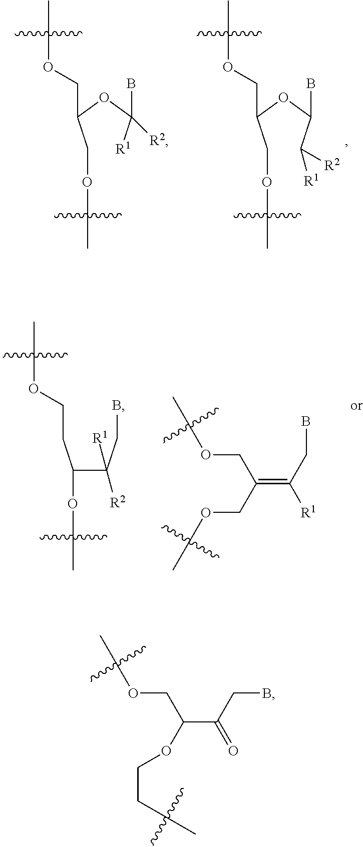

wherein B is a modified or unmodified nucleobase, R₁ and RZ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

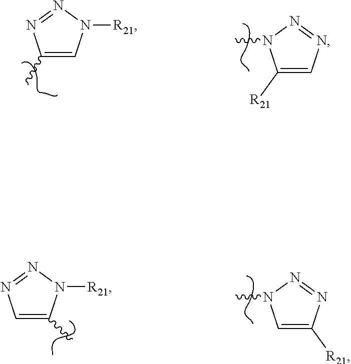

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alkali metal or transition metal with an overall charge of +1; and Y is O, S, or NR′, where R′ is hydrogen, optionally substituted aliphatic. Preferably this modification is at the 5′ terminal of the iRNA.

In certain embodiments, the oligonucleotide comprises at least two regions of at least two contiguous monomers of the above formula. In certain embodiments, the oligonucleotide comprises a gapped motif. In certain embodiments, the oligonucleotide comprises at least one region of from about 8 to about 14 contiguous β-D-2′-deoxyribofuranosyl nucleosides. In certain embodiments, the oligonucleotide comprises at least one region of from about 9 to about 12 contiguous β-D-2′-deoxyribofuranosyl nucleosides.

In certain embodiments, the oligonucleotide comprises at least one (e.g., 1, 2, 3, 4, 5, 6, 7, 8, 9, 10, 11, 12, 13, 14, 15 or more) comprises at least one (S)-cEt monomer of the formula:

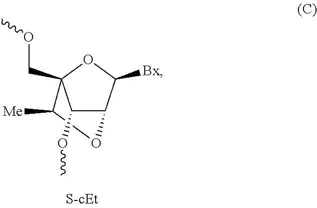

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—).

As discussed above, described herein is the

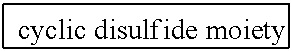

that is introduced to one or more of the phosphorous-containing internucleotide linkage groups of an oligonucleotide as a temporary protecting group. The remaining phosphorous-containing internucleotide linkage groups can also be modified using the methods described below.

Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S-CH₂—O-5′), formacetal (3′-O—CH₂-O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O-C5′), thioethers (C3′-S-C5′), thioacetamido (C3′—N(H)—C(═O)—CH₂—S—C5′, C3′-O—P(O)—O—SS-C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phosphotriesters, aminoalkylphosphotriesters, alkyl-phosphonaters (e.g., methyl-phosphonate), selenophosphates, phosphoramidates (e.g., N-alkylphosphoramidate), and boranophosphonates.

In some embodiments, the oligonucleotide further comprises at least one (e.g., 1, 2, 3, 4, 5, 6, 7, 8, 9, 10, 11, 12, 13, 14, 15 or more and up to including all) modified or nonphosphodiester linkages. In some embodiments, the oligonucleotide further comprises at least one (e.g., 1, 2, 3, 4, 5, 6, 7, 8, 9, 10, 11, 12, 13, 14, 15 or more and up to including all) phosphorothioate linkages.

The oligonucleotide can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backbone-extended pyrrolidine PNA (bepPNA) nucleoside surrogates. A preferred surrogate is a PNA surrogate.

The oligonucleotide described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the oligonucleotide are all such possible isomers, as well as their racemic and optically pure forms.

### Nucleic Acid Modifications (Terminal Modifications)

In some embodiments, the oligonucleotide further comprises a phosphate or phosphate mimic at the 5′-end of the antisense strand. In one embodiment, the phosphate mimic is a 5′-vinyl phosphonate (VP).

In some embodiments, the 5′-end of the antisense strand does not contain a 5′-vinyl phosphonate (VP).

Ends of the iRNA agent can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligonucleotide, this array can substitute for a hairpin loop in a hairpin-type oligonucleotide.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligonucleotide comprises the modification

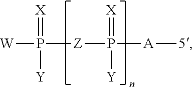

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O—5′); 5′-triphosphate ((HO)₂(O)P—O-(HO)(O)P—O—P(HO)(O)—O-5′); 5′-monothiophosphate (phosphorothioate; (HO)₂(S)P-O-5′); 5′-monodithiophosphate (phosphorodithioate; (HO)(HS)(S)P-O-5′), 5′-phosphorothiolate ((HO)₂(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ); 5′-guanosine cap (7-methylated or non-methylated) (7m-G-O-5′-(HO)(O)P—O-(HO)(O)P—O—P(HO)(O)—O—5′); 5′-adenosine cap (Appp), and any modified or unmodified nucleotide cap structure (N-O-5′-(HO)(O)P—O-(HO)(O)P—O-P(HO)(O)—O—5′). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The oligonucleotide, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

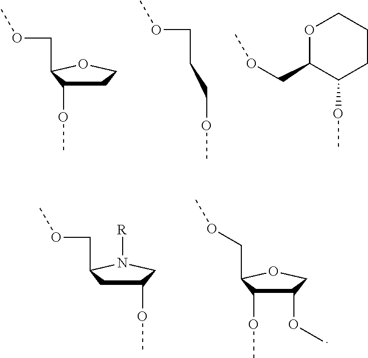

Exemplified sugar modifications are:

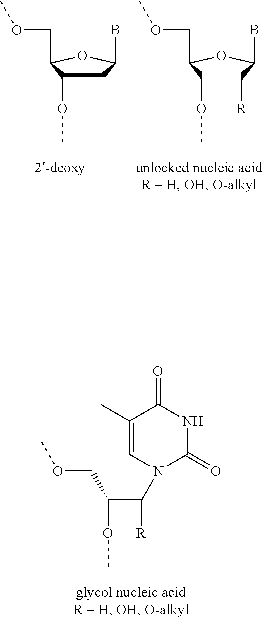

The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

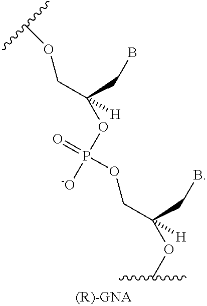

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the oligonucleotide, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In certain embodiments, the antisense strand includes one thermally destabilizing nucleotide that is not a terminal nucleotide and not a cleavage region nucleotide. Optionally, the thermally destabilizing nucleotide is a glycol nucleic acid (GNA). Optionally, the thermally destabilizing nucleotide is an unlocked nucleic acid (UNA). Optionally, the thermally destabilizing nucleotide is a 2′-5′ linked ribonucleotide (3′-RNA).

Further examples of thermally destabilizing nucleotide include,

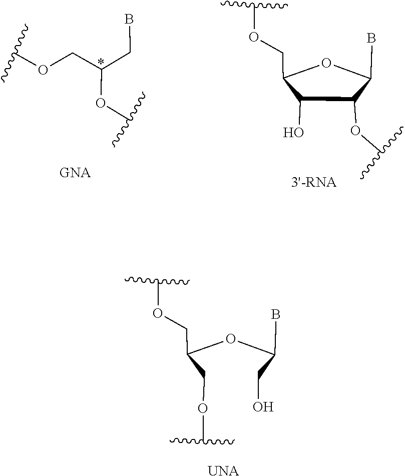

or a stereoisomer thereof, wherein B is a modified or unmodified nucleobase and asterisk represents either R, S or racemic (e.g., S-GNA). In another embodiment, a thermally destabilizing nucleotide includes a nucleobase mismatch between the antisense and the sense strand; for example, the sense strand may have a mismatch to the antisense strand while the latter remains matched at the same position to a target mRNA.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

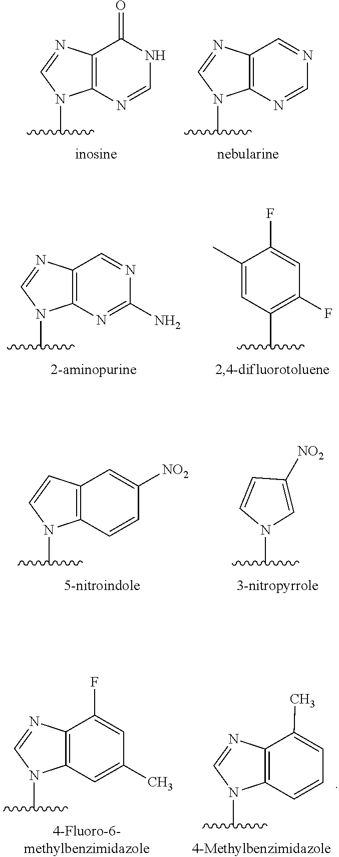

Exemplary phosphate modifications known to decrease the thermal stability of dsRNA duplexes compared to natural phosphodiester linkages are:

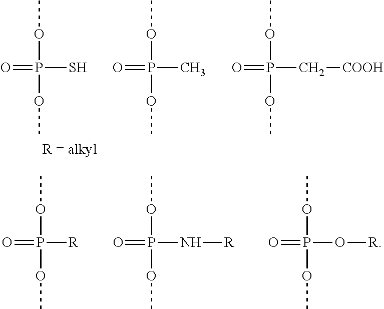

In some embodiments, the oligonucleotide can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the oligonucleotide can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In some embodiments, one or more targeting ligands are connected to the modified phosphate prodrug compound via any one of R₂, R₃, R₄, R₅, R₆, R₇, R₈, and R9 of the

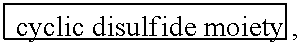

optionally via one or more linkers/tethers.

Introduction of the tar ng ligands into an oligonucleotide via a

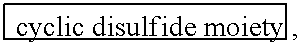

on either the sense or antisense strand or both the sense and antisense strands, are illustrated in Scheme 16 in Example 10 below. These targeting ligands can be cleaved off with the

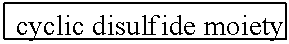

after the siRNA oligonucleotide enters into cytosol.

In some embodiments, the targeting ligand is selected from the group consisting of an antibody, a ligand-binding portion of a receptor, a ligand for a receptor, an aptamer, a carbohydrate-based ligand, a fatty acid, a lipoprotein, folate, thyrotropin, melanotropin, surfactant protein A, mucin, glycosylated polyaminoacids, transferrin, bisphosphonate, polyglutamate, polyaspartate, a lipophilic moiety that enhances plasma protein binding, a cholesterol, a steroid, bile acid, vitamin B12, biotin, a fluorophore, and a peptide.

In certain embodiments, at least one ligand is a carbohydrate-based ligand targeting a liver tissue. In one embodiment, the carbohydrate-based ligand is selected from the group consisting of galactose, multivalent galactose, N-acetyl-galactosamine (GalNAc), multivalent GalNAc, mannose, multivalent mannose, lactose, multivalent lactose, N-acetyl-glucosamine (GlcNAc), multivalent GlcNAc, glucose, multivalent glucose, fucose, and multivalent fucose.

In certain embodiments, at least one ligand is a lipophilic moiety. In one embodiment, the lipophilicity of the lipophilic moiety, measured by log K_{ow}, exceeds 0, or the hydrophobicity of the compound, measured by the unbound fraction in the plasma protein binding assay of the compound, exceeds 0.2.

In one embodiment, the lipophilic moiety contains a saturated or unsaturated C₄-C₃₀ hydrocarbon chain, and an optional functional group selected from the group consisting of hydroxyl, amine, carboxylic acid, sulfonate, phosphate, thiol, azide, and alkyne. For instance, the lipophilic moiety contains a saturated or unsaturated C₆-C₁₈ hydrocarbon chain.

Additional lipophilic moieties and additional details regarding lipophilicity of the lipophilic moiety and hydrophobicity of the oligonucleotide can be found in PCT Application No. PCT/US20/59399, entitled “Extrahepatic Delivery,” filed on Nov. 6, 2020, the content of which is incorporated herein by reference in its entirety.

In certain embodiments, at least one ligand targets a receptor which mediates delivery to a CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

In certain embodiments, at least one ligand targets a receptor which mediates delivery to an ocular tissue. In one embodiment, the targeting ligand is selected from the group consisting of trans-retinol, RGD peptide, LDL receptor ligand, and carbohydrate based ligands.

The targeting ligands can also be introduced into the oligonucleotide directly (independent (i.e., not through the

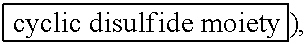

In some embodiments, the oligonucleotide contains at east e targeting ligand at the 5′-end, 3′-end, and/or internal position(s) of the antisense strand.

In some embodiments, the oligonucleotide contains at least one targeting ligand at the 5′-end, 3′-end, and/or internal position(s) of the sense strand.

In some embodiments, the oligonucleotide contains at least one

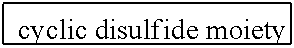

at the 5′-end, 3′-end, and/or internal position(s) of the antisense strand, and at least one targeting ligand at the 5′-end, 3′-end, and/or internal position(s) of the sense strand.

In one embodiment, the oligonucleotide contains at least one

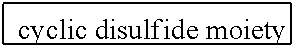

at the 5′-end of the antisense strand, and at least one targeting ligand at the 3′-end of the sense strand.

In some embodiments, one or more targeting ligands are connected to the modified phosphate prodrug compound (via the

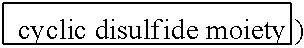

via one or more linkers/tethers, as described below.

In some embodiments, one or more targeting ligands are connected to the oligonucleotide directly (i.e., not through the

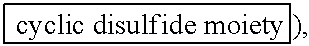

via one or more linkers/tethers, as described below.

### Linkers/Tethers

Linkers/Tethers are connected to the modified phosphate prodrug compound at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)-) group on the linker/tether, which may serve as a connection point for the modified phosphate prodrug compound. Non-limited examples of linkers/tethers (underlined) include TAP-(CH₂)ₙNH-; TAP-C(O)(CH₂)ₙNH-; TAP-NR″″(CH₂)ₙNH-, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—: or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND): TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)—C(O)(LIGAND); TAP-C(O(CH₂)ₙ—C(O)O(LIGAND); TAP-C(O)—O(LIGAND); TAP-C(O—CH₂)ₙ—NH—C(O(LIGAND): TAP-C(O)—(CH₂)ₙ(LIGAND); TAP-C(O)—NH(LIGAND); TAP-C(O)(LIGAND); TAP-(CH₂)ₙ—C(O)(LIGAND); TAP-(CH₂)ₙ—C(O)O(LIGAND); TAP-(CH₂)(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)—SH, TAP-C(O)(CH₂)ₙSH. TAP-(CH₂)ₙ—(CH═CH₂), or TAP-C(O)(CH₂)(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O) OC₆F₅; or TAP-NR″″(CH₂)ₙC(O) OC₆F₅, in which n is 1-11 and R″″ is C₁-C₆ alkyl; or -(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether

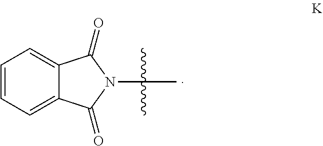 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O-P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S-. A preferred embodiment is —O—P(O)(OH)—O-. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)-. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH-). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers/Tethers

The linkers can also include biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include:

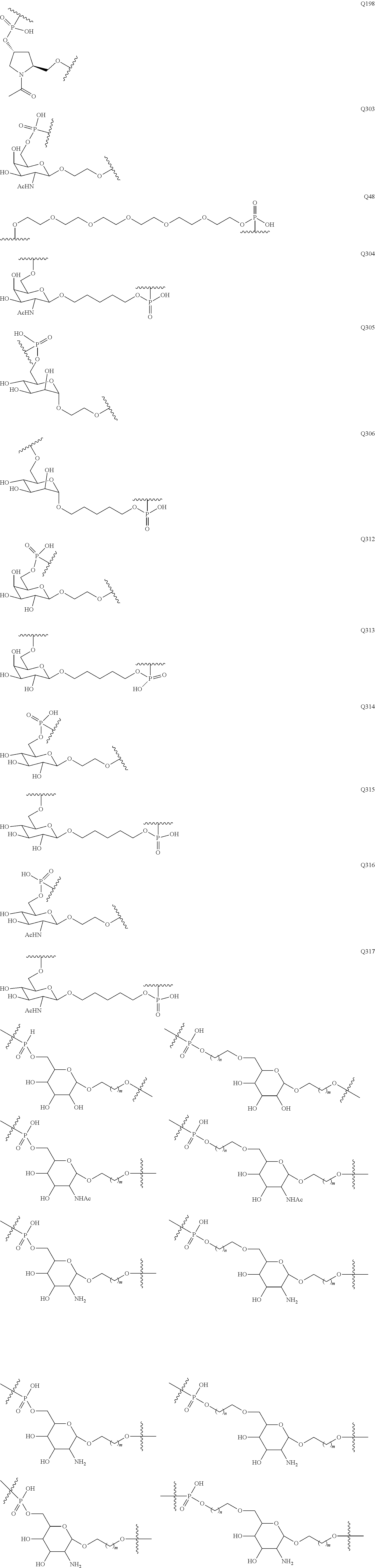

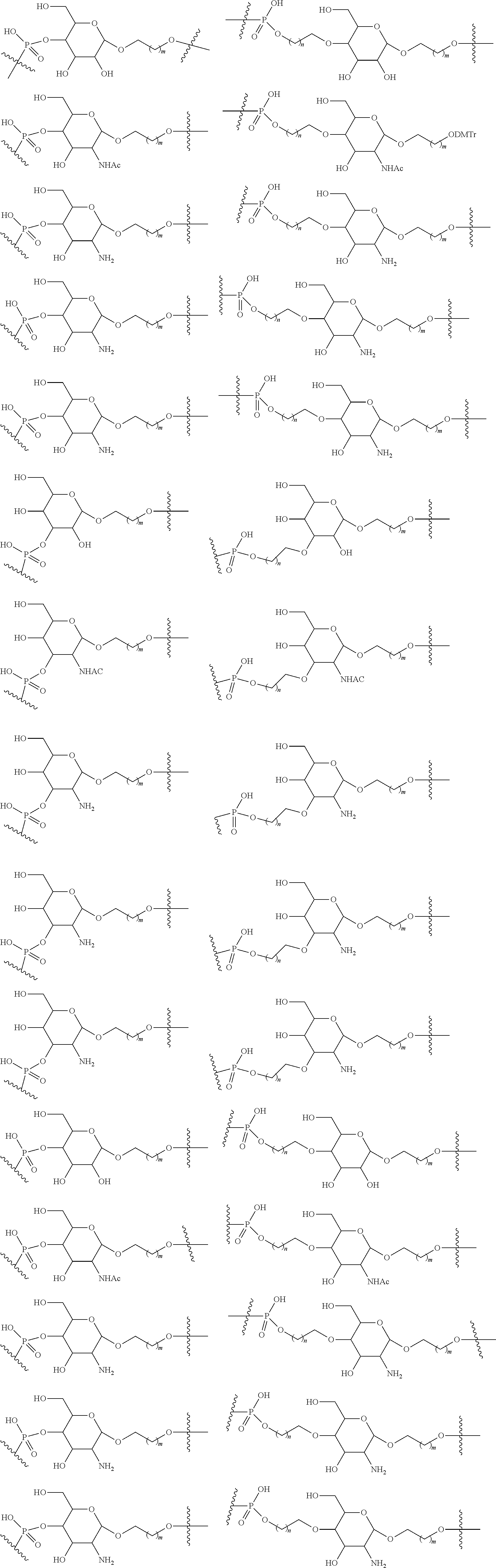

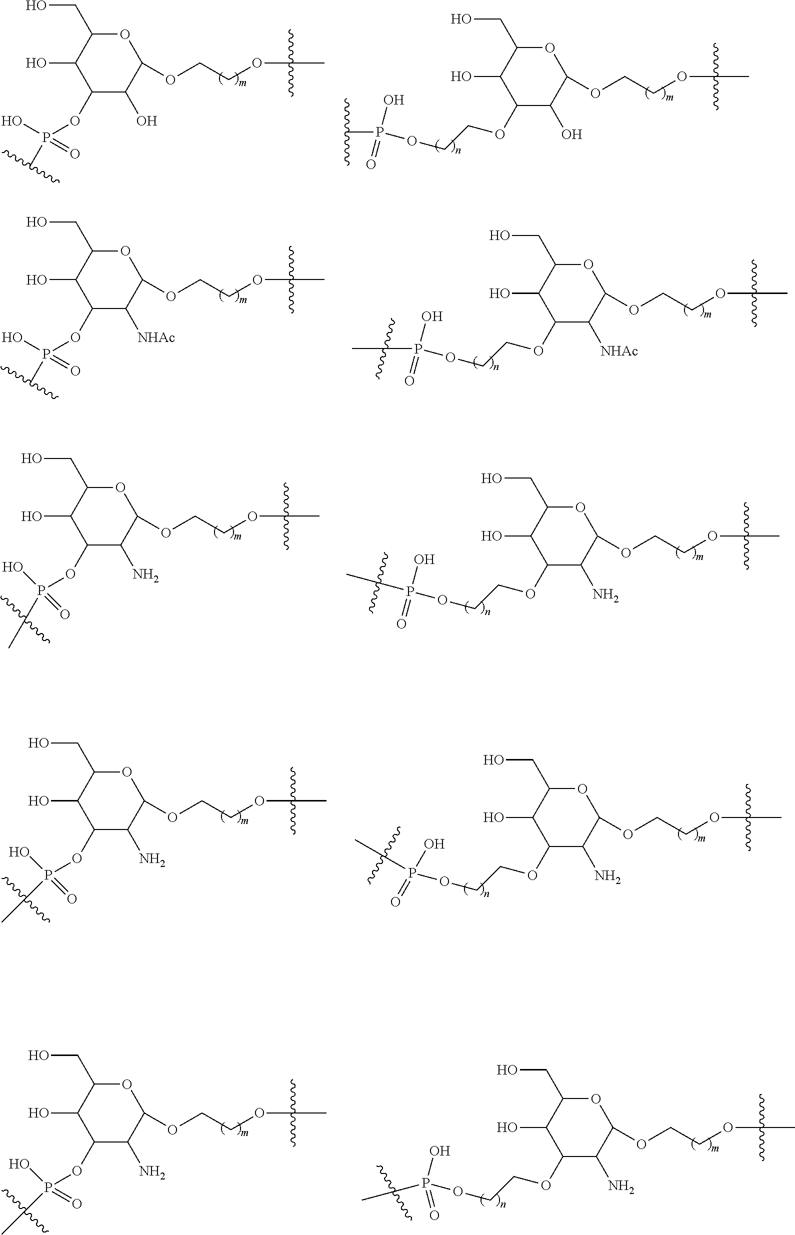

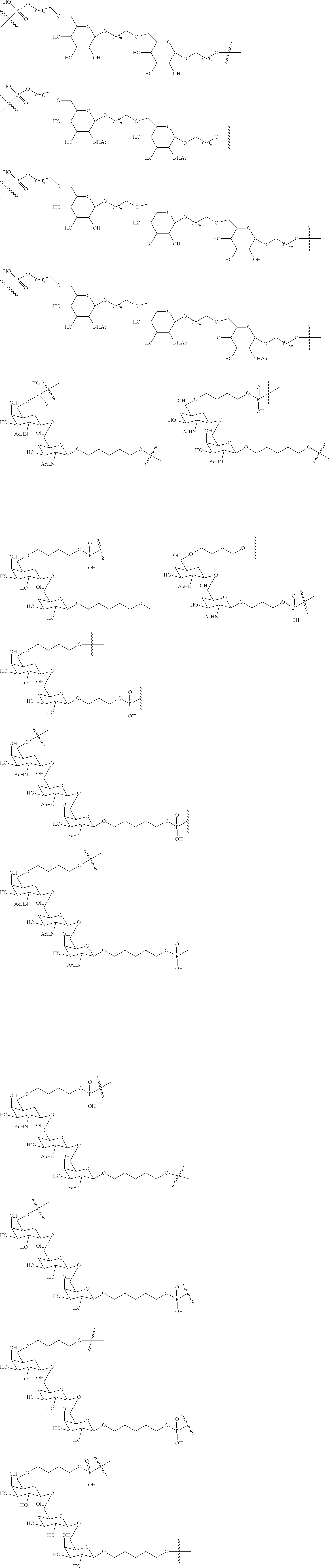

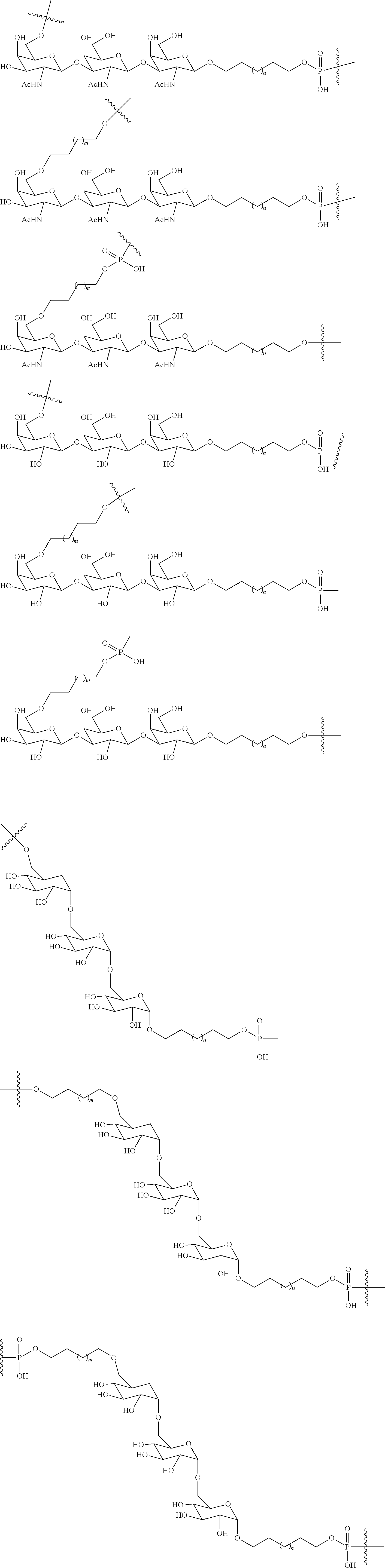

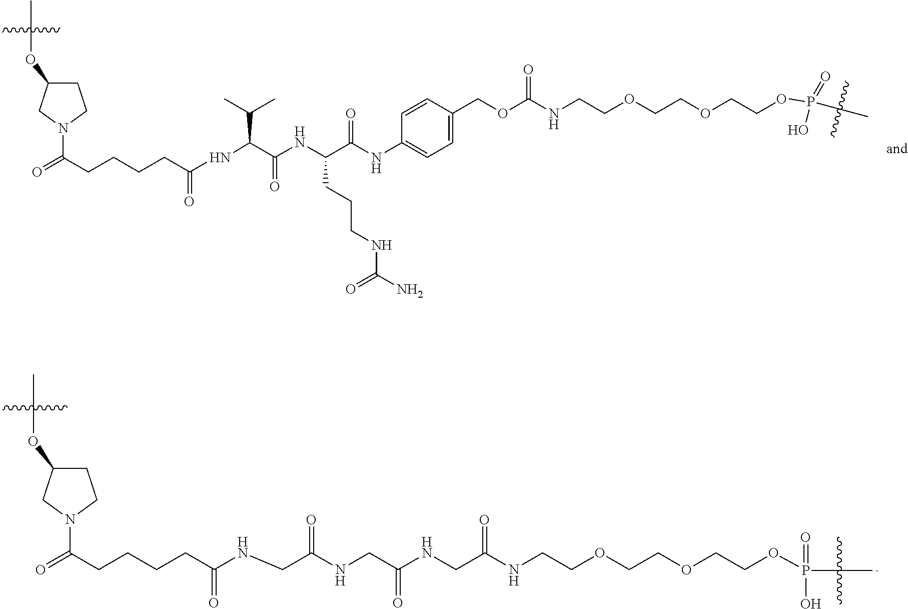 [SMILES: *O[C@H]1CCN(C(=O)CCCCC(=O)N[C@H](C(=O)N[C@@H](CCCNC(N)=O)C(=O)Nc2ccc(COC(=O)NCCOCCOCCOP(*)(=O)O)cc2)C(C)C)C1 ; *O[C@H]1CCN(C(=O)CCCCC(=O)NCC(=O)NCC(=O)NCC(=O)NCCOCCOCCOP(*)(=O)O)C1]

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### Carrier

In some embodiments, one or more targeting ligands are connected to the modified phosphate prodrug compound (via the

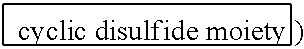

via one or more carriers, as described herein, and optionally via one or more linkers/tethers, as described above,

In some embodiments, one or more targeting ligands are connected to the oligonucleotide directly (i.e., not through the

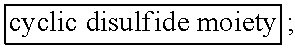

via one or more carriers, as described herein, and optionally via one or more linkers/tethers, as described above.

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

The carrier can replace one or more nucleotide(s) of the iRNA agent.

In some embodiments, the carrier replaces one or more nucleotide(s) in the internal position(s) of the iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand. The targeting ligand can be directly attached to the carrier or indirectly attached to the carrier by an intervening linker/tether, as described above.

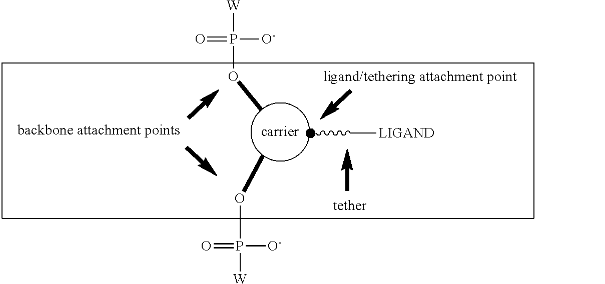

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁸ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂-, wherein one bond is connected to the carrier and one to a backbone atom, e.g., a linking oxygen or a central phosphorus atom.

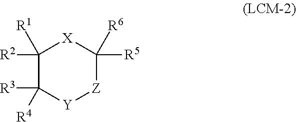

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is -C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X^{5″})(X^{5′″}) in which (X^{5′}), (X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy; n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D)

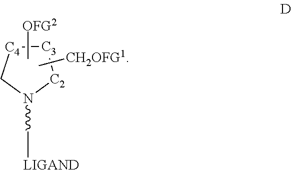

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to CA. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to CA. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

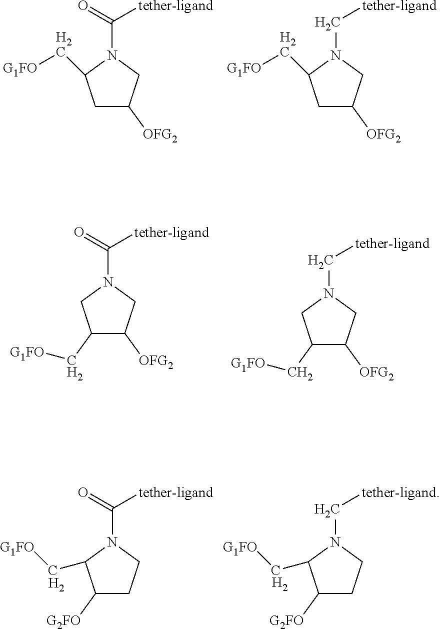

In certain embodiments, the carrier may be based on the piperidine ring system (E), e.g., X is N(CO)R⁷ or NR⁷, Y is CR⁹R¹⁰, and Z is CR¹¹R¹²

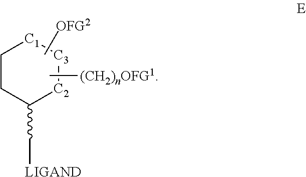

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E).-(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3;-(CH₂)ₙOFG¹ may be attached to C-3 and OFG² may be attached to C-2;-(CH₂)ₙOFG¹ may be attached to C-3 and OFG² may be attached to C4; or -(CH₂)ₙOFG¹ may be attached to C4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

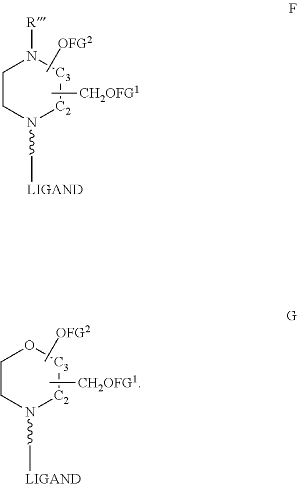

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

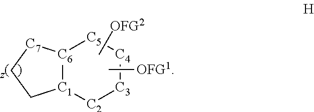

OFG¹ is preferably attached to a primary carbon, e.g., an exocyclic methylene group (n=1) or ethylene group (n=2) connected to one of C-2, C-3, C4, or C-5 [—(CH₂)ₙOFG¹ in H]. OFG² is preferably attached directly to one of C-2, C-3, C4, or C-5 (—OFG² in H).-(CH₂)ₙOFG¹ and OFG² may be disposed in a geminal manner on the ring, i.e., both groups may be attached to the same carbon, e.g., at C-2, C-3, C4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2;-(CH₂)ₙOFG¹ may be attached to C-3 and OFG² may be attached to C4; or -(CH₂)ₙOFG¹ may be attached to C4 and OFG² may be attached to C-3;-(CH₂)ₙOFG¹ may be attached to C4 and OFG² may be attached to C-5; or -(CH₂)ₙOFG¹ may be attached to C-5 and OFG² may be attached to C4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

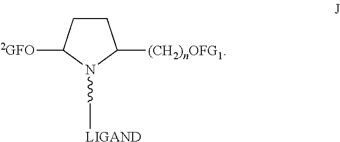

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

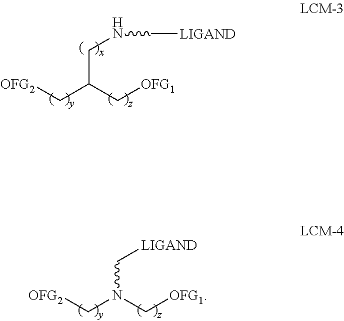

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the oligonucleotide comprises one or more targeting ligands conjugated to the 5′ end of the sense strand or the 5′ end of the antisense strand, optionally via a carrier and/or linker/tether.

In some embodiments, the oligonucleotide comprises one or more targeting ligands conjugated to the 3′ end of the sense strand or the 3′ end of the antisense strand, optionally via a carrier and/or linker/tether.

In some embodiments, the oligonucleotide comprises one or more targeting ligands conjugated to both ends of the sense strand, optionally via a carrier and/or linker/tether.

In some embodiments, the oligonucleotide comprises one or more targeting ligands conjugated to both ends of the antisense strand, optionally via a carrier and/or linker/tether.

In some embodiments, the oligonucleotide comprises one or more targeting ligands conjugated to internal position(s) of the sense or antisense strand, optionally via a carrier and/or linker/tether.

In some embodiments, one or more targeting ligands are conjugated to the ribose, nucleobase, and/or at the internucleotide linkages. In some embodiments, one or more targeting ligands are conjugated to the ribose at the 2′ position, 3′ position, 4′ position, and/or 5′ position of the ribose. In some embodiments, one or more targeting ligands are conjugated at the nucleobase of natural (such as A, T, G, C, or U) or modified as defined herein. In some embodiments, one or more targeting ligands are conjugated at the phosphate or modified phosphate groups as defined herein.

In some embodiments, the oligonucleotide comprises one or more targeting ligands conjugated to the 5′ end or 3′ end of the sense strand, and one or more same or different targeting ligands conjugated to the 5′ end or 3′ end of the antisense strand,

In some embodiments, at least one targeting ligand is located on one or more terminal positions of the sense strand or antisense strand. In one embodiment, at least one targeting ligand is located on the 3′ end or 5′ end of the sense strand. In one embodiment, at least one targeting ligand is located on the 3′ end or 5′ end of the antisense strand.

In some embodiments, at least one targeting ligand is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, at least one targeting ligand is located on one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the targeting ligand is located on one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, at least one targeting ligand is located on one or more positions of at least one end of the duplex region, which include all positions within the duplex region, but not include the overhang region or the carrier that replaces the terminal nucleotide on the 3′ end of the sense strand.

In one embodiment, at least one targeting ligand is located on the sense strand within the first five, four, three, two, or first base pairs at the 5′-end of the antisense strand of the duplex region.

In one embodiment, at least one targeting ligand (e.g., a lipophilic moiety) is located on one or more internal positions on at least one strand, except the cleavage site region of the sense strand, for instance, the targeting ligand (e.g., a lipophilic moiety) is not located on positions 9-12 counting from the 5′-end of the sense strand, for example, the targeting ligand (e.g., a lipophilic moiety) is not located on positions 9-11 counting from the 5′-end of the sense strand. Alternatively, the internal positions exclude positions 11-13 counting from the 3′-end of the sense strand.

In one embodiment, at least one targeting ligand (e.g., a lipophilic moiety) is located on one or more internal positions on at least one strand, which exclude the cleavage site region of the antisense strand. For instance, the internal positions exclude positions 12-14 counting from the 5′-end of the antisense strand.

In one embodiment, at least one targeting ligand (e.g., a lipophilic moiety) is located on one or more internal positions on at least one strand, which exclude positions 11-13 on the sense strand, counting from the 3′-end, and positions 12-14 on the antisense strand, counting from the 5′-end.

In one embodiment, one or more targeting ligands (e.g., a lipophilic moiety) are located on one or more of the following internal positions: positions 4-8 and 13-18 on the sense strand, and positions 6-10 and 15-18 on the antisense strand, counting from the 5′end of each strand.

In one embodiment, one or more targeting ligands (e.g., a lipophilic moiety) are located on one or more of the following internal positions: positions 5, 6, 7, 15, and 17 on the sense strand, and positions 15 and 17 on the antisense strand, counting from the 5′end of each strand.

### Target Genes

Without limitations, target genes for siRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the siRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLI1 fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides an olignucleotide that modulates a micro-RNA.

### Targeting CNS

In some embodiments, the invention provides an oligonucleotide that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, the invention provides an oligonucleotide that targets TARDBP for ALS, MAPT (Tau) for Frontotemporal Dementia, and HTT for Huntington Disease.

In some embodiments, the invention provides an oligonucleotide that targets SNCA for Parkinson Disease, FUS for ALS, ATXN3 for Spinocerebellar Ataxia 3, ATXN1 for SCA1, genes for SCA7 and SCA8, ATN1 for DRPLA, MCCP2 for XLMR, PRNP for Prion Diseases, recessive CNS disorders: Lafora Disease, DMPK for DM1 (CNS and Skeletal Muscle), and TTR for hATTR (CNS, ocular and systemic).

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

More detailed descriptions about these CNS targeting receptors and related diseases may be found in PCT Application No. PCT/US20/59399, entitled “Extrahepatic Delivery,” filed on Nov. 6, 2020, the content of which is incorporated herein by reference in its entirety.

In some embodiments, the invention provides an oligonucleotide that target genes for diseases including, but are not limited to, age-related macular degeneration (AMD) (dry and wet), birdshot chorioretinopathy, dominant retinitis pigmentosa 4, Fuch's dystrophy, hATTR amyloidosis, hereditary and sporadic glaucoma, and stargardt's disease.

In some embodiments, the oligonucleotide targets VEGF for wet (or exudative) AMD.

In some embodiments, the oligonucleotide targets C3 for dry (or nonexudative) AMD.

In some embodiments, the oligonucleotide targets CFB for dry (or nonexudative) AMD.

In some embodiments, the oligonucleotide targets MYOC for glaucoma.

In some embodiments, the oligonucleotide targets ROCK2 for glaucoma.

In some embodiments, the oligonucleotide targets ADRB2 for glaucoma.

In some embodiments, the oligonucleotide targets CA2 for glaucoma.

In some embodiments, the oligonucleotide targets CRYGC for cataract.

In some embodiments, the oligonucleotide targets PPP3CB for dry eye syndrome.

### Ligands

In certain embodiments, the oligonucleotide is further modified by covalent attachment of one or more conjugate groups. In general, conjugate groups modify one or more properties of the attached compound of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligonucleotide. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the oligonucleotide further comprises a targeting ligand that targets a receptor which mediates delivery to a specific CNS tissue. These targeting ligands can also be conjugated in combination with a lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

In some embodiments, the oligonucleotide further comprises a targeting ligand that targets a receptor which mediates delivery to a specific ocular tissue. These targeting ligands can also be conjugated in combination with a lipophilic moiety to enable specific ocular delivery (e.g., intravitreal delivery) and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH (SEQ ID. NO: 328) or Cyclo(-Arg-Gly-Asp-D-Phe-Cys (SEQ ID. NO: 329); LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligonucleotides described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

(GALA)
(SEQ ID NO: 330)
AALEALAEALEALAEALEALAEAAAAGGC;
(EALA)
(SEQ ID NO: 331)
AALAEALAEALAEALAEALAEALAAAAGGC;
(SEQ ID NO: 332)
ALEALAEALEALAEA;
(INF-7)
(SEQ ID NO: 333)
GLFEAIEGFIENGWEGMIWDYG;
(Inf HA-2)
(SEQ ID NO: 334)
GLFGAIAGFIENGWEGMIDGWYG;
(diINF-7)
(SEQ ID NO: 335)
GLFEAIEGFIENGWEGMIDGWYGCGLFEAIEGFIENGWEGMID GWYGC;
(diINF-3)
(SEQ ID NO: 336)
GLFEAIEGFIENGWEGMIDGGCGLFEAIEGFIENGWEGMIDGGC;
(GLF)
(SEQ ID NO: 337)
GLFGALAEALAEALAEHLAEALAEALEALAAGGSC;
(GALA-INF3)
(SEQ ID NO: 338)
GLFEAIEGFIENGWEGLAEALAEALEALAAGGSC;
(INF-5, n is norleucine)
(SEQ ID NO: 339)
GLF EAI EGFI ENGW EGnI DG K GLF EAI EGFI ENGW EGnI DG;
(JTS-1)
(SEQ ID NO: 340)
LFEALLELLESLWELLLEA;
(ppTG1)
(SEQ ID NO: 341)
GLFKALLKLLKSLWKLLLKA;
(ppTG20)
(SEQ ID NO: 342)
GLFRALLRLLRSLWRLLLRA;
(KALA)
(SEQ ID NO: 343)
WEAKLAKALAKALAKHLAKALAKALKACEA;
(HA)
(SEQ ID NO: 344)
GLFFEAIAEFIEGGWEGLIEGC;
(Melittin)
(SEQ ID NO: 345)
GIGAVLKVLTTGLPALISWIKRKRQQ;
H₅WYG;
(SEQ ID NO: 346)
and
(SEQ ID NO: 347)
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin) (SEQ ID NO: 348); GRKKRRQRRRPPQC (Tat fragment 48-60) (SEQ ID NO: 349); GALFLGWLGAAGSTMGAWSQPKKKRKV (signal sequence based peptide) (SEQ ID NO: 350); LLIILRRRIRKQAHAHSK (PVEC) (SEQ ID NO: 351); GWTLNSAGYLLKINLKALAALAKKIL (transportan) (SEQ ID NO: 352); KLALKLALKALKAALKLA (amphiphilic model peptide) (SEQ ID NO: 353); RRRRRRRRR (Arg9)(SEQ ID NO:354); KFFKFFKFFK (Bacterial cell wall permeating peptide) (SEQ ID NO: 355); LLGDFFRKSKEKIGKEFKRIVQRIKDFLRNLVPRTES (LL-37) (SEQ ID NO: 356); SWLSKTAKKLENSAKKRISEGIAIAIQGGPR (cecropin P1) (SEQ ID NO: 357); ACYCRIPACIAGERRYGTCIYQGRLWAFCC (α-defensin) (SEQ ID NO: 358); DHYNCVSSGGQCLYSACPIFTKIQGTCYRGKAKCCK (β-defensin) (SEQ ID NO: 359); RRRPRPPYLPRPRPPPFFPPRLPPRIPPGFPPRFPPRFPGKR-NH2 (PR-39) (SEQ ID NO: 360); ILPWKWPWWPWRR-NH2 (indolicidin) (SEQ ID NO: 361); AAVALLPAVLLALLAP (RFGF) (SEQ ID NO: 362); AALLPVLLAAP (RFGF analogue) (SEQ ID NO: 363); and RKCRIVVIRVCR (bactenecin) (SEQ ID NO: 364).

Exemplary cationic groups include, but are not limited to, protonated amino groups, derived from e.g., O-AMINE (AMINE═NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE═NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)_{Q}CH₂CH₂-AMINE (AMINE═NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃(GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligonucleotides that comprise a number of phosphorothioate intersugar linkages are also known to bind to serum protein, thus short oligonucleotides, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the compound of the invention (e.g., a compound of the invention or linker). In some embodiments, the ligand can be incorporated via coupling to a “precursor” monomer after said “precursor” monomer has been incorporated into a component of the compound of the invention (e.g., a compound of the invention or linker). For example, a monomer having, e.g., an amino-terminated tether (i.e., having no associated ligand), e.g., monomer-linker-NH₂ can be incorporated into a component of the compounds of the invention (e.g., a compound of the invention or linker). In a subsequent operation, i.e., after incorporation of the precursor monomer into a component of the compounds of the invention (e.g., a compound of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the oligonucleotide. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Exemplary carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligonucleotides with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the oligonucleotide via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254, 469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; contents of which are herein incorporated in their entireties by reference.

In some embodiments, the oligonucleotide further comprises a targeting ligand that targets a liver tissue. In some embodiments, the targeting ligand is a carbohydrate-based ligand. In one embodiment, the targeting ligand is a GalNAc conjugate.

In some embodiments, the carbohydrate-based ligand is any one of the ligands listed in Table 2, Table 2A, Table 3, Table 3A, Table 4, or Table 4A of WO2015/006740, which is incorporated herein by reference in its entirety.

In some embodiments, the linkers including branched linkers such as a bivalent or trivalent branched linker for attaching these carbohydrate-based ligands include the linker(s) listed in Table 1 or Table 1A and the spacer(s) listed in Table 5 of WO2015/006740, which is incorporated herein by reference in its entirety.

In some embodiments, the GalNAc-based conjugate is a GalNAc analog containing a S or N atom, or a —CH₂— group in the glycosidic linkage to change a metagolically labile glycosidic linkage to a metabolically stable glycosidic linkage, e.g., having “O” in the glycosidic linkage being replaced by S or N atom, or a —CH₂— group, as shown in the scheme below.

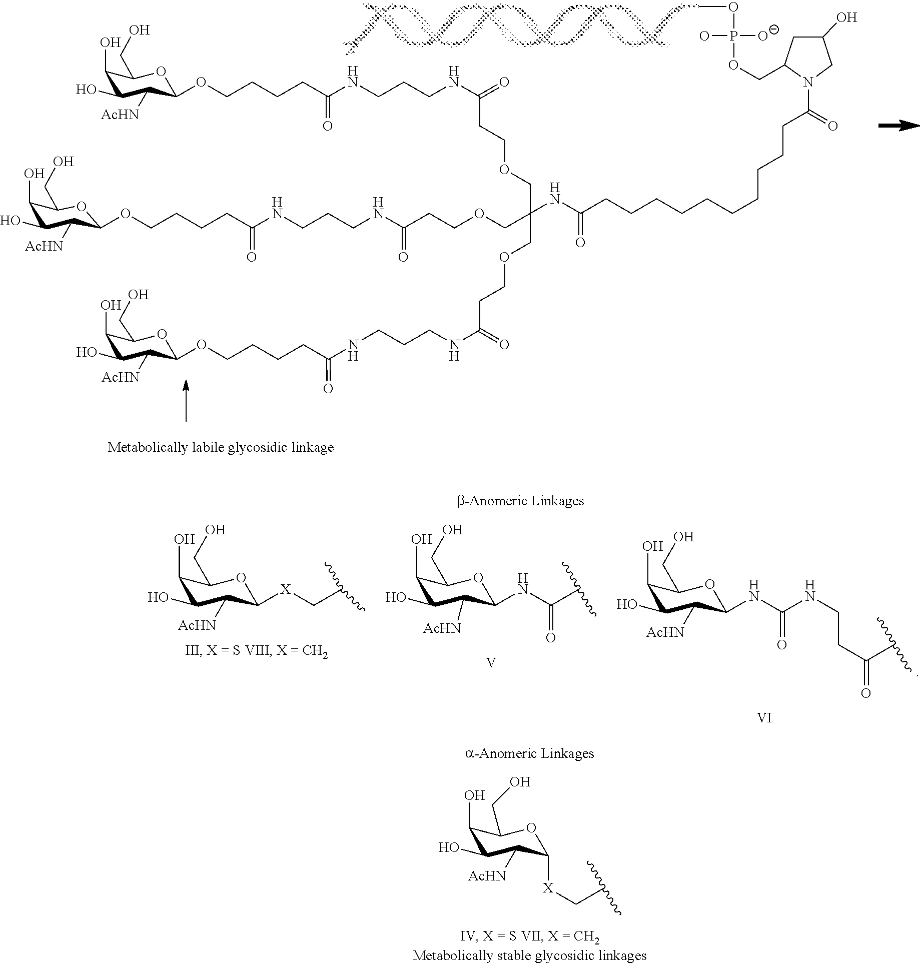

See the synthesis procedures of these GalNAc analog in Kandasamy et al., “Metabolically Stable Anomeric Linkages Containing GalNAc—siRNAConjugates: An Interplay among ASGPR, Glycosidase, and RISC Pathywas,” J. Med. Chem. 66:2506-23 (2023), which is incorporated by reference in its entirety.

In some embodiments, the GalNAc-based conjugate is a GalNAc analog having one of the following structures:

[chemical structure]

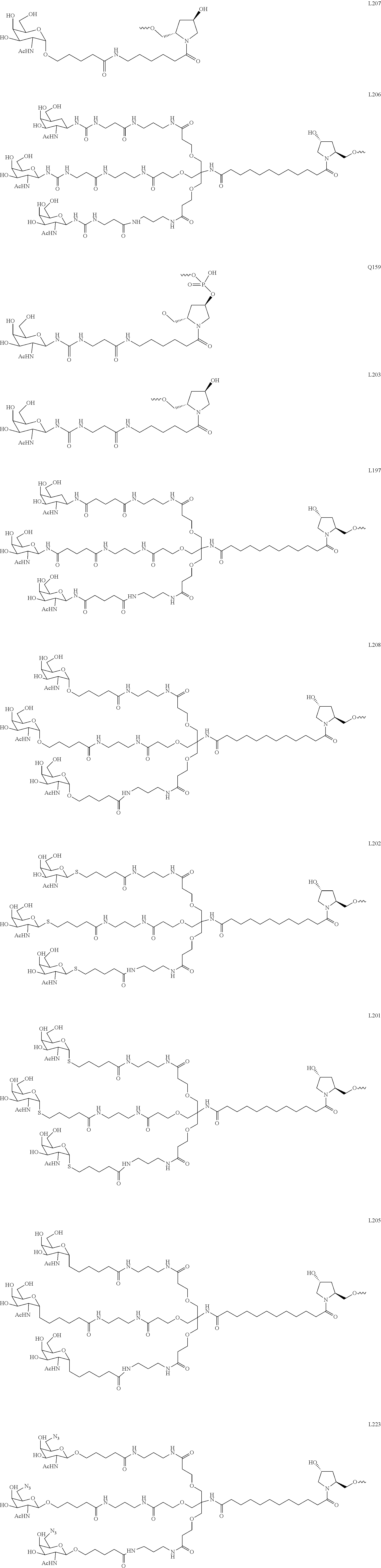

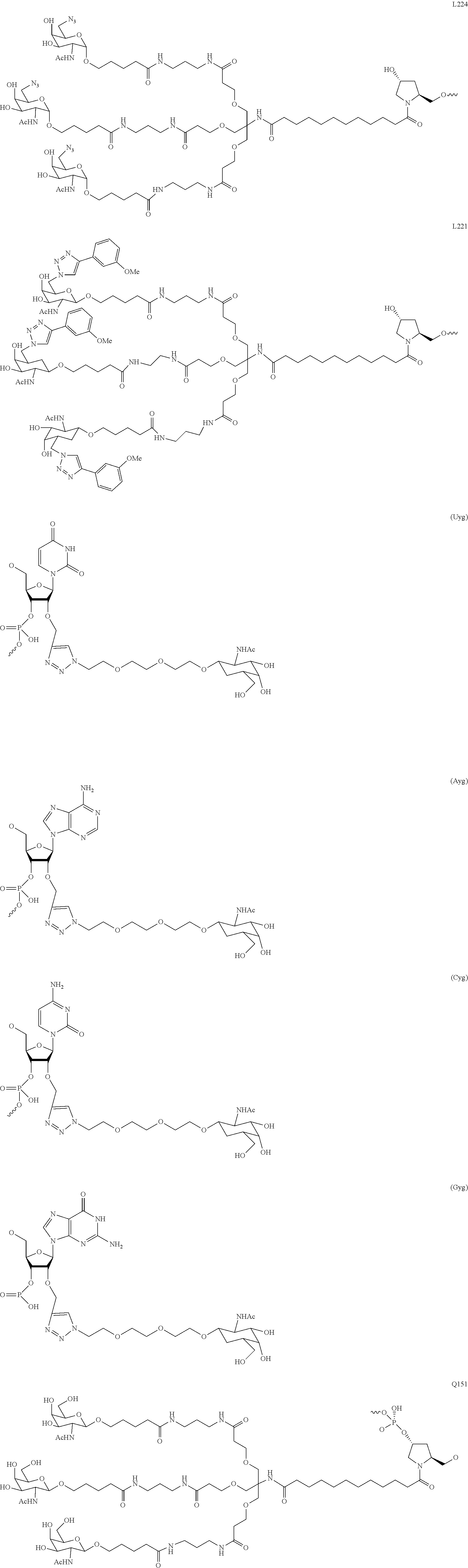

The GalNAc analogs listed in the above table may be prepared using the methods described in WO2015/006740, which is incorporated herein by reference in its entirety.

In some embodiments, the GalNAc-based conjugate is a GalNAc analog having one of the following structures:

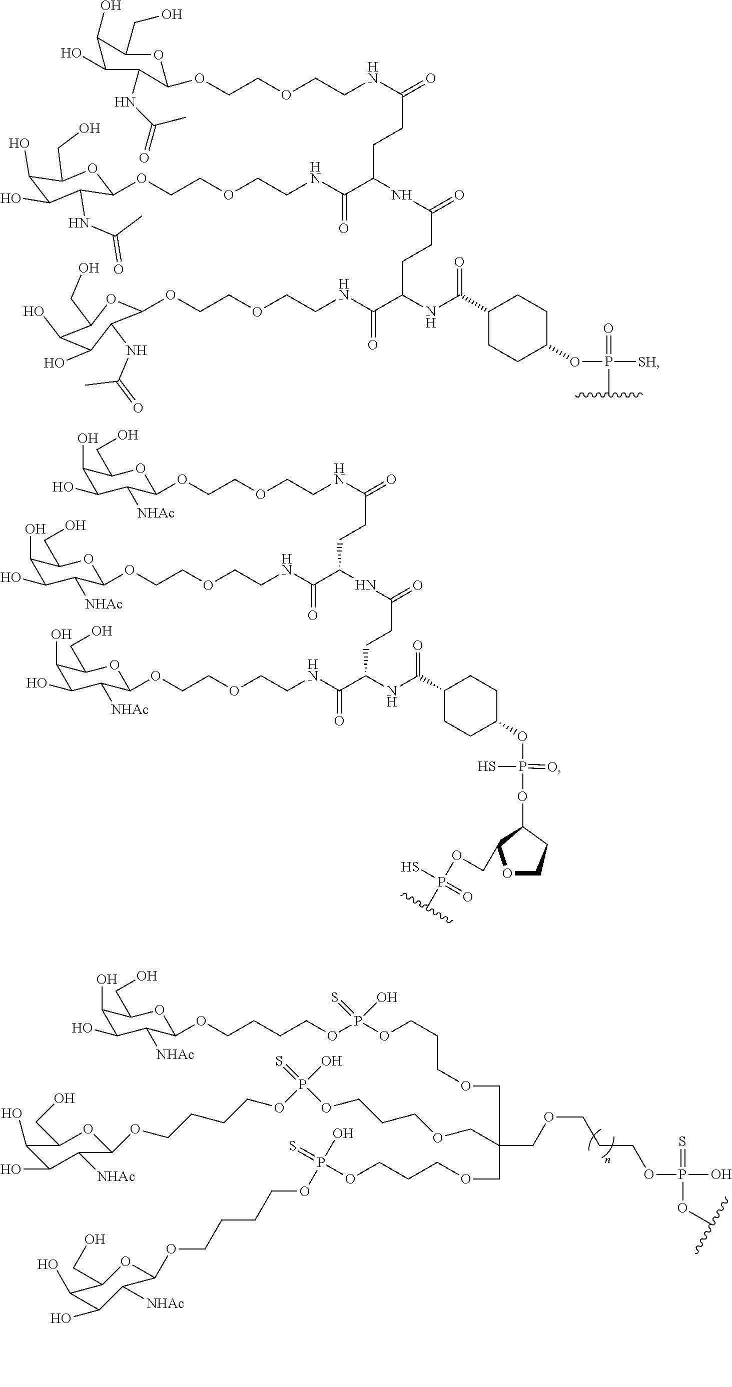

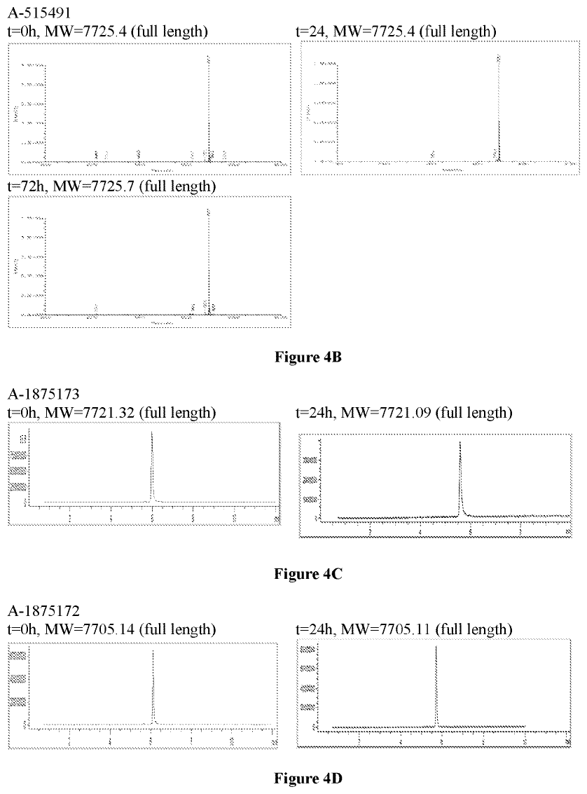

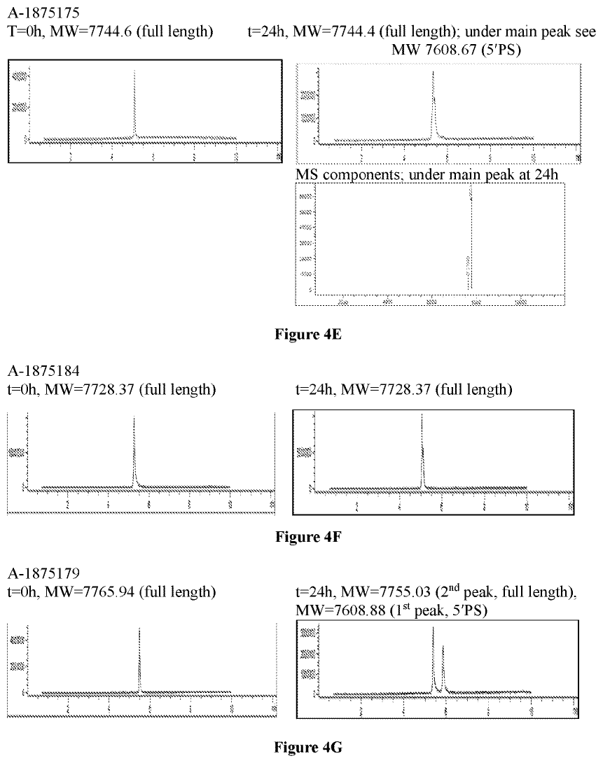

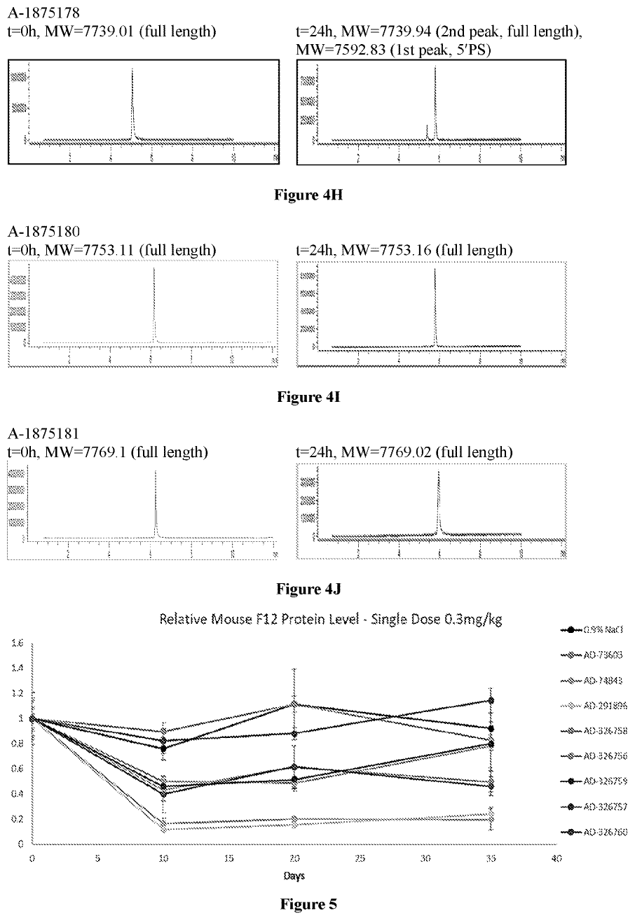

(wherein n=0-10 (e.g., 1 or 4). See FIGS. 4A and 4B of US2021/0123048A1, which is incorporated herein by reference in its entirety),

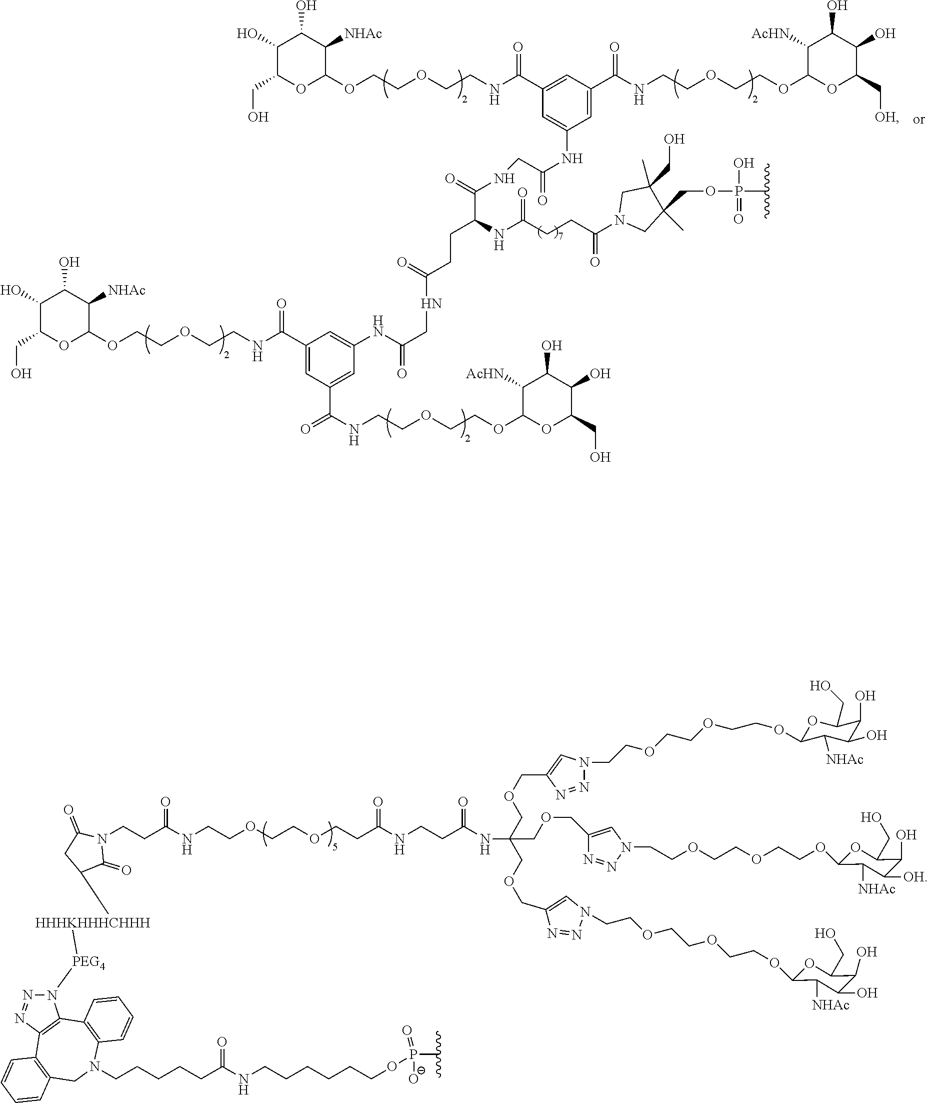

In certain embodiments, the oligonucleotide further comprises a ligand having a structure shown below:

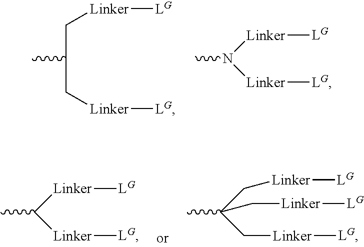

wherein:
L^{G} is independently for each occurrence a ligand, e.g., carbohydrate, e.g. monosaccharide, disaccharide, trisaccharide, tetrasaccharide, polysaccharide; and
Z′, Z″, Z′″ and Z″″ are each independently for each occurrence O or S.

In certain embodiments, the oligonucleotide comprises a ligand of Formula (II), (III), (IV) or (V):

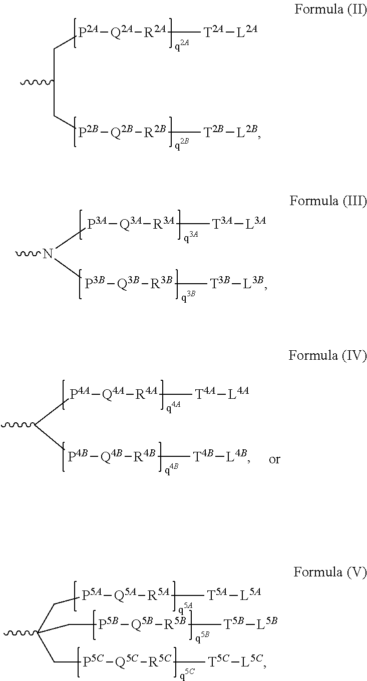

wherein:
q^{2A}, q^{2B}, q^{3A}, q^{3B}, q4^{A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);
T^{B} and T^{B′} are each independently for each occurrence absent, CO, NH, O, S, OC(O), OC(O)O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(Rₐ)C(O), —C(O)—CH(R^{a})—NH-, CO, CH═N-O,

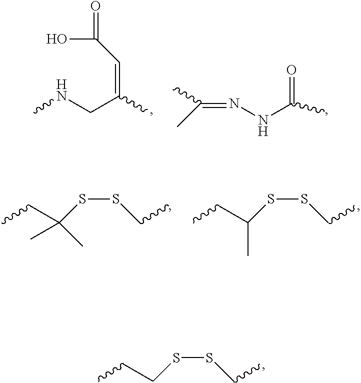

or heterocyclyl;
L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence O or S;
p represents independently for each occurrence 0-20.

As discussed above, because the ligand can be conjugated to the oligonucleotide via a linker or carrier, and because the linker or carrier can contain a branched linker, the oligonucleotide can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valences. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In certain embodiments, at least one ligand conjugated to the oligonucleotide is a transferrin receptor (TfR) ligand, such as a TfR1 ligand.

In certain embodiments, at least one ligand conjugated to the oligonucleotide is an integrin ligand (e.g., an integrin αvβ6 ligand, or an integrin αVβ3 ligand).

In some embodiments, the integrin ligand conjugated to the oligonucleotide is an integrin αVβ3 ligand. For instance, the integrin ligand is an integrin αVβ3 ligand having the structure of

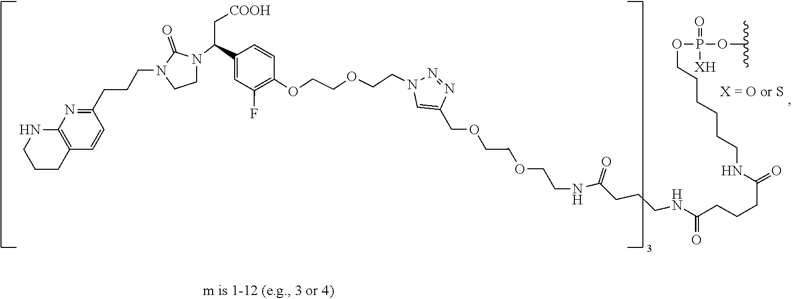

wherein [symbol] represents the bond to an oligonucleotide (e.g., the 5′-carbon at the 5′-end, the 3-carbon at the Y-end, or a 2′-carbon at one or more internal nucleotides. In one embodiment, the integrin ligand is an internal ligand variant having a structure of

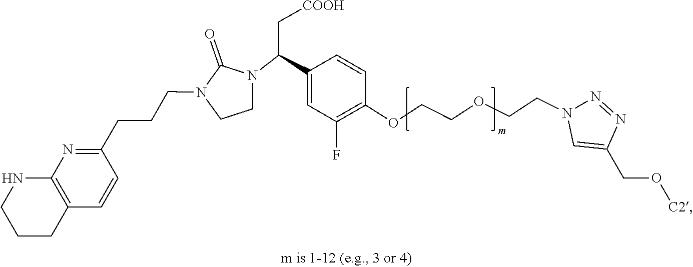

wherein C2′ is 2′-carbon of one or more internal nucleotides (e.g., 1, 2 or 3 internal nucleotides).

In some embodiments, the integrin ligand conjugated to the oligonucleotide is an integrin αVβ6 ligand.

In some embodiments, the integrin ligand conjugated to the oligonucleotide is an αvβ6 integrin ligand comprising:

R-G¹-D-L-Xaa¹-Xaa²-L-Xaa³-Xaa⁴-L-R¹ (Formula VIII) (SEQ ID NO: 365)
wherein:
R is L-arginine;
G¹ is L-glycine or N-methyl glycine;
D is L-aspartic acid (L-aspartate);
L is L-leucine;
Xaa¹ is L-alanine;
Xaa² is L-α-amino-butyric acid (Abu);
Xaa³ is L-citrulline (Cit);
Xaa⁴ is α-amino-isobutyric acid (Aib); and
R¹ is optional and, if present, includes polyethylene glycol and/or a linking group.

In some embodiments, in Formula VIII above, R¹ comprises a polyethylene glycol having 2-20 ethylene oxide units. In some embodiments, in Formula VIII above, the αvβ6 integrin ligand comprises the sequence of Ac-RGDLAAbuLCitAibL (SEQ ID NO:366).

In some embodiments, in Formula VIII above, the αvβ6 integrin ligand comprises an N-terminal cap. The N-terminal cap may be selected from the group consisting of: CH₃CO, CH₃CH₂CO, CH₃(CH₂)₂CO, (CH₃)₂CHCO, CH₃(CH₂)₃CO, (CH₃)₂CHCH₂CO, CH₃CH₂CH(CH₃)CO, (CH₃)₃CCO, CH₃(CH₂)₄CO, CH₃SO₂, CH₃CH₂SO₂, CH₃(CH₂)₂O₂, (CH₃)₂CHSO₂, CH₃(CH₂)₃SO₂, (CH₃)₂CHCH₂SO₂, CH₃CH₂CH(CH₃)SO₂, (CH₃)₃CSO₂, PhCO, PhSO₂, alkyl group having 1, 2, 3, 4, 5, 6, 7, 8, 9, or 10 carbon atoms, methyl, ethyl, propyl, butyl, pentyl, NH₂NH, PEG, guanidinyl, CH₃OCH₂CH₂OCH₂CH₂CO, CH₃O(CH₂CH₂O)₂CH₂CH₂CO, CH₃O(CH₂CH₂O)₃CH₂CH₂CO, CH₃O(CH₂CH₂O)₄CH₂CH₂CO, CH₃O(CH₂CH₂O)₅CH₂CH₂CO, CH₃OCH₂CH₂OCH₂CO, CH₃O(CH₂CH₂O)₂CH₂CO, CH₃O(CH₂CH₂O)₃CH₂CO, CH₃O(CH₂CH₂O)₄CH₂CO, CH₃O(CH₂CH₂O)₅CH₂CO, CH₃OCH₂CH₂OCO, CH₃O(CH₂CH₂O)₂CO, CH₃O(CH₂CH₂O)₃CO, CH₃O(CH₂CH₂O)₄CO, CH₃O(CH₂CH₂O)₅CO, HOCH₂CH₂OCH₂CH₂CO, HO(CH₂CH₂O)₂CH₂CH₂CO, HO(CH₂CH₂O)₃CH₂CH₂CO, HO(CH₂CH₂O)₄CH₂CH₂CO, HO(CH₂CH₂O)₅CH₂CH₂CO, HOCH₂CH₂OCH₂CO, HO(CH₂CH₂O)₂CH₂CO, HO(CH₂CH₂O)₃CH₂CO, HO(CH₂CH₂O)₄CH₂CO, HO(CH₂CH₂O)₅CH₂CO, HOCH₂CH₂OCO, HO(CH₂CH₂O)₂CO, HO(CH₂CH₂O)₃CO, HO(CH₂CH₂O)₄CO, HO(CH₂CH₂O)₅CO, CH₃CH₂OCH₂CH₂OCH₂CH₂CO, CH₃CH₂O(CH₂CH₂O)₂CH₂CH₂CO, CH₃CH₂O(CH₂CH₂O)₃CH₂CH₂CO, CH₃CH₂O(CH₂CH₂O)₄CH₂CH₂CO, CH₃CH₂O(CH₂CH₂O)₅CH₂CH₂CO, CH₃CH₂OCH₂CH₂OCH₂CO, CH₃CH₂O(CH₂CH₂O)₂CH₂CO, CH₃CH₂O(CH₂CH₂O)₃CH₂CO, CH₃CH₂O(CH₂CH₂O)₄CH₂CO, CH₃CH₂O(CH₂CH₂O)₅CH₂CO, CH₃CH₂OCH₂CH₂OCO, CH₃CH₂O(CH₂CH₂O)₂CO, CH₃CH₂O(CH₂CH₂O)₃CO, CH₃CH₂O(CH₂CH₂O)₄CO, CH₃CH₂O(CH₂CH₂O)₅CO, CH₃OCH₂CH₂CO, HOCH₂CH₂CO, and CH₃CH₂OCH₂CH₂CO. In one embodiment, the N-terminal cap is CH₃CO.

In one embodiment, the integrin ligand is an integrin αVβ6 ligand having the structure of

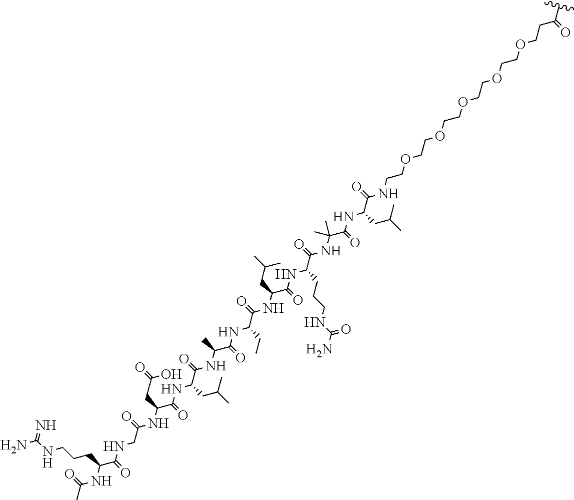 [SMILES: *C(=O)CCOCCOCCOCCOCCOCCNC(=O)[C@H](CC(C)C)NC(=O)C(C)(C)NC(=O)[C@H](CCCNC(N)=O)NC(=O)[C@H](CC(C)C)NC(=O)[C@H](CC)NC(=O)[C@H](C)NC(=O)[C@H](CC(C)C)NC(=O)[C@H](CC(=O)O)NC(=O)CNC(=O)[C@H](CCCNC(=N)N)NC(C)=O]

(SEQ ID NO: 367), wherein [symbol] represents the bond to an oligonucleotide (e.g., a 5′-C or a 5′-O at the 5′-end, a 3′-C or 3′-O at the 3′-end, or a 2′-C or 2′-O at one or more internal nucleotides). In one embodiment, the integrin ligand comprises Ac-Arg-Gly-Asp-Leu-Ala-Abu-Leu-Cit-Aib-Leu (SEQ ID NO: 366).

In one embodiment, the integrin ligand conjugated to the oligonucleotide is an αvβ36 integrin ligand comprising:

Ac-Arg-Gly-Asp-Leu-Ala-Abu-Leu-Cit-Aib-Leu-N(H)—[CH₂CH₂O]ₙ—CH₂CH₂C(O)—*, (SEQ ID NO: 367), wherein:
Ac is an acetyl group;
Abu is a-Aminobutyric acid (homoalanine);
Aib is 2-Aminoisobutyric acid (α-aminoisobutyric acid or 2-methylalanine);
Cit is citrulline;
n is 1-10 (e.g., 5); and
* represents the bond to an oligonucleotide (e.g., a 5′-C at the 5′-end, a 3′-C at the 3′-end, or a 2′-C at an internal nucleotide.

Additional embodiments of integrin αVβ6 ligands, particularly relating to the aforementioned Formula VIII, that can be conjugated to the oligonucleotide described herein include those described in U.S. Pat. No. 11,180,529, which is incorporated herein by reference in its entirety.

In some embodiments, the integrin ligand conjugated to the oligonucleotide is an αvβ6 integrin ligand comprising:

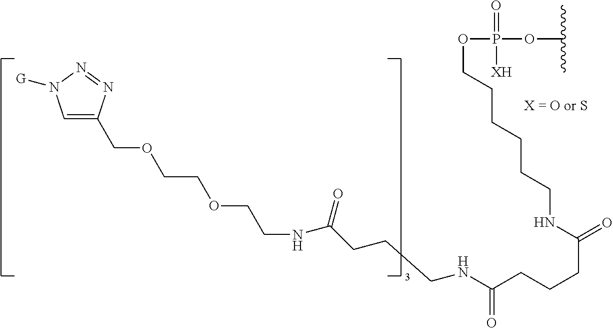

wherein each G may be selected from the group consisting of:

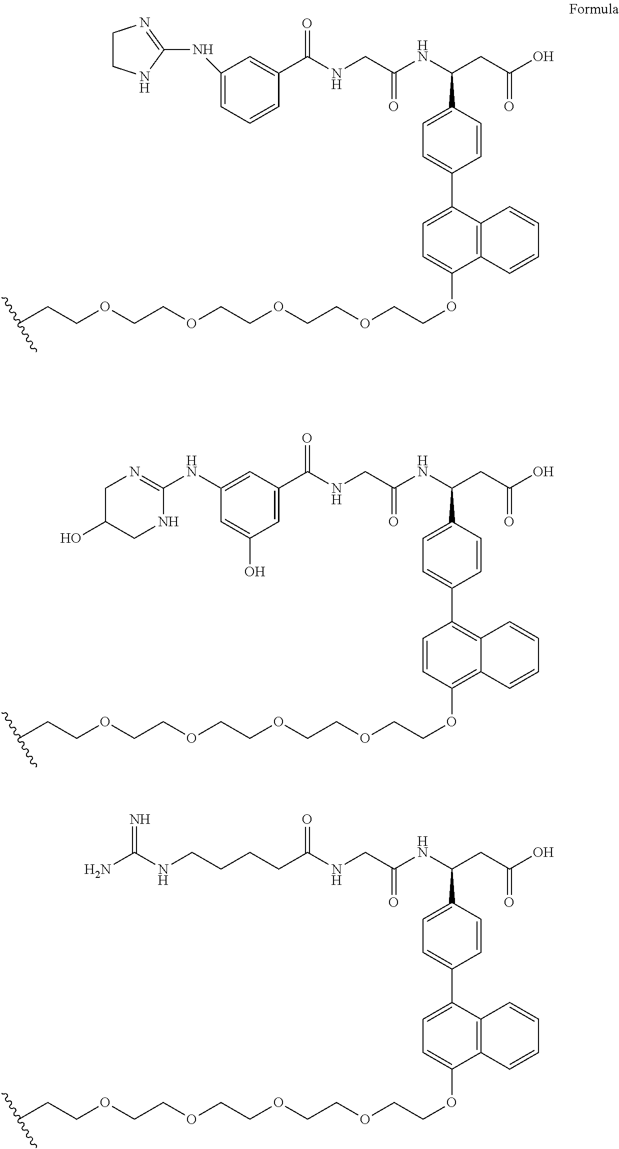 [SMILES: *CCOCCOCCOCCOCCOc1ccc(-c2ccc([C@H](CC(=O)O)NC(=O)CNC(=O)c3cccc(NC4=NCCN4)c3)cc2)c2ccccc12 ; *CCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12 ; *CCOCCOCCOCCOCCOc1ccc(-c2ccc([C@H](CC(=O)O)NC(=O)CNC(=O)CCCCNC(=N)N)cc2)c2ccccc12]

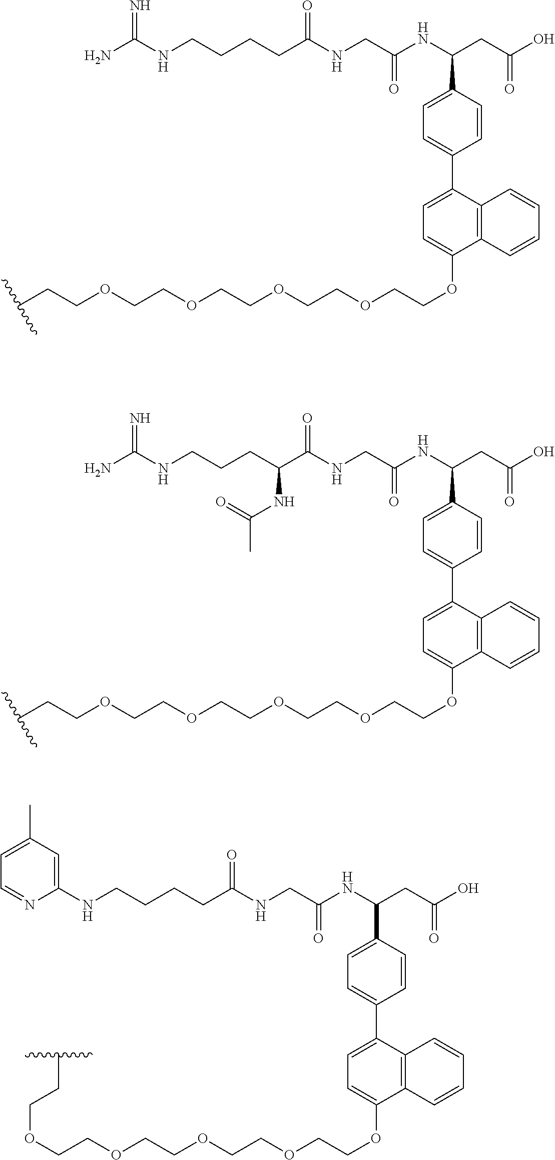 [SMILES: *CCOCCOCCOCCOCCOc1ccc(-c2ccc([C@H](CC(=O)O)NC(=O)CNC(=O)CCCCNC(=N)N)cc2)c2ccccc12 ; *CCOCCOCCOCCOCCOc1ccc(-c2ccc([C@H](CC(=O)O)NC(=O)CNC(=O)[C@H](CCCNC(=N)N)NC(C)=O)cc2)c2ccccc12 ; *CCOCCOCCOCCOCCOc1ccc(-c2ccc([C@H](CC(=O)O)NC(=O)CNC(=O)CCCCNc3cc(C)ccn3)cc2)c2ccccc12]

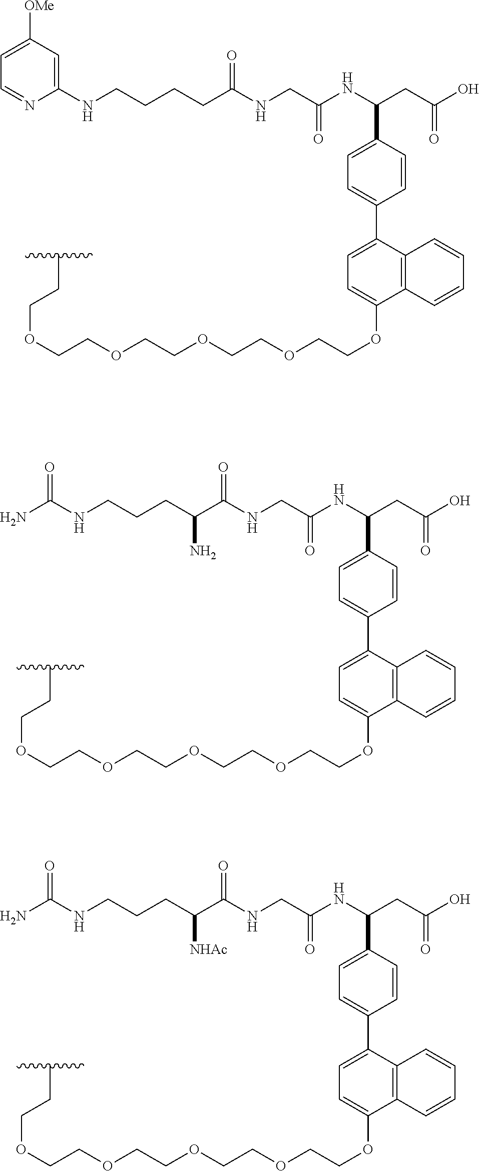 [SMILES: *CCOCCOCCOCCOCCOc1ccc(-c2ccc([C@H](CC(=O)O)NC(=O)CNC(=O)CCCCNc3cc(OC)ccn3)cc2)c2ccccc12 ; *CCOCCOCCOCCOCCOc1ccc(-c2ccc([C@H](CC(=O)O)NC(=O)CNC(=O)[C@@H](N)CCCNC(N)=O)cc2)c2ccccc12 ; *CCOCCOCCOCCOCCOc1ccc(-c2ccc([C@H](CC(=O)O)NC(=O)CNC(=O)[C@H](CCCNC(N)=O)NC(C)=O)cc2)c2ccccc12]

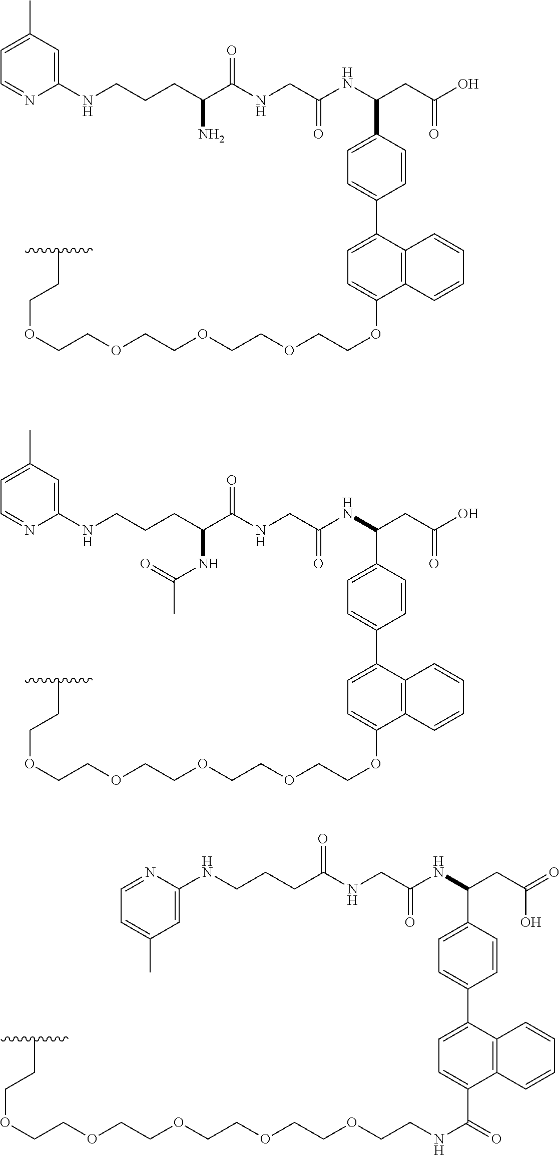

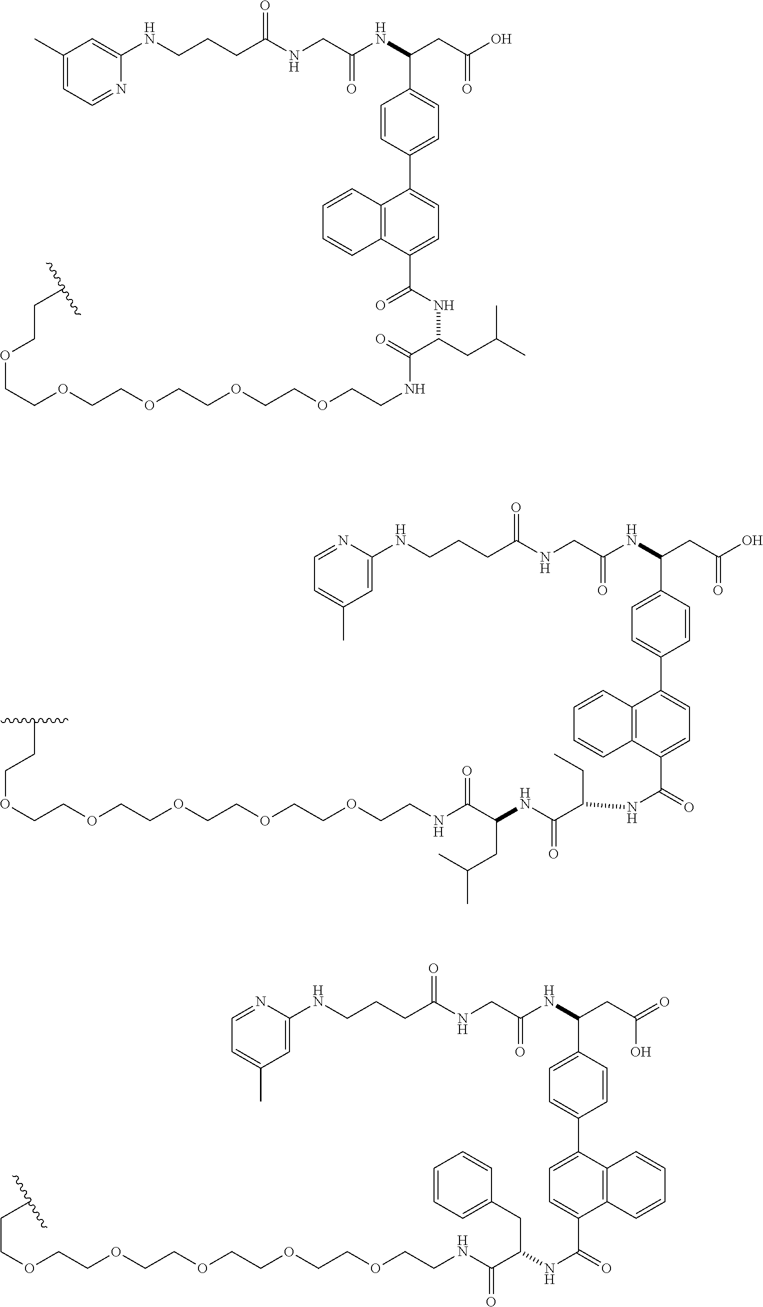

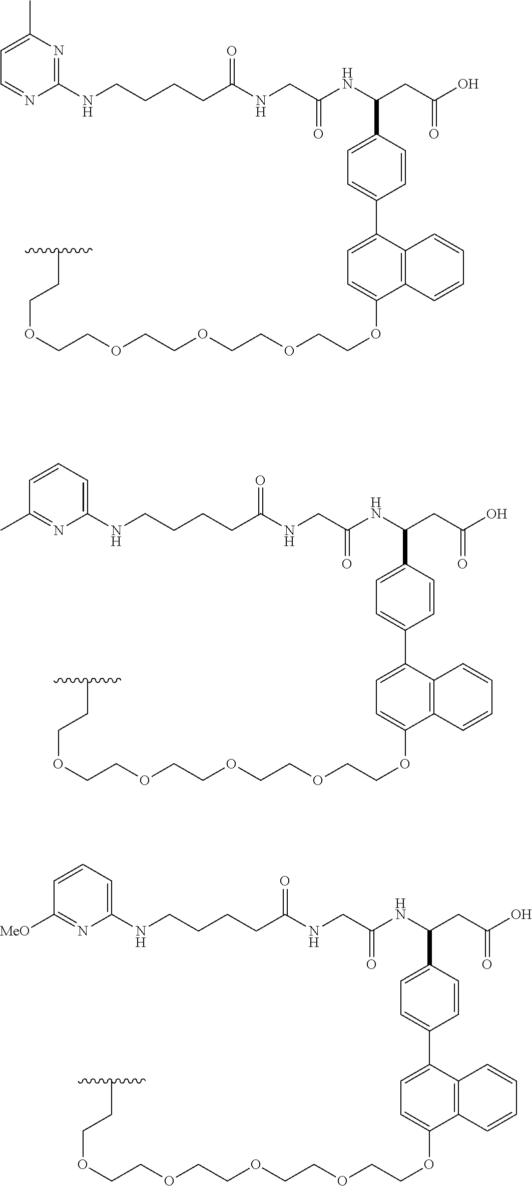 [SMILES: *CCOCCOCCOCCOCCOc1ccc(-c2ccc([C@H](CC(=O)O)NC(=O)CNC(=O)CCCCNc3nccc(C)n3)cc2)c2ccccc12 ; *CCOCCOCCOCCOCCOc1ccc(-c2ccc([C@H](CC(=O)O)NC(=O)CNC(=O)CCCCNc3cccc(C)n3)cc2)c2ccccc12 ; *CCOCCOCCOCCOCCOc1ccc(-c2ccc([C@H](CC(=O)O)NC(=O)CNC(=O)CCCCNc3cccc(OC)n3)cc2)c2ccccc12]

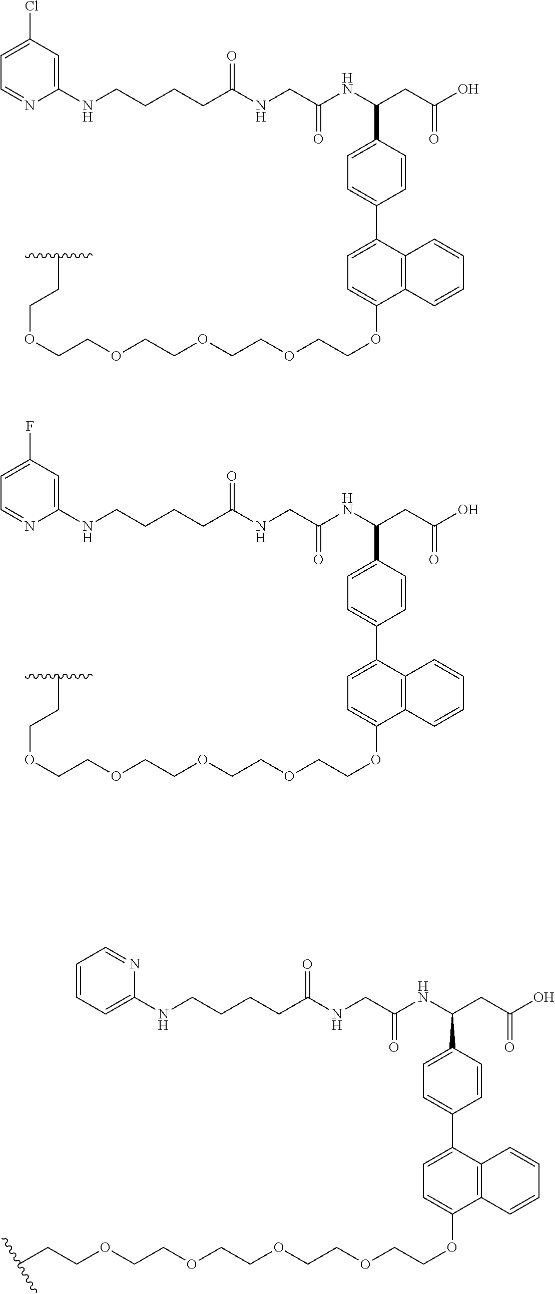 [SMILES: *CCOCCOCCOCCOCCOc1ccc(-c2ccc([C@H](CC(=O)O)NC(=O)CNC(=O)CCCCNc3cc(Cl)ccn3)cc2)c2ccccc12 ; *CCOCCOCCOCCOCCOc1ccc(-c2ccc([C@H](CC(=O)O)NC(=O)CNC(=O)CCCCNc3cc(F)ccn3)cc2)c2ccccc12 ; *CCOCCOCCOCCOCCOc1ccc(-c2ccc([C@H](CC(=O)O)NC(=O)CNC(=O)CCCCNc3ccccn3)cc2)c2ccccc12]

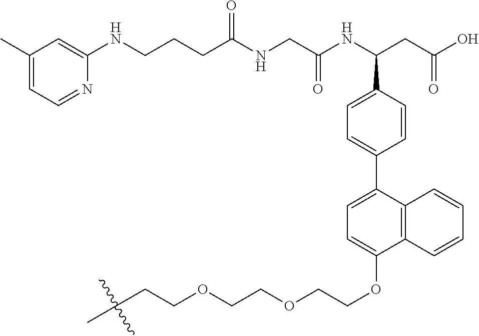 [SMILES: *CCOCCOCCOc1ccc(-c2ccc([C@H](CC(=O)O)NC(=O)CNC(=O)CCCNc3cc(C)ccn3)cc2)c2ccccc12]

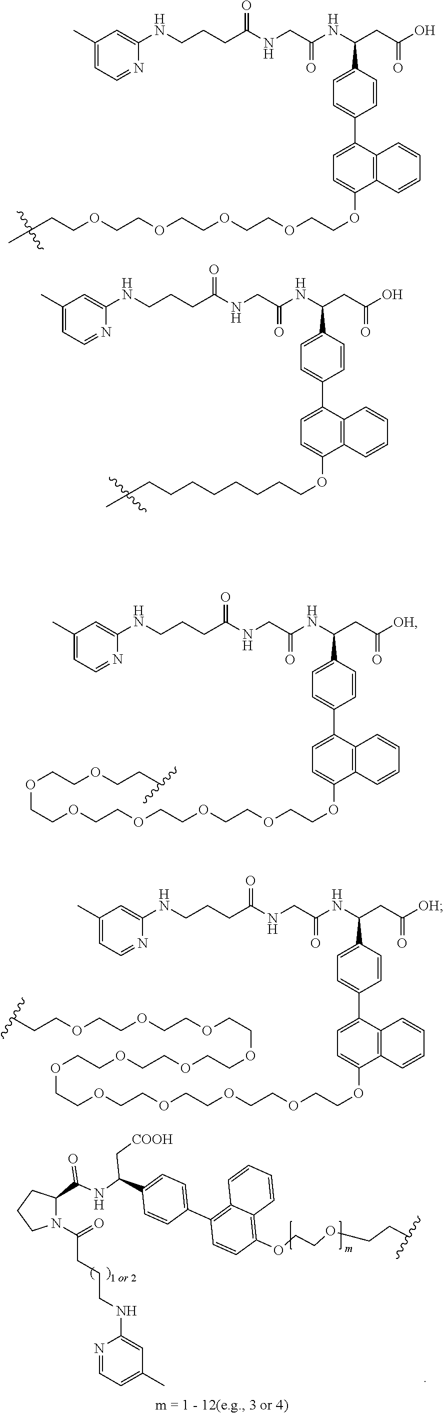

For example, the integrin ligand conjugated to the oligonucleotide described herein can have the structure of:

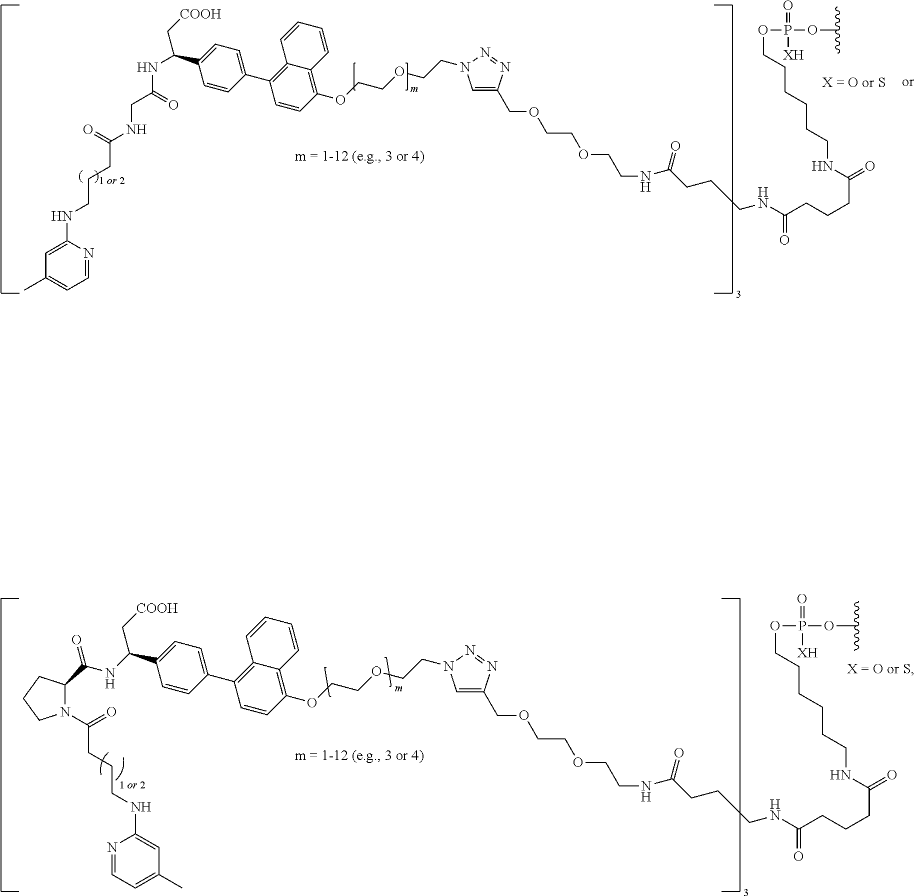

wherein [symbol] represents the bond to an oligonucleotide (e.g., a 5′-C at the 5′-end, a 3′-C at the 3′-end, or a 2′-C at one or more internal nucleotides.

The preceding can be prepared through azide-alkyne click chemistry between a alkyne-terminated compound of the formula

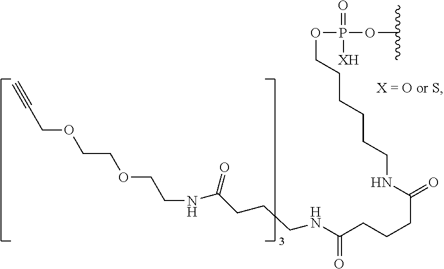

with an azide compound such as,

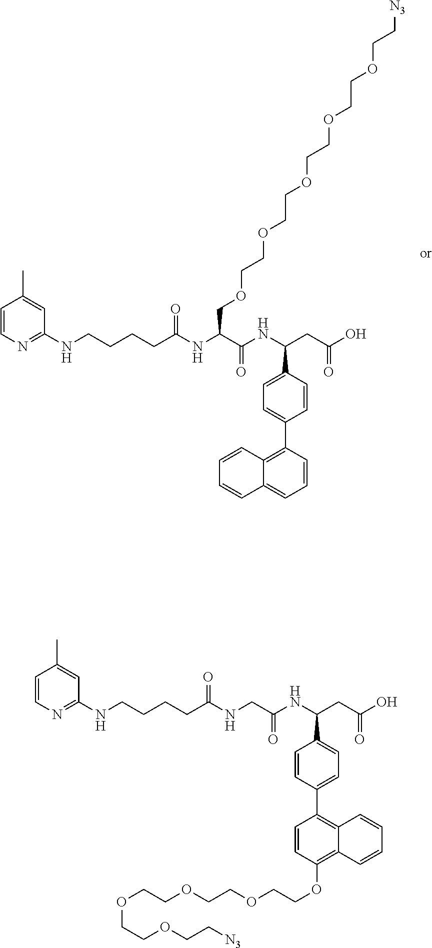 [SMILES: Cc1ccnc(NCCCCC(=O)N[C@@H](COCCOCCOCCOCCOCCN=[N+]=[N-])C(=O)N[C@@H](CC(=O)O)c2ccc(-c3cccc4ccccc34)cc2)c1 ; Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

Additional embodiments of integrin αVβ6 ligands that can be conjugated to the oligonucleotide described herein include those described in PCT Publication No. WO 2022/056266, which is incorporated herein by reference in its entirety.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradant can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNAs.

In an alternative functional assay, a candidate dssiRNA homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase IL, can be monitored as an indicator that the agent is inhibiting expression. For example, cleavage of c-mos mRNA by a dssiRNA would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The siRNAs described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA could be complementary to sequences within homologous genes, e.g., oncogenes or tumor suppressor genes, of the non-human mammal and the human. By determining the toxicity of the siRNA in the non-human mammal, one can extrapolate the toxicity of the siRNA in a human. For a more strenuous toxicity test, the siRNA can be complementary to a human and more than one, e.g., two or three or more, non-human animals.

The methods described herein can be used to correlate any physiological effect of an siRNA on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA and a drug that affects the uptake of the siRNA into the cell. The drug can be administered before, after, or at the same time that the siRNA is administered. The drug can be covalently or non-covalently linked to the siRNA. The drug can be, for example, a lipopolysaccharide, an activator of p38 MAP kinase, or an activator of NF-κB. The drug can have a transient effect on the cell. The drug can increase the uptake of the siRNA into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

siRNA Production

An siRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilot II reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove endsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9; and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

Making iRNA Agents Conjugated to a Targeting Ligand

In some embodiments, the targeting ligand conjugated to the iRNA agent via a nucleobase, sugar moiety, or internucleosidic linkage.

Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a targeting ligand is conjugated to a nucleobase, the preferred position is one that does not interfere with hybridization, i.e., does not interfere with the hydrogen bonding interactions needed for base pairing. In one embodiment, the targeting ligand may be conjugated to a nucleobase via a linker containing an alkyl, alkenyl or amide linkage.

Conjugation to sugar moieties of nucleosides can occur at any carbon atom. Exemplary carbon atoms of a sugar moiety that a targeting ligand can be attached to include the 2′, 3′, and 5′ carbon atoms. A targeting ligand can also be attached to the 1′ position, such as in an abasic residue. In one embodiment, the targeting ligand may be conjugated to a sugar moiety, via a 2′-O modification, with or without a linker.

Internucleosidic linkages can also bear targeting ligands. For phosphorus-containing linkages (e.g., phosphodiester, phosphorothioate, phosphorodithioate, phosphoroamidate, and the like), the targeting ligand can be attached directly to the phosphorus atom or to an O, N, or S atom bound to the phosphorus atom. For amine- or amide-containing internucleosidic linkages (e.g., PNA), the targeting ligand can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligonucleotides with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant targeting ligand, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, a targeting ligand having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the targeting ligand has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the targeting ligand having the phosphoramidite group may include phosphitylation of a free hydroxyl to generate the phosphoramidite group.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif.), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### Pharmaceutical Compositions

In one aspect, the invention features a pharmaceutical composition that includes an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) including a nucleotide sequence complementary to a target RNA, e.g., substantially and/or exactly complementary. The target RNA can be a transcript of an endogenous human gene. In one embodiment, the siRNA (a) is 19-25 nucleotides long, for example, 21-23 nucleotides, (b) is complementary to an endogenous target RNA, and, optionally, (c) includes at least one 3′ overhang 1-5 nt long. In one embodiment, the pharmaceutical composition can be an emulsion, microemulsion, cream, jelly, or liposome.

In one example the pharmaceutical composition includes an iRNA (an siRNA) mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes an iRNA (an siRNA), e.g., a double-stranded siRNA, or ssiRNA (e.g., a precursor, e.g., a larger siRNA which can be processed into a ssiRNA, or a DNA which encodes an siRNA, e.g., a double-stranded siRNA, or ssiRNA, or precursor thereof) admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a form suitable for oral delivery. In one embodiment, oral delivery can be used to deliver an siRNA composition to a cell or a region of the gastro-intestinal tract, e.g., small intestine, colon (e.g., to treat a colon cancer), and so forth. The oral delivery form can be tablets, capsules or gel capsules. In one embodiment, the siRNA of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an iRNA (an siRNA) and a delivery vehicle. In one embodiment, the siRNA is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA, e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a rectal dosage form. In one embodiment, the rectal dosage form is an enema. In another embodiment, the rectal dosage form is a suppository.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a vaginal dosage form. In one embodiment, the vaginal dosage form is a suppository. In another embodiment, the vaginal dosage form is a foam, cream, or gel.

In one aspect, the invention features a pharmaceutical composition including an iRNA (an siRNA), e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) in a pulmonary or nasal dosage form. In one embodiment, the siRNA is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell, comprising contacting said cell with the oligonucleotide. In one embodiment, the cell is an extrahepatic cell.

Another aspect of the invention relates to a method of reducing the expression of a target gene in a subject, comprising administering to the subject the oligonucleotide.

Another aspect of the invention relates to a method of treating a subject having a CNS disorder, comprising administering to the subject a therapeutically effective amount of the double-stranded iRNA agent of the invention, thereby treating the subject. Exemplary CNS disorders that can be treated by the method of the invention include Alzheimer, amyotrophic lateral sclerosis (ALS), frontotemporal dementia, Huntington, Parkinson, spinocerebellar, prion, and lafora.

The oligonucleotide can be delivered to a subject by a variety of routes, depending on the type of genes targeted and the type of disorders to be treated. In some embodiments, the oligonucleotide is administered extrahepatically, such as an ocular administration (e.g., intravitreal administration) or an intrathecal or intracerebroventricular administration.

In one embodiment, the oligonucleotide is administered intrathecally or intracerebroventricularly. By intrathecal or intracerebroventricular administration of the double-stranded iRNA agent, the method can reduce the expression of a target gene in a brain or spine tissue, for instance, cortex, cerebellum, cervical spine, lumbar spine, and thoracic spine.

In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, and TTR. To reduce the expression of these target genes in the subject, the oligonucleotide can be administered to the eye(s) directly (e.g., intravitreally). By intravitreal administration of the double-stranded iRNA agent, the method can reduce the expression of the target gene in an ocular tissue.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. A composition that includes a iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular or intracerebroventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular or intracerebroventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

In one embodiment, the administration of the iRNA (siRNA), e.g., a double-stranded siRNA, or ssiRNA, composition is parenteral, e.g., intravenous (e.g., as a bolus or as a diffusible infusion), intradermal, intraperitoneal, intramuscular, intrathecal, intraventricular, intracerebroventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the is delivered by intrathecal injection (i.e. injection into the spinal fluid which bathes the brain and spinal cord tissue). Intrathecal injection of iRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally or intracerebroventricularly injected iRNA agents may vary from one target gene to another target gene and the appropriate amount that has to be applied may have to be determined individually for each target gene. Typically, this amount ranges between 10 μg to 2 mg, preferably 50 μg to 1500 μg, more preferably 100 μg to 1000 μg.

Rectal Administration. The invention also provides methods, compositions, and kits, for rectal administration or delivery of siRNAs described herein.

Accordingly, an iRNA (an siRNA), e.g., a double-stranded siRNA, or ssiRNA, (e.g., a precursor, e.g., a larger siRNA which can be processed into a ssiRNA, or a DNA which encodes a an siRNA, e.g., a double-stranded siRNA, or ssiRNA, or precursor thereof) described herein, e.g., a therapeutically effective amount of a siRNA described herein, e.g., a siRNA having a double stranded region of less than 40, and, for example, less than 30 nucleotides and having one or two 1-3 nucleotide single strand 3′ overhangs can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the siRNA is by means of an enema. The siRNA of the enema can be dissolved in a saline or buffered solution. The rectal administration can also by means of a suppository, which can include other ingredients, e.g., an excipient, e.g., cocoa butter or hydropropylmethylcellulose.

Ocular Delivery. The iRNA agents described herein can be administered to an ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

In certain embodiments, the double-stranded iRNA agents may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the double-stranded iRNA agents may be administered into the eye, for example the vitreous chamber of the eye, by intravitreal injection, such as with pre-filled syringes in ready-to-inject form for use by medical personnel.

For ophthalmic delivery, the double-stranded iRNA agents may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the double-stranded iRNA agents. Viscosity building agents, such as hydroxymethyl cellulose, hydroxyethyl cellulose, methylcellulose, polyvinylpyrrolidone, or the like may be added to the pharmaceutical compositions to improve the retention of the double-stranded iRNA agents.

To prepare a sterile ophthalmic ointment formulation, the double-stranded iRNA agents is combined with a preservative in an appropriate vehicle, such as mineral oil, liquid lanolin, or white petrolatum. Sterile ophthalmic gel formulations may be prepared by suspending the double-stranded iRNA agents in a hydrophilic base prepared from the combination of, for example, CARBOPOL®-940 (BF Goodrich, Charlotte, N.C.), or the like, according to methods known in the art.

Topical Delivery. Any of the siRNAs described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the siRNA composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. In some embodiments, an siRNA, e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal. Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

Pulmonary Delivery. Any of the siRNAs described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication. Pulmonary delivery is effective not only for disorders which directly affect pulmonary tissue, but also for disorders which affect other tissue. siRNAs can be formulated as a liquid or nonliquid, e.g., a powder, crystal, or aerosol for pulmonary delivery.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. A composition that includes an siRNA, e.g., a double-stranded siRNA, or ssiRNA, (e.g., a precursor, e.g., a larger siRNA which can be processed into a ssiRNA, or a DNA which encodes an siRNA, e.g., a double-stranded siRNA, or ssiRNA, or precursor thereof) can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 μm. Usually the particle size distribution is between about 0.1 μm and about 5 μm in diameter, sometimes about 0.3 μm to about 5 μm.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral or Nasal Delivery. Any of the siRNAs described herein can be administered orally, e.g., in the form of tablets, capsules, gel capsules, lozenges, troches or liquid syrups. Further, the composition can be applied topically to a surface of the oral cavity.

Any of the siRNAs described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue siRNAs can be formulated as a liquid or nonliquid, e.g., a powder, crystal, or for nasal delivery. As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

For ease of exposition the formulations, compositions and methods in this section are discussed largely with regard to modified siRNAs. It may be understood, however, that these formulations, compositions and methods can be practiced with other siRNAs, e.g., unmodified siRNAs, and such practice is within the invention. Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of iRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

An aspect of the invention also relates to a method of delivering an oligonucleotide into the CNS by intrathecal or intracerebroventricular delivery, or into an ocular tissue by ocular delivery, e.g., an intravitreal delivery.

Some embodiments relates to a method of reducing the expression of a target gene in a subject, comprising administering to the subject the oligonucleotide described herein. In one embodiment, the oligonucleotide is administered intrathecally or intracerebroventricularly (to reduce the expression of a target gene in a brain or spine tissue). In one embodiment, the oligonucleotide is administered ocularly, e.g., intravitreally, (to reduce the expression of a target gene in an ocular tissue).

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention and are not intended to limit the invention.

TABLE I
Abbreviations of nucleotide monomers used in nucleic acid sequence representation.
It will be understood that these monomers, when present in an oligonucleotide, are mutually
linked by 5′-3′-phosphodiester bonds; and it is understood that when the nucleotide contains a
2′-fluoro modification, then the fluoro replaces the hydroxy at that position in the parent
nucleotide (i.e., it is a 2′-deoxy-2′-fluoronucleotide). The abbreviations are understood to
omit the 3′-phosphate (i.e. they are 3′-OH) when placed at the 3′-terminal position of an
oligonucleotide

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine -3′-phosphorothioate |
| Us | uridine -3′-phosphorothioate |
| N | any nucleotide, modified or unmodified |
| a | 2′-O-methyladenosine-3′-phosphate |
| as | 2′-O-methyladenosine-3′- phosphorothioate |
| c | 2′-O-methylcytidine-3′-phosphate |
| cs | 2′-O-methylcytidine-3′ - phosphorothioate |
| g | 2′-O-methylguanosine-3′-phosphate |
| gs | 2′-O-methylguanosine-3′- phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| L96 | N-[tris(GalNAc-alkyl)-amidodecanoyl)]-4-hydroxyprolinol |
| | (Hyp-(GalNAc-alkyl)3) |
| | 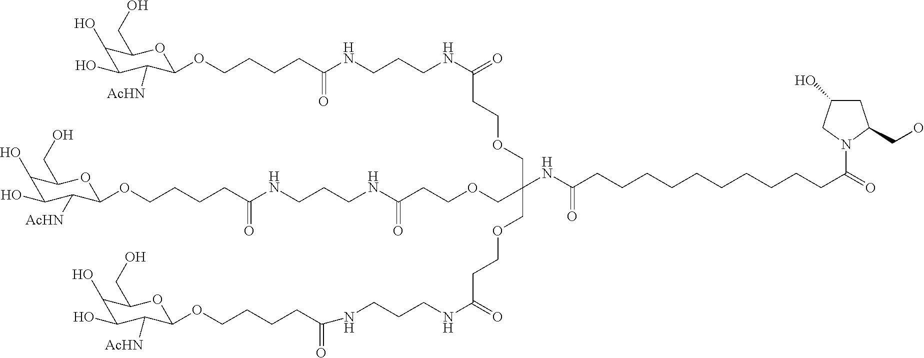 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| | (2S,4R)-1-[29-[[2-(acetylamino)-2-deoxy-β-D-galactopyranosyl]oxy]- |
| | 14,14-bis[[3-[[3-[5-[[2-(acetylamino)-2-deoxy-β-D- |
| | galactopyranosyl]oxy]-1-oxopentyl]amino]propyl]amino]-3- |
| | oxopropoxy]methyl]-1,12,19,25-tetraoxo-16-oxa-13,20,24- |
| | triazanonacos-1-yl]-4-hydroxy-2-hydroxymethylpyrrolidine |
| aL96 | 3′-terminal L-96 modified nucleotide (L96 modified a, u, c, g, or t, |
| uL96 | respectively at a 3′-terminal. E.g., uL96: |
| cL96 | 2′-O-methyluridine-3′-phosphate ((2S,4R)-1-[29-[[2-(acetylamino)-2- |
| gL96 | deoxy-B-D-galactopyranosyl]oxy]-14,14-bis [3-[[3-[5-[2- |
| tL96 | (acetylamino)-2-deoxy-β-D-galactopyranosyl]oxy]-1- |
| | oxopentyl]amino]propyl]amino]-3-oxopropoxy]methyl]-1,12,19,25- |
| | tetraoxo-16-oxa-13,20,24-triazanonacos-1-yl]-4-hydroxy-2- |
| | pyrrolidinyl)methyl ester |
| | 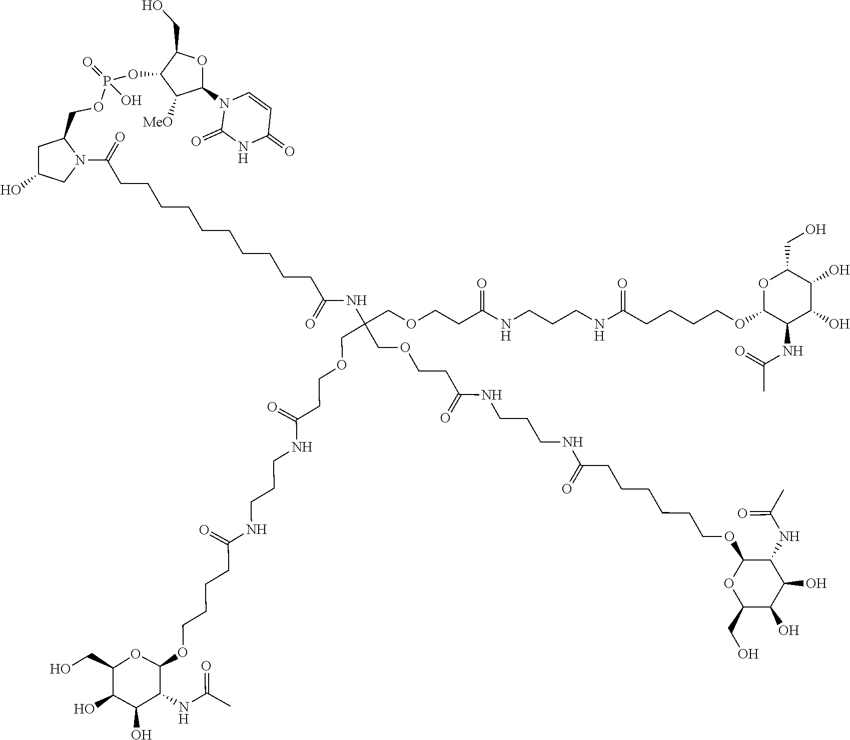 [SMILES: CO[C@@H]1[C@H](OP(=O)(O)OC[C@@H]2C[C@@H](O)CN2C(=O)CCCCCCCCCCC(=O)NC(COCCC(=O)NCCCNC(=O)CCCCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)[C@@H](CO)O[C@H]1n1ccc(=O)[nH]c1=O] |
| Y34 | 2-hydroxymethyl-tetrahydrofurane-4-methoxy-3-phosphate (abasic 2′- |
| | OMe furanose) |
| | 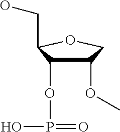 |
| Y44 | inverted abasic DNA (2-hydroxymethyl-tetrahydrofurane-5-phosphate) |
| | 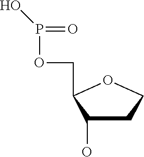 |
| L10 | N-(cholesterylcarboxamidocaproyl)-4-hydroxyprolinol (Hyp-C6-Chol) |
| | 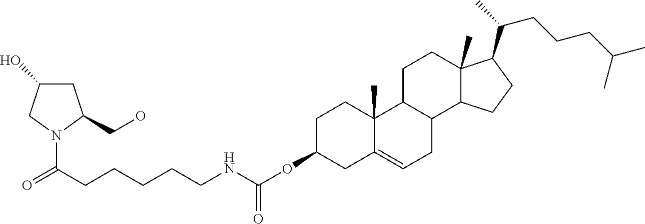 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3 -phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3 -phosphorothioate |
| dT | 2′-deoxythymidine-3′-phosphate |
| dTs | 2′-deoxythymidine-3 -phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′ -phosphate |
| (A2p) | adenosine-2′-phosphate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |
| s | Phosphorothioate |

TABLE II
Abbreviations of the cyclic disulfide modified phosphate prodrugs

| Abbv. | Structure | Name (as a 5′-modification) |
|---|---|---|
| (PdArlcbs) | 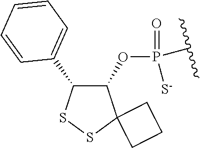 [SMILES: *P(=O)([S-])O[C@H]1[C@@H](c2ccccc2)SSC12CCC2] | 5′-(cis-3-phenyl-1,2-dithiaspiro[4.3] octan-4-yl thiophosphate) |
| | (+/−) cis | |
| (PdAr1chs) | 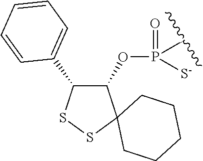 | 5′-(cis-3-phenyl-1,2-dithiaspiro[4.5] decan-4-yl thiophosphate) |
| | (+/−) cis | |
| (PdArlcps) | 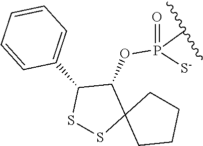 [SMILES: *P(=O)([S-])O[C@H]1[C@@H](c2ccccc2)SSC12CCCC2] | 5′-(cis-3-phenyl-1,2-dithiaspiro[4.4] nonan-4-ylthiophosphate) |
| | (+/−) cis | |
| (PdArlpn1) | 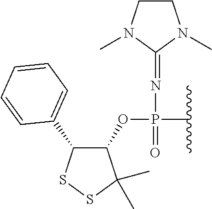 [SMILES: *P(=O)(N=C1N(C)CCN1C)O[C@H]1[C@@H](c2ccccc2)SSC1(C)C] | 5′-((cis-3,3-dimethyl-5-phenyl-1,2- dithiolan-4-yl) (1,3- dimethylimidazolidin-2- ylidene)phosphoramidate) |
| | (+/−) cis | |
| (PdArlpn2) | 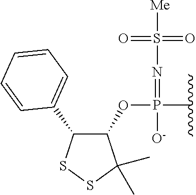 [SMILES: *P([O-])(=NS(C)(=O)=O)O[C@H]1[C@@H](c2ccccc2)SSC1(C)C] | 5′-(cis-5-phenyl-3,3-dimethyl-1,2- dithiolan-4-yl (N-methylsulfonyl) phosphimidate) |
| | (+/−) cis | |
| (PdArls) | 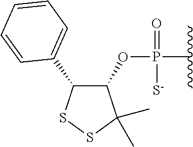 [SMILES: *P(=O)([S-])O[C@H]1[C@@H](c2ccccc2)SSC1(C)C] | 5′-(cis-5-phenyl-3,3-dimethyl-1,2- dithiolan-4-yl thiophosphate) |
| | (+/−) cis | |
| (PdAr2s) | 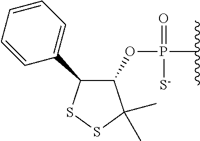 [SMILES: *P(=O)([S-])O[C@H]1[C@H](c2ccccc2)SSC1(C)C] | 5′-(trans-5-phenyl-3,3-dimethyl-1,2- dithiolan-4-yl thiophosphate) |
| | (+/−) trans | |
| (PdAr3s) | 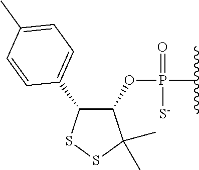 [SMILES: *P(=O)([S-])O[C@H]1[C@@H](c2ccc(C)cc2)SSC1(C)C] | 5′-(cis-5-(4-methylphenyl)-3,3-dimethyl- 1,2-dithiolan-4-yl thiophosphate) |
| | (+/−) cis | |
| (PdAr4s) | 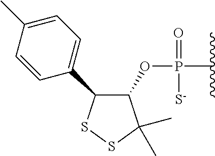 [SMILES: *P(=O)([S-])O[C@H]1[C@H](c2ccc(C)cc2)SSC1(C)C] | 5′-(trans-5-(4-methylphenyl)-3,3-dimethyl- 1,2-dithiolan-4-yl thiophosphate) |
| | (+/−) trans) | |
| (PdAr5s) | 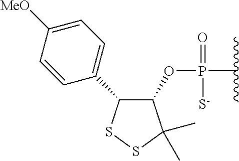 [SMILES: *P(=O)([S-])O[C@H]1[C@@H](c2ccc(OC)cc2)SSC1(C)C] | 5′-(cis-5-(4-methoxyphenyl)-3,3-dimethyl- 1,2-dithiolan-4-yl thiophosphate) |
| | (+/−) cis | |
| (PdAr6s) | 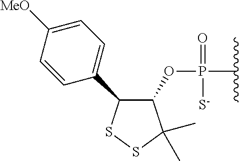 [SMILES: *P(=O)([S-])O[C@H]1[C@H](c2ccc(OC)cc2)SSC1(C)C] | 5′-(trans-5-(4-methoxyphenyl)-3,3- dimethyl-1,2-dithiolan-4-yl thiophosphate) |
| | (+/−) trans | |
| (PdAr7s) | 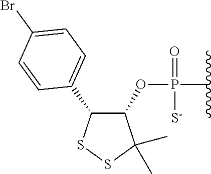 [SMILES: *P(=O)([S-])O[C@H]1[C@@H](c2ccc(Br)cc2)SSC1(C)C] | 5′-(cis-5-(4-bromophenyl)-3,3-dimethyl- 1,2-dithiolan-4-yl thiophosphate) |
| | (+/−) cis | |
| (PdAr9s) | 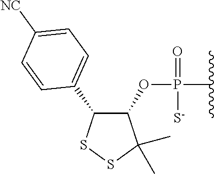 [SMILES: *P(=O)([S-])O[C@H]1[C@@H](c2ccc(C#N)cc2)SSC1(C)C] | 5′-(cis-5-(4-cyanophenyl)-3,3-dimethyl- 1,2-dithiolan-4-yl thiophosphate) |
| | (+/−) cis | |
| (PdAr11s) | 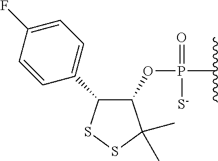 [SMILES: *P(=O)([S-])O[C@H]1[C@@H](c2ccc(F)cc2)SSC1(C)C] | 5′-(cis-5-(4-fluorophenyl)-3,3-dimethyl- 1,2-dithiolan-4-yl thiophosphate) |
| | (+/−) cis | |
| (PdAr13s) | 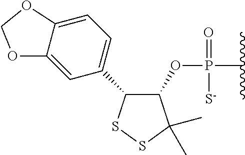 [SMILES: *P(=O)([S-])O[C@H]1[C@@H](c2ccc3c(c2)OCO3)SSC1(C)C] | 5′-(cis-5-(benzo[1,3]dioxol-6-yl)-3,3- dimethyl-1,2-dithiolan-4-yl thiophosphate) |
| | (+/−) cis | |
| (PdAr15s) | 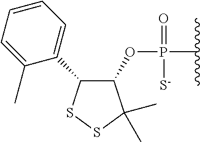 [SMILES: *P(=O)([S-])O[C@H]1[C@@H](c2ccccc2C)SSC1(C)C] | 5′-(cis-5-(2-methylphenyl)-3,3-dimethyl- 1,2-dithiolan-4-ylthiophosphate) |
| | (+/−) cis | |
| (PdAr17s) | 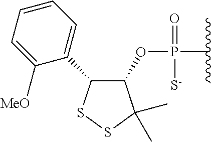 [SMILES: *P(=O)([S-])O[C@H]1[C@@H](c2ccccc2OC)SSC1(C)C] | 5′-(cis-5-(2-methoxyphenyl)-3,3-dimethyl- 1,2-dithiolan-4-yl thiophosphate) |
| | (+/−) cis | |
| (PdAr19s) | 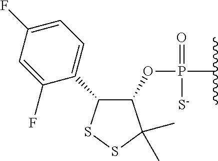 [SMILES: *P(=O)([S-])O[C@H]1[C@@H](c2ccc(F)cc2F)SSC1(C)C] | 5′-(cis-5-(2,4-difluorophenyl)-3,3-dimethyl- 1,2-dithiolan-4-yl thiophosphate) |
| | (+/−) cis | |
| (PdAr21s) | 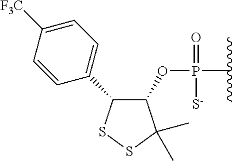 [SMILES: *P(=O)([S-])O[C@H]1[C@@H](c2ccc(C(F)(F)F)cc2)SSC1(C)C] | 5′-(cis-5-(4-trifluoromethylphenyl)-3,3- dimethyl-1,2-dithiolan-4-yl thiophosphate) |
| | (+/−) cis | |
| (PdAr23s) | 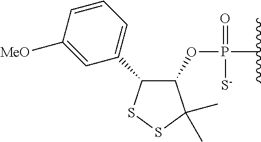 [SMILES: *P(=O)([S-])O[C@H]1[C@@H](c2cccc(OC)c2)SSC1(C)C] | 5′-(cis-5-(3-methoxyphenyl)-3,3-dimethyl- 1,2-dithiolan-4-yl thiophosphate) |
| | (+/−) cis | |
| (Pdmd1) | 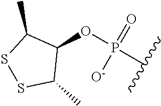 | 5′-((trans-dimethyl-1,2-dithiolan-4-yl) phosphate) |
| | (+/−) trans | |
| (Pdmd1s) | 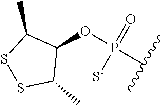 | 5′-((trans-dimethyl-1,2-dithiolan-4-yl) thiophosphate) |
| | (+/−) trans | |
| (Pmeds) | 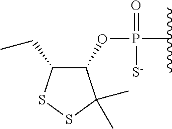 [SMILES: *P(=O)([S-])O[C@H]1[C@@H](CC)SSC1(C)C] | 5′-(cis-5-ethyl-3,3-dimethyl-1,2-dithiolan-4-yl thiophosphate) |
| | (+/−) cis | |
| (Pmids) | 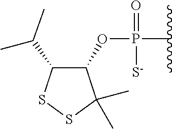 [SMILES: *P(=O)([S-])O[C@H]1[C@@H](C(C)C)SSC1(C)C] | 5′-(cis-5-isopropyl-3,3-dimethyl-1,2- dithiolan-4-y1 thiophosphate) |
| | (+/−) cis | |
| (Pmmdpn1) | 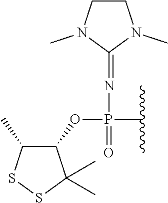 | 5′-((cis-3,3,5-trimethyl-1,2-dithiolan-4-yl) (1,3-dimethylimidazolidin-2-ylidene) phosphoramidate) |
| | (+/−) cis | |
| (Pmmdpn2) | 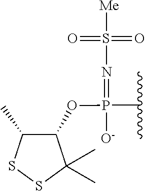 [SMILES: *P([O-])(=NS(C)(=O)=O)O[C@H]1[C@@H](C)SSC1(C)C] | 5′-((cis-3,3,5-trimethyl-1,2-dithiolan-4-yl) (N- methylsulfonyl) phosphimidate) |
| | (+/−) cis | |
| (Pmmd) | 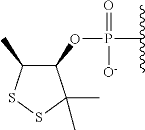 [SMILES: *P(=O)([O-])O[C@@H]1[C@H](C)SSC1(C)C] | 5′-(cis-3,3,5-trimethyl-1,2-dithiolan-4-yl phosphate) |
| | (+/−) cis | |
| (Pmmds) | 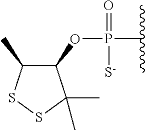 [SMILES: *P(=O)([S-])O[C@@H]1[C@H](C)SSC1(C)C] | 5′-(cis-3,3,5-trimethyl-1,2-dithiolan-4-yl thiophosphate) |
| | (+/−) cis | |
| (+cPmmds) | 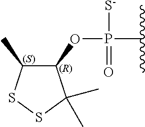 | 5′-(3,3,5S-trimethyl-1,2-dithiolan-4R-yl thiophosphate) |
| (−cPmmds) | 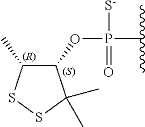 | 5′-(3,3,5R-trimethyl-1,2-dithiolan-4S-yl thiophosphate) |
| (+tPmmds) | 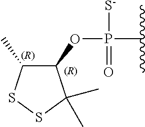 | 5′-(3,3,5R-trimethyl-1,2-dithiolan-4R- yl thiophosphate) |
| (tPmmds) | 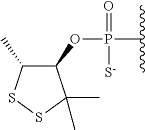 [SMILES: *P(=O)([S-])O[C@@H]1[C@@H](C)SSC1(C)C] | 5′-(trans-3,3,5-trimethyl-1,2-dithiolan-4- yl thiophosphate) |
| | (+/−) trans | |
| (−tPmmds) | 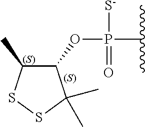 | 5′-(3,3,5S-trimethyl-1,2-dithiolan-4S-yl thiophosphate) |
| (Ptmd) | 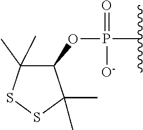 | 5′-(3,3,5,5-tetramethyl-1,2-dithiolan-4-yl phosphate) |
| (Ptmds) | 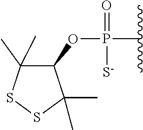 | 5′-(3,3,5,5-tetramethyl-1,2-dithiolan-4-yl thiophosphate) |
| PS | 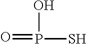 | 5′-thiophosphate |
| Q51 | 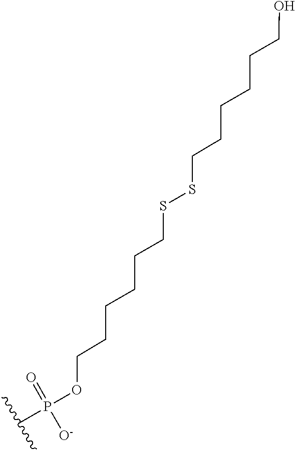 [SMILES: *P(=O)([O-])OCCCCCCSSCCCCCCO] | 5′-(14-hydroxy-7,8-dithiatetradecyl phosphate) |
| (Pmd) | 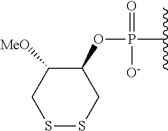 [SMILES: *P(=O)([O-])O[C@H]1CSSC[C@@H]1OC] | 5′-(trans-5-methoxy-1,2-dithian-4-yl phosphate) |
| | (+/−) trans | |
| (Pmds) | 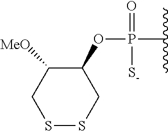 | 5′-(trans-5-methoxy-1,2-dithian- 4-yl thiophosphate) |
| | (+/−) trans | |
| (Pd) | 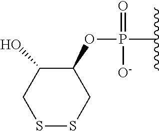 [SMILES: *P(=O)([O-])O[C@H]1CSSC[C@@H]1O] | 5′-(trans-5-hydroxy-1,2-dithian-4-yl phosphate) |
| | (+/−) trans | |
| (Pds) | 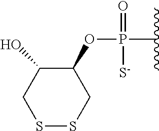 [SMILES: *P(=O)([S-])O[C@H]1CSSC[C@@H]1O] | 5′-(trans-5-hydroxy-1,2-dithian- 4-yl thiophosphate) |
| | (+/−) trans | |
| (Cymd) | 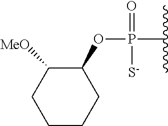 [SMILES: *P(=O)([S-])O[C@H]1CCCC[C@@H]1OC] | 5′-(trans-2-methoxycyclohex-1-yl phosphate) |
| | (+/−) trans | |
| (Cymds) | 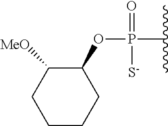 [SMILES: *P(=O)([S-])O[C@H]1CCCC[C@@H]1OC] | 5′-(trans-2-methoxycyclohex-1-yl thiophosphate) |
| | (+/−) trans | |
| (Ufms) | 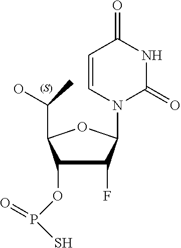 | 2′-deoxy-2′-fluoro-5′-(S)-methyluridine 3′- phosphorothioate |
| (upn9) | 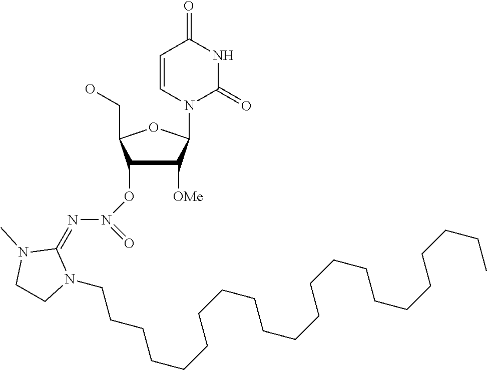 | 2′-O-methyluridine 3′-(1-methyl-3- docosanylimidazolidin- 2-ylidene)phosphoramidate |
| (URs) | 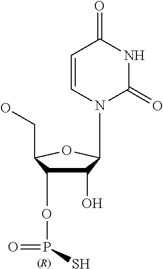 | uridine 3′-(R)-phosphorothioate |

### Example 1. Synthesis of Cyclic Disulfide Modified Phosphate Prodrug Derivatives

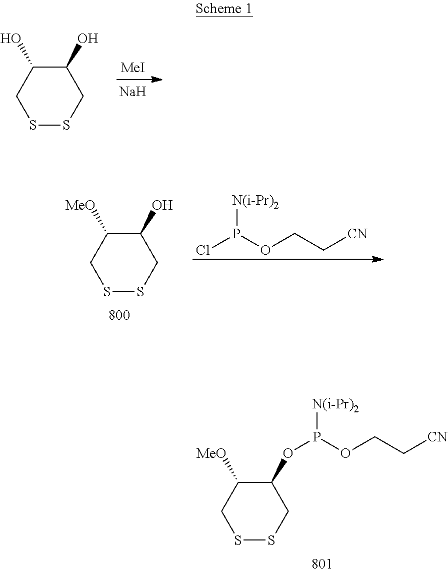

Compound 800: Trans-1,2-dithiane-4,5-diol (3.04 g, 20 mmol), was dissolved in anhydrous THF (30 mL) under inert atmosphere and cooled in a water/ice bath. Sodium hydride, 60% dispersion in oil (0.84 g, 21 mmol) was added and the mixture was stirred for 30 minutes. Iodomethane (3.7 mL, 60 mmol) was added and the mixture was allowed to slowly warm to room temperature overnight. The reaction mixture was concentrated under vacuum to a colorless liquid. The product was isolated by silica gel flash chromatography of crude (3.66 g) with isocratic 30% ethyl acetate in hexane (1:9 to 1:1 gradient). Obtained 1.11 g (33%) of 800 as yellowish oil. ¹H NMR, (400 MHz, DMSO-d₆) δ 5.29 (d, J=4.8 Hz, 1H); 3.44 (septet, J=4.8 Hz, 1H); 3.30 (dd, J=3.6, 13.2 Hz, 1H); 3.11-3.03 (m, 2H); 2.74 (dd, J=10.0, 13.6 Hz, 1H); 2.67 (dd, J=10.0, 13.6 Hz, 1H).

Compound 801: 2-cyanoethyl N,N-diisopropylchlorophosphoramidite (1.80 mL, 8 mmol) was added to a stirred solution of carbinol 800 (1.07 g, 6.4 mmol) and DIEA (1.40 mL, 8 mmol) in anhydrous ethyl acetate (30 mL) under Ar atmosphere. The mixture was stirred at room temperature for 1 hour and quenched. The organic phase was separated, washed twice with 5% NaCl, saturated NaCl, dried over anhydrous sodium sulfate, and the crude residue was purified over a column of silica gel with isocratic 25% ethyl acetate containing 1% of TEA in hexane to afford 1.88 g (80%) of pure amidite 801 as a yellowish oil. ¹H NMR (400 MHz, CD₃CN): δ 3.94-3.74 (m, 2.5H); 3.74-3.55 (m, 2.5H); 3.39 (s, 1.5H); 3.38 (s, 1.5H); 3.36-3.15 (m, 3H); 2.91 (dd, J=9.2, 13.6 Hz, 1H); 2.85-2.77 (m, 1H); 2.72-2.59 (m, 2H); 1.24-1.13 (m, 12H). ¹³C NMR (101 MHz, CD₃CN) δ 119.64; 119.61; 59.84; 59.67; 59.11; 58.91; 58.38; 58.10; 57.64; 44.10; 43.98; 25.03; 24.96; 24.95; 24.92; 24.90; 24.84; 24.76; 21.09; 21.03. ³¹P NMR (202 MHz, CD₃CN): δ 150.68; 150.37.

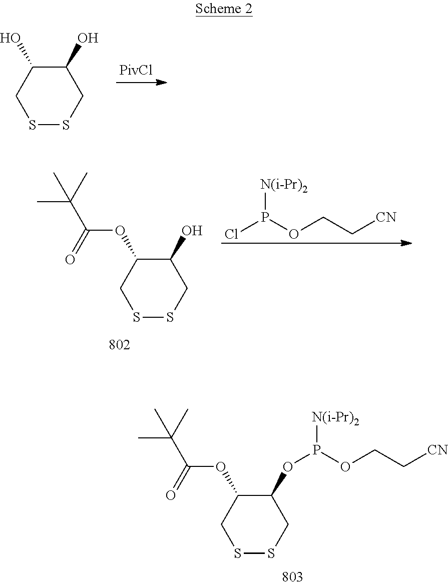

Compound 802: Trans-1,2-dithiane-4,5-diol (5.0 g, 32.8 mmol), was dissolved in pyridine (160 mL) under inert atmosphere and cooled in a water/ice bath. Trimethylacetyl chloride (14.2 mL, 98.5 mmol) was added over 5 minutes, the mixture was warmed to room temperature and stirred for 1.5 hours. The mixture was concentrated under vacuum, re-dissolved in ethyl acetate (100 mL), washed with 5% NaCl (2×100 mL), saturated NaCl (1×100 mL), dried over Na₂SO₄, filtered, and concentrated to an oil. The product was purified by silica gel flash chromatography, 120 g silica column, with ethyl acetate:hexane (1:4 to 1:2 gradient). The product-containing fractions were concentrated, chased with acetonitrile (2×), and dried under high vacuum. Compound 802 was obtained as a white solid, 66% yield (5.12 g). ¹H NMR, (500 MHz, DMSO-d₆) δ 5.43 (d, J=5.9 Hz, 1H), 4.67-4.60 (m, 1H), 3.64-3.55 (m, 1H), 3.14 (dd, J=13.2, 3.9 Hz, 2H), 2.90-2.80 (m, 2H), 1.15 (s, 9H). ¹³C NMR (126 MHz, DMSO-d₆) δ 176.54, 38.28, 26.79.

Compound 803: Compound 802 (3.0 g, 12.7 mmol) was dissolved in anhydrous ethyl acetate (60 mL) under an inert atmosphere. N,N-diisopropylethylamine (2.9 mL, 16.5 mmol) and 2-cyanoethyl N,N-diisopropylchlorophosphoramidite (3.7 mL, 16.5 mmol) were added and the mixture was stirred at room temperature for 2 hours. The reaction mixture was quenched, washed with 5% NaCl (3×200 mL), saturated NaCl (1×100 mL), dried over anhyd. Na₂SO₄, filtered, and concentrated. The product was purified by silica gel flash chromatography, 80 g silica column, using isocratic ethyl acetate (+0.5% triethylamine):hexane (1:10). The product-containing fractions were concentrated in vacuum, chased with acetonitrile (2×), and dried in high vacuum. Compound 803 was isolated as a colorless oil, 77% yield (4.24 g). ¹H NMR, (500 MHz, Acetonitrile-d₃) δ 4.88-4.75 (m, 1H), 4.03-3.92 (m, 1H), 3.89-3.68 (m, 2H), 3.67-3.56 (m, 2H), 3.46-3.32 (m, 1H), 3.03-2.84 (m, 2H), 2.71-2.59 (m, 2H), 1.25-1.11 (m, 21H). ¹³C NMR (101 MHz, Acetonitrile-d₃) δ 178.02, 119.47, 59.71, 59.51, 59.25, 59.05, 44.29, 44.16, 44.09, 43.96, 39.59, 27.55, 27.52, 25.10, 25.04, 25.03, 24.96, 24.90, 24.82, 21.13, 21.10, 21.06, 21.03. ³¹P NMR (202 MHz, Acetonitrile-d₃) δ 151.22, 148.72.

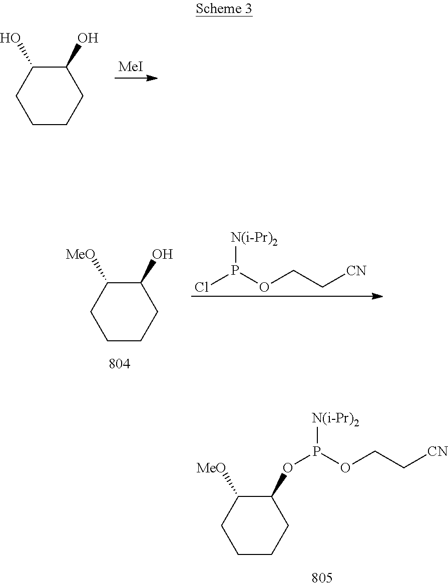

Compound 804: Trans-1,2-cyclohexanediol (10.1 g, 87.0 mmol), was dissolved in THF (120 mL) under inert atmosphere and cooled in a water/ice bath. Sodium hydride, 60% dispersion in oil (3.96 g, 91.4 mmol) was added and the reaction was stirred for 1.5 hours. Iodomethane (16.3 mL, 261.1 mmol) was added and the reaction was allowed to slowly warm to room temperature over 17 hours. The reaction mixture was concentrated under vacuum to a colorless liquid. The product was isolated by silica gel flash chromatography, 220 g silica column, using ethyl acetate:hexane (1:9 to 1:1 gradient). The product-containing fractions were concentrated and chased with dichloromethane (2×). Compound 804 was obtained as a colorless oil, 14% yield (1.6 g). ¹H NMR, (400 MHz, DMSO-d₆) δ 4.57 (d, J=4.2 Hz, 1H), 3.30-3.22 (m, 4H), 2.89-2.79 (m, 1H), 1.93-1.85 (m, 1H), 1.77-1.66 (m, 1H), 1.62-1.48 (m, 2H), 1.17-1.00 (m, 4H). ¹³C NMR (126 MHz, DMSO-d₆) δ 83.29, 71.52, 56.35, 32.64, 28.30, 23.14, 23.03.

Compound 805: Compound 804 (0.51 g, 3.9 mmol) was dissolved in anhydrous ethyl acetate (20 mL) under inert atmosphere. N,N-diisopropylethylamine (1.0 mL, 5.9 mmol) and 2-cyanoethyl N,N-diisopropylchlorophosphoramidite (1.3 mL, 5.9 mmol) were added, and the mixture was stirred at room temperature for 3 hours. The mixture was quenched and diluted with ethyl acetate (60 mL). The organic phase was separated, washed with 5% NaCl (3×150 mL), saturated NaCl (1×150 mL), dried over anhydrous Na₂SO₄, filtered, and concentrated in vacuum. The product was purified by silica gel flash chromatography over a 24 g silica column quenched with triethylamine (10 mL) using ethyl acetate:hexane (1:9 to 1:2 gradient). The product-containing fractions were concentrated, chased with acetonitrile (2×), and dried under high vacuum. Compound 805 was obtained as a colorless oil, 79% yield (1.02 g). ¹H NMR, (400 MHz, Acetonitrile-d₃) δ 3.87-3.54 (m, 5H), 3.32 (d, J=2.4 Hz, 3H), 3.14-3.02 (m, 1H), 2.70-2.57 (m, 2H), 2.02-1.83 (m, 2H), 1.66-1.55 (m, 2H), 1.47-1.21 (m, 4H), 1.21-1.13 (m, 12H). ¹³C NMR (126 MHz, Acetonitrile-d₃) δ 83.00, 82.75, 76.03, 75.55, 59.64, 59.50, 59.13, 58.98, 57.46, 56.97, 44.05, 44.03, 43.95, 43.93, 32.77, 32.42, 29.34, 29.17, 25.07, 25.05, 25.01, 25.00, 24.87, 24.83, 24.81, 24.77, 23.89, 23.76, 23.63, 23.60, 21.19, 21.13, 21.08. ³¹P NMR (162 MHz, Acetonitrile-d₃) δ 148.85, 148.56.

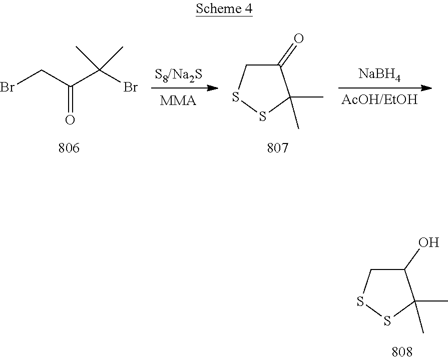

Compound 807: A suspension of sodium sulfide nonahydrate (4.08 g, 17 mmol) and elemental sulfur (1.09 g, 34 mmol) in MMA (N-methylacetamide) (35 mL) was stirred at 30° C. overnight to form homogeneous yellowish solution. A solution of dibromoketone 806 (3.45 g, 14 mmol) in MMA (10 mL) was added dropwise for ~30 minutes while maintaining bath temperature at 30° C. The mixture was stirred at 30° C. for additional 2 hours, cooled to room temperature, and quenched by addition of 5% aqeuous NaCl (200 mL). The mixture was extracted with ethyl acetate, and the organic phase was separated, washed with 5% aqeuous NaCl, saturated NaCl, and dried over anhydrous sodium sulfate. The solvent was evaporated in vacuum to afford crude residue (2.17 g) that was purified over a column of silica gel with 5% of ethyl acetate in hexane to afford 0.97 g (47%) of pure dimethyl ketone 807. ¹H NMR (400 MHz, CDCl3): δ 3.58 (s, 2H); 1.52 (s, 6H). ¹³C NMR (126 MHz, CDCl3): δ 210.5; 55.8; 41.7; 23.7.

Compound 808: Sodium borohydride (122 mg, 3.2 mmol) was added to a cooled (−78° C.) and stirred solution of ketone 807 (0.94 g, 6.4 mmol) and acetic acid (0.37 mL, 6.4 mmol) in dry ethanol (15 mL) under Ar atmosphere. The mixture was stirred at −78° C. for 2 hours, the cooling bath was removed, and the mixture was quenched by addition of saturated ammonium chloride (15 mL) and ethyl acetate (20 mL). The mixture was allowed to warm up to room temperature and water (8 mL) was added to dissolve solids. The organic phase was separated, washed consecutively with 15% aqeuous NaCl, saturated sodium bicarbonate, saturated NaCl, and dried over anhydrous sodium sulfate. The solvent was removed in vacuum to afford crude product (0.93 g) that was purified over a column of silica gel with gradient of 10 to 30% of Ethyl acetate in hexane to afford 0.66 g (70%) of 808 as slowly crystallizing yellowish oil. ¹H NMR (500 MHz, CD₃CN): δ 4.10-4.05 (m, 1H); 3.39 (dd, J=5.5, 11.0 Hz, 1H); 3.17 (d, J=8 Hz, 1H); 3.03 (dd, J=4.0, 11.0 Hz, 1H); 1.41 (s, 3H); 1.37 (s, 3H). C13 NMR (126 MHz, CDCl3): δ 82.7; 65.0; 43.4; 26.6; 21.4.

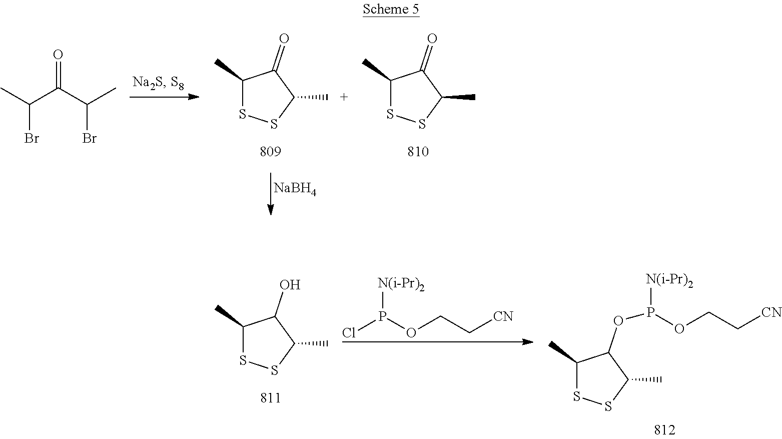

Compound 809 and 810: N-Methylacetamide (100 mL) was heated to 30° C. under inert atmosphere. Disodiumsulfide-nonahydrate (8.38 g, 34.8 mmol) and sulfur (2.24 g, 69.7 mmol) were added and the suspension was stirred at 35° C. for 24 hours to dissolve solids. Solution of 2,4-dibromo-3-pentanone (8.47 g, 34.8 mmol) in N-methylacetamine (10 mL) and was added slowly over 20 minutes. The mixture was stirred at 30° C. for 20 hours and quenched by slowly pouring to a stirred solution of 5% NaCl (400 mL). The mixture was diluted with ethyl acetate (400 mL), and the organic layer was separated, washed with 5% NaCl (1×300 mL), saturated NaCl (1×300 mL), dried over anhydrous Na₂SO₄, filtered, and concentrated. Oily residue was diluted in hexane (200 mL) and stirred for 18 hours. Solids were removed by filtration and the filtrate was concentrated to an oil. The product was purified by silica gel flash chromatography, 120 g silica column, using ethyl acetate:hexane (0 to 10% gradient). Early eluting compound 809 was isolated as a yellow liquid, 31% yield (1.31 g). Later eluting compound 810 was isolated as a yellow oil, 9% yield (0.38 g, 4:1 mix of 810 to 809). Compound 809: ¹H NMR (400 MHz, DMSO-d₆) δ 3.84 (q, J=6.9 Hz, 2H), 1.35 (d, J=6.9 Hz, 6H). Compound 810: ¹H NMR (400 MHz, DMSO-d₆) δ 3.93 (q, J=7.0 Hz, 2H), 1.37 (d, J=7.0 Hz, 6H).

Compound 811: Ketone 809 (1.02 g, 6.75 mmol) was dissolved in ethanol (15 mL) under inert atmosphere and cooled to −78° C. Acetic acid (0.39 mL, 6.75 mmol) was added, followed by sodium borohydride (130 mg, 3.37 mmol). The mixture was stirred for 5 hours, and a second portion of sodium borohydride (130 mg, 3.37 mmol) was added. The mixture was stirred for additional 2 hours, quenched with saturated NH₄Cl (10 mL), and allowed to warm to room temperature. Ethyl acetate (20 mL), saturated NH₄Cl (10 mL), and water (10 mL) were added, and the mixture was stirred at room temperature overnight. The organic layer was separated, washed with 1:1 saturated NH₄Cl:water (1×20 mL), saturated NaHCO₃ (1×25 mL), and saturated NaCl (1×25 mL), dried over Na₂SO₄, filtered, and concentrated. The product was purified by silica gel flash chromatography, 24 g silica column, using ethyl acetate:hexane (1:15 to 1:9 gradient). The product-containing fractions were concentrated, chased with dichloromethane (2×), and dried under high vacuum overnight. Compound 811 was obtained as a colorless oil, 42% yield (427 mg). ¹H NMR, (400 MHz, DMSO-d₆) δ 5.35 (d, J=5.6 Hz, 1H), 3.93 (q, J=5.1 Hz, 1H), 3.62-3.53 (m, 1H), 3.44-3.35 (m, 1H), 1.28 (t, J=6.9 Hz, 6H).

Compound 812: Compound 811 (0.40 g, 2.66 mmol) was dissolved in anhydrous ethyl acetate (13 mL) under inert atmosphere. N,N-diisopropylethylamine (0.70 mL, 4.0 mmol) and 2-cyanoethyl N,N-diisopropylchlorophosphoramidite (0.95 mL, 4.0 mmol) were added, and the mixture was stirred at room temperature for 1.5 hours. The mixture was quenched, washed with 5% NaCl (3×40 mL), saturated NaCl (1×40 mL), dried over Na₂SO₄, filtered, and concentrated. The product was purified by silica gel flash chromatography, 24 g silica column, using ethyl acetate (+1% triethylamine):hexane (1:15 to 1:9 gradient). The product-containing fractions were concentrated under vacuum, chased with acetonitrile (2×), and dried under high vacuum. Compound 812 was isolated as a yellow oil, 20% yield (182 mg). ¹H NMR, (500 MHz, Acetonitrile-d₃) δ 4.28-4.19 (m, 1H), 3.89-3.57 (m, 6H), 2.73-2.61 (m, 2H), 1.42-1.35 (m, 6H), 1.22-1.16 (m, 12H). ³¹P NMR (202 MHz, Acetonitrile-d₃) δ 150.85, 150.47.

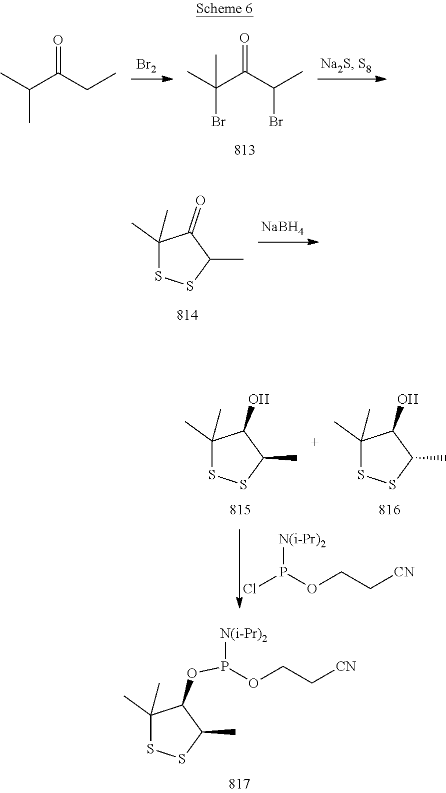

Compound 813: 2-Methyl-3-pentanone (23.3 g, 233 mmol) was dissolved in diethyl ether (100 mL) under inert atmosphere. A separately prepared solution of bromine (25.7 mL, 465 mmol) in dichloromethane (50 mL) (12 drops) was added to the ketone solution and stirred for 1 minute to initiate the reaction. The mixture was cooled in an ice/water bath, and the bromine solution (65 mL) was added dropwise to cooled and stirred ketone solution over 3 hours. The ice bath was removed, and the mixture was stirred at room temperature for 20 minutes. The mixture was diluted with diethyl ether (300 mL) and added portion-wise to a stirred aqueous solution of 5% NaCl (300 mL) and stirred 10 minutes. The organic layer was washed with 5% NaCl (2×500 mL), 5% Na₂S₂O₅ (1×450 mL) and saturated NaCl (1×500 mL), dried over Na₂SO₄, filtered, and concentrated. Compound 813 was isolated as a light-yellow liquid, 95% yield (57.1 g). ¹H NMR, (500 MHz, DMSO-d₆) δ 5.41 (q, J=6.6 Hz, 1H), 1.98 (s, 3H), 1.87 (s, 3H), 1.74 (d, J=6.6 Hz, 3H). ¹³C NMR (126 MHz, DMSO-d₆) δ 198.94, 64.58, 41.06, 29.35, 28.59, 22.10.

Compound 814: N-Methylacetamide (300 mL) was heated at 33° C. under inert atmosphere. Disodiumsulfide-nonahydrate (27.9 g, 116 mmol) and sulfur (7.46 g, 233 mmol) were added and the suspension was stirred at 35° C. for 24 hours to dissolve solids. The reaction was cooled to 30° C. and a solution of Compound 813 (30 g, 116 mmol) in N-methylacetamide (20 mL) was added slowly over 15 minutes. The mixture was stirred at 30° C. for 22 hours and quenched by slowly pouring to a stirred solution of 5% NaCl (1200 mL). The mixture was diluted with ethyl acetate (1200 mL), and washed with 5% NaCl (3×1200 mL) and saturated NaCl (1×800 mL). The organic layer was dried over Na₂SO₄, filtered, and concentrated. The oily residue was diluted in hexane (600 mL) and stirred for 18 hours. Solids were removed by decanting and the supernatant was concentrated to an oil. The product was purified by silica gel flash chromatography, 220 g silica column, using dichloromethane:hexane (1:9 to 1:8 gradient). The product-containing fractions were concentrated and chased with dichloromethane (2×). Compound 814 was isolated as a yellow oil, 32% yield (6.1 g). ¹H NMR, ELN0021-16-7 (400 MHz, DMSO-d₆) δ 3.98 (q, J=7.0 Hz, 1H), 1.45 (d, J=9.0 Hz, 6H), 1.37 (d, J=7.0 Hz, 3H). ¹³C NMR (126 MHz, DMSO-d₆) δ 211.58, 56.27, 50.15, 24.69, 24.11, 16.00.

Compound 815 and 816: Compound 814 (2.3 g, 14.2 mmol) was dissolved in ethanol (35 mL) under inert atmosphere and cooled to −78° C. Sodium borohydride (531 mg, 4.17 mmol) was added, and the reaction mixture was stirred for 15 minutes, warmed to room temperature, and stirred for additional 3 hours. The mixture was cooled to −78° C. and quenched with saturated NH₄Cl (10 mL). Ethyl acetate (50 mL), saturated NH₄Cl (35 mL), and water (20 mL) were added and the mixture was stirred at room temperature overnight. The organic layer was separated, washed with 1:1 saturated NH₄Cl:water (1×50 mL), saturated NaHCO₃ (1×50 mL), saturated NaCl (1×50 mL), dried over Na₂SO₄, filtered, and concentrated. The product was isolated by silica gel flash chromatography, 80 g silica column, using ethyl acetate:hexane (1:20 to 1:9 gradient). The product-containing fractions were concentrated and chased with dichloromethane (2×). Early eluting Compound 815 was isolated as a yellow solid, 40% yield (0.93 g). Later eluting Compound 816 was isolated as a yellow oil, 10% yield (0.23 g). Compound 815: ¹H NMR, (500 MHz, DMSO-d₆) δ 5.13 (d, J=6.9 Hz, 1H), 3.88-3.82 (m, 1H), 3.76 (dd, J=6.9, 4.5 Hz, 1H), 1.37 (d, J=6.5 Hz, 6H), 1.28 (d, J=6.8 Hz, 3H). ¹³C NMR (101 MHz, DMSO-d₆) δ 83.39, 63.88, 51.88, 28.15, 22.83, 15.24. Compound 816: ¹H NMR, (400 MHz, DMSO-d₆) δ 5.67 (d, J=6.3 Hz, 1H), 3.36 (dd, J=8.4, 6.2 Hz, 1H), 3.27-3.19 (m, 1H), 1.37 (d, J=6.5 Hz, 3H), 1.31 (d, J=3.9 Hz, 6H). ¹³C NMR (101 MHz, DMSO-d₆) δ 88.45, 57.99, 48.86, 25.74, 21.63, 19.17.

Compound 817: Compound 815 (0.2 g, 1.2 mmol) was dissolved in anhydrous ethyl acetate (6 mL) under an inert atmosphere. N,N-Diisopropylethylamine (0.32 mL, 1.8 mmol) and 2-cyanoethyl N,N-diisopropylchlorophosphoramidite (0.41 mL, 1.8 mmol) were added, and the mixture was stirred at room temperature for 3 hours. The reaction was quenched, diluted with ethyl acetate (20 mL), washed with 5% NaCl (3×40 mL), saturated NaCl (1×40 mL), dried over Na₂SO₄, filtered, and concentrated. The product was isolated by silica gel flash chromatography on a standard 24 g silica column quenched with triethylamine (10 mL), using gradient of ethyl acetate in hexane (1:9 to 1:2). The product-containing fractions were concentrated, chased with acetonitrile (2×), and dried under high vacuum. Compound 817 was obtained as a slowly crystallizing yellow oil, 54% yield (0.24 g). ¹H NMR (500 MHz, Acetonitrile-d₃) δ 4.19-4.10 (m, 1H), 4.01-3.64 (m, 5H), 2.75-2.63 (m, 2H), 1.52 (s, 3H), 1.50-1.37 (m, 6H), 1.27-1.20 (m, 12H). ³¹P NMR (202 MHz, Acetonitrile-d₃) δ 152.39, 150.75.

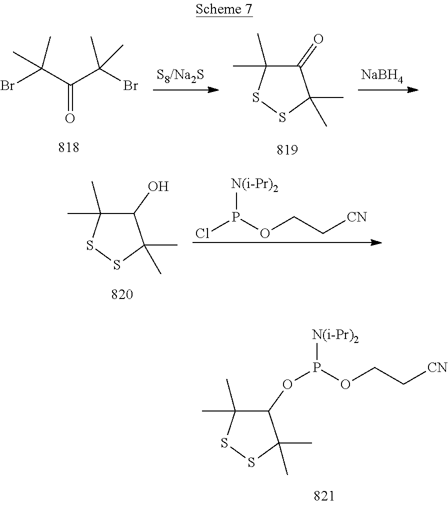

Compound 819: A suspension of sodium sulfide nonahydrate (4.08 g, 17 mmol) and elemental sulfur (1.09 g, 34 mmol) in MMA (N-methylacetamide) (35 mL) was stirred at 30° C. overnight to form homogeneous yellow solution. A solution of dibromoketone 818 (2.4 mL, 14 mmol) in MMA (10 mL) was added dropwise for ~20 minutes while maintaining bath temperature at 30° C. The mixture was stirred at 30° C. for additional 3 hours, cooled to room temperature, and quenched by addition of 5% aqeuous NaCl (200 mL). The mixture was extracted with ethyl acetate, the organic phase was separated, washed with 5% aqeuous NaCl, saturated NaCl, and dried over anhydrous sodium sulfate. The solvent was evaporated in vacuum to afford crude residue (2.49 g) that was purified over a column of silica gel with gradient of 0 to 10% of ethyl acetate in hexane to afford 1.18 g (48%) of pure tetramethyl ketone 819. ¹H NMR (400 MHz, CD3CN): δ 1.50 (s, 12H). ¹³C NMR (126 MHz, CD3CN): δ 215.4; 58.6; 25.7.

Compound 820: Sodium borohydride (420 mg, 11 mmol) was added portion wise over period of 3 hours to a cooled (0° C.) and stirred solution of ketone 819 (0.78 g, 4.4 mmol) and acetic acid (0.5 mL, 8.7 mmol) in dry ethanol (15 mL) under Ar atmosphere. The mixture was stirred at 0° C. for additional 3 hours, the cooling bath was removed, and the mixture was quenched by addition of saturated ammonium chloride (30 mL) and ethyl acetate (10 mL). The mixture was allowed to warm up to room temperature, water (5 mL) was added to dissolve solids, and the mixture was stirred vigorously in the presence of air for 48 hours. The organic phase was separated, washed with saturated NaCl, and dried over anhydrous sodium sulfate. The solvent was removed in vacuum to afford crude product (0.84 g) that was purified over a column of silica gel with gradient 5 to 20% of ethyl acetate in hexane to afford 0.65 g (83%) of 820 as slowly crystallizing yellowish liquid. ¹H NMR (500 MHz, CD₃CN): δ 3.50 (d, J=6.5 Hz, 1H); 3.43 (d, J=7.0 Hz, 1H); 1.44 (s, 6H); 1.36 (s, 6H).

Compound 821: 2-cyanoethyl N,N-diisopropylchlorophosphoramidite (0.45 mL, 2 mmol) was added to a cooled (0° C.) and stirred solution of tetramethyl carbinol 820 (0.27 g, 1.5 mmol) and N, N-diisopropylethylamine (0.35 mL, 2 mmol) in anhydrous ethyl acetate (7 mL) under Ar atmosphere. The cooling bath was removed, the mixture was stirred at room temperature for 24 hours, cooled to 0° C. and quenched by addition of saturated solution of sodium bicarbonate. The organic phase was separated, dried over anhyd. sodium sulfate, and the crude residue was purified over a column of silica gel with gradient of dichloromethane in hexane 35 to 100% to afford 0.37 g (65%) of pure amidite 821 as a yellowish oil. ¹H NMR (400 MHz, CD₃CN): δ 3.89-3.80 (m, 1H); 3.77 (d, J=12.4 Hz, 1H); 3.73-3.59 (m, 3H); 2.65 (t, J=6.0 Hz, 2H); 1.55 (s, 3H); 1.50 (s, 3H); 1.44 (s, 3H); 1.42 (s, 3H); 1.22 (d, J=6.8 Hz, 6H); 1.18 (d, J=6.8 Hz, 6H). ³¹P NMR (202 MHz, CD₃CN): δ 150.8.

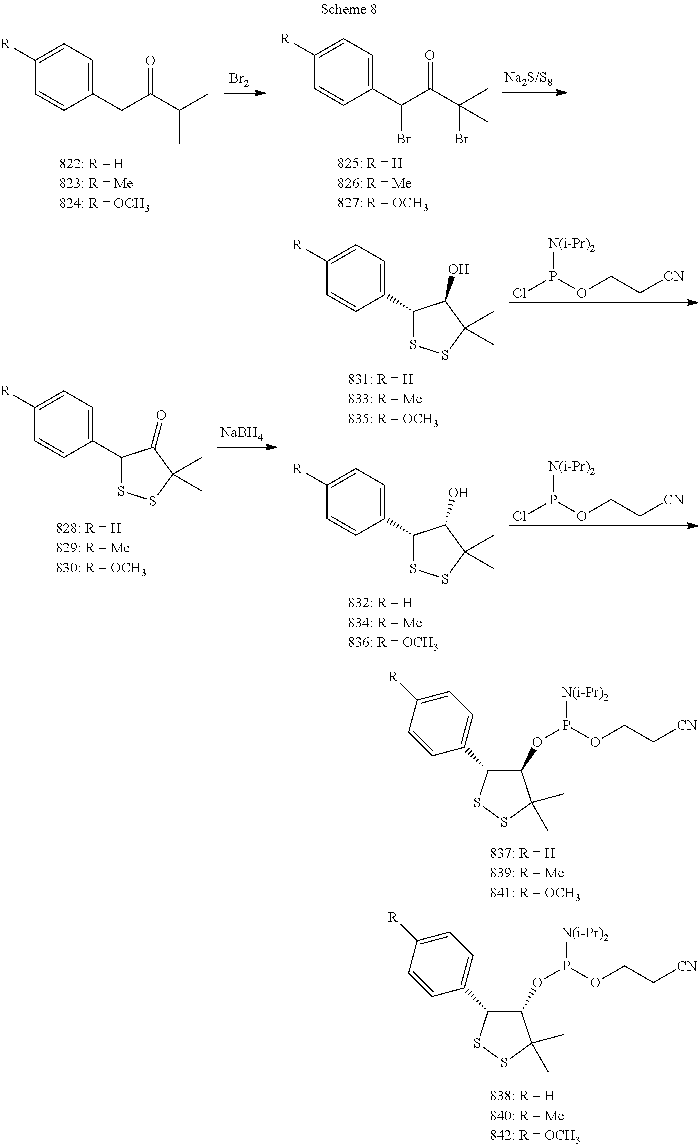

Compound 825: 3-methyl-1-phenyl-2-butanone, Compound 822 (4.0 g, 24.7 mmol) was dissolved in anhydrous diethyl ether (20 mL) under argon atmosphere. To the ketone solution was added 3 drops of a solution of bromine (7.9 g, 2.5 mL, 49.3 mmol) and DCM (10 mL) to initiate the reaction. Once the reaction changed from orange to colorless, the remaining bromine solution was added dropwise over a period of one hour. The reaction was stirred for additional 2 hours, then diluted with diethyl ether (100 mL), and charged to a stirring solution of 5% NaCl (100 mL) portion-wise. The organic layer was then washed with 5% NaCl (3×100 mL), 5% Na₂S₂O₅ (1×100 mL), and saturated NaCl (1×100 mL). The organic layer was dried over Na₂SO₄, filtered, and concentrated. The brownish crude residue was purified over a column of silica gel with gradient of 2% to 7% ethyl acetate in hexane to afford pure compound 825 as a white solid, 96% yield (7.6 g). ¹H NMR (400 MHz, DMSO) δ 7.67-7.63 (m, 2H), 7.41-7.33 (m, 3H), 6.60 (s, 1H), 1.99 (s, 3H), 1.79 (s, 3H).

Compound 826: 1-(4-methyl)-3-methylbutan-2-one, Compound 823 (4.51 g, 25.6 mmol) was dissolved in anhydrous diethyl ether (20 mL) under argon atmosphere. To the ketone solution was added 10 drops of a solution of bromine (8.18 g, 2.62 mL, 51.2 mmol) and DCM (15 mL) to initiate the reaction. Once the reaction changed from orange to lighter orange, the remaining bromine solution was added dropwise over a period of one hour. The reaction was stirred for additional 2 hours, then diluted with diethyl ether (100 mL), and charged to a stirring solution of 5% NaCl (100 mL) portion-wise. The organic layer was then washed with 5% NaCl (3×100 mL), 5% Na₂S₂O₅ (1×100 mL), and saturated NaCl (1×100 mL). The organic layer was dried over Na₂SO₄, filtered, and concentrated. The brownish crude residue was purified over a column of silica gel with gradient of 3% to 20% ethyl acetate in hexane to afford pure compound 826 as a white solid, 79% yield (6.77 g). ¹H NMR (500 MHz, DMSO) δ 7.53 (d, J=8.2 Hz, 2H), 7.19 (d, J=7.7 Hz, 2H), 6.57 (s, 1H), 2.28 (s, 3H), 1.97 (s, 3H), 1.78 (s, 3H).

Compound 827:1-(4-methoxyphenyl)-3-methylbutan-2-one, Compound 824 (4.82 g, 25.1 mmol) was dissolved in anhydrous diethyl ether (20 mL) under argon atmosphere. To the ketone solution was added 3 drops of a solution of bromine (8.01 g, 2.6 mL, 50.1 mmol) and DCM (15 mL) to initiate the reaction. Once the reaction changed from orange to lighter orange, the remaining bromine solution was added dropwise over a period of one hour. The reaction was stirred for additional 2 hours, then diluted with diethyl ether (100 mL), and charged to a stirring solution of 5% NaCl (100 mL) portion-wise. The organic layer was then washed with 5% NaCl (3×100 mL), 5% Na₂S₂O₅ (1×100 mL), and saturated NaCl (1×100 mL). The organic layer was dried over Na₂SO₄, filtered, and concentrated. The brownish crude residue was purified over a column of silica gel with gradient of 0% to 15% ethyl acetate in hexane to afford pure compound 827 as a white solid, 91% yield (8.0 g). H NMR (600 MHz, DMSO) δ 7.59 (d, J=6.8 Hz, 2H), 6.95 (d, J=6.8 Hz, 2H), 6.61 (s, 1H), 3.76 (s, 3H), 1.97 (s, 3H), 1.79 (s, 3H).

Compound 828: To a reactor containing N-methylacetamide (40 mL), heated to 33° C., was charged sodium sulfide nonahydrate (6.0 g, 25 mmol) and sulfur (1.6 g, 50 mmol). This suspension was stirred overnight at 35° C. to dissolve the solids. The mixture was cooled to 30° C., then Compound 825 (4.0 g, 25.0 mmol) was added. The reaction was stirred for 3 hours, then quenched by adding to a stirring solution of 5% NaCl (200 mL). The mixture was extracted with ethyl acetate (100 mL) and washed with 5% NaCl (2×150 mL) and saturated NaCl (1×150 mL). The organic layer was dried over Na₂SO₄, filtered, and concentrated. The crude product was diluted with hexane (100 mL), and the precipitated residual sulfur was removed by vacuum filtration. The filtrate was concentrated to afford a crude residue which was purified over a column of silica gel with gradient of 4% to 10% ethyl acetate in hexane to afford pure compound 828 as a yellow solid, 89% yield (2.49 g). H NMR (500 MHz, DMSO) δ 7.44-7.25 (m, 6H), 5.28 (s, 1H), 1.62 (s, 3H), 1.52 (s, 3H). ¹³C NMR (101 MHz, DMSO) δ 210.06, 135.77, 129.16, 128.75, 128.32, 58.97, 56.66, 24.56, 24.16.

Compound 829: To a reactor containing N-methylacetamide (40 mL), heated to 33° C., was charged sodium sulfide nonahydrate (6.0 g, 25 mmol) and sulfur (1.6 g, 50 mmol). This suspension was stirred overnight at 35° C. to dissolve the solids. The mixture was cooled to 30° C., then Compound 826 (4.0 g, 25.0 mmol) was added. The reaction was stirred for 3 hours, then quenched by adding to a stirring solution of 5% NaCl (200 mL). The mixture was extracted with ethyl acetate (100 mL) and washed with 5% NaCl (2×150 mL) and saturated NaCl (1×150 mL). The organic layer was dried over Na₂SO₄, filtered, and concentrated. The crude product was diluted with hexane (100 mL), and the precipitated residual sulfur was removed by vacuum filtration. The filtrate was concentrated to afford a crude residue which was purified over a column of silica gel with gradient of 4% to 10% ethyl acetate in hexane to afford pure compound 829 as a yellow solid, 53% yield (1.51 g). ¹H NMR (500 MHz, DMSO) δ 7.17 (s, 4H), 5.23 (s, 1H), 2.28 (s, 3H), 1.62 (s, 3H), 1.50 (s, 3H). ¹³C NMR (101 MHz, DMSO) δ 210.21, 137.81, 132.70, 129.32, 129.09, 58.91, 56.59, 24.65, 24.19, 20.71.

Compound 830: To a reactor containing N-methylacetamide (30 mL), heated to 33° C., was charged sodium sulfide nonahydrate (4.71 g, 19.6 mmol) and sulfur (1.26 g, 39.2 mmol). This suspension was stirred overnight at 35° C. to dissolve the solids. The mixture was cooled to 30° C., then Compound 827 (3.43 g, 9.8 mmol) was added. The reaction was stirred for 3 hours, then quenched by adding to a stirring solution of 5% NaCl (150 mL). The mixture was extracted with ethyl acetate (100 mL) and washed with 5% NaCl (2×150 mL) and saturated NaCl (1×150 mL). The organic layer was dried over Na₂SO₄, filtered, and concentrated. The crude product was diluted with hexane (100 mL), and the precipitated residual sulfur was removed by vacuum filtration. The filtrate was concentrated to afford a crude residue which was purified over a column of silica gel with gradient of 0% to 15% ethyl acetate in hexane to afford pure compound 830 as a yellow solid, 78% yield (1.75 g). H NMR (600 MHz, DMSO) δ 7.21 (d, J=6.9 Hz, 2H), 6.94 (d, J=6.9 Hz, 2H), 5.24 (s, 1H), 3.75 (s, 3H), 1.63 (s, 3H), 1.50 (s, 3H). ¹³C NMR (151 MHz, DMSO) δ 210.87, 159.73, 131.03, 127.99, 114.71, 59.26, 56.98, 55.65, 25.23, 24.72.

Compounds 831 and 832: Compound 828 (2.32 g, 10.34 mmol) was dissolved in ethanol (25 mL) under argon in an oven dried flask, and was then cooled to −78° C. Acetic acid (0.62 g, 0.60 mL, 10.34 mmol) was charged, followed by NaBH₄ (0.39 g, 10.34 mmol). The reaction was stirred at −78° C. for 10 minutes, at 0° C. for an hour, and then at room temperature overnight. The reaction was cooled to 0° C., and an additional aliquot of NaBH₄ (0.10 g, 2.59 mmol) was added. The reaction was stirred at 0° C. for 5 hours, and then quenched by slow addition of saturated NH₄Cl (15 mL). The mixture was diluted with ethyl acetate (80 mL), saturated NH₄Cl (40 mL) and water (35 mL). The mixture was stirred for 18 hours at room temperature. The organic layer was washed with 1:1 saturated NH₄Cl:water (1×50 mL), saturated NaHCO₃ (1×50 mL), and saturated NaCl (1×50 mL). The organic layer was dried over Na₂SO₄, filtered, and concentrated. The crude yellow residue was purified over a column of silica gel with gradient of 6% to 10% ethyl acetate in hexane to afford early eluting compound 832 (racemic mixture) as a yellow solid (1.45 g, 62% yield) and late eluting compound 831 (racemic mixture) as a yellow solid (0.13 g, 6% yield). Compound 831: ¹H NMR (500 MHz, DMSO) δ 7.51-7.42 (m, 2H), 7.38-7.24 (m, 3H), 5.68 (d, J=6.7 Hz, 1H), 4.29 (d, J=8.7 Hz, 1H), 3.93 (dd, J=8.6, 6.6 Hz, 1H), 1.46 (s, 3H), 1.38 (s, 3H). ¹³C NMR (101 MHz, DMSO) δ 140.17, 128.44, 128.15, 127.59, 89.28, 58.61, 57.01, 25.32, 21.12. Compound 832: ¹H NMR (500 MHz, DMSO) δ 7.56-7.46 (m, 2H), 7.33-7.22 (m, 3H), 5.23 (d, J=7.2 Hz, 1H), 5.00 (d, J=3.8 Hz, 1H), 3.94 (dd, J=6.9, 3.8 Hz, 1H), 1.52 (s, 3H), 1.44 (s, 3H). ¹³C NMR (101 MHz, DMSO) δ 136.49, 130.01, 127.66, 127.40, 83.58, 65.70, 61.53, 28.68, 23.27.

Compounds 833 and 834: Compound 829 (1.25 g, 5.24 mmol) was dissolved in ethanol (13 mL) under argon in an oven dried flask, and was then cooled to −78° C. Acetic acid (0.31 g, 0.30 mL, 5.24 mmol) was charged, followed by NaBH₄ (0.20 g, 5.24 mmol). The reaction was stirred at −78° C. for 10 minutes, at 0° C. for an hour, and then at room temperature overnight. The reaction was cooled to 0° C., and an additional aliquot of NaBH₄ (0.05 g, 1.31 mmol) was added. The reaction was stirred at 0° C. for 5 hours, and then quenched by slow addition of saturated NH₄Cl (10 mL). The mixture was diluted with ethyl acetate (50 mL), saturated NH₄Cl (25 mL) and water (20 mL). The mixture was stirred for 18 hours at room temperature. The organic layer was washed with 1:1 saturated NH₄Cl:water (1×50 mL), saturated NaHCO₃ (1×50 mL), and saturated NaCl (1×50 mL). The organic layer was dried over Na₂SO₄, filtered, and concentrated. The crude yellow residue was purified over a column of silica gel with gradient of 5% to 20% ethyl acetate in hexanes to afford early eluting compound 834 (racemic mixture) as a yellow solid (0.87 g, 69% yield) and late eluting compound 833 (racemic mixture) as a yellow solid (0.052 g, 4% yield). Compound 833: ¹H NMR (600 MHz, DMSO) δ 7.37-7.32 (m, 2H), 7.16 (d, J=7.6 Hz, 2H), 5.66 (d, J=6.7 Hz, 1H), 4.26 (d, J=8.6 Hz, 1H), 3.91 (dd, J=8.8, 6.5 Hz, 1H), 2.29 (s, 3H), 1.45 (s, 3H), 1.38 (s, 3H). Compound 834: ¹H NMR (600 MHz, DMSO) δ 7.44-7.36 (m, 2H), 7.13-7.08 (m, 2H), 5.24-5.16 (m, 1H), 4.97 (d, J=3.1 Hz, 1H), 3.93-3.86 (m, 1H), 2.28 (s, 3H), 1.52 (s, 3H), 1.44 (s, 3H). ¹³C NMR (151 MHz, DMSO) δ 137.11, 133.81, 130.38, 128.75, 84.03, 66.08, 61.83, 29.28, 23.84, 21.17.

Compound 835 and 836: Compound 830 (1.9 g, 7.5 mmol) was suspended in ethanol (20 mL) under argon in an oven dried flask, and then cooled to −78° C. Acetic acid (0.45 g, 0.43 mL, 7.5 mmol) was charged, followed by NaBH₄ (0.28 g, 7.5 mmol). The reaction was stirred at −78° C. for 10 minutes, at 0° C. for an hour, and then at room temperature overnight. The reaction was cooled to 0° C., and an additional aliquot of NaBH₄ (0.05 g, 1.31 mmol) was added. The reaction was stirred at 0° C. for 5 hours, then quenched by slow addition of saturated NH₄Cl (40 mL) and water (35 mL). The mixture was stirred for 48 hours. The organic layer was washed with 1:1 saturated NH₄Cl:water (1×50 mL), saturated NaHCO₃ (1×50 mL), and saturated NaCl (1×50 mL). The organic layer was dried over Na₂SO₄, filtered, and concentrated. The crude yellow residue was purified over a column of silica gel with 3:48.5:48.5 diethyl ether:DCM:hexanes to afford early eluting compound 836 (racemic mixture) as a yellow solid (1.0 g, 52% yield) and late eluting compound 835 (racemic mixture) as a yellow solid (0.07 g, 4% yield). Compound 835: ¹H NMR (600 MHz, DMSO) δ 7.37 (d, J=8.7 Hz, 2H), 6.91 (d, J=8.7 Hz, 2H), 5.66 (d, J=6.7 Hz, 1H), 4.27 (d, J=8.8 Hz, 1H), 3.89 (dd, J=8.8, 6.6 Hz, 1H), 3.74 (s, 3H), 1.43 (s, 3H), 1.38 (s, 3H). ¹³C NMR (151 MHz, DMSO) δ 159.21, 131.67, 128.54, 113.60, 83.93, 66.04, 61.41, 55.54, 29.36, 23.88. Compound 836: ¹H NMR (600 MHz, DMSO) δ 7.45 (d, J=8.7 Hz, 2H), 6.86 (d, J=8.8 Hz, 2H), 5.27 (d, J=7.2 Hz, 1H), 4.97 (d, J=3.7 Hz, 1H), 3.85 (d, J=3.3 Hz, 1H), 3.73 (s, 3H), 1.51 (s, 3H), 1.43 (s, 3H). ¹³C NMR (151 MHz, DMSO) δ 159.29, 132.02, 129.81, 114.37, 89.34, 58.74, 57.19, 55.62, 26.10, 21.89.

Compound 837: Compound 831 (0.1 g, 0.44 mmol) was dissolved in anhydrous ethyl acetate (1 mL) under an inert atmosphere. N,N-Diisopropylethylamine (0.15 mL, 0.88 mmol) and 2-cyanoethyl N,N-diisopropylchlorophosphoramidite (0.15 mL, 0.66 mmol) were added and the mixture was stirred at room temperature for 18 hours. The reaction was quenched, diluted with ethyl acetate (20 mL), washed with 5% NaCl (3×20 mL) and saturated NaCl (1×40 mL), dried over Na₂SO₄, filtered, and concentrated. The crude residue was purified over a column of silica gel with gradient of 5% to 30% ethyl acetate in hexanes to afford pure compound 837 as a yellow oil, 77% yield (0.15 g). ¹H NMR (600 MHz, CD₃CN) δ 7.55-7.50 (m, 2H), 7.41-7.30 (m, 3H), 4.57-4.43 (m, 2H), 3.68-3.49 (m, 3H), 3.27-3.13 (m, 1H), 2.560-2.56 (m, 1H), 2.26-2.20 (m, 1H), 1.62-1.56 (m, 6H), 1.16 (d, J=6.8 Hz, 3H), 1.10 (d, J=6.8 Hz, 3H), 1.05 (d, J=6.8 Hz, 3H), 0.97 (d, J=6.8 Hz, 3H). ¹³C NMR (151 MHz, CD₃CN) δ 140.19, 139.67, 129.22, 129.17, 129.14, 129.07, 128.60, 128.46, 92.49, 92.40, 92.22, 92.15, 59.68, 59.65, 59.62, 59.20, 59.16, 58.38, 58.25, 58.19, 58.05, 43.55, 43.47, 43.40, 43.31, 26.16, 25.94, 25.91, 24.37, 24.32, 24.29, 24.26, 24.20, 24.14, 22.59, 22.00, 20.39, 20.34, 20.14, 20.09. ³¹P NMR (243 MHz, CD₃CN) S 150.08, 148.64.

Compound 839: Compound 833 (0.05 g, 0.21 mmol) was dissolved in anhydrous ethyl acetate (0.5 mL) under an inert atmosphere. N,N-Diisopropylethylamine (0.07 mL, 0.42 mmol) and 2-cyanoethyl N,N-diisopropylchlorophosphoramidite (0.07 mL, 0.31 mmol) were added and the mixture was stirred at room temperature for 18 hours. The reaction was quenched, diluted with ethyl acetate (10 mL), washed with 5% NaCl (3×15 mL) and saturated NaCl (1×15 mL), dried over Na₂SO₄, filtered, and concentrated. The crude residue was purified over a column of silica gel with gradient of 5% to 30% ethyl acetate in hexanes to afford pure compound 839 as a yellow oil, 46% yield (0.042 g). ¹H NMR (600 MHz, CD₃CN) δ 7.42-7.37 (m, 2H), 7.24-7.14 (m, 2H), 4.54-4.40 (m, 2H), 3.68-3.48 (m, 3H), 3.25-3.11 (m, 1H), 2.59-2.57 (m, 1H), 2.34 (d, J=12.2 Hz, 3H), 2.26-2.20 (m, 1H), 1.61-1.56 (m, 5H), 1.16 (d, J=6.8 Hz, 3H), 1.10 (d, J=6.8 Hz, 3H), 1.05 (d, J=6.8 Hz, 3H), 0.97 (d, J=6.8 Hz, 3H). ¹³C NMR (151 MHz, CD₃CN) δ 138.49, 138.43, 136.85, 136.42, 129.70, 129.67, 129.09, 129.08, 92.42, 92.31, 92.12, 92.04, 59.74, 59.71, 59.43, 59.40, 59.09, 58.99, 58.44, 58.30, 58.17, 58.03, 43.59, 43.51, 43.41, 43.32, 26.31, 26.05, 26.02, 24.38, 24.33, 24.30, 24.29, 24.26, 24.15, 24.09, 22.71, 22.12, 20.71, 20.70, 20.39, 20.34, 20.08, 20.03. ³¹P NMR (243 MHz, CD₃CN) δ 150.32, 148.64.

Compound 841: Compound 835 (0.042 g, 0.16 mmol) was dissolved in anhydrous ethyl acetate (0.5 mL) under an inert atmosphere. N,N-Diisopropylethylamine (0.04 mL, 0.25 mmol) and 2-cyanoethyl N,N-diisopropylchlorophosphoramidite (0.05 mL, 0.25 mmol) were added, and the mixture was stirred at room temperature for 18 hours. The reaction was quenched, diluted with ethyl acetate (10 mL), washed with 5% NaCl (3×15 mL) and saturated NaCl (1×15 mL), dried over Na₂SO₄, filtered, and concentrated. The crude residue was purified over a column of silica gel with gradient of 5% to 30% ethyl acetate in hexane to afford pure compound 841 as a yellow oil, 44% yield (0.033 g). ¹H NMR (600 MHz, CD₃CN) δ 7.46-7.40 (m, 2H), 6.97-6.87 (m, 2H), 4.52-4.37 (m, 2H), 3.80 (d, J=11.7 Hz, 3H), 3.70-3.19 (m, 4H), 2.59 (t, J=5.9 Hz, 1H), 2.31-2.27 (m, 1H), 1.63-1.55 (m, 6H), 1.16 (d, J=6.8 Hz, 3H), 1.11 (d, J=6.8 Hz, 3H), 1.06 (d, J=6.8 Hz, 3H), 0.98 (d, J=6.8 Hz, 3H). ³¹P NMR (243 MHz, CD₃CN) δ 150.16, 148.78.

Compound 838: Compound 832 (0.40 g, 1.77 mmol) was dissolved in anhydrous ethyl acetate (9 mL) under an inert atmosphere. N,N-Diisopropylethylamine (0.4 mL, 2.65 mmol) and 2-cyanoethyl N,N-diisopropylchlorophosphoramidite (0.59 mL, 2.65 mmol) were added and the mixture was stirred at room temperature for 18 hours. The reaction was quenched, diluted with ethyl acetate (40 mL), washed with 5% NaCl (3×80 mL) and saturated NaCl (1×80 mL), dried over Na₂SO₄, filtered, and concentrated. The crude residue was purified over a column of silica gel with gradient of 5% to 30% ethyl acetate in hexane to afford pure compound 838 as a yellow oil, 80% yield (0.60 g). ¹H NMR (400 MHz, CD₃CN) δ 7.48-7.42 (m, 2H), 7.34-7.21 (m, 3H), 5.01 (d, J=5.6 Hz, 1H), 4.36-4.31 (m, 1H), 3.76-3.66 (m, 1H), 3.62-3.43 (m, 3H), 2.64-2.55 (m, 2H), 1.66 (s, 3H), 1.61 (s, 3H), 1.04 (d, J=6.8 Hz, 6H), 0.94 (d, J=6.8 Hz, 6H). ³¹P NMR (162 MHz, CD₃CN) δ 150.93.

Compound 840: Compound 834 (0.61 g, 2.53 mmol) was dissolved in anhydrous ethyl acetate (10 mL) under an inert atmosphere. N,N-Diisopropylethylamine (0.66 mL, 3.8 mmol) and 2-cyanoethyl N,N-diisopropylchlorophosphoramidite (0.85 mL, 3.8 mmol) were added and the mixture was stirred at room temperature for 18 hours. The reaction was quenched, diluted with ethyl acetate (50 mL), washed with 5% NaCl (3×50 mL) and saturated NaCl (1×50 mL), dried over Na₂SO₄, filtered, and concentrated. The crude residue was purified over a column of silica gel with gradient of 5% to 30% ethyl acetate in hexane to afford pure compound 840 as a yellow oil, 83% yield (0.93 g). ¹H NMR (600 MHz, CD₃CN) δ 7.43-7.32 (m, 2H), 7.19-7.10 (m, 2H), 5.06-4.97 (m, 1H), 4.36-4.29 (m, 1H), 3.77-3.23 (m, 4H), 2.67-2.39 (m, 2H), 2.36-2.27 (m, 3H), 1.70-1.55 (m, 6H), 1.13-0.93 (m, 12H). ¹³C NMR (151 MHz, CD₃CN) δ 138.08, 137.85, 134.25, 133.76, 131.16, 130.77, 129.34, 128.85, 119.28, 86.81, 86.73, 86.00, 85.94, 64.40, 62.96, 62.94, 61.09, 58.37, 58.28, 58.24, 58.13, 43.69, 43.60, 43.59, 43.51, 27.99, 27.56, 27.54, 24.59, 24.52, 24.47, 24.43, 24.39, 24.31, 24.27, 24.22, 23.83, 23.80, 20.73, 20.69, 20.56, 20.51, 20.41, 20.36. ³¹P NMR (243 MHz, CD₃CN) δ 151.40, 149.14.

Compound 842: Compound 836 (0.50 g, 1.95 mmol) was dissolved in anhydrous ethyl acetate (8 mL) under an inert atmosphere. N,N-Diisopropylethylamine (0.51 mL, 2.9 mmol) and 2-cyanoethyl N,N-diisopropylchlorophosphoramidite (0.65 mL, 2.9 mmol) were added and the mixture was stirred at room temperature for 18 hours. The reaction was quenched, diluted with ethyl acetate (50 mL), washed with 5% NaCl (3×50 mL) and saturated NaCl (1×50 mL), dried over Na₂SO₄, filtered, and concentrated. The crude residue was purified over a column of silica gel with gradient of 5% to 30% ethyl acetate in hexane to afford pure compound 842 as a yellow oil, 77% yield (0.68 g). ¹H NMR (600 MHz, CD₃CN) δ 7.48-7.35 (m, 2H), 6.92-6.83 (m, 2H), 5.05-4.98 (m, 1H), 4.34-4.24 (m, 1H), 3.84-3.26 (m, 7H), 2.66-2.42 (m, 2H), 1.71-1.54 (m, 6H), 1.15-0.95 (m, 12H). ¹³C NMR (151 MHz, CD₃CN) δ 159.94, 159.84, 132.42, 132.06, 131.44, 129.25, 128.73, 119.30, 114.13, 113.56, 86.68, 86.60, 85.84, 85.78, 64.31, 62.83, 62.79, 62.46, 62.44, 60.71, 58.39, 58.31, 58.25, 58.16, 55.49, 55.48, 43.71, 43.62, 43.57, 43.49, 27.95, 27.51, 27.49, 24.55, 24.51, 24.50, 24.44, 24.40, 24.38, 24.33, 24.33, 24.28, 23.77, 23.75, 22.94, 20.56, 20.52, 20.46, 20.40. ³¹P NMR (243 MHz, CD₃CN) δ 151.42, 149.09.

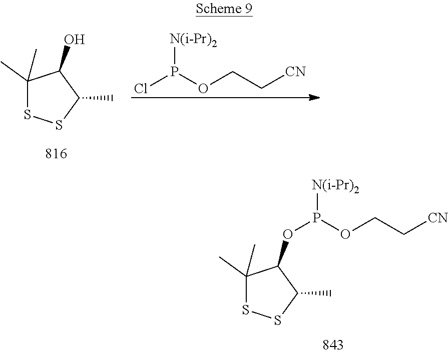

Compound 843: Compound 816 (0.4 g, 2.4 mmol) was dissolved in anhydrous ethyl acetate (12 mL) under an inert atmosphere. N,N-Diisopropylethylamine (0.41 g, 3.2 mmol) was added followed by addition of 2-cyanoethyl N,N-diisopropylchlorophosphoramidite (0.75 g, 3.2 mmol), and the mixture was stirred at room temperature for 3 hours. The reaction was quenched with a solution of saturated sodium bicarbonate and ethyl acetate. The organic phase was dried over anhydrous Na₂SO₄, filtered, and concentrated under reduced pressure to give a crude residue which was purified by silica gel flash chromatography to afford pure Compound 843 as a yellow oil, 89% yield (0.79 g). ¹H NMR (400 MHz, CD₃CN) δ 3.91-3.42 (m, 6H), 2.69-2.61 (m, 2H), 1.52-1.43 (m, 9H), 1.23-1.15 (m, 12H). ¹³C NMR (101 MHz, CD₃CN) δ 119.62, 92.89, 92.76, 92.58, 92.46, 60.15, 60.10, 59.76, 59.74, 59.11, 59.07, 58.91, 58.87, 52.19, 52.15, 51.96, 44.17, 44.15, 44.05, 44.02, 26.90, 26.68, 26.63, 25.10, 25.02, 24.96, 24.94, 24.88, 23.39, 22.91, 21.04, 20.96, 19.92, 19.81, 19.75. ³¹P NMR (162 MHz, CD₃CN) δ 150.00, 149.81.

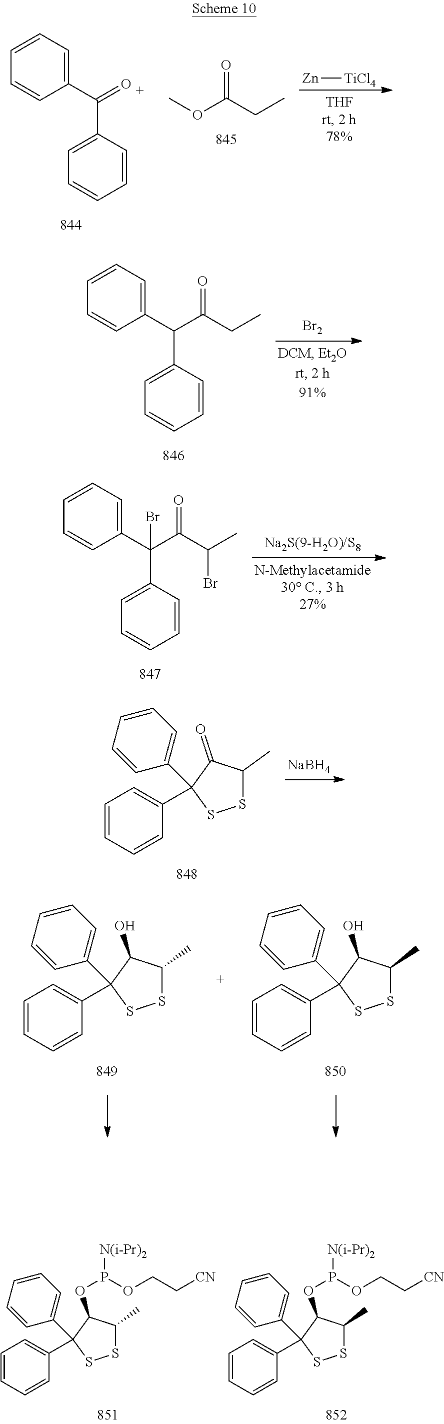

Ketone 846: To a 1 L three-neck flask equipped with a reflux condenser were added methyl propionate 845 (6.48 g, 73.5 mmol), diphenylmethanone 844 (6.70 g, 36.8 mmol) and zinc powder (9.62 g, 147 mmol) under argon atmosphere. Anhydrous THF (180 mL) was added to the mixture with stirring. The suspension was cooled to 0-5° C. in ice-water bath, and titanium (IV) chloride (13.95 g, 8.1 mL, 73.5 mmol) was slowly added to the mixture. The dark-blue suspension was stirred for 2 hours at 25° C. followed by heating at 50° C. for 6 hours. The mixture was cooled to room temperature, and 1M HCl (800 mL) was added. The mixture was stirred at room temperature for 10 minutes and extracted with ethyl acetate (250 mL×3). The organic layers were combined, washed with aqueous NaCl, and dried over MgSO₄. After the solid was filtered and solvent was removed in vacuum, the residue was purified by flash column chromatography on silica gel (330 g, 120 mL/min, gradient of 30% DCM to 60% of DCM in hexanes) to give compound 846: (6.44 g, 78%). H NMR (600 MHz, DMSO-d₆) δ 0.93 (t, 3H, J=6 Hz), 2.56 (q, 2H, J=6 Hz), 5.39 (s, 1H), 7.23-7.27 (m, 6H), 7.31-7.34 (m, 4H). ¹³C NMR (126 MHz, DMSO-d₆) δ 8.5, 35.8, 62.8, 127.3, 129.0, 129.3, 139.6, 209.4.

Dibromo-ketone 847: 1,1-Diphenyl-butan-2-one (846) (7.2 g, 32.1 mmol) was dissolved in anhydrous diethyl ether (45 mL) under argon atmosphere. 12 drops of a solution of bromine (12.8 g, 80.3 mmol) in anhydrous DCM (15 mL) were added to initiate reaction. Once the reaction mixture solution changed color from orange to almost colorless, the remaining bromine solution was added dropwise over a period of 35 minutes. The reaction mixture was stirred for an additional 2 hours, diluted with diethyl ether (120 mL), and slowly, portion-wise poured to a stirring solution of 5% NaCl (150 mL). The organic layer was separated, washed with 5% NaCl (2×150 mL), 5% sodium meta-bisulfite (1×150 mL), and saturated NaCl (1×150 mL). The organic layer was dried over Na₂SO₄, filtered, and concentrated under vacuum to give yellowish liquid which slowly solidified upon cooling to give compound 847: 95% purity, 11.2 g (91%). ¹H NMR (600 MHz, DMSO-d₆) δ 1.69 (d, 3H, J=5 Hz), 4.99 (q, 2H, J=5 Hz), 7.30-7.32 (m, 4H), 7.41-7.48 (m, 6H). ¹³C NMR (126 MHz, DMSO-d₆) δ 24.5, 44.4, 76.1, 128.9, 129.1, 129.5, 129.8, 130.0, 130.1, 137.6, 138.5, 198.7.

Cyclic ketone 848: To a 100 mL RBF containing N-methylacetamide (15 mL) and heated to 33° C. was added sodium sulfide nonahydrate (1.20 g, 5.0 mmol) and sulfur (320 mg, 10 mmol). The suspension was stirred for 24 hours at 35° C. to dissolve the solids. The reaction mixture was cooled to 30° C., and a solution of 847 (1.27 g, 3.33 mmol) in N-methylacetamide (3 mL) was added slowly, dropwise to the reaction mixture. The reaction was stirred at 30° C. for 3 hours, and quenched by pouring to a stirred solution of 5% NaCl (60 mL). The mixture was extracted with ethyl acetate (50 mL), washed with 5% NaCl (3×40 mL) and saturated NaCl (1×40 mL). The organic layer was separated and dried over Na₂SO₄, filtered, and concentrated under vacuum to a yellow oil which was triturated with hexanes (80 mL) to precipitate solid sulfur that was removed by filtration. The resulted filtrate was concentrated under vacuum to give yellow oil which was purified by flash column chromatography on silica gel (40 g, 20 mL/min, elution with gradient of 5% to 35% of ethyl acetate in hexanes). The product-containing fractions were combined and concentrated under vacuum to afford compound 848 as a yellow oil: 265 mg (27%). ¹H NMR (600 MHz, DMSO-d₆) δ 1.08 (d, 3H, J=5 Hz), 4.33 (q, 1H, J=5 Hz), 7.30-7.39 (m, 8H), 7.49-7.51 (m, 2H). ¹³C NMR (126 MHz, DMSO-d₆) δ 11.3, 51.6, 68.24, 125.9, 126.1, 126.3, 126.4, 126.8, 126.9, 137.7, 140.9, 205.5.

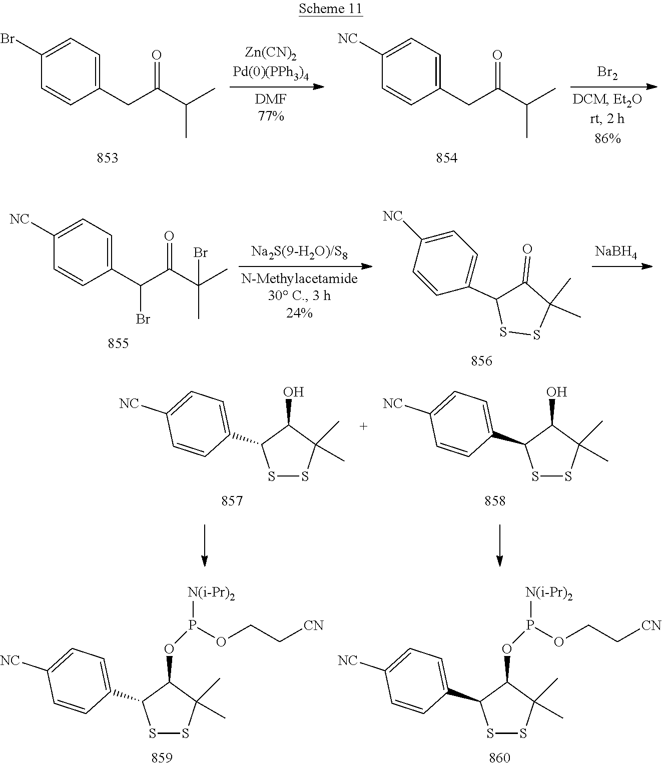

Ketone 854: To an oven-dried 100 mL round bottom flask were added 1-(4-bromophenyl)-3-methyl-butan-2-one (853) (3.50 g, 14.5 mmol), palladium (0) tetrakis-triphenylphosphine (1.34 g, 1.2 mmol) and zinc cyanide (1.70 g, 14.5 mmol). Anhydrous DMF (35 mL) was added, the reaction mixture was then degassed and heated at 90° C. under argon atmosphere overnight. The mixture was cooled, diluted with 150 mL of EtOAc and washed with ammonium hydroxide (2M, 150 mL×2), followed by saturated NaHCO₃ (140 mL) and saturated NaCl (100 mL). The organic layer was separated, dried over sodium sulfate, filtered, and concentrated under vacuum to give 3.22 g of crude residue. The crude residue was purified by flash column chromatography on silica gel (220 g, 60 mL/min, gradient of 20% to 35% of ethyl acetate in hexanes) to afford colorless oil which slowly solidified to furnish compound 854 as a white solid: (2.13 g, 77%). ¹H NMR (600 MHz, CDCl₃) δ 1.17 (d, 6H, J=5 Hz), 2.74 (m, 1H), 3.84 (s, 2H), 7.31-7.33 (m, 2H), 7.62-7.64 (m, 2H). ¹³C NMR (126 MHz, CDCl₃) δ 18.2, 41.0, 47.1, 110.9, 118.8, 130.4, 132.3, 139.8, 210.2.

Dibromo-ketone 855: 1-(4-cyanophenyl)-3-methyl-butan-2-one 854 (2.10 g, 11.2 mmol) was dissolved in anhydrous diethyl ether (12 mL) under argon atmosphere. 12 Drops of a solution of bromine (3.85 g, 24.1 mmol) in anhydrous DCM (5 mL) was added to initiate the reaction. Once the reaction mixture solution changed color from orange to almost colorless, the remaining bromine solution was added dropwise over a period of 25 minutes. The reaction was then stirred for an additional 2 hours, diluted with diethyl ether (40 mL) and slowly, portion wise poured to a stirring solution of 5% NaCl (55 mL). The organic layer was separated, washed with 5% NaCl (55 mL×2), 5% sodium meta-bisulfite (1×55 mL), and saturated NaCl (1×55 mL), dried over Na₂SO₄, filtered, and concentrated under vacuum to give compound 855 (~95% purity) as a yellow liquid which slowly solidified upon cooling: 3.35 g (86%). ¹H NMR (600 MHz, CDCl₃) δ 11.79 (s, 3H), 2.05 (s, 3H), 6.04 (s, 1H), 7.59 (d, 2H, J=8 Hz), 7.73 (d, 2H, J=8 Hz). ¹³C NMR (126 MHz, CDCl₃) δ 27.2, 28.5, 41.5, 62.2, 111.1, 116.3, 128.2, 130.5, 138.9, 194.1.

Cyclic ketone 856: To a 100 mL RBF containing N-methylacetamide (15 mL) and heated to 33° C. was added sodium sulfide nonahydrate (1.20 g, 5.0 mmol) and sulfur (320 mg, 10 mmol). The suspension was stirred for 24 hours at 35° C. to dissolve solids. The reaction mixture was cooled to 30° C., and a solution of 855 (1.27 g, 3.33 mmol) in N-methylacetamide (3 mL) was added slowly, dropwise to the reaction mixture. The reaction was stirred at 30° C. for 3 hours and quenched by pouring to a stirred solution of 5% NaCl (60 mL). The mixture was extracted with ethyl acetate (50 mL), washed with 5% NaCl (3×40 mL) and saturated NaCl (1×40 mL). The organic layer was separated and dried over Na₂SO₄, filtered, and concentrated under vacuum to a yellow oil which was triturated with hexanes (80 mL) to precipitate solid sulfur that was removed by filtration. The resulted filtrate was concentrated under vacuum to give yellow oil which was purified by flash column chromatography on silica gel (40 g, 20 mL/min, elution with gradient of 5% to 35% of ethyl acetate in hexanes). The product-containing fractions were combined and concentrated under vacuum to afford 856 as a yellow oil: 265 mg (27%). ¹H NMR (600 MHz, DMSO-d₆) δ 1.56 (s, 3H), 1.60 (s, 3H), 5.50 (s, 1H), 7.52-7.54 (m, 2H), 7.86-7.88 (m, 2H). ¹³C NMR (126 MHz, DMSO-d₆) δ 24.5, 24.8, 57.5, 58.3, 111.6, 119.0, 130.5, 133.2, 142.0, 209.7.

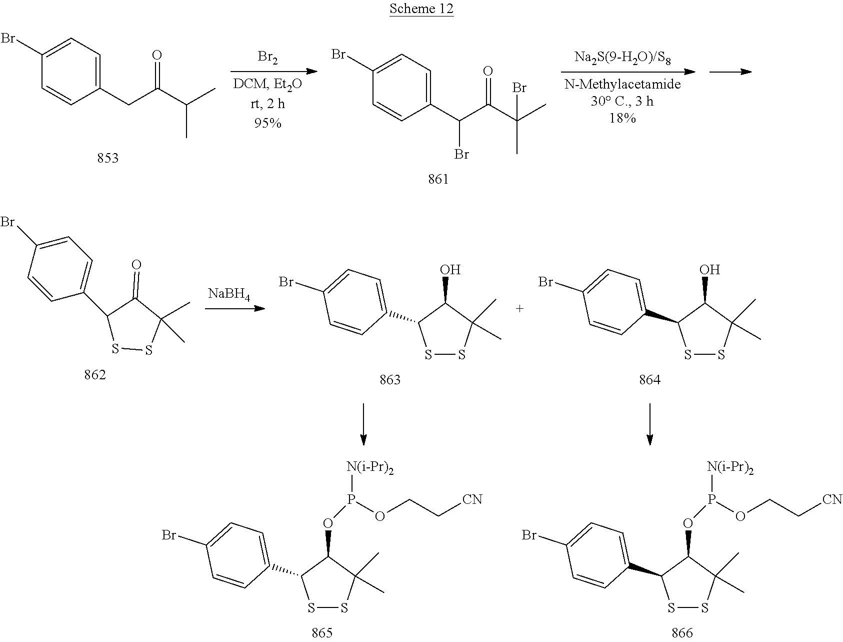

Dibromo-ketone 861: 1-(4-bromophenyl)-3-methyl-butan-2-one 853 (2.00 g, 8.3 mmol) was dissolved in anhydrous diethyl ether (12 mL) under argon atmosphere. 12 Drops of a solution of bromine (3.98 g, 24.9 mmol) in anhydrous DCM (6 mL) were added to initiate reaction. Once the reaction mixture changed color from orange to almost colorless, the remaining bromine solution was added dropwise over a period of 25 minutes. The mixture was stirred for an additional 2 hours, diluted with diethyl ether (40 mL), and slowly, portion-wise poured to a stirring solution of 5% NaCl (60 mL). The organic layer was separated, washed with 5% NaCl (60 mL×2), 5% sodium meta-bisulfite (60 mL×1) and saturated NaCl (60 mL×1), dried over Na₂SO₄, filtered, and concentrated under vacuum to give compound 861 (~95% purity) as a yellow liquid which slowly solidified upon cooling: 3.15 g (95%). ¹H NMR (600 MHz, CDCl₃) δ 1.74 (s, 3H), 1.98 (s, 3H), 6.00 (s, 1H), 7.17-7.45 (m, 4H). ¹³C NMR (126 MHz, CDCl₃) δ 29.3, 30.5, 44.5, 63.8, 123.6, 130.9, 132.0, 134.9, 196.6.

Cyclic ketone 862: To a 100 mL round bottom flask containing N-methylacetamide (14 mL) and heated to 33° C. was added sodium sulfide nonahydrate (1.00 g, 4.2 mmol) and sulfur (0.268 g, 8.4 mmol). The suspension was stirred for 24 hours at 35° C. to dissolve the solids. The mixture was cooled to 30° C., and a solution of compound 861 (1.11 g, 2.8 mmol) in N-methylacetamide (3 mL) was added slowly dropwise. The reaction mixture was stirred for 3 hours at 30° C. and quenched by pouring to a stirring solution of 5% NaCl (50 mL). The mixture was extracted with ethyl acetate (50 mL), and the organic layer was separated, washed with 5% NaCl (3×40 mL) and saturated NaCl (1×40 mL). The organic layer was dried over Na₂SO₄, filtered, and concentrated under vacuum to a yellow oil which was triturated with hexanes (80 mL) to precipitate sulfur that was removed by filtration. The filtrate was concentrated under vacuum to afford a crude residue as a yellow oil which was purified by flash column chromatography on silica gel (40 g, 20 mL/min, elution with gradient of 5% to 35% of ethyl acetate in hexanes). The product-containing fractions were combined and concentrated under vacuum to afford compound 862 (~80% purity) as a yellow oil: (156 mg, 18%). ¹H NMR (600 MHz, CDCl₃) δ 1.60 (s, 3H), 1.69 (s, 3H), 4.75 (s, 1H), 7.19-7.20 (m, 2H), 7.51-7.55 (m, 2H).

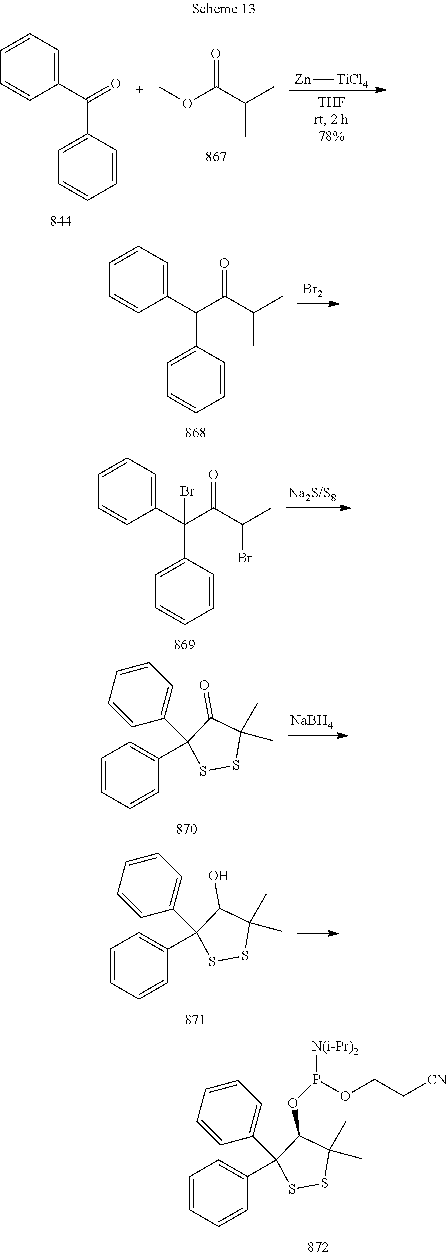

Ketone 868: To a 1 L three-neck flask equipped with a reflux condenser were added methyl 2-methylpropionic ester compound 867 (10.2 g, 100 mmol), diphenylmethanone compound 844 (9.10 g, 49.9 mmol) and zinc powder (13.06 g, 199.8 mmol) under argon atmosphere. Anhydrous THF (200 mL) was added with stirring, the suspension was cooled to 0-5° C. in ice-water bath, and titanium (IV) chloride (18.9 g, 10.9 mL, 100 mmol) was slowly added. The dark-blue suspension was stirred for 2 hours at 25° C. and then heated at 50° C. overnight. The reaction mixture was cooled to room temperature and 1M HCl (800 mL) was added. The mixture was stirred at room temperature for 10 minutes, and extracted with ethyl acetate (300 mL×3). The organic layers were combined, washed with aqueous NaCl, and dried over MgSO₄. After the solid was filtered and the solvent was removed in vacuum, the crude residue was purified by flash column chromatography on silica gel (330 g, 120 mL/min, gradient 30% to 60% of DCM in hexanes) to give compound 868: (8.87 g, 74%). ¹H NMR (600 MHz, CDCl₃) δ 1.15 (d, 6H, J=6 Hz), 2.83 (m, 1H), 5.33 (s, 1H), 7.25-7.29 (m, 6H), 7.32-7.35 (m, 4H). ¹³C NMR (126 MHz, CDCl₃) δ 18.6, 41.0, 62.2, 127.1, 128.7, 129.0, 138.6, 212.1.

### Example 2. Synthesis of Oligonucleotides Containing Modified Phosphate Prodrug at the 5′ End of the Oligonucleotide

All oligonucleotides were synthesized as described here, or as otherwise described in Table 7. Oligonucleotides were synthesized at 1 or 10 μmol scale using standard solid-phase oligonucleotide protocols, with 500-Å controlled pore glass (CPG) solid supports from Prime Synthesis and commercially available amidites from ChemGenes. The phosphoramidite solutions were 0.15 M in anhydrous acetonitrile with 15% THF as a co-solvent for 2′-O-methyl uridine, 2′-O-methyl cytidine, and the modified phosphate prodrugs. The modified phosphate prodrug monomers were coupled either on synthesizer or manually. For manual coupling, activator (0.25M 5-ethylthio-1H-tetrazole (ETT) in anhydrous ACN) was added followed by equal volume of prodrug solution. Solution was mixed for 20 minutes. Following coupling, the column was put on an ABI for oxidation or sulfurization. Oxidation (0.02M iodine in THF/pyridine/water) or sulfurization solution (0.1M 3-(dimethylaminomethylidene)amino-3H-1,2,4-dithiazole-3-thione (DDTT) in pyridine) was delivered to column for one minute or 30 seconds, respectively, and then held in solution for 10 minutes. This process was repeated for sulfurization. After completion of the solid-phase syntheses (SPS), the CPG solid support was washed with anhydrous acetonitrile and dried with argon. Oligonucleotides were deprotected by incubation with 5% diethylanolamine (DEA) in ammonia at room temperature for 2 hours.

Crude oligonucleotides were purified using strong anion exchange through a TSKgel SuperQ-5PW(20) resin with phosphate buffers (pH=8.5) containing sodium bromide at 65° C. The appropriate fractions were pooled and desalted via SEC.

TABLE 7
Single oligonucleotide strands having modified phosphate prodrug at 5′ end

| | | | | | SEQ |
|---|---|---|---|---|---|
| | | Synthesizer/ | | | ID |
| Oligo ID | Sequence | scale | Deprotection | Other | NO: |
| A-515432.1 | (Pmd)dTdTdTdTdTdTd | ABI394/ | 5% DEA in | | 1 |
| | TdTdTdT | 1 μmol | ammonia, 2 h, RT | | |
| A-515433.1 | (Pmds)dTdTdTdTdTdTd | ABI394/ | 5% DEA in | | 2 |
| | TdTdTdT | 1 μmol | ammonia, 2 h, RT | | |
| A-515488.2 | (Pmd)usCfsacuUfuAfUf | Mermade192/ | 5% DEA in | | 3 |
| | ugagUfuUfcugugscsc | 1 μmol | ammonia, 24 h, | | |
| | | | 30° C. | | |
| A-515489.1 | (Pmd)uCfacuUfuAfUfug | Mermade192/ | 5% DEA in | | 4 |
| | agUfuUfcugugscsc | 1 μmol | ammonia, 24 h | | |
| | | | 30° C. | | |
| A-515490.1 | (Pmds)usCfsacuUfuAfU | Mermade192/ | 5% DEA in | | 5 |
| | fugagUfuUfcugugscsc | 1 μmol | ammonia, 24 h | | |
| | | | 30° C. | | |
| A-515490.3 | (Pmds)usCfsacuUfuAfU | Mermade12/ | 5% DEA in | | 6 |
| | fugagUfuUfcugugscsc | 25 μmol | ammonia, 24 h | | |
| | | | 30° C. | | |
| A-515491.1 | (Pmds)uCfacuUfuAfUfu | Mermade192/ | 5% DEA in | | 7 |
| | gagUfuUfcugugscsc | 1 μmol | ammonia, 24 h | | |
| | | | 30° C. | | |
| A-515491.3 | (Pmds)usCfsacuUfuAfU | Mermade12/ | 5% DEA in | | 8 |
| | fugagUfuUfcugugscsc | 25 μmol | ammonia, 24 h | | |
| | | | 30° C. | | |
| A-784093.1 | (Pmds)uUfauaGfagcaaga | Mermade12/ | 5% DEA in | | 9 |
| | AfcAfcuguususu | 10 μmol | ammonia, 24 h, | | |
| | | | 30° C. | | |
| A-780495.1 | (Pmds)uUfuagAfgUfGfa | Mermade12/ | 5% DEA in | | 10 |
| | ggaUfuAfaaaugsasg | 10 μmol | ammonia, 2 h, | | |
| | | | 30° C. | | |
| A-1875172.1 | (Cymd)usCfsacuUfuAfU | Precursor | 5% DEA in | Labile amidites (Pac | 11 |
| | fugagUfuUfcugugscsc | (up to | ammonia, 2 h, | protected) for 2′- | |
| | | prodrug) on | RT | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | Purification at 50° C. | |
| | | prodrug | | | |
| A-1875173.1 | (Cymds)usCfsacuUfuAf | Precursor | 5% DEA in | Labile amidites (Pac | 12 |
| | UfugagUfuUfcugugscsc | (up to | ammonia, 2 h, | protected) for 2′- | |
| | | prodrug) on | RT | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | Purification at 50° C. | |
| | | prodrug | | | |
| A-1875174.1 | (Pd)usCfsacuUfuAfUfug | Precursor | 5% DEA in | Labile amidites (Pac | 13 |
| | agUfuUfcugugscsc | (up to | ammonia, 2 h at | protected) for 2′- | |
| | | prodrug) on | RT, 5 h at 65° C. | OMeA, 2′-OMe G, | |
| | | Akta | | 2′-F A, 2′-F G | |
| | | Oligopilot/ | | Cap A; 5% | |
| | | 12 mL, | | phenoxyacetic | |
| | | Manual | | anhydride in THF | |
| | | coupling | | Purification at 50° C. | |
| | | prodrug | | | |
| A-1875175.1 | (Pds)usCfsacuUfuAfUfu | Precursor | 5% DEA in | Labile amidites (Pac | 14 |
| | gagUfuUfcugugscsc | (up to | ammonia, 2 h at | protected) for 2′- | |
| | | prodrug) on | RT, 5 h at 65° C. | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | Purification at 50° C. | |
| | | prodrug | | | |
| A-1875176.1 | (Pdmd1)usCfsacuUfuAf | Precursor | 5% DEA in | Labile amidites (Pac | 15 |
| | UfugagUfuUfcugugscsc | (up to | ammonia, 2 h, | protected) for 2′- | |
| | | prodrug) on | RT | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | Unstable amidite, | |
| | | prodrug | | unsuccessful | |
| | | | | coupling | |
| A-1875177.1 | (Pdmd1s)usCfsacuUfaA | Precursor | 5% DEA in | Labile amidites (Pac | 16 |
| | fUfugagUfuUfcugugscsc | (up to | ammonia, 2 h, | protected) for 2′- | |
| | | prodrug) on | RT | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | Unstable amidite, | |
| | | prodrug | | unsuccessul | |
| | | | | coupling | |
| A-1875178.1 | (Pmmd)usCfsacuUfuAf | Precursor | 5% DEA in | Labile amidites (Pac | 17 |
| | UfugagUfuUfcugugscsc | (up to | ammonia, 2 h, | protected) for 2′- | |
| | | prodrug) on | RT | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | Purification at 50° C. | |
| | | prodrug | | | |
| A-1875179.1 | (Pmmds)usCfsacuUfuAf | Precursor | 5% DEA in | Labile amidites (Pac | 18 |
| | UfugagUfuUfcugugscsc | (up to | RT | OMeA, 2′-OMe G, | |
| | | prodrug) on | | 2′-F A, 2′-F G | |
| | | Akta | | Cap A: 5% | |
| | | Oligopilot/ | | phenoxyacetic | |
| | | 12 mL, | | anhydride in THF | |
| | | Manual | | | |
| | | coupling of | | | |
| | | prodrug | | | |
| A-1875180.1 | (Ptmd)usCfsacuUfuAfUf | Precursor | 5% DEA in | Labile amidites (Pac | 19 |
| | ugagUfuUfcugugscsc | (up to | ammonia, 2 h, | protected) for 2′- | |
| | | prodrug) on | RT | OMeA, 2′-OMe G, | |
| | | Akta | | 2′-F A, 2′-F G | |
| | | Oligopilot/ | | Cap A: 5% | |
| | | 12 mL, | | phenoxyacetic | |
| | | coupling of | | anhydride in THF | |
| | | prodrug | | 50% n-1 | |
| | | | | Purification at 50° C. | |
| A-1875181.1 | (Ptmds)usCfsacuUfuAf | Precursor | 5% DEA in | Labile amidites (Pac | 20 |
| | UfugagUfuUfcugugscsc | (up to | ammonia, 1 h at | protected) for 2′- | |
| | | prodrug) on | 65° C., 16 h at | OMeA, 2′-OMe G, | |
| | | Akta | 30° C. | 2′-F A, 2′-F G | |
| | | Oligopilot/ | | Cap A: 5% | |
| | | 12 mL, | | phenoxyacetic | |
| | | Manual | | anhydride in THF | |
| | | coupling of | | 50% n-1 | |
| | | prodrug | | Purification at 50° C. | |
| A-2058840.1 | (Cymds)uCfacuUfuAfUf | Precursor | 5% DEA in | | 21 |
| | ugagUfuUfcugugscsc | (up to | ammonia, 1 h at | | |
| | | prodrug) on | 65° C., 16 h at | | |
| | | ABI394/ | 30° C. | | |
| | | 10 μmol, | | | |
| | | Manual | | | |
| | | coupling of | | | |
| | | prodrug | | | |
| A-2058841.1 | (Pds)uCfacuUfuAfUfuga | Precursor | 5% DEA in | | 22 |
| | gUfuUfcugugscsc | (up to | ammonia, 10 h | | |
| | | prodrug) on | at 65° C., 30 h at | | |
| | | ABI394/ | 30° C. | | |
| | | 10 μmol, | | | |
| | | Manual | | | |
| | | coupling of | | | |
| | | prodrug | | | |
| A-2058842.1 | (Pmmds)uCfacuUfuAfU | Precursor | 5% DEA in | | 23 |
| | fugagUfuUfcugugscsc | (up to | ammonia, 1 h at | | |
| | | prodrug) on | 65° C., 16 h at | | |
| | | ABI394/ | 30° C. | | |
| | | 10 μmol, | | | |
| | | Manual | | | |
| | | coupling of | | | |
| | | prodrug | | | |
| A-2058843.1 | (Ptmds)uCfacuUfuAfUf | Precursor | 5% DEA in | Double coupling | 24 |
| | ugagUfuUfcugugscsc | (up to | ammonia, 1 h at | | |
| | | prodrug) on | 65° C., 16 h at | | |
| | | ABI394/ | 30° C. | | |
| | | 10 μmol, | | | |
| | | Manual | | | |
| | | coupling of | | | |
| | | prodrug | | | |

Upper case letter followed with f- 2′-deoxy-2′-fluoro (2′-F) sugar modification; lower case letter- 2′-O-methyl
(2′-OMe) sugar modification; s- phosphorothioate (PS) linkage; VP- vinyl phosphonate; the prodrugs-

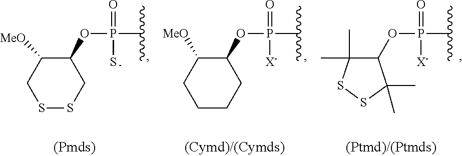

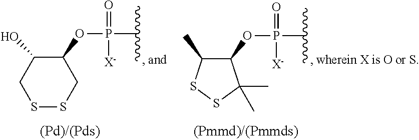

The siRNA duplexes having cyclic disulfide phosphate modifications at 5′-end of the antisense strand were synthesized and listed in Table 8.

TABLE 8
siRNAs having modified phosphate prodrug at the 5′-end of the antisense strand

| | | | | | | | SEQ |
|---|---|---|---|---|---|---|---|
| Duplex ID | Oligo ID | Strand | Target | OligoSeq | Molecular Weight | Molecular Weight Found | ID NO: |
| AD-73603 | A-147464 | sense | F12 | csascagaAfaCfUfCfaauaaagugaL96 | 8801.65 | 8797.12 | 25 |
| | A-147465 | antis | F12 | usCfsacuUfuAfUfugagUfuUfcugugscsc | 7514.87 | 7511.02 | 26 |
| AD-291896 | A-147464 | sense | F12 | csascagaAfaCfUfCfaauaaagugaL96 | 8801.65 | 8797.12 | 27 |
| | A-447595 | antis | F12 | VPuCfacuUfuAfUfugagUfuUfcugugscsc | 7558.74 | 7555.10 | 28 |
| AD-326760 | A-147464 | sense | F12 | csascagaAfaCfUfCfaauaaagugaL96 | 8801.65 | 8797.13 | 29 |
| | A-515491 | antis | F12 | (Pmds)uCfacuUfuAfUfugagUfuUfcugugscsc | 7728.03 | 7723.02 | 30 |
| AD-1023150 | A-147464 | sense | F12 | csascagaAfaCfUfCfaauaaagugaL96 | 8801.65 | 8797.13 | 31 |
| | A-1875172 | antis | F12 | (Cymd)usCfsacuUfuAfUfugagUfuUfcugugscsc | 7707.02 | 7703.08 | 32 |
| AD-1023155 | A-147464 | sense | F12 | csascagaAfaCfUfCfaauaaagugaL96 | 8801.65 | 8797.13 | 33 |
| | A-1875173 | antis | F12 | (Cymds)usCfsacuUfuAfUfugagUfuUfcugugscsc | 7723.08 | 7719.03 | 34 |
| AD-1023151 | A-147464 | sense | F12 | csascagaAfaCfUfCfaauaaagugaL96 | 8801.65 | 8797.13 | 35 |
| | A-1875174 | antis | F12 | (Pd)usCfsacuUfuAfUfugagUfuUfcugugscsc | 7729.06 | 7724.97 | 36 |
| AD-1023156 | A-147464 | sense | F12 | csascagaAfaCfUfCfaauaaagugaL96 | 8801.65 | 8797.13 | 37 |
| | A-1875175 | antis | F12 | (Pds)usCfsacuUfuAfUfugagUfuUfcugugscsc | 7745.12 | 7740.95 | 38 |
| AD-1023154 | A-147464 | sense | F12 | csascagaAfaCfUfCfaauaaagugaL96 | 8801.65 | 8797.13 | 39 |
| | A-1875178 | antis | F12 | (Pmmd)usCfsacuUfuAfUfugagUfuUfcugugscsc | 7741.11 | 7737.01 | 40 |
| AD-1023152 | A-147464 | sense | F12 | csascagaAfaCfUfCfaauaaagugaL96 | 8801.65 | 8797.13 | 41 |
| | A-1875179 | antis | F12 | (Pmmds)usCfsacuUfuAfUfugagUfuUfcugugscsc | 7757.18 | 7752.99 | 42 |
| AD-1023153 | A-147464 | sense | F12 | csascagaAfaCfUfCfaauaaagugaL96 | 8801.65 | 8797.13 | 43 |
| | A-1875180 | antis | F12 | (Ptmd)usCfsacuUfuAfUfugagUfuUfcugugscsc | 7755.14 | 7751.03 | 44 |
| AD-1023157 | A-147464 | sense | F12 | csascagaAfaCfUfCfaauaaagugaL96 | 8801.65 | 8797.13 | 45 |
| | A-1875181 | antis | F12 | (Ptmds)usCfsacuUfuAfUfugagUfuUfcugugscsc | 7771.20 | 7767.01 | 46 |
| AD-1144829 | A-147464 | sense | F12 | csascagaAfaCfUfCfaauaaagugaL96 | 8801.65 | 8797.13 | 47 |
| | A-2058840 | antis | F12 | (Cymds)uCfacuUfuAfUfugagUfuUfcugugscsc | 7690.95 | 7687.10 | 48 |
| AD-1144831 | A-147464 | sense | F12 | csascagaAfaCfUfCfaauaaagugaL96 | 8801.65 | 8797.13 | 49 |
| | A-2058841 | antis | F12 | (Pds)uCfacuUfuAfUfugagUfuUfcugugscsc | 7712.99 | 7709.00 | 50 |
| AD-1144832 | A-147464 | sense | F12 | csascagaAfaCfUfCfaauaaagugaL96 | 8801.65 | 8797.13 | 51 |
| | A-2058842 | antis | F12 | (Pmmds)uCfacuUfuAfUfugagUfuUfcugugscsc | 7725.05 | 7721.04 | 52 |
| AD-1144830 | A-147464 | sense | F12 | csascagaAfaCfUfCfaauaaagugaL96 | 8801.65 | 8797.13 | 53 |
| | A-2058843 | antis | F12 | (Ptmds)uCfacuUfuAfUfugagUfuUfcugugscsc | 7739.07 | 7735.05 | 54 |

Upper case letter followed with f- 2′-F sugar modification; lower case letter- 2′-OMe sugar modification;
s- phosphorothioate (PS) linkage; VP- vinyl phosphonate; the prodrugs are the same as in Table 7 above; the ligand-
L96

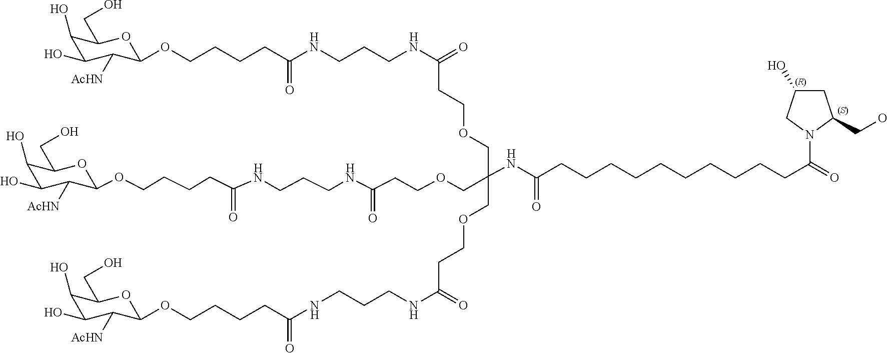

### Example 3. In Vitro Evaluation of siRNA Duplexes Containing Modified Phosphate Prodrugs at the 5′ End

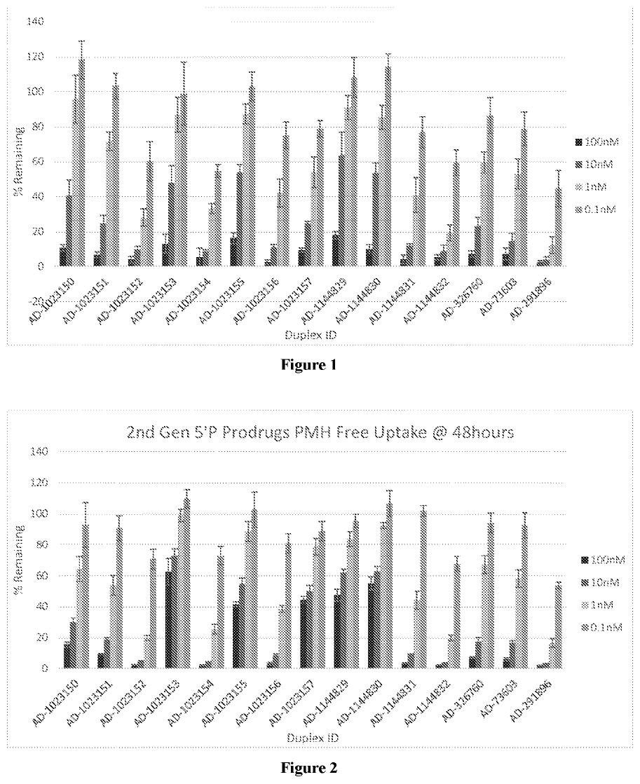

Transfection procedure: siRNA duplexes containing the modified phosphate prodrugs at the 5′ end (Table 9) were transfected in primary mouse hepatocytes with RNAiMAX at 0.1, 1, 10, and 100 nm concentrations and analyzed 24 hours post-transfection. Percent F12 message remaining was determined by qPCR. The results were plotted against the control, as shown in FIG. 1.

Free uptake procedure: siRNA duplexes containing the modified phosphate prodrugs at the 5′ end (Table 9) were incubated with primary mouse hepatocytes at 0.1, 1, 10, and 100 nm concentrations in cell culture medium and analyzed after 48 hours. Percentage of F12 message remaining was determined by qPCR. The results were plotted against the control, as shown in FIG. 2.

TABLE 9
siRNA duplexes used for in vitro evaluation

| | | SEQ | | SEQ |
|---|---|---|---|---|
| Duplex | Sense Strand Sequence | ID | Antisense Strand Sequence | ID |
| ID | (5′-3′) | NO | (5′-3′) | NO |
| AD- | csascagaAfaCfUfCfaauaaagu | 55 | (Cymd)usCfsacuUfuAfUfugagU | 56 |
| 1023150 | gaL96 | | fuUfcugugscsc | |
| AD- | csascagaAfaCfUfCfaauaaagu | 55 | (Pd)usCfsacuUfuAfUfugagUfuU | 57 |
| 1023151 | gaL96 | | fcugugscsc | |
| AD- | csascagaAfaCfUfCfaauaaagu | 55 | (Pmmds)usCfsacuUfuAfUfugag | 58 |
| 1023152 | gaL96 | | UfuUfcugugscsc | |
| AD- | csascagaAfaCfUfCfaauaaagu | 55 | (Ptmd)usCfsacuUfuAfUfugagUf | 59 |
| 1023153 | gaL96 | | uUfcugugscsc | |
| AD- | csascagaAfaCfUfCfaauaaagu | 55 | (Pmmd)usCfsacuUfuAfUfugagU | 60 |
| 1023154 | gaL96 | | fuUfcugugscsc | |
| AD- | csascagaAfaCfUfCfaauaaagu | 55 | (Cymds)usCfsacuUfuAfUfugag | 61 |
| 1023155 | gaL96 | | UfuUfcugugscsc | |
| AD- | csascagaAfaCfUfCfaauaaagu | 55 | (Pds)usCfsacuUfuAfUfugagUfu | 62 |
| 1023156 | gaL96 | | Ufcugugscsc | |
| AD- | csascagaAfaCfUfCfaauaaagu | 55 | (Ptmds)usCfsacuUfuAfUfugagU | 63 |
| 1023157 | gaL96 | | fuUfcugugscsc | |
| AD- | csascagaAfaCfUfCfaauaaagu | 55 | (Cymds)uCfacuUfuAfUfugagUf | 64 |
| 1144829 | gaL96 | | uUfcugugscsc | |
| AD- | csascagaAfaCfUfCfaauaaagu | 55 | (Ptmds)uCfacuUfuAfUfugagUfu | 65 |
| 1144830 | gaL96 | | Ufcugugscsc | |
| AD- | csascagaAfaCfUfCfaauaaagu | 55 | (Pds)uCfacuUfuAfUfugagUfuUf | 66 |
| 1144831 | gaL96 | | cugugscsc | |
| AD- | csascagaAfaCfUfCfaauaaagu | 55 | (Pmmds)uCfacuUfuAfUfugagUf | 67 |
| 1144832 | gaL96 | | uUfcugugscsc | |
| AD- | csascagaAfaCfUfCfaauaaagu | 55 | (Pmds)uCfacuUfuAfUfugagUfu | 68 |
| 326760 | gaL96 | | Ufcugugscsc | |
| AD- | csascagaAfaCfUfCfaauaaagu | 55 | usCfsacuUfuAfUfugagUfuUfcug | 69 |
| 73603 | gaL96 | | ugscsc | |
| AD- | csascagaAfaCfUfCfaauaaagu | 55 | VPuCfacuUfuAfUfugagUfuUfcu | 70 |
| 291896 | gaL96 | | gugscsc | |

Upper case letter followed with f-2′-F sugar modification; lower case letter-2′-OMe sugar modification; s-phosphorothioate (PS) linkage; VP-vinyl phosphonate; the prodrugs and ligands are the same as in Table 8 above.

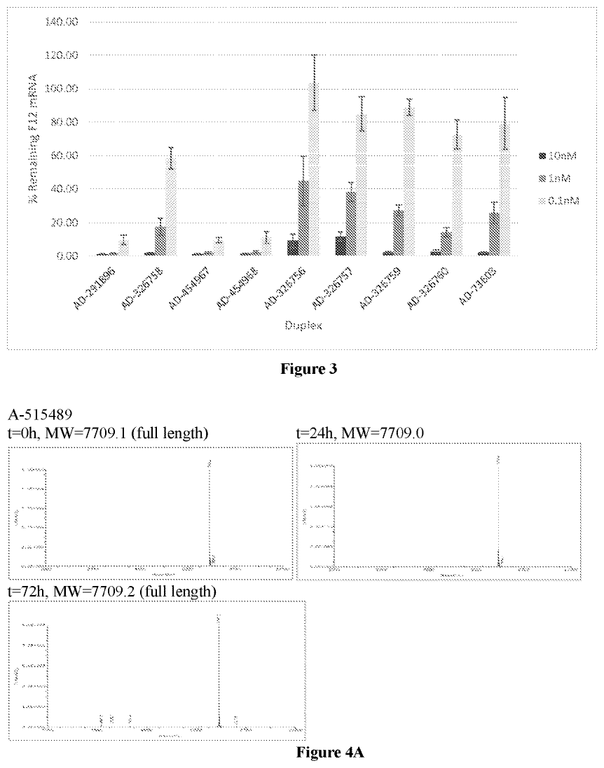

Transfection procedure: siRNA duplexes containing the modified phosphate prodrugs at the 5′ end were transfected in primary mouse hepatocytes with RNAiMAX at 0.1, 1, and 10 nm concentrations and analyzed 24 hours post-transfection. Percent F12 message remaining was determined by qPCR. The results were plotted against the control, as shown in FIG. 3.

TABLE 10
siRNA duplexes used for in vitro evaluation in FIG. 3

| | | | | | | Molecular | SEQ |
|---|---|---|---|---|---|---|---|
| Duplex | Oligo | | | | Molecular | Weight | ID |
| ID | ID | Strand | Target | Oligo Seq | Weight | found | NO: |
| AD- | A- | sense | F12 | csascagaAfaCfUfCf | 8801.656 | 8797.128 | 71 |
| 73603 | 147464 | | | aauaaagugaL96 | | | |
| | A- | antis | F12 | usCfsacuUfuAfUfug | 7514.875 | 7511.025 | 72 |
| | 147465 | | | agUfuUfcugugscsc | | | |
| AD- | A- | sense | F12 | csascagaAfaCfUfCf | 8801.656 | 8797.128 | 73 |
| 291896 | 147464 | | | aauaaagugaL96 | | | |
| | A- | antis | F12 | VPuCfacuUfuAfUfu | 7558.743 | 7555.05 | 74 |
| | 447595 | | | gagUfuUfcugugscsc | | | |
| AD- | A- | sense | F12 | csascagaAfaCfUfCf | 8801.656 | 8797.128 | 75 |
| 326758 | 147464 | | | aauaaagugaL96 | | | |
| | A- | antis | F12 | uCfacuUfuAfUfuga | 7482.744 | 7479.071 | 76 |
| | 515487 | | | gUfuUfcugugscsc | | | |
| AD- | A- | sense | F12 | csascagaAfaCfUfCf | 8801.656 | 8797.128 | 77 |
| 326756 | 147464 | | | aauaaagugaL96 | | | |
| | A- | antis | F12 | (Pmd)usCfsacuUfu | 7744.095 | 7738.993 | 78 |
| | 515488 | | | AfUfugagUfuUfcug | | | |
| | | | | ugscsc | | | |
| AD- | A- | sense | F12 | csascagaAfaCfUfCf | 8801.656 | 8797.128 | 79 |
| 326757 | 147464 | | | aauaaagugaL96 | | | |
| | A- | antis | F12 | (Pmd)uCfacuUfuAf | 7711.964 | 7707.039 | 80 |
| | 515489 | | | UfugagUfuUfcugug | | | |
| | | | | SCSC | | | |
| AD- | A- | sense | F12 | csascagaAfaCfUfCf | 8801.656 | 8797.128 | 81 |
| 326759 | 147464 | | | aauaaagugaL96 | | | |
| | A- | antis | F12 | (Pmds)usCfsacuUfu | 7760.161 | 7754.97 | 82 |
| | 515490 | | | AfUfugagUfuUfcug | | | |
| | | | | ugscsc | | | |
| AD- | A- | sense | F12 | csascagaAfaCfUfCf | 8801.656 | 8797.128 | 83 |
| 326760 | 147464 | | | aauaaagugaL96 | | | |
| | A- | antis | F12 | (Pmds)uCfacuUfuAf | 7728.03 | 7723.016 | 84 |
| | 515491 | | | UfugagUfuUfcugug | | | |
| | | | | SCSC | | | |
| AD- | A- | sense | F12 | csascagaAfaCfUfCf | 8801.656 | 8797.128 | 85 |
| 454968 | 147464 | | | aauaaagugaL96 | | | |
| | A- | antis | F12 | PSusCfsacuUfuAfU | 7611.928 | 7606.969 | 86 |
| | 815956 | | | fugagUfuUfcugugsc | | | |
| | | | | SC | | | |
| AD- | A- | sense | F12 | csascagaAfaCfUfCf | 8801.656 | 8797.128 | 87 |
| 454967 | 147464 | | | aauaaagugaL96 | | | |
| | A- | antis | F12 | PSuCfacuUfuAfUfu | 7579.797 | 7575.015 | 88 |
| | 815957 | | | gagUfuUfcugugscsc | | | |

Upper case letter followed with f-2′-F sugar modification; lower case letter-2′-OMe sugar modification; s-phosphorothioate (PS) linkage; the structures for the prodrug Pmds and ligand L96 are the same as in Table 8 above.

### Example 4. DTT Reduction Assay to Check the Cleavability of the Cyclic Phosphate Prodrug Analogues

Reduction of the 5′-cyclic modified phosphate prodrugs by dithiothreitol (DTT) to 5′ phosphate or 5′ phosphorothioate was explored by incubating 100 9M modified oligonucleotide (23-nt length) with 100 mM DTT in 7×PBS. The amount of full-length (non-reduced) oligonucleotide was observed via LCMS analysis (either Agilent Single-Quad MS or Novatia HTSC) up to 72 hours post incubation.

TABLE 11
MS data from Novatia LCMS

| | | SEQ | % Full | | % Full | | % Full | |
|---|---|---|---|---|---|---|---|---|
| Oligo | | ID | length | MW | length | MW at | length | MW |
| ID | Sequence | NO: | at 0 h | at 0 h | at 0 h | 24 h | at 0 h | at 72 h |
| A- | (Pmd)uCfacuUfuAfUfuga | 89 | 100 | 7709.1 | 100 | 7709.0 | 100 | 7709.2 |
| 515489 | gUfuUfcugugscsc | | | | | | | |
| A- | (Pmds)uCfacuUfuAfUfug | 90 | 100 | 7725.4 | 100 | 7725.4 | 100 | 7725.7 |
| 515491 | agUfuUfcugugscsc | | | | | | | |

TABLE 12
MS data from Agilent LCMS

| | | SEQ | % Full- | | % Full | | MW |
|---|---|---|---|---|---|---|---|
| Oligo | | ID | length | MW | length | MW | (5′ PS) |
| ID | Sequence | NO: | at 0 h | at 0 h | at 24 h | at 24 h | at 24 h |
| A- | (Cymd)usCfsacuUfuAf | 91 | 100 | 7721.32 | 100 | 7721.09 | |
| 1875173 | UfugagUfuUfcugugscsc | | | | | | |
| A- | (Cymds)usCfsacuUfuAf | 92 | 100 | 7705.13 | 100 | 7705.11 | |
| 1875172 | UfugagUfuUfcugugscsc | | | | | | |
| A- | (Pd)usCfsacuUfuAfUfug | 93 | 100 | 7744.6 | <100 | 7744.4 | 7608.67 |
| 1875175 | agUfuUfcugugscsc | | | | | | (~10-20%) |
| A- | (Pds)usCfsacuUfuAfUfu | 94 | 100 | 7728.37 | 100 | 7728.37 | |
| 1875174 | gagUfuUfcugugscsc | | | | | | |
| A- | (Pmmd)usCfsacuUfuAf | 95 | 100 | 7608.88 | 58 | 7755.03 | 7608.88 |
| 1875179 | UfugagUfuUfcugugscsc | | | | | | (42%) |
| A- | (Pmmds)usCfsacuUfuAf | 96 | 100 | 7739.01 | 14 | 7739.94 | 7592.83 |
| 1875178 | UfugagUfuUfcugugscsc | | | | | | (86%) |
| A- | (Ptmd)usCfsacuUfuAfU | 97 | 100 | 7753.11 | 100 | 7753.16 | |
| 1875180 | fugagUfuUfcugugscsc | | | | | | |
| A- | (Ptmds)usCfsacuUfuAf | 98 | 100 | 7769.1 | 100 | 7769.02 | |
| 1875181 | UfugagUfuUfcugugscsc | | | | | | |

Upper case letter followed with f-2′-F sugar modification; lower case letter-2′-OMe sugar modification; s-phosphorothioate (PS) linkage; the structures for the prodrugs are the same as in Table 8 above.

The representative LCMS spectra of the oligonucleotides tested in the DTT reduction assay are shown in FIGS. 4A-J.

### Example 5. Glutathione Assay to Check the Cleavability of Cyclic Prodrug Analogues

Modified oligonucleotide (11-nt or 23-nt length) was added at 100 μM to a solution of 250 μg (6.25 U/mL) glutathione-S-transferase from equine liver (GST) (Sigma Cat. No. G6511) and 0.1 mg/mL NADPH (Sigma Cat. No. 481973) in 0.1 M Tris pH 7.2. Glutathione (GSH) (MP Biomedicals, Inc. Cat. No. 101814 #) was added to the mixture for a final concentration of 10 mM. Immediately after addition of GSH, sample was injected onto a Dionex DNAPac PA200 column (4×250 mm) at 30° C. and run on an anion exchange gradient of 35-65% (20 mM Sodium Phosphate, 10-15% CH₃CN, 1M Sodium Bromide pH11) at 1 mL/min for 6.5 minutes.

Glutathione-mediated cleavage kinetics were monitored every hour for 24 hours. The area under the main peak for each hour was normalized to the area from the 0 h time point (first injection). First-order decay kinetics were used to calculate half-lives. A control sequence containing modified oligonucleotide (23-nt length) with 5′ Thiol modifier C6 (Glen Research Cat. No. 10-1936-02) between N6 and N7 was run each day of assay run. A second control sequence containing modified oligonucleotide (23-nt length) with the same 5′ thiol modifier C6 at N1 was also run once per set of sequences. Half-lives were reported relative to half-life of control sequence. Glutathione and GST were prepared as stocks of 100 mM and 10 mg/mL in water, respectively, and aliquoted into 1 mL tubes and stored at −80° C. A new aliquot was used for every day the assay was run.

TABLE 13
Half-life of oligonucleotides 5′ modified phosphate prodrug after incubating with glutathione

| | | SEQ ID | Half-Life |
|---|---|---|---|
| Oligo ID | Sequence | NO: | (h) |
| A-515432 | (Pmd)dTdTdTdTdTdTdTdTdTdT | 99 | >24 |
| A-515433 | (Pmds)dTdTdTdTdTdTdTdTdTdT | 100 | >24 |
| A-801703 | Q51uUfaUfaGfaGfcAfagaAfcAfcUfgUfuuu | 101 | <1 |
| (Control 1) | | | |
| A-801704 | uUfaUfaGfQ51GfcAfagaAfcAfcUfgUfuuu | 102 | 4.2 |
| (Control 2) | | | |
| A-1875173 | (Cymd)usCfsacuUfaAfUfugagUfuUfcugugscsc | 103 | >24 |
| A-1875172 | (Cymds)usCfsacuUfuAfUfugagUfuUfcugugscsc | 104 | >24 |
| A-1875175 | (Pd)usCfsacuUfuAfUfugagUfuUfcugugscsc | 105 | >24 |
| A-1875174 | (Pds)usCfsacuUfuAfUfugagUfuUfcugugscsc | 106 | >24 |
| A-1875179 | (Pmmd)usCfsacuUfuAfUfugagUfuUfcugugscsc | 107 | >24 |
| A-1875178 | (Pmmds)usCfsacuUfuAfUfugagUfuUfcugugscsc | 108 | >24 |
| A-1875180 | (Ptmd)usCfsacuUfuAfUfugagUfuUfcugugscsc | 109 | >24 |
| A-1875181 | (Ptmds)usCfsacuUfuAfUfugagUfuUfcugugscsc | 110 | >24 |

Upper case letter followed with f- 2′-deoxy-2′-fluoro (2′-F) sugar modification; lower case
letter- 2′-O-methyl (2′-OMe) sugar modification; s- phosphorothioate (PS) linkage; the
structures for the prodrug are the same as in Table 9 above; Q51-

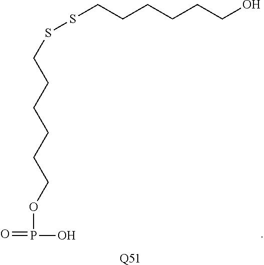

### Example 6. In Vivo Evaluation of siRNA Duplexes Containing Modified Phosphate Prodrugs at the 5′ End

In vivo study procedure: C57bl6 female mice (n=3/group) were dosed with 0.3 mg/mL of siRNA duplex containing the modified phosphate prodrugs at the 5′ end in Table 14. Serum was collected at days 11, 22 and 35 days pos-dose and analyzed via ELISA to determine relative F12 protein levels. The results are shown in FIG. 5.

TABLE 14
siRNA duplexes used in the in vivo evaluation shown in FIG. 5

| | | | | | SEQ | | Molecular |
|---|---|---|---|---|---|---|---|
| Duplex | Oligo | | | | ID | Molecular | Weight |
| ID | ID | Strand | Target | Oligo Seq | NO: | Weight | found |
| AD- | A- | sense | F12 | csascagaAfaCfUfCfaaua | 111 | 8801.656 | 8797.128 |
| 73603 | 147464 | | | aagugaL96 | | | |
| | A- | antis | F12 | usCfsacuUfuAfUfugag | 112 | 7514.875 | 7511.025 |
| | 147465 | | | UfuUfcugugscsc | | | |
| AD- | A- | sense | F12 | csascagaAfaCfUfCfaaua | 113 | 8801.656 | 8797.128 |
| 74843 | 147464 | | | aagugaL96 | | | |
| | A- | antis | F12 | VPusCfsacuUfuAfUfug | 114 | 7591.874 | 7587.005 |
| | 150219 | | | agUfuUfcugugscsc | | | |
| AD- | A- | sense | F12 | csascagaAfaCfUfCfaaua | 115 | 8801.656 | 8797.128 |
| 291896 | 147464 | | | aagugaL96 | | | |
| | A- | antis | F12 | VPuCfacuUfuAfUfugag | 116 | 7558.743 | 7555.05 |
| | 447595 | | | UfuUfcugugscsc | | | |
| AD- | A- | sense | F12 | csascagaAfaCfUfCfaaua | 117 | 8801.656 | 8797.128 |
| 326758 | 147464 | | | aagugaL96 | | | |
| | A- | antis | F12 | uCfacuUfuAfUfugagUf | 118 | 7482.744 | 7479.071 |
| | 515487 | | | uUfcugugscsc | | | |
| AD- | A- | sense | F12 | csascagaAfaCfUfCfaaua | 119 | 8801.656 | 8797.128 |
| 326756 | 147464 | | | aagugaL96 | | | |
| | A- | antis | F12 | (Pmd)usCfsacuUfuAfUf | 120 | 7744.095 | 7738.993 |
| | 515488 | | | ugagUfuUfcugugscsc | | | |
| AD- | A- | sense | F12 | csascagaAfaCfUfCfaaua | 121 | 8801.656 | 8797.128 |
| 326757 | 147464 | | | aagugaL96 | | | |
| | A- | antis | F12 | (Pmd)uCfacuUfuAfUfu | 122 | 7711.964 | 7707.039 |
| | 515489 | | | gagUfuUfcugugscsc | | | |
| AD- | A- | sense | F12 | csascagaAfaCfUfCfaaua | 123 | 8801.656 | 8797.128 |
| 326759 | 147464 | | | aagugaL96 | | | |
| | A- | antis | F12 | (Pmds)usCfsacuUfuAfU | 124 | 7760.161 | 7754.97 |
| | 515490 | | | fugagUfuUfcugugscsc | | | |
| AD- | A- | sense | F12 | csascagaAfaCfUfCfaaua | 125 | 8801.656 | 8797.128 |
| 326760 | 147464 | | | aagugaL96 | | | |
| | A- | antis | F12 | (Pmds)uCfacuUfuAfUfu | 126 | 7728.03 | 7723.016 |
| | 515491 | | | gagUfuUfcugugscsc | | | |

Upper case letter followed with f-2′-F sugar modification; lower case letter-2′-OMe sugar modification; s-phosphorothioate (PS) linkage; the structures for the prodrug Pmds and ligand L96 are the same as in Table 8 above.

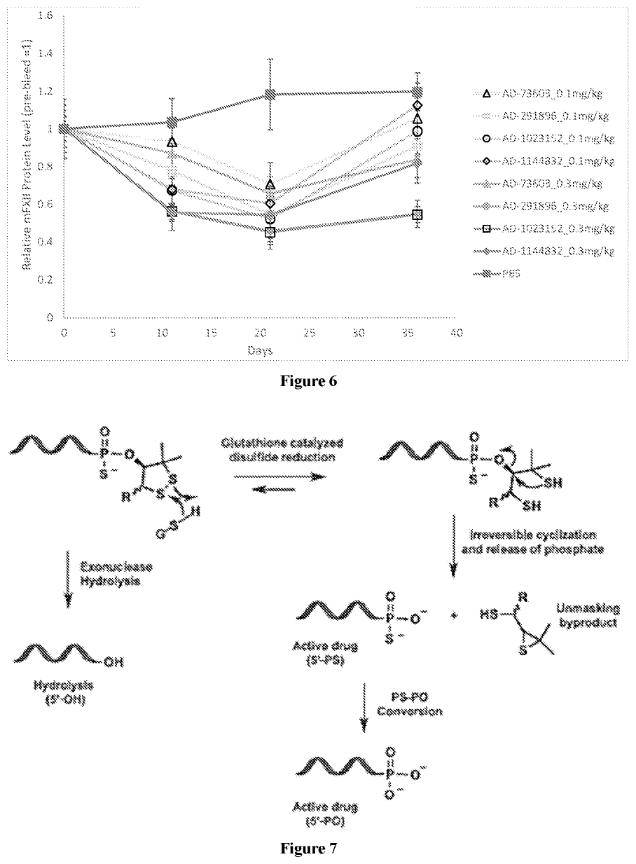

In vivo study procedure: C57bl6 female mice (n=3/group) were dosed with either 0.1 mg/mL or 0.3 mg/mL of siRNA duplex containing the modified phosphate prodrugs at the 5′ end in Table 15. Serum was collected at days 11, 22 and 35 days pos-dose and analyzed via ELISA to determine relative F12 protein levels. The results are shown in FIG. 6.

TABLE 15
siRNA Duplexes used for in vivo evaluation in FIG. 6

| Duplex | Sense Strand Sequence | SEQ ID | Antisense Strand Sequence | SEQ ID |
|---|---|---|---|---|
| ID | (5′-3′) | NO: | (5′-3′) | NO: |
| AD- | csascagaAfaCfUfCfaauaa | 127 | usCfsacuUfuAfUfugagUfu | 128 |
| 73603 | agugaL96 | | Ufcugugscsc | |
| AD- | csascagaAfaCfUfCfaauaa | 127 | VPuCfacuUfuAfUfugagUf | 129 |
| 291896 | agugaL96 | | uUfcugugscsc | |
| AD- | csascagaAfaCfUfCfaauaa | 127 | (Pmmds)usCfsacuUfuAfUf | 130 |
| 1023152 | agugaL96 | | ugagUfuUfcugugscsc | |
| AD- | csascagaAfaCfUfCfaauaa | 127 | (Pmmds)uCfacuUfuAfUfu | 131 |
| 1144832 | agugaL96 | | gagUfuUfcugugscsc | |

Upper case letter followed with f-2′-F sugar modification; lower case letter-2′-OMe sugar modification; s-phosphorothioate (PS) linkage; the structures for the prodrug Pmds and ligand L96 are the same as in Table 8 above.

### Example 7. In Vivo Metabolic Stability and Determination of 5′-Phosphate

Metabolic stability study procedure: C57bl6 female mice (n=2/group) Mice were dosed with 10 mg/kg siRNA duplex containing the modified phosphate prodrugs at the 5′ end in Table 16. Livers were collected 5 days post-dose and analyzed via LC-MS.

TABLE 16
siRNA Duplexes used for metabolic evaluation

| Duplex | Sense Strand Sequence | SEQ ID | Antisense Strand Sequence | SEQ ID |
|---|---|---|---|---|
| ID | (5′-3′) | NO: | (5′-3′) | NO: |
| AD- | csascagaAfaCfUfCfaauaaagug | 132 | (Pmds)uCfacuUfuAfUfuga | 133 |
| 326760 | aL96 | | gUfuUfcugugscsc | |
| AD- | csascagaAfaCfUfCfaauaaagug | 132 | (Pmmds)usCfsacuUfuAfUf | 134 |
| 1023152 | aL96 | | ugagUfuUfcugugscsc | |
| AD- | csascagaAfaCfUfCfaauaaagug | 132 | (Cymds)usCfsacuUfuAfUf | 135 |
| 1023155 | aL96 | | ugagUfuUfcugugscsc | |
| AD- | csascagaAfaCfUfCfaauaaagug | 132 | (Pds)usCfsacuUfuAfUfuga | 136 |
| 1023156 | aL96 | | gUfuUfcugugscsc | |
| AD- | csascagaAfaCfUfCfaauaaagug | 132 | (Ptmds)usCfsacuUfuAfUfu | 137 |
| 1023157 | aL96 | | gagUfuUfcugugscsc | |

Upper case letter followed with f-2′-F sugar modification; lower case letter-2′-OMe sugar modification; s-phosphorothioate (PS) linkage; the structures for the prodrugs are the same as in Table 8 above.

The results of the metabolic stability study are shown in Tables 17 and 18.

TABLE 17
siRNA metabolites found after in vivo study

| | | Metabolites (%) | | | | | | | | | |
|---|---|---|---|---|---|---|---|---|---|---|---|
| Duplex | Strand | | | | | | | | | | (pd)N1 + |
| ID | (5′→3′) | FL | (pd)N1 | N3N4 | N5N6 | N13N14 | N18N19 | N20N21 | N21L96 | N22N23 | N22N23 |
| AD- | Sense | 98 | | | | | 1 | 1 | | | |
| 326760 | Antisense | 72 | | | | 1 | | | | 28 | |
| AD- | Sense | 97 | | | | | | 1 | 2 | | |
| 1023155 | Antisense | 70 | 6 | | | | | | | 21 | 2 |
| AD- | Sense | 100 | | | | | | | | | |
| 1023156 | Antisense | 64 | 12 | | | | | | | 18 | 6 |
| AD- | Sense | 96 | | | | | 1 | 1 | 2 | | |
| 1023152 | Antisense | 72 | | | 2 | | | | | 25 | 1 |
| AD- | Sense | 90 | | 2 | | | 2 | 2 | 4 | | |
| 1023157 | Antisense | 71 | | | 2 | | | | | 27 | |

Sense (5′→3′) = N1N2N3N4N5N6N7N8N9N10N11N12N13N14N15N16N17N18N19N20N21L96
Antisense (5′→3′) = (pd)N1N2N3N4N5N6N7N8N9N10N11N12N13N14N15N16N17N18N19N20N21N22N23
FL = (Full Length Strand)
(pd) = prodrug

TABLE 18
% of 5′-phosphate, thiophosphate, or hydroxy
group found after 5 days in mice on antisense strand

| Duplex ID | 5′PO (%) | 5′PS (%) | 5′OH (%) |
|---|---|---|---|
| AD-326760 | 0 | 0 | 5 |
| AD-1023155 | 0 | 0 | 8 |
| AD-1023156 | 0 | 0 | 18 |
| AD-1023152 | 1.6 | 1.3 | 1 |
| AD-1023157 | 0 | 0 | 0 |

The possible in vivo cytosolic unmasking mechanism of the 5′ cyclic disulfide modified phosphate prodrugs to reveal 5′-phosphate is shown in FIG. 7.

### Example 8: In Vivo Evaluation of siRNA Duplexes Containing Modified Phosphate Prodrugs at the 5′ End in CNS

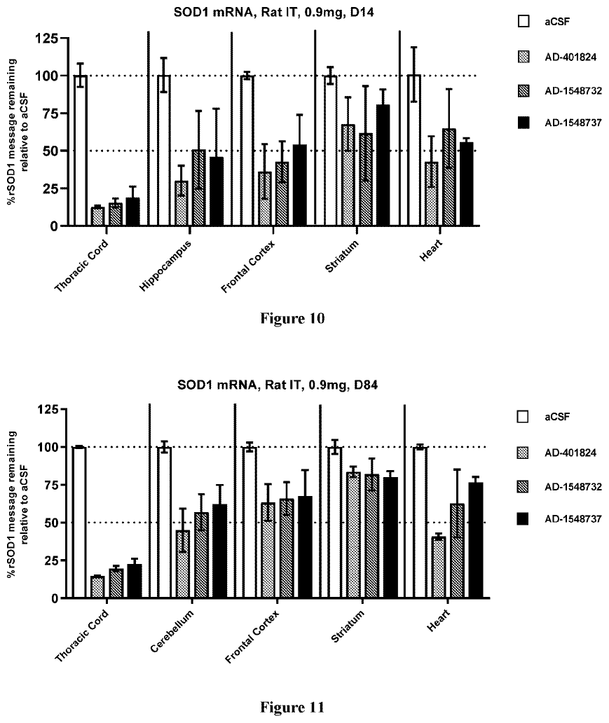

In vivo study procedure for CNS (IT—Intrathecal administration): Female rat (n=3/group) were dosed with 0.1 mg/rat, 0.3 mg/rat, or 0.9 mg/rat of SOD1 siRNA duplex containing the modified phosphate prodrugs at the 5′ end in Table 19. Brain was collected after 14 or 84 days of IT administration and dissected into different regions for qPCR analysis to determine the relative SOD1 mRNA levels. The results are shown in FIGS. 5-13.

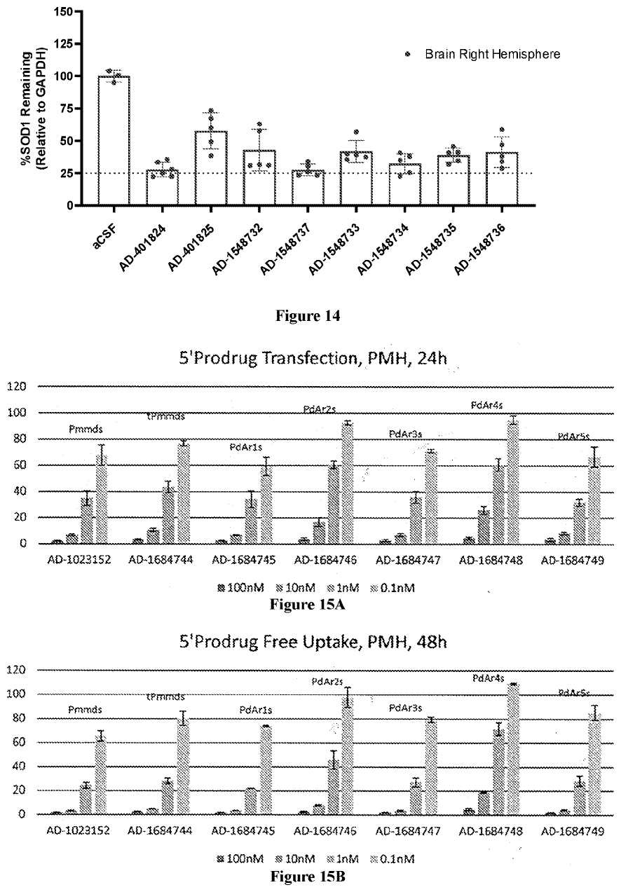

In vivo study procedure for CNS (ICV—intracranial ventricular administration): C57bl6 female mice (n=4/group) were dosed with 100 pig of SOD1 siRNA duplex containing the modified phosphate prodrugs at the 5′ end in Table 19. Brain was collected after 7 days of ICV administration and the right hemisphere was used for qPCR analysis to determine the relative SOD mRNA levels. The results are shown in FIG. 14.

TABLE 19
siRNAs for CNS studies

| Duplex | | | | | SEQ | Molecular | Exact |
|---|---|---|---|---|---|---|---|
| Id | Oligo Id | Strand | Target | Oligo Sequence | ID NO: | Weight | Mass |
| 401824 | 637448 | sense | SOD1 | csasuuu(Uhd)AfaUfCfCfuc | 138 | 7043.98 | 7040.25 |
| | | | | acucuasasa | | | |
| | 444402 | antis | | VPusUfsuagAfgUfGfagga | 139 | 7851.16 | 7847.15 |
| | | | | UfuAfaaaugsasg | | | |
| 401825 | 637448 | sense | SOD1 | csasuuu(Uhd)AfaUfCfCfuc | 140 | 7043.98 | 7040.25 |
| | | | | acucuasasa | | | |
| | 268862 | antis | | usUfsuagAfgUfGfaggaUfu | 141 | 7775.16 | 7771.18 |
| | | | | Afaaaugsasg | | | |
| 1548732 | 637448 | sense | SOD1 | csasuuu(Uhd)AfaUfCfCfuc | 142 | 7043.98 | 7040.25 |
| | | | | acucuasasa | | | |
| | 2717577 | antis | | (Pmmds)usUfsuagAfgUfGf | 143 | 8017.47 | 8013.14 |
| | | | | aggaUfuAfaaaugsasg | | | |
| 1548737 | 637448 | sense | SOD1 | csasuuu(Uhd)AfaUfCfCfuc | 144 | 7043.98 | 7040.25 |
| | | | | acucuasasa | | | |
| | 2717578 | antis | | (tPmmds)usUfsuagAfgUfG | 145 | 8017.47 | 8013.14 |
| | | | | faggaUfuAfaaaugsasg | | | |
| 1548733 | 637448 | sense | SOD1 | csasuuu(Uhd)AfaUfCfCfuc | 146 | 7043.98 | 7040.25 |
| | | | | acucuasasa | | | |
| | 2717579 | antis | | (Pds)usUfsuagAfgUfGfagg | 147 | 8005.41 | 8001.11 |
| | | | | aUfuAfaaaugsasg | | | |
| 1548734 | 637448 | sense | SOD1 | csasuuu(Uhd)AfaUfCfCfuc | 148 | 7043.98 | 7040.25 |
| | | | | acucuasasa | | | |
| | 2717580 | antis | | (Pmmds)UsUfsuagAfgUfG | 149 | 8003.44 | 7999.13 |
| | | | | faggaUfuAfaaaugsasg | | | |
| 1548735 | 637448 | sense | SOD1 | csasuuu(Uhd)AfaUfCfCfuc | 150 | 7043.98 | 7040.25 |
| | | | | acucuasasa | | | |
| | 2717581 | antis | | (Pmmds)Us(Ufms)uagAfg | 151 | 8017.47 | 8013.14 |
| | | | | UfGfaggaUfuAfaaaugsasg | | | |
| 1548736 | 637448 | sense | SOD1 | csasuuu(Uhd)AfaUfCfCfuc | 152 | 7043.98 | 7040.25 |
| | | | | acucuasasa | | | |
| | 2717582 | antis | | (Pmmds)(URs)(Ufms)uagA | 153 | 8013.14 | 8013.14 |
| | | | | fgUfGfaggaUfuAfaaaugsas | | | |
| | | | | g | | | |
| 1700788 | 637448 | sense | SOD1 | csasuuu(Uhd)AfaUfCfCfuc | 154 | 7043.98 | 7040.25 |
| | | | | acucuasasa | | | |
| | 3019034 | antis | | (PdAr1s)usUfsuagAfgUfGf | 155 | 8079.53 | 8075.16 |
| | | | | aggaUfuAfaaaugsasg | | | |
| 1700790 | 637448 | sense | SOD1 | csasuuu(Uhd)AfaUfCfCfuc | 156 | 7043.98 | 7040.25 |
| | | | | acucuasasa | | | |
| | 3019035 | antis | | (PdAr3s)usUfsuagAfgUfGf | 157 | 8093.56 | 8089.17 |
| | | | | aggaUfuAfaaaugsasg | | | |
| 1700789 | 637448 | sense | SOD1 | csasuuu(Uhd)AfaUfCfCfuc | 158 | 7043.98 | 7040.25 |
| | | | | acucuasasa | | | |
| | 3019036 | antis | | (PdAr5s)usUfsuagAfgUfGf | 159 | 8109.56 | 8105.17 |
| | | | | aggaUfuAfaaaugsasg | | | |

Upper case letter followed with f- 2′-deoxy-2′-fluoro (2′-F) sugar modification; lower case letter-
2′-O-methyl (2′-OMe) sugar modification; s- phosphorothioate (PS) linkage; Uhd: 2′-O-hexadecyl
uridine (2′-C16); VP- vinyl phosphonate; the prodrugs:

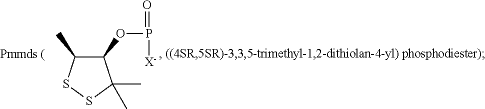

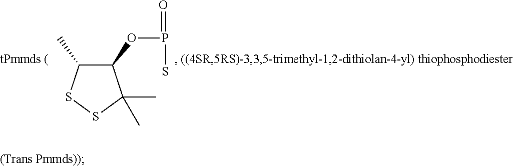

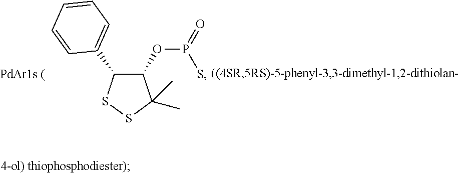

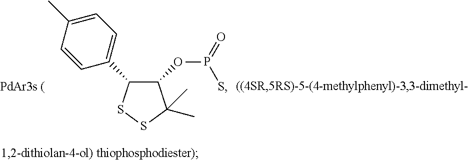

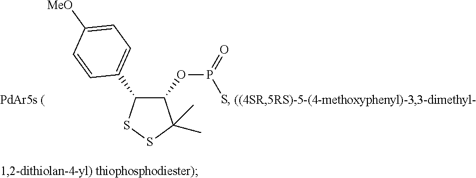

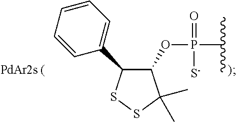

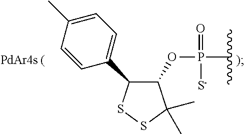

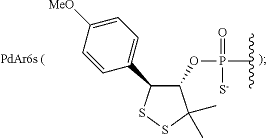

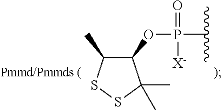

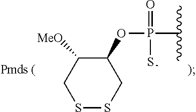

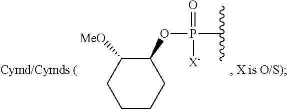

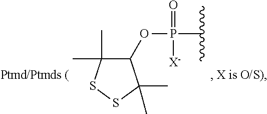

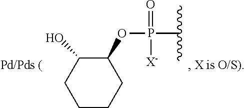

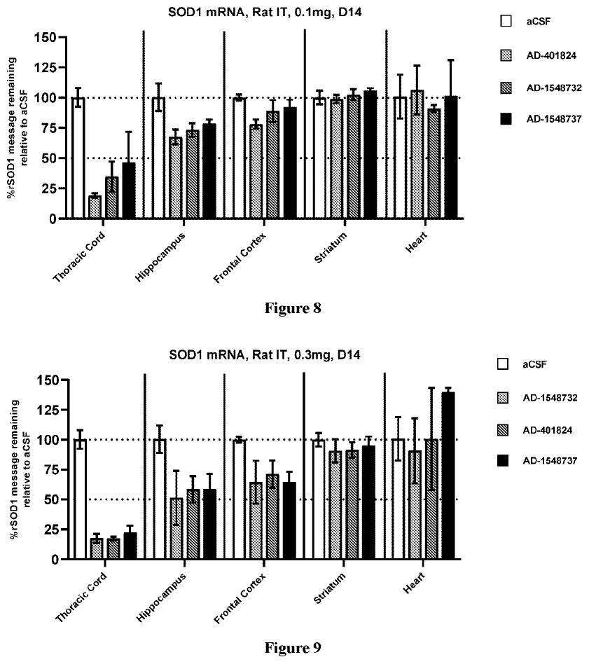

As shown in FIGS. 8-11, the siRNA duplex containing Pmmds and tPmmds prodrugs at the 5′ end displayed similar activity and duration as the siRNA duplex containing 5′-VP control in CNS tissues.

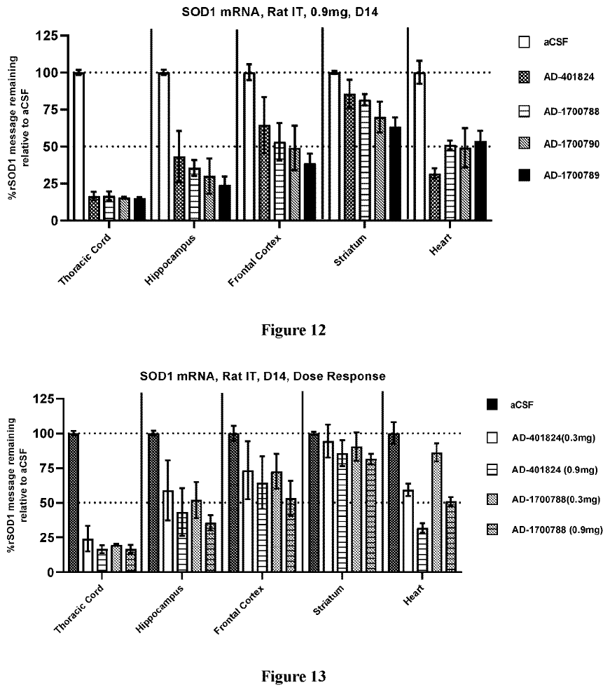

As shown in FIG. 12-13, the siRNA duplex containing PdAr1s, PdAr3s, and PdAr5s prodrugs at the 5′ end displayed better or at least comparable activity as compared to the siRNA duplex containing 5′-VP control in CNS tissues.

Overall, metabolically stable 5′-phosphate mimic such as 5′-VP could improve siRNA activity in extrahepatic tissues with less efficient endogenous 5′-phosphorylation of modified siRNA. The novel 5′ modified phosphate prodrug described herein showed stability in plasma and endosomal environment. These 5′ modified phosphate prodrugs unmasked in cytosol to reveal f natural 5′-phosphate (or phosphorothioate) required for efficient RISC loading. The siRNAs containing novel 5′ modified phosphate prodrugs at the 5′ end displayed activity comparable to or even better than that of siRNAs containing a stable 5′-phosphate mimic design, such as 5′-VP.

For instance, the activity of the siRNAs containing the following list of 5′ modified phosphate prodrugs,

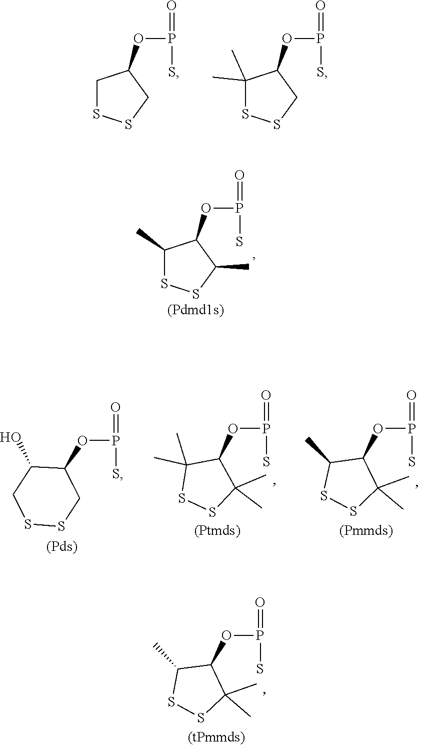

were generally comparable to the activity of siRNAs containing 5′-VP. The siRNAs containing the following list of 5′ modified phosphate prodrugs,

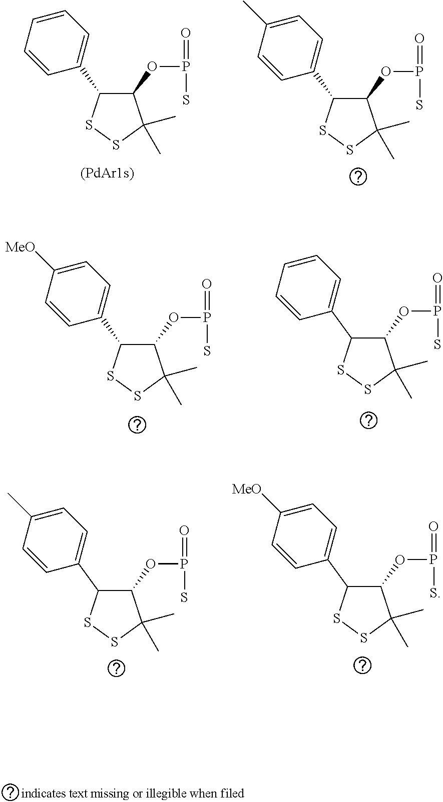

generally have an improved stability than that of siRNAs containing 5′-VP and have a better or comparable activity than that of siRNAs containing 5′-VP.

### Example 9. Introduction of the Modified Phosphate Prodrugs for Masking Internucleotide Phosphate Linkages to Mask the Charge

Different cyclic phosphate prodrug derivatives can be introduced to the phosphate group as a temporary protecting group to any internucleotide phosphate group on either the sense or antisense strand or both the sense and antisense strands, as shown in the Schemes 14-19.

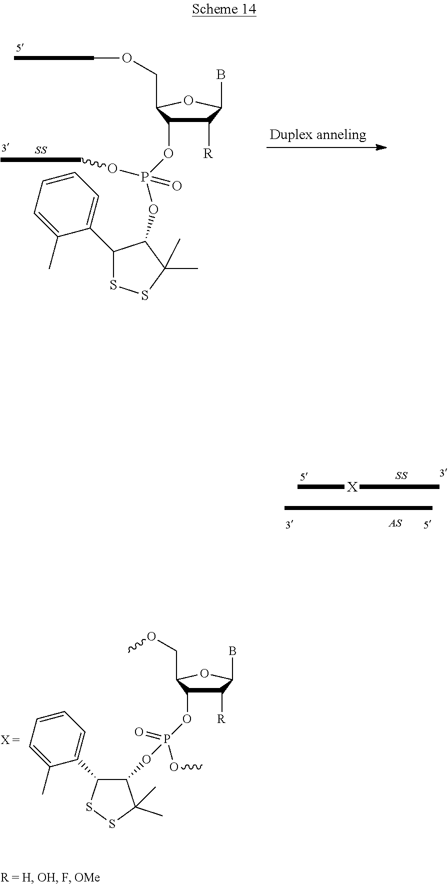

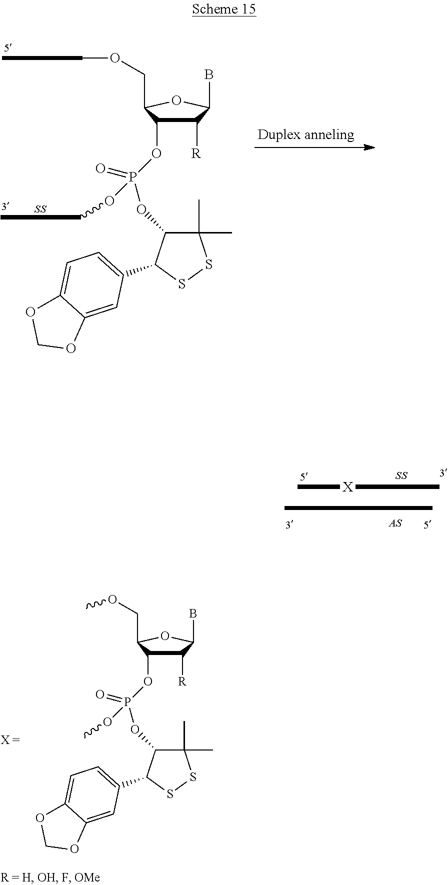

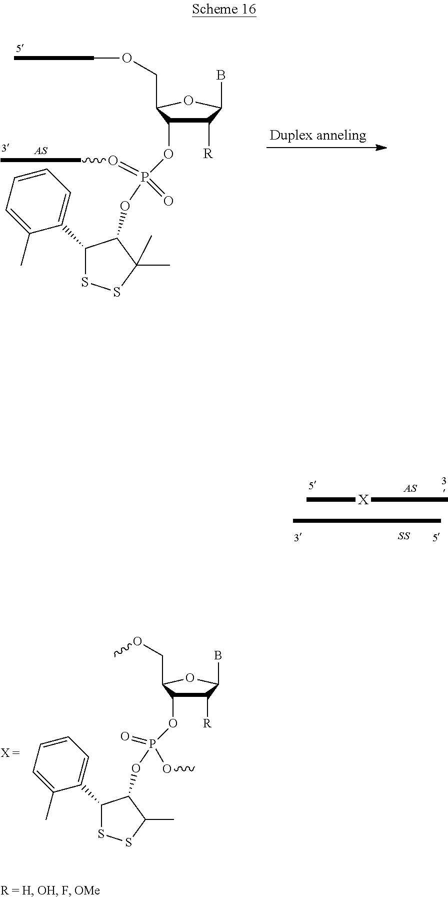

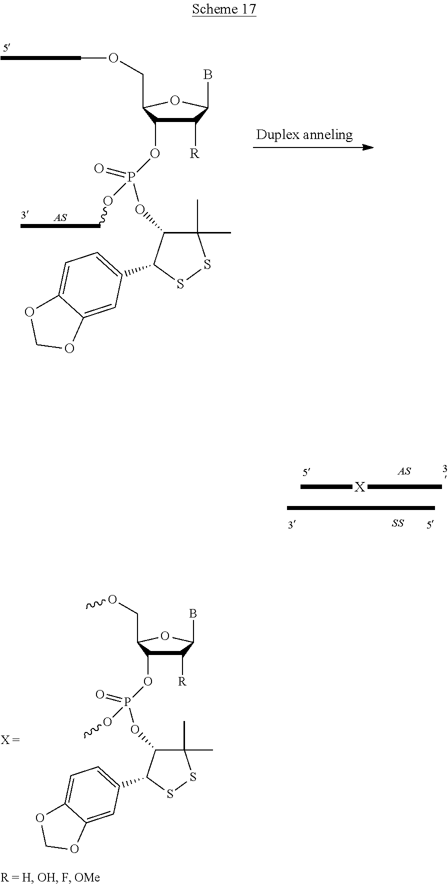

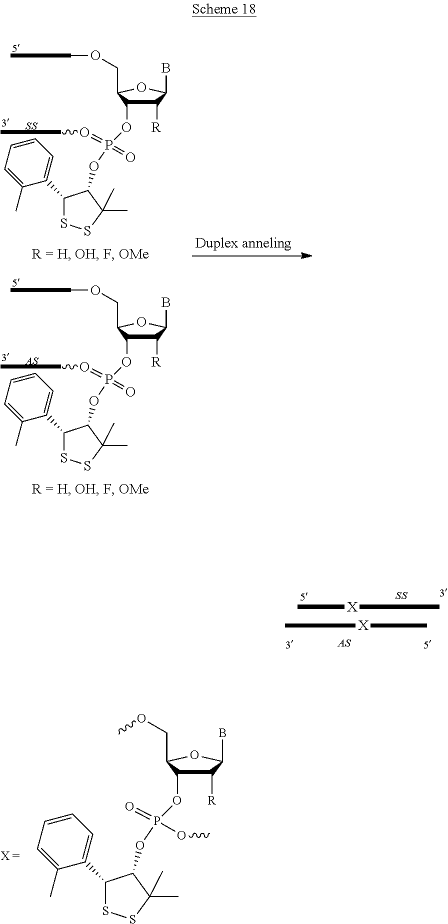

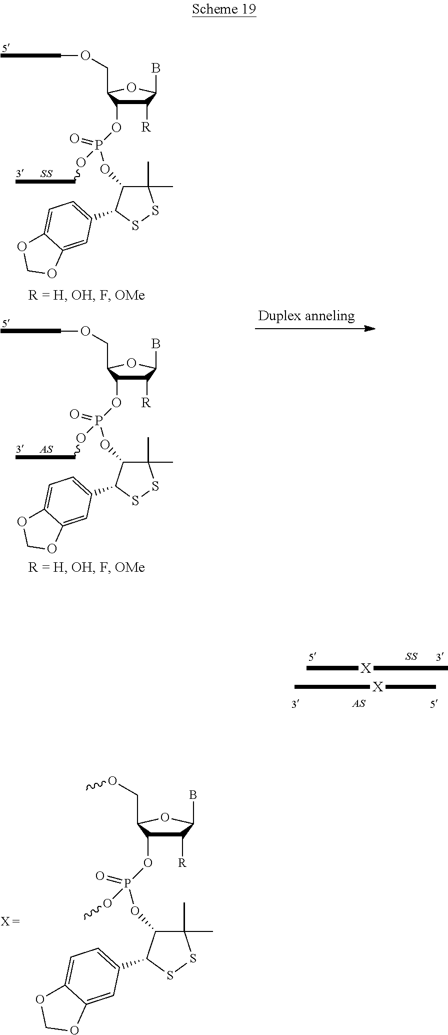

### Example 10. Using the Modified Phosphate Prodrugs to Generate Cleavable siRNA Conjugates Having Different Targeting Ligands

Different targeting ligands can be introduced into a siRNA duplex through the cyclic phosphate derivatives as shown in Scheme 20. These derivatives will be cleaved off after the siRNA enters into cytosol.

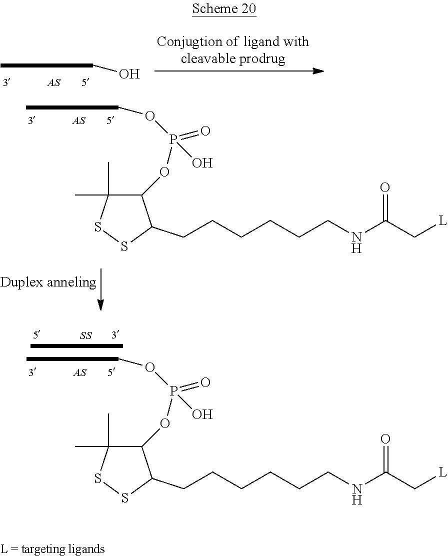

### Example 11. Synthesis of Cyclic-Disulfide Modified Phosphate Prodrug Derivatives

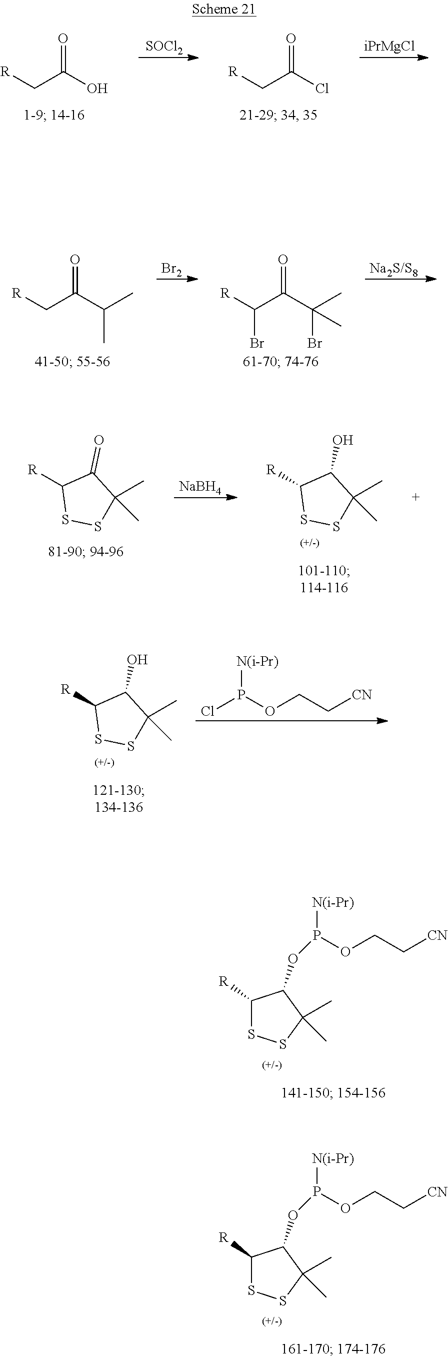

General procedure—Compound 21 (R=4-FC₆H₄): 4-fluorphenylacetic acid 1 (5.0 g, 32.4 mmol) was dissolved in anhydrous toluene (20 mL) under inert atmosphere and cooled in a water/ice bath. Thionyl chloride (7.1 mL, 97.3 mmol) was added dropwise, then stirred for 30 minutes before being warmed to room temperature. The reaction was stirred for an additional 18 hours, concentrated in vacuum, the residue was co-evaporated with toluene, and dried in high vacuum. Compound 21 was obtained as a yellow oil, (5.25 g) 94% yield. ¹H NMR (600 MHz, CDCl₃) δ 7.24 (dd, J=8.6, 5.3 Hz, 2H), 7.06 (t, J=8.6 Hz, 2H), 4.12 (s, 2H).

Compound 24 (R=2-MeC₆H₄): Compound 24 was prepared analogously from Compound 4 (R=2-MeC₆H₄) (10.5 g, 69.9 mmol) with a 93% yield. ¹H NMR (CDCl₃) δ 2.35 (s, 3H), 4.19 (s, 2H), 7.21-7.31 (m, 4H). ¹³C NMR (CDCl₃) δ 17.6, 49.3, 124.6, 126.7, 128.4, 128.7, 128.8, 135.1, 169.8.

Compound 25 (R=2-MeOC₆H₄): Compound 25 was prepared analogously from Compound 5 (R=2-MeOC₆H₄) (10.5 g, 63.2 mmol) with a 93% yield. ¹H NMR (CDCl₃) δ 3.87 (s, 3H), 4.16 (s, 2H), 6.93-6.94 (m, 1H), 6.96-6.99 (m, 1H), 7.19-7.20 (m, 1H), 7.34-7.37 (m, 1H). ¹³C NMR (CDCl₃) δ 48.1, 55.5, 110.7, 120.7, 120.9, 131.0, 157.5, 171.8.

Compound 26 (R=2,4-F₂C₆H₃): Compound 26 was prepared analogously from Compound 6 (R=2,4-F₂C₆H₃) (5.0 g, 29 mmol) with a 94% yield. ¹H NMR (600 MHz, CDCl₃) δ 7.23 (td, J=8.4, 6.1 Hz, 1H), 6.94-6.81 (m, 2H), 4.16 (d, J=1.3 Hz, 2H). ¹³C NMR (CDCl₃) δ 45.8, 104.3, 111.8, 115.1, 132.2, 162.0, 163.9, 170.8.

Compound 27 (R=2,4-(MeO)₂C₆H₃): Compound 27 was prepared analogously from Compound 7 (R=2,4-(MeO)₂C₆H₃) (6.9 g) with a 99% yield. ¹H NMR (600 MHz, CDCl₃) δ 7.08-7.04 (m, 1H), 6.50-6.44 (m, 2H), 4.05 (s, 2H), 3.81 (s, 6H).

Compound 28 (R=3,4-(CH₂O₂)C₆H₃): Compound 28 was prepared analogously from Compound 8 (R=3,4-(CH₂O₂)C₆H₃) (6.5 g) with a 99% yield. ¹H NMR (600 MHz, CDCl₃) δ 6.81-6.70 (m, 3H), 5.97 (s, 2H), 4.04 (s, 2H).

Compound 29 (R=2,4,6-Me₃C₆H₂): Compound 29 was prepared analogously from Compound 9 (R=2,4,6-Me₃C₆H₂) (10.0 g, 56 mmol) with a 99% yield. ¹H NMR (600 MHz, CDCl₃) δ 6.89 (s, 2H), 4.18 (s, 2H), 2.27 (d, J=5.5 Hz, 9H). ¹³C NMR (151 MHz, CDCl₃) δ 20.1, 21.0, 47.2, 126.6, 129.2, 137.0, 137.9, 171.9.

Compound 34 (R=3-MeOC₆H₅): Compound 34 was prepared analogously from 14 (R=3-MeOC₆H₅) (20.0 g). 96% yield. ¹H NMR (600 MHz, DMSO) δ 7.26-7.18 (m, 1H), 6.86-6.77 (m, 3H), 3.73 (s, 3H), 3.53 (s, 2H); ¹³C NMR (151 MHz, DMSO) δ 170.8, 157.5, 134.7, 127.6, 119.9, 113.4, 110.3, 53.3, 39.0.

Compound 35 (R=4-CF₃C₆H₅): Compound 35 was prepared analogously from 15 (R=4-CF₃C₆H₅) (15.0 g, 73.5 mmol). 90% yield. ¹H NMR (600 MHz, CDCl₃) δ 7.67 (d, J=8.0 Hz, 2H), 7.43 (d, J=8.0 Hz, 2H), 4.24 (s, 2H).

General procedure—Compound 41 (R=4-FC₆H₄): Compound 21 (R=4-FC₆H₄) (5 g, 29.0 mmol) was dissolved in anhydrous tetrahydrofuran (120 mL) under inert atmosphere. Copper (I) bromide (4.2 g, 29.0 mmol) was added, the suspension was stirred for 5 minutes, then cooled in a water/ice bath. Isopropyl magnesium chloride (2M in THF, 16 mL, 31.9 mmol) was added dropwise to the stirred solution over 20 minutes. The reaction was allowed to slowly warm to room temperature and stirred for 5 hours. The mixture was quenched with saturated aqueous ammonium chloride and diluted with ethyl acetate. The organic layer was separated, washed with aq. 5% NaCl, saturated NaCl, dried over Na₂SO₄, filtered, and concentrated to afford a crude oil. The crude product was purified by silica gel flash chromatography. Compound 41 was obtained as a light-yellow oil, (4.2 g) 80% yield. ¹H NMR (600 MHz, DMSO-D6) δ 7.23-7.18 (m, 2H), 7.15-7.10 (m, 2H), 3.84 (s, 2H), 2.73 (heptet, J=6.9 Hz, 1H), 1.04 (d, J=7.0 Hz, 6H). MS (ESI+APCI), calculated for C₁₁H₁₃FO [M] exact mass m/z=180.10.

Compound 44 (R=2-MeC₆H₄): Compound 44 was prepared analogously from compound 24 (R=2-MeC₆H₄), (5.40 g, 32 mmol). 68% yield, ¹H NMR (600 MHz, CDCl₃) δ 1.15 (d, 6H), 2.25 (s, 3H), 2.73-2.78 (m, 1H), 3.79 (s, 2H), 4.75 (s, 1H), 7.12-7.21 (m, 4H). ¹³C NMR (151 MHz, CDCl₃) δ 18.5, 19.7, 40.0, 46.0, 126.1, 127.2, 130.4, 133.3, 136.9, 212.0. MS (ESI+APCI), calculated for C₁₂H₆O [M+H]⁺ exact mass m/z=177.12, found 177.2.

Compound 45 (R=2-MeOC₆H₄): Compound 45 was prepared analogously from compound 25 (R=2-MeOC₆H₄), (5.50 g, 26 mmol). 75% yield, ¹H NMR (600 MHz, CDCl₃) δ 1.14 (d, J=5 Hz, 6H), 2.75 (m, 1H), 3.76 (s, 2H), 3.81 (s, 3H), 6.88-6.89 (m, 1H), 6.93-6.95 (m, 1H), 7.13-7.15 (m, 1H), 7.25-7.28 (m, 1H). ¹³C NMR (151 MHz, CDCl₃) δ 18.4, 40.0, 42.4, 55.3, 110.4, 120.6, 123.9, 128.3, 131.2, 157.3, 212.5. MS (ESI+APCI), calculated for C12H₆O₂[M+H]⁺ exact mass m/z=193.12, found 193.2.

Compound 46 (R=2,4-F₂C₆H₃): Compound 46 was prepared analogously from compound 26 (R=2,4-F₂C₆H₃), (5.0 g, 26 mmol). 73% Yield. ¹H NMR (600 MHz, DMSO-d) δ 7.30 (td, J=8.6, 6.6 Hz, 1H), 7.18 (ddd, J=10.3, 9.4, 2.6 Hz, 1H), 7.03 (tdd, J=8.5, 2.7, 1.0 Hz, 1H), 3.90 (d, J=1.4 Hz, 2H), 2.76 (hept, J=6.9 Hz, 1H), 1.07 (d, J=6.9 Hz, 6H). ¹³C NMR (151 MHz, CDCl₃) δ 16.5, 101.8, 109.5, 117.3, 131.5, 158.1, 158.8, 159.8, 160.5, 208.4. MS (ESI+APCI), calculated for C₁₁H₁₂F₂O [M] exact mass m/z=198.09.

Compound 47 (R=2,4-(MeO)₂C₆H₃): Compound 47 was prepared analogously from compound 27 (R=2,4-(MeO)₂C₆H₃), (3.6 g). 52% yield. ¹H NMR (600 MHz, DMSO-d) δ 7.01 (d, J=8.2 Hz, 1H), 6.52 (d, J=2.4 Hz, 1H), 6.46 (dd, J=8.2, 2.4 Hz, 1H), 3.75 (s, 3H), 3.70 (s, 3H), 3.62 (s, 2H), 2.67 (hept, J=6.9 Hz, 1H), 1.01 (d, J=6.9 Hz, 6H). MS (ESI+APCI), calculated for C₁₃H₁₈O₃ [M] exact mass m/z=222.13.

Compound 48 (R=3,4-(CH₂O₂)C₆H₃): Compound 48 was prepared analogously from compound 28 (R=3,4-(CH₂O₂)C₆H₃) (5.2 g). 79% yield. ¹H NMR (600 MHz, DMSO-d) δ 6.83 (d, J=7.9 Hz, 1H), 6.75 (d, J=1.7 Hz, 1H), 6.64 (dd, J=7.9, 1.6 Hz, 1H), 5.98 (s, 2H), 3.73 (s, 2H), 2.71 (hept, J=6.9 Hz, 1H), 1.02 (d, J=6.9 Hz, 6H). MS (ESI+APCI), calculated for C₁₂H₁₄O₃[M] exact mass m/z=206.09.

Compound 49 (R=2,4,6-Me₃C₆H₂): Compound 49 was prepared analogously from compound 29 (R=2,4,6-Me₃C₆H₂) (5.00 g, 25.4 mmol). 90% yield. ¹H NMR (600 MHz, DMSO-d₆) δ 6.80 (s, 2H), 3.84 (s, 2H), 2.78 (hept, J=6.9 Hz, 1H), 2.20 (s, 3H), 2.08 (s, 6H), 1.07 (d, J=6.9 Hz, 6H). ¹³C NMR (151 MHz, CDCl₃) δ 18.7, 20.3, 20.9, 40.2, 41.7, 128.9, 129.1, 136.3, 136.7, 212.0. MS (ESI+APCI), calculated for C₁₄H₂₀O [M] exact mass m/z=204.15.

Compound 54 (R=3-MeOC₆H₅): Compound 54 was prepared analogously from compound 34 (R=3-MeOC₆H₅) (5.5 g, 26.2 mmol). 62% yield. ¹H NMR (600 MHz, DMSO-d₆) δ δ 7.25-7.18 (m, 1H), 6.80 (ddd, J=8.2, 2.6, 1.0 Hz, 1H), 6.78-6.72 (m, 2H), 3.79 (s, 2H), 3.73 (s, 3H), 2.73 (hept, J=6.9 Hz, 1H), 1.02 (d, J=6.9 Hz, 6H). ¹³C NMR (151 MHz, DMSO) δ 211.8, 159.6, 137.0, 129.7, 122.3, 115.8, 112.3, 55.4, 47.1, 18.5. MS (ESI+APCI), calculated for C12H₆O₂[M+H]⁺ exact mass m/z=193.12, found 193.2.

Compound 55 (R=4-CF₃C₆H₅): Compound 55 was prepared analogously from compound 35 (R=4-CF₃C₆H₅) (9.00 g, 40.4 mmol). 71% yield. ¹H NMR (600 MHz, DMSO-d₆) δ δ 7.71-7.63 (m, 2H), 7.44-7.37 (m, 2H), 3.99 (s, 2H), 2.76 (hept, J=6.9 Hz, 1H), 1.07 (d, J=6.9 Hz, 6H). ¹³C NMR (151 MHz, DMSO) δ 211.3, 140.6, 131.1, 127.6, 125.9, 125.4, 124.0, 122.2, 46.4, 18.4. MS (ESI+APCI), calculated for C₁₂H₁₃F₃O [M] exact mass m/z=230.09.

Compound 43: To an oven-dried 100 mL round bottom flask were added commercial 1-(4-bromophenyl)-3-methyl-butan-2-one (42) (3.50 g, 14.5 mmol), palladium (0) tetrakis-triphenylphosphine (1.34 g, 1.2 mmol) and zinc cyanide (1.70 g, 14.5 mmol). Anhydrous DMF (35 mL) was added, the reaction mixture was then degassed and heated at 90° C. under argon atmosphere overnight. The mixture was cooled to rt, diluted with 150 mL of EtOAc and washed with ammonium hydroxide (2M, 150 mL×2), followed by satd. NaHCO₃ (140 mL) and satd. NaCl (100 mL). The organic layer was separated, dried over sodium sulfate, filtered, and concentrated in vacuum to give 3.22 g of crude residue. The latter was purified by flash column chromatography on silica gel (220 g, 60 mL/min, gradient of 20% to 35% of ethyl acetate in hexanes) to afford colorless oil which slowly solidified to furnish compound 43 as a white solid: (2.13 g, 77%). ¹H NMR (600 MHz, CDCl₃) δ 1.17 (d, 6H, J=5 Hz), 2.74 (m, 1H), 3.84 (s, 2H), 7.31-7.33 (m, 2H), 7.62-7.64 (m, 2H). ¹³C NMR (151 MHz, CDCl₃) δ 18.2, 41.0, 47.1, 110.9, 118.8, 130.4, 132.3, 139.8, 210.2. MS (ESI+APCI), calculated for C₁₂H₁₃NO [M] exact mass m/z=187.10.

General procedure—Compound 61 (R=4-FC₆H₄): Compound 41(4.0 g, 22 mmol) was dissolved in diethyl ether (20 mL) under inert atmosphere. A small amount (12 drops) of a separately prepared solution of bromine (2.3 mL, 46 mmol) in dichloromethane (10 mL) was added to the ketone solution and stirred for a few minutes to initiate the reaction which was indicated by discoloration of the mixture. The mixture was cooled in an ice/water bath and the rest of bromine solution in DCM was added dropwise to cooled and stirred ketone solution for 1 hour. The ice bath was removed, and the mixture was stirred at room temperature for 2 hours. The mixture was diluted with diethyl ether (100 mL), poured portion-wise to a stirred aqueous solution of 5% aq. NaCl, and stirred for 10 minutes. The organic layer was separated, washed with 5% NaCl (2×), 5% Na₂S₂O₅ (1×), saturated NaCl (1×), dried over Na₂SO₄, filtered, and concentrated in vacuum. The residue was dried in high vacuum to afford compound 61 as a greyish solid, (7.4 g) 98% yield. ¹H NMR (600 MHz, DMSO) δ 7.77-7.70 (m, 2H), 7.28-7.23 (m, 2H), 6.67 (s, 1H), 2.01 (s, 3H), 1.82 (s, 3H). MS (ESI+APCI), calculated for C₁₁H₁₁Br₂FO [M] exact mass m/z=335.92.

Compound 62 (R=4-BrC₆H₄): Compound 62 was prepared analogously from compound 42 (4.50 g, 19 mmol). 90% yield, ¹H NMR (600 MHz, DMSO-d6) δ 1.82 (s, 3H), 2.01 (s, 3H), 6.65 (s, 1H), 7.63 (s, 4H). 13C NMR (151 MHz, CDCl₃) δ 29.0, 29.5, 45.6, 65.0, 123.1, 131.7, 132.3, 136.1, 197.4. MS (ESI+APCI), calculated for C₁₁H₁₁Br₃O [M] exact mass m/z=395.84.

Compound 63 (R=4-NCC₆H₄): Compound 63 was prepared analogously from compound 43 (2.10 g, 11 mmol). 86% yield, ¹H NMR (600 MHz, CDCl₃) δ 1.79 (s, 3H), 2.05 (s, 3H), 6.04 (s, 1H), 7.59 (d, 2H, J=8 Hz), 7.73 (d, 2H, J=8 Hz). ¹³C NMR (151 MHz, CDCl₃) δ 27.2, 28.5, 41.5, 62.2, 111.1, 116.3, 128.2, 130.5, 138.9, 194.1. MS (ESI+APCI), calculated for C₁₂H₁₁Br₂NO [M] exact mass m/z=342.92.

Compound 64 (R=2-MeC₆H₄): Compound 64 was prepared analogously from compound 44 (R=2-MeC₆H₄) (2.00 g, 11 mmol). 91% Yield, ¹H NMR (600 MHz, DMSO-d₆) δ 1.73 (s, 3H), 1.98 (s, 3H), 2.47 (s, 3H), 6.55 (s, 1H), 7.24-7.30 (m, 3H), 7.43 (d, J=5 Hz, 1H). ¹³C NMR (151 MHz, DMSO-d₆) δ 19.4, 29.8, 30.1, 46.6, 64.3, 127.3, 128.7, 129.9, 131.8, 134.3, 136.7, 197.7. MS (ESI+APCI), calculated for C₁₂H₁₄Br₂O [M] exact mass m/z=331.94.

Compound 65 (R=2-MeOC₆H₄): Compound 65 was prepared analogously from compound 45 (R=2-MeOC₆H₄) (11 mmol). 95% yield, ¹H NMR (600 MHz, DMSO-d6) δ 1.71 (s, 3H), 1.87 (s, 3H), 3.79 (s, 3H), 6.07 (s, 1H), 6.93-7.01 (m, 2H), 7.24-7.32 (m, 2H). ¹³C NMR (151 MHz, DMSO-d₆) δ 27.8, 28.2, 53.7, 62.4, 65.0, 109.4, 118.7, 126.0, 126.5, 127.7, 154.2, 201.6. MS (ESI+APCI), calculated for C₁₂H₁₄Br₂O₂ [M] exact mass m/z=347.94.

Compound 66 (R=2,4-F₂C₆H₃): Compound 66 was prepared analogously from compound 46 (R=2,4-F₂C₆H₃) (1.25 g, 3.6 mmol). 95% yield. ¹H NMR (CDCl₃) δ 1.77 (s, 3H), 1.99 (s, 3H), 6.38 (s, 1H), 6.73-7.78 (m, 1H), 6.85-6.89 (m, 1H), 7.78-7.84 (m, 1H). MS (ESI+APCI), calculated for C₁₁H₁₀Br₂F₂O [M] exact mass m/z=353.91.

Compound 68 (R=3,4-(CH₂O₂)C₆H₃): Compound 68 was prepared analogously from compound 48 (R=3,4-(CH₂O₂)C₆H₃) (7.1 g). 81% yield. ¹H NMR (600 MHz, DMSO-d6) δ 7.20 (d, J=1.8 Hz, 1H), 7.16 (dd, J=8.1, 1.9 Hz, 1H), 6.92 (d, J=8.1 Hz, 1H), 6.59 (s, 1H), 6.05 (dd, J=8.1, 1.1 Hz, 2H), 1.97 (s, 3H), 1.81 (s, 3H). MS (ESI+APCI), calculated for C₁₂H₁₂Br₂O₃ [M] exact mass m/z=361.92.

Compound 70 (R=Me): Compound 70 was prepared analogously from compound 50 (R=Me) (23.3 g, 233 mmol). 81% yield (57.1 g). ¹H NMR, (500 MHz, DMSO-d₆) δ 5.41 (q, J=6.6 Hz, 1H), 1.98 (s, 3H), 1.87 (s, 3H), 1.74 (d, J=6.6 Hz, 3H). ¹³C NMR (126 MHz, DMSO-d₆) δ 198.94, 64.58, 41.06, 29.35, 28.59, 22.10. MS (ESI+APCI), calculated for C₆H₁₀Br₂O [M] exact mass m/z=255.91.

Compound 74 (R=3-MeOC₆H₅): Compound 74 was prepared analogously from compound 54 (R=3-MeOC₆H₅) (6.0 g, 31.2 mmol). 84% yield. ¹H NMR (CDCl₃) δ 7.32 (t, J=7.9 Hz, 1H), 7.25-7.19 (m, 2H), 6.94 (ddd, J=8.3, 2.6, 0.9 Hz, 1H), 6.59 (s, 1H), 3.76 (s, 3H), 1.98 (s, 3H), 1.81 (s, 3H). MS (ESI+APCI), calculated for C₁₂H₁₄Br₂O₂ [M] exact mass m/z=347.94.

Compound 75 (R=4-CF₃C₆H₅): Compound 75 was prepared analogously from compound 55 (R=4-CF₃C₆H₅) (1.25 g, 3.62 mmol). 98% yield. ¹H NMR (600 MHz, DMSO-d6) δ 7.95-7.88 (m, 2H), 7.84-7.76 (m, 2H), 6.75 (s, 1H), 2.04 (s, 3H), 1.84 (s, 3H). ¹³C NMR (151 MHz, DMSO-d6) δ 197.4, 141.3, 130.5, 129.8, 126.2, 125.3, 123.5, 65.2, 44.9, 29.4, 28.9. MS (ESI+APCI), calculated for C₁₂H₁₁Br₂F₃O [M] exact mass m/z=385.91.

Compound 76 (R=Et): Compound 76 was prepared analogously from commercial compound 56 (R=Et) (4.0 g, 35.0 mmol). 97% yield. ¹H NMR (600 MHz, DMSO-d6) δ 4.91 (dd, J=8.0, 6.3 Hz, 1H), 2.21-2.11 (m, 2H), 2.09 (s, 3H), 1.10 (t, J=7.3 Hz, 3H). ¹³C NMR (151 MHz, DMSO-d6) δ 198.0, 64.0, 47.3, 31.0, 29.2, 28.2, 12.2. MS (ESI+APCI), calculated for C₇H₁₂Br₂O [M] exact mass m/z=269.93.

Compound 77 (R=i-Pr): Compound 77 was prepared analogously from commercial compound 57 (R=i-Pr) (4.42 g, 35.0 mmol). 100% yield. ¹H NMR (600 MHz, DMSO-d6) δ 4.91 (d, J=8.4 Hz, 1H), 2.21 (d, J=8.4 Hz, 1H), 2.03 (s, 3H); 1.90 (s, 3H); 1.14 (d, J=6.6 Hz, 3H); 0.98 (d, J=6.6 Hz, 3H). ¹³C NMR (151 MHz, DMSO-d6) δ 198.4, 65.2, 54.2, 31.8, 30.7, 29.6, 20.5, 20.4. MS (ESI+APCI), calculated for C₈H₁₄Br₂O [M] exact mass m/z=283.94.

General procedure—Compound 81 (R=4-FC₆H₄): N-Methylacetamide (75 mL) was heated at 33° C. under inert atmosphere. Disodiumsulfide-nonahydrate (10.0 g, 41 mmol) and sulfur (2.7 g, 83 mmol) were added, and the suspension was stirred at 35° C. for 24 hours to dissolve solids. The mixture was cooled to 30° C. and compound 61(7.0 g, 21 mmol) was added. The mixture was stirred at 30° C. for 22 hours and quenched by slowly pouring to a stirred solution of 5% NaCl (300 mL). The mixture was diluted with ethyl acetate, washed with 5% NaCl (3×) and saturated NaCl (1×). Organic layer was dried over Na₂SO₄, filtered, and concentrated. Oily residue was diluted in hexanes and stirred for 18 hours. Solids were removed by decanting and the supernatant was concentrated to an oil. The crude product was purified by silica gel flash chromatography. Compound 81 was isolated as a yellow oil (2.0 g), 40% yield. ¹H NMR (600 MHz, DMSO-d6) δ 7.38-7.33 (m, 2H), 7.26-7.20 (m, 2H), 5.36 (s, 1H), 1.63 (s, 3H), 1.53 (s, 3H). MS (ESI+APCI), calculated for C₁₁H₁₁FOS₂ [M] exact mass m/z=242.02.

Compound 82 (R=4-BrC₆H₄): Compound 82 was prepared analogously from compound 62 (R=4-BrC₆H₄) (1.55 g, 2.8 mmol). 45% yield, ¹H NMR (600 MHz, DMSO-d₆) δ 1.53 (s, 3H), 1.61 (s, 3H), 5.36 (s, 1H), 7.27-7.28 (m, 2H), 7.59-7.61 (m, 2H). ¹³C NMR (151 MHz, DMSO-d₆) δ 24.6, 25.0, 57.2, 58.5, 122.2, 131.8, 132.3, 135.8, 210.3. MS (ESI+APCI), calculated for C₁₁H₁₁BrOS₂ [M] exact mass m/z=301.94.

Compound 83 (R=4-NCC₆H₄): Compound 83 was prepared analogously from compound 63 (1.51 g, 4.4 mmol). 38% yield, ¹H NMR (600 MHz, DMSO-d₆) δ 1.56 (s, 3H), 1.60 (s, 3H), 5.50 (s, 1H), 7.52-7.54 (m, 2H), 7.86-7.88 (m, 2H). ¹³C NMR (151 MHz, DMSO-d₆) δ 24.5, 24.8, 57.5, 58.3, 111.6, 119.0, 130.5, 133.2, 142.0, 209.7. MS (ESI+APCI), calculated for C₁₂H₁₁NOS₂ [M+H]⁺ exact mass m/z=249.03, found 249.0.

Compound 84 (R=2-MeC₆H₄): Compound 84 was prepared analogously from compound 64 (0.95 g, 4.0 mmol). 40% yield, ¹H NMR (600 MHz, DMSO) δ 7.32-7.18 (m, 3H), 7.11-7.04 (m, 1H), 5.35 (s, 1H), 2.36 (s, 3H), 1.66 (s, 3H), 1.54 (s, 3H). ¹³C NMR (151 MHz, DMSO) δ 211.0, 138.0, 134.67, 131.1, 129.5, 128.9, 126.9, 57.4, 57.3, 25.2, 24.7, 19.7. MS (ESI+APCI), calculated for C₁₂H₁₄OS₂ [M+H]⁺ exact mass m/z=239.05, found 239.0.

Compound 85 (R=2-MeOC₆H₄): Compound 85 was prepared analogously from compound 65 (1.50 g, 2.8 mmol). 33% yield, ¹H NMR (600 MHz, DMSO-d₆) δ 1.48 (s, 3H), 1.68 (s, 3H), 3.76 (s, 3H), 5.27 (s, 1H), 6.94-6.97 (m, 1H), 7.04-7.06 (m, 1H), 7.20-7.21 (m, 1H), 7.35-7.38 (m, 1H). ¹³C NMR (151 MHz, DMSO-d₆) δ 24.8, 26.0, 56.1, 56.3, 57.1, 112.3, 121.2, 123.8, 130.8, 131.7, 157.4, 210.6. MS (ESI+APCI), calculated for C₁₁H₁₄O₂S₂ [M] exact mass m/z=254.04.

Compound 86 (R=2,4-F₂C₆H₃): Compound 86 was prepared analogously from compound 66 (R=2,4-F₂C₆H₃) (1.25 g, 3.5 mmol). 21% Yield. ¹H NMR (600 MHz, CDCl₃) δ 1.59 (s, 3H), 1.74 (s, 3H), 5.03 (s, 1H), 6.85-6.93 (m, 2H), 7.20-7.24 (m, 1H). ¹³C NMR (151 MHz, CDCl₃) δ 24.6, 25.5, 54.0, 56.8, 104.5, 112.0, 118.8, 131.7, 160.3, 162.1, 163.9, 209.6. MS (ESI+APCI), calculated for C₁₁H₁₀F₂OS₂ [M−H]⁻ exact mass m/z=259.01, found 259.0.

Compound 88 (R=3,4-(CH₂O₂)C₆H₃): Compound 88 was prepared analogously from compound 68 (1.7 g). 38% yield. ¹H NMR (600 MHz, DMSO-d₆) δ 6.92 (d, J=8.0 Hz, 1H), 6.83-6.77 (m, 2H), 6.04 (s, 2H), 5.23 (s, 1H), 1.63 (s, 3H), 1.50 (s, 3H). MS (ESI+APCI), calculated for C₁₂H₁₂O₃S₂ [M] exact mass m/z=268.02.

Compound 90 (R=Me): Compound 90 was prepared analogously from compound 70 (R=Me) (30 g, 116 mmol). 32% Yield (6.1 g). ¹H NMR, (400 MHz, DMSO-d₆) δ 3.98 (q, J=7.0 Hz, 1H), 1.45 (d, J=9.0 Hz, 6H), 1.37 (d, J=7.0 Hz, 3H). ¹³C NMR (126 MHz, DMSO-d₆) δ 211.58, 56.27, 50.15, 24.69, 24.11, 16.00. MS (ESI+APCI), calculated for C₆H₁₀OS₂ [M] exact mass m/z=162.02.

Compound 94 (R=3-MeOC₆H₅): Compound 94 was prepared analogously from compound 74 (R=3-MeOC₆H₅) (1.25 g, 3.5 mmol). 21% Yield. ¹H NMR (600 MHz, CDCl₃) δ 1.59 (s, 3H), 1.74 (s, 3H), 5.03 (s, 1H), 6.85-6.93 (m, 2H), 7.20-7.24 (m, 1H). ¹³C NMR (151 MHz, CDCl₃) δ 24.6, 25.5, 54.0, 56.8, 104.5, 112.0, 118.8, 131.7, 160.3, 162.1, 163.9, 209.6. MS (ESI+APCI), calculated for C₁₂H₁₄O₂S₂ [M+H]⁺ exact mass m/z=255.04, found 255.0.

Compound 95 (R=4-CF₃C₆H₅): Compound 95 was prepared analogously from compound 75 (R=4-CF₃C₆H₅) (3.2 g, 8.25 mmol). 45% yield. ¹H NMR (600 MHz, CDCl₃) δ 7.77 (d, J=7.2 Hz, 2H), 7.56 (d, J=7.2 Hz, 2H), 5.50 (s, 1H), 1.62 (s, 3H), 1.56 (s, 3H). ¹³C NMR (151 MHz, DMSO) δ 210.0, 141.1, 130.4, 129.4, 129.2, 127.2, 126.2, 125.4, 123.6, 58.3, 57.4, 24.9, 24.6. MS (ESI+APCI), calculated for C₁₂H₁₁F₃OS₂ [M+H]⁺ exact mass m/z=293.03, found 293.0.

Compound 96 (R=Et): Compound 96 was prepared analogously from compound 76 (R=Et) (5.6 g, 20.6 mmol). 69% yield. ¹H NMR (600 MHz, DMSO) δ 3.96 (dd, J=8.9, 4.3 Hz, 1H), 1.95 (dqd, J=14.2, 7.5, 4.3 Hz, 1H), 1.69 (ddq, J=14.6, 8.9, 7.3 Hz, 1H), 1.47 (s, 3H), 1.44 (s, 3H), 0.98 (t, J=7.4 Hz, 3H). ¹³C NMR (151 MHz, DMSO) δ 211.6, 57.9, 57.2, 24.6, 24.5, 24.5, 12.3. MS (ESI+APCI), calculated for C₇H₁₂OS₂ [M−H]⁻ exact mass m/z=175.03, found 175.0.

Compound 97 (R=i-Pr): Compound 97 was prepared analogously from compound 77 (R=i-Pr) (9.15 g, 32 mmol). 35% yield. ¹H NMR (600 MHz, DMSO) δ 4.02 (d, J=4.2 Hz, 1H); 2.36 (quintet, J=4.2 Hz, 1H); 1.46 (s, 3H); 1.43 (s, 3H); 1.04 (d, J=7.2 Hz, 1H); 0.88 (d, J=7.2 Hz, 1H). ¹³C NMR (151 MHz, DMSO) δ 211.0, 63.1, 57.4, 30.0, 24.5, 24.2, 21.8, 18.6. MS (ESI+APCI), calculated for C₈H₁₄OS₂ [M−H]⁺ exact mass m/z=189.04, found 189.1.

General procedure—Compound 102 (R=4-BrC₆H₄): Ketone 82 (1.0 g, 3.3 mmol) was dissolved in ethanol (11 mL) under inert atmosphere and cooled to −78° C. Acetic acid (198 mg, 3.29 mmol) was added, followed by sodium borohydride (124 mg, 3.3 mmol), the. The mixture was warmed up to 0° C., stirred at 0° C. for one hour, followed by stirring at room temperature for 15 hours. A second portion of sodium borohydride (32 mg, 0.82 mmol) was added after the reaction mixture was cooled to 0° C. again. The reaction mixture was stirred for additional 4 hours at room temperature, cooled to 0° C., quenched with small amount of saturated NH₄Cl, and allowed to warm up to room temperature. Ethyl acetate (50 mL), saturated NH₄Cl (20 mL), and water (16 mL) were added, and the mixture was stirred vigorously at room temperature for 24 hours in the presence of air. The organic layer was separated, washed with 1:1 saturated NH₄Cl:water (1×), saturated NaHCO₃(1×), and saturated NaCl (1×), dried over Na₂SO₄, filtered, and concentrated. The crude product was purified by silica gel flash chromatography to obtain pure compound 102. (388 mg, 38%) ¹H NMR (600 MHz, DMSO-d₆) δ 1.43 (s, 3H), 1.52 (s, 3H), 3.93 (dd, J=7.1, 3.9 Hz, 1H), 5.02 (d, J=3.9 Hz, 1H), 5.34 (d, J=7.1 Hz, 1H), 7.47-7.52 (m, 4H). ¹H NMR (600 MHz, DMSO-d₆+D₂O) δ 1.42 (s, 3H), 1.51 (s, 3H), 3.92 (d, J=3.9 Hz, 1H), 5.00 (d, J=3.9 Hz, 1H), 7.45-7.50 (m, 4H). ¹³C NMR (151 MHz, DMSO-d₆) δ 23.4, 28.7, 61.0, 66.4, 121.1, 131.0, 132.7, 136.6, 142.0, 209.7. MS (ESI+APCI), calculated for C₁₁H₁₃BrOS₂ [M] exact mass m/z=303.96.

Compound 101 (R=4-FC₆H₄): Compound 101 was prepared analogously from compound 81(1.95 g, 8.05 mmol). 75% Yield, ¹H NMR (600 MHz, DMSO) δ 7.60-7.54 (m, 2H), 7.17-7.11 (m, 2H), 5.33 (d, J=7.2 Hz, 1H), 5.04 (d, J=3.8 Hz, 1H), 3.91 (dd, J=7.2, 3.8 Hz, 1H), 1.53 (s, 3H), 1.44 (s, 3H). MS (ESI+APCI), calculated for C₁₁H₁₃FOS₂ [M] exact mass m/z=244.04.

Compound 103 (R=4-NCC₆H₄): Compound 103 was prepared analogously from compound 83 (0.42 g, 1.7 mmol). 42% Yield, ¹H NMR (600 MHz, DMSO-d₆) δ 1.43 (s, 3H), 1.54 (s, 3H), 4.01 (dd, 1H), 5.12 (d, 1H), 5.40 (d, 1H), 7.71-7.72 (m, 2H), 7.78-7.79 (m, 2H). ¹³C NMR (151 MHz, DMSO-d₆) δ 23.2, 28.3, 61.1, 66.7, 84.2, 110.5, 119.4, 131.6, 132.0, 142.0, 143.5. MS (ESI+APCI), calculated for C₁₂H₁₃NOS₂ [M] exact mass m/z=251.04.

Compound 104 (R=2-MeC₆H₄): Compound 104 was prepared analogously from compound 84 (0.95 g, 4.0 mmol). 53% Yield, ¹H NMR (600 MHz, DMSO-d₆) δ 7.73-7.66 (m, 1H), 7.21-7.11 (m, 3H), 5.16 (dd, J=7.7, 5.4 Hz, 2H), 4.00 (dd, J=7.0, 3.8 Hz, 1H), 2.35 (s, 3H), 1.55 (s, 3H), 1.46 (s, 3H). ¹³C NMR (151 MHz, DMSO-d₆) δ 136.2, 134.9, 131.2, 130.3, 127.6, 125.7, 81.9, 66.3, 58.8, 29.2, 23.9, 19.8. MS (ESI+APCI), calculated for C₁₂H₁₆OS₂ [M] exact mass m/z=240.06.

Compound 105 (R=2-MeOC₆H₄): Compound 105 was prepared analogously from compound 85 (1.78 g, 7.0 mmol). 50% Yield, ¹H NMR (600 MHz, DMSO-d₆) δ 7.64 (dd, J=7.6, 1.8 Hz, 1H), 7.26 (ddt, J=9.1, 7.4, 1.8 Hz, 1H), 6.99 (dt, J=8.3, 1.5 Hz, 1H), 6.93 (td, J=7.5, 1.2 Hz, 1H), 5.30 (d, J=3.7 Hz, 1H), 5.12 (d, J=7.3 Hz, 1H), 3.97 (dd, J=7.0, 3.6 Hz, 1H), 3.81 (s, 3H), 1.52 (s, 3H), 1.44 (s, 3H). ¹³C NMR (151 MHz, DMSO-d₆) δ 156.8, 131.9, 129.0, 124.5, 120.3, 111.0, 82.1, 66.3, 56.2, 55.5, 29.1, 23.8. MS (ESI+APCI), calculated for C12H₁₆O₂S₂ [M+H]⁺ exact mass m/z=257.06, found 257.0.

Compound 106 (R=2,4-F2C₆H₄): Compound 106 was prepared analogously from compound 86 (0.64 g, 2.5 mmol). 45% Yield, ¹H NMR (600 MHz, DMSO-d₆) δ 7.77 (m, 1H), 7.22 (m, 1H), 7.09 (m, 1H), 5.40 (dd, J=7.0, 1.4 Hz, 1H), 5.15 (d, J=4.2 Hz, 1H), 4.00 (dd, J=7.1, 4.2 Hz, 1H), 1.53 (s, 3H), 1.44 (s, 3H). MS (ESI+APCI), calculated for C₁₁H₁₂F₂OS₂ [M] exact mass m/z=262.03.

Compound 108 (R=3,4-(CH₂O₂)C₆H₃): Compound 108 was prepared analogously from compound 88 (1.65 g, 6.1 mmol). 74% Yield, ¹H NMR (600 MHz, DMSO) δ 7.15 (d, J=1.7 Hz, 1H), 6.98 (dd, J=8.1, 1.7 Hz, 1H), 6.83 (d, J=8.0 Hz, 1H), 5.99 (s, 2H), 5.28 (d, J=7.4 Hz, 1H), 4.97 (d, J=3.7 Hz, 1H), 3.86 (dd, J=7.2, 3.7 Hz, 1H), 1.51 (s, 3H), 1.43 (s, 3H). MS (ESI+APCI), calculated for C₁₂H₁₄O₃S₂ [M] exact mass m/z=270.04.

Compound 114 (R=3-MeOC₆H₄): Compound 114 was prepared analogously from compound 94 (1.75 g, 6.9 mmol). 41% Yield, ¹H NMR (600 MHz, DMSO-d₆) δ 7.84 (t, J=7.9 Hz, 1H), 7.72-7.64 (m, 2H), 7.44 (ddd, J=8.2, 2.6, 0.9 Hz, 1H), 5.61 (d, J=3.5 Hz, 1H), 4.57 (dd, J=9.4, 3.5 Hz, 1H), 4.37 (s, 3H), 3.51 (s, 1H), 2.17 (s, 3H), 2.07 (s, 3H). ¹H NMR (DMSO-d₆+D₂O) δ 7.83 (t, J=7.9 Hz, 1H), 7.71-7.63 (m, 2H), 7.43 (ddd, J=8.3, 2.6, 0.9 Hz, 1H), 5.60 (d, J=3.5 Hz, 1H), 4.56 (d, J=3.5 Hz, 1H), 4.36 (s, 3H), 2.15 (s, 3H), 2.06 (s, 3H). ¹³C NMR (151 MHz, DMSO-d₆) δ δ 159.2, 138.4, 129.1, 122.7, 116.1, 113.5, 84.1, 66.2, 62.0, 55.5, 29.3, 23.8. MS (ESI+APCI), calculated for C12H₁₆O₂S₂ [M+H]⁺ exact mass m/z=257.06, found 257.0.

Compound 115 (R=4-CF₃C₆H₃): Compound 115 was prepared analogously from compound 95 (1.35 g, 4.6 mmol). 47% Yield, ¹H NMR (600 MHz, DMSO-d₆) δ 7.75 (d, J=8.1 Hz, 2H), 7.68 (d, J=8.2 Hz, 2H), 5.39 (d, J=7.1 Hz, 1H), 5.13 (d, J=3.9 Hz, 1H), 4.00 (dd, J=7.1, 4.0 Hz, 1H), 1.55 (s, 3H), 1.44 (s, 3H). ¹³C NMR (151 MHz, DMSO-d₆) δ 142.4, 131.3, 128.5, 128.3, 127.5, 125.0, 123.9, 84.1, 66.6, 61.1, 28.5, 23.3. MS (ESI+APCI), calculated for C₁₂H₁₃F₃OS₂ [M] exact mass m/z=294.04.

Compound 116 (R=Et): Compound 116 was prepared analogously from compound 96 (1.25 g, 7.1 mmol). 56% Yield, ¹H NMR (600 MHz, DMSO-d₆) δ 5.11 (dd, J=7.2, 0.5 Hz, 1H), 3.84 (dd, J=7.3, 4.0 Hz, 1H), 3.69 (ddd, J=8.5, 6.1, 4.0 Hz, 1H), 1.92-1.80 (m, 1H), 1.65-1.55 (m, 1H), 1.38 (d, J=10.2 Hz, 6H), 0.94 (t, J=7.4 Hz, 3H). ¹³C NMR (151 MHz, DMSO-d₆) δ 82.6, 65.0, 60.8, 28.4, 23.6, 23.2, 14.0. MS (ESI+APCI), calculated for C₇H₁₄OS₂ [M] exact mass m/z=178.05.

Compound 117 (R=i-Pr): Compound 117 was prepared analogously from compound 97 (2.09 g, 11 mmol). 36% Yield, ¹H NMR (600 MHz, DMSO-d₆) δ 4.99 (d, J=7.1 Hz, 1H), 3.85 (dd, J=7.8, 3.0 Hz, 1H), 3.48 (dd, J=10.2, 3.0 Hz, 1H), 2.10-2.01 (m, 1H); 1.39 (s, 3H), 1.38 (s, 3H), 1.01 (d, J=6.6 Hz, 3H), 0.96 (d, J=6.6 Hz, 3H). ¹³C NMR (151 MHz, DMSO-d₆) δ 81.7, 67.4, 66.3, 30.0, 28.4, 23.4, 23.3, 22.5. MS (ESI+APCI), calculated for C₈H₁₆OS₂ [M] exact mass m/z=192.06.

Compounds 110 and 130 (R=Me): Compounds 110 and 130 were prepared analogously from compound 90 (R=Me) (2.3 g, 14.2 mmol). Isomers separated by column chromatography on silica gel with ethylacetate-hexanes eluents. 110: Yield 40% (0.93 g). ¹H NMR, (500 MHz, DMSO-d₆) δ 5.13 (d, J=6.9 Hz, 1H), 3.88-3.82 (m, 1H), 3.76 (dd, J=6.9, 4.5 Hz, 1H), 1.37 (d, J=6.5 Hz, 6H), 1.28 (d, J=6.8 Hz, 3H). ¹³C NMR (101 MHz, DMSO-d₆) δ 83.39, 63.88, 51.88, 28.15, 22.83, 15.24. MS (ESI+APCI), calculated for C₆H₁₂OS₂ [M] exact mass m/z=164.03. 130: Yield 10% (0.23 g) ¹H NMR, (400 MHz, DMSO-d₆) δ 5.67 (d, J=6.3 Hz, 1H), 3.36 (dd, J=8.4, 6.2 Hz, 1H), 3.27-3.19 (m, 1H), 1.37 (d, J=6.5 Hz, 3H), 1.31 (d, J=3.9 Hz, 6H). ¹³C NMR (101 MHz, DMSO-d₆) δ 88.45, 57.99, 48.86, 25.74, 21.63, 19.17. MS (ESI+APCI), calculated for C₆H₁₂OS₂ [M] exact mass m/z=164.03.

General procedure—Compound 142 (R=4-BrC₆H₄): Compound 102 (0.66 g, 1.3 mmol) was dissolved in anhydrous ethyl acetate (18 mL) under inert atmosphere. N,N-diisopropylethylamine (0.35 mL, 2 mmol) followed by 2-cyanoethyl N,N-diisopropylchlorophosphoramidite (0.44 mL, 2 mmol) were added and the mixture was stirred at room temperature overnight. The mixture was quenched with satd. NaHCO₃, the organic phase was separated, washed with satd. NaCl (1×), dried over Na₂SO₄, filtered, and concentrated. The crude product was purified by silica gel flash chromatography to obtain pure compound 142 (202 mg, 18%). ¹H NMR (600 MHz, CD₃CN) δ 0.97 (d, 6H, J=5 Hz), 1.07 (d, 6H, J=5 Hz), 1.63 (s, 3H), 1.67 (s, 3H), 2.63 (m, 2H), 3.52 (m, 2H), 3.61 (m, 1H), 3.76 (m, 1H), 4.36 (m, 1H), 5.02 (d, 1H), 7.39 (d, 2H, J=5 Hz), 7.48 (d, 2H, J=5 Hz). ¹³C NMR (151 MHz, CD₃CN) δ 20.5, 24.2, 27.6, 43.6, 58.2, 62.1, 64.7, 85.9, 119.3, 121.7, 131.6, 132.9, 136.5. ³¹P NMR (243 MHz, CD₃CN) δ 149.2, 151.9. MS (ESI+APCI), calculated for C₂₀H₃₀BrN₂O₂PS₂ [M] exact mass m/z=504.07.

Compound 141 (R=4-FC₆H₄): Compound 141 was prepared analogously from compound 101 (0.60 g, 2.5 mmol). 43% Yield, ¹H NMR (600 MHz, CD₃CN) δ 7.60-7.45 (m, 2H), 7.07 (m, 2H), 5.10-5.02 (m, 1H), 4.38-4.29 (m, 1H), 3.99-3.23 (m, 4H), 2.68-2.59 (m, 1H), 2.57-2.43 (m, 1H), 1.70-1.49 (m, 6H), 1.14-0.94 (m, 10H). ³¹P NMR (243 MHz, CD3CN) δ 151.8, 149.1. MS (ESI+APCI), calculated for C₂₀H₃₀FN₂O₂PS₂ [M] exact mass m/z=444.15.

Compound 143 (R=4-NCC₆H₄): Compound 143 was prepared analogously from compound 103 (1.3 mmol). 27% Yield, ¹H NMR (600 MHz, CD₃CN) δ 0.92 (d, 6H, J=5 Hz), 1.06 (d, 6H, J=5 Hz), 1.64 (s, 3H), 1.66 (s, 3H), 2.64 (m, 2H), 3.50 (m, 2H), 3.62 (m, 1H), 3.73 (m, 1H), 4.43 (m, 1H), 5.10 (d, 1H), 7.63-7.71 (m, 4H). ¹³C NMR (151 MHz, CD₃CN) δ 20.5, 24.2, 27.5, 43.6, 58.3, 62.2, 65.0, 86.1, 111.6, 119.2, 131.8, 132.0, 132.1, 132.5, 143.1. ³¹P NMR (243 MHz, CD₃CN) δ 149.2, 152.0. MS (ESI+APCI), calculated for C₂₁H₃₀N₃O₂PS₂ [M+H]⁺ exact mass m/z=452.15, found 452.2.

Compound 144 (R=2-MeC₆H₄): Compound 144 was prepared analogously from compound 104 (1.5 mmol). 57% Yield, ¹H NMR (600 MHz, CD₃CN) δ 7.66-7.57 (m, 1H), 7.25-7.11 (m, 3H), 5.22 (d, J=5.3 Hz, 1H), 4.47-4.34 (m, 1H), 3.74 (m, 1H), 3.63-3.46 (m, 3H), 3.41-3.26 (m, 1H), 2.63 (t, J=5.8 Hz, 2H), 2.41 (s, 3H), 1.71 (s, 3H), 1.65 (s, 3H), 1.04 (d, J=6.8 Hz, 6H), 0.93 (d, J=6.8 Hz, 6H). ³¹P NMR (243 MHz, CD₃CN) S 150.1, 149.4. MS (ESI+APCI), calculated for C₂₁H₃₃N₂O₂PS₂ [M] exact mass m/z=440.17.

Compound 145 (R=2-MeOC₆H₄): Compound 145 was prepared analogously from compound 105 (2.3 mmol). 48% Yield, ¹H NMR (600 MHz, CD₃CN) δ 7.55 (m, 1H), 7.28 (m, 1H), 6.98-6.87 (m, 2H), 5.36 (d, J=4.8 Hz, 1H), 4.47 (dd, J=12.7, 4.8 Hz, 1H), 3.77-3.69 (m, 1H), 3.58 (m, 1H), 3.50 (m, 1H), 3.42-3.21 (m, 1H), 2.66-2.58 (m, 1H), 1.66 (s, 3H), 1.64 (d, J=1.7 Hz, 3H), 1.11-0.98 (m, 6H), 0.93 (d, J=6.8 Hz, 6H. ³¹P NMR (243 MHz, CD₃CN) δ 150.3, 149.6. MS (ESI+APCI), calculated for C₂₁H₃₃N₂O₃PS₂ [M+H]⁺ exact mass m/z=457.17, found 457.2.

Compound 146 (R=2,4-F2C₆H₄): Compound 146 was prepared analogously from compound 106 (0.41 g, 1.6 mmol). 37% Yield, ¹H NMR (600 MHz, CD₃CN) δ 7.68 (td, J=8.6, 6.5 Hz, 1H), 7.01-6.90 (m, 2H), 5.27 (d, J=5.7 Hz, 1H), 4.40 (dd, J=13.1, 5.7 Hz, 1H), 3.77 (m, 1H), 3.62 (m, 1H), 3.51 (m, 2H), 2.69-2.59 (m, 2H), 1.67 (s, 3H), 1.64 (s, 3H), 1.07 (d, J=6.8 Hz, 6H), 0.94 (d, J=6.8 Hz, 6H). ¹³C NMR (151 MHz, CD₃CN) δ 163.8, 162.2, 161.7, 160.0, 134.4, 134.3, 120.8, 120.7, 119.2, 112.0, 103.7, 84.7, 65.04, 58.4, 58.2, 54.7, 43.7, 43.6, 27.9, 27.5, 24.4, 24.3, 24.2, 24.1, 22.0, 20.5. ³¹P NMR (243 MHz, CD₃CN) S 151.3, 149.4. MS (ESI+APCI), calculated for C₂₀H₂₉F₂N₂O₂PS₂ [M+H]⁺ exact mass m/z=463.14, found 463.2.

Compound 148 (R=3,4-(CH₂O₂)C₆H₃): Compound 148 was prepared analogously from compound 108 (0.60 g, 2.2 mmol). 81% Yield, ¹H NMR (600 MHz, CD₃CN) δ 7.12-6.74 (m, 3H), 6.00-5.90 (m, 2H), 4.98 (d, J=5.7 Hz, 1H), 4.29 (d, J=5.7 Hz, 1H), 3.79-3.31 (m, 4H), 2.63 (d, J=1.2 Hz, 2H), 1.69-1.49 (m, 7H), 1.16-0.99 (m, 12H). ³¹P NMR (243 MHz, CD₃CN) δ 151.5, 149.1. MS (ESI+APCI), calculated for C₂₁H₃₁N₂O₄PS₂ [M] exact mass m/z=470.15.

Compound 154 (R=3-MeOC₆H₄): Compound 154 was prepared analogously from compound 114 (0.50 g, 1.9 mmol). 40% Yield, ¹H NMR (600 MHz, CD₃CN) δ 7.26-7.18 (m, 1H), 7.13-6.94 (m, 2H), 6.88-6.80 (m, 1H), 5.05-4.97 (m, 1H), 4.37 (ddd, J=12.9, 7.8, 6.0 Hz, 1H), 3.78 (s, 3H), 3.76-3.71 (m, 1H), 3.64-3.57 (m, 1H), 3.52 (m, 2H), 2.66-2.59 (m, 2H), 1.68 (s, 3H), 1.63 (s, 3H), 1.06 (dd, J=6.8, 4.8 Hz, 6H), 0.96 (d, J=6.8 Hz, 6H). ¹³C NMR (151 MHz, CD₃CN) δ 159.9, 138.3, 129.7, 123.0, 119.3, 118.25, 117.9, 116.7, 113.4, 86.16, 86.0, 64.6, 63.3, 58.3, 58.1, 55.3, 43.5, 28.0, 24.7, 24.4, 24.3, 24.2, 20.5. ³¹P NMR (243 MHz, CD₃CN) δ 151.2, 149.2. MS (ESI+APCI), calculated for C₂₁H₃₃N₂O₃PS₂ [M+H]⁺ exact mass m/z=457.17, found 457.2.

Compound 155 (R=4-CF₃C₆H₄): Compound 155 was prepared analogously from compound 115 (0.58 g, 2.0 mmol). 37% Yield, ¹H NMR (600 MHz, CD₃CN) δ 7.65 (m, 4H), 5.13 (d, J=5.9 Hz, 1H), 4.42 (ddd, J=13.1, 5.9, 0.8 Hz, 1H), 3.81-3.70 (m, 1H), 3.60 (m, 1H), 3.51 (m, 2H), 2.67-2.58 (m, 2H), 1.68 (s, 3H), 1.65 (s, 3H), 1.05 (d, J=6.8 Hz, 6H), 0.90 (d, J=6.8 Hz, 6H). ¹³C NMR (151 MHz, CD₃CN) δ 142.0, 131.5, 129.6, 125.5, 124.10, 119.30, 86.1, 64.9, 62.3, 58.2, 43.6, 27.5, 24.3, 24.2, 24.1, 20.5. ³¹P NMR (243 MHz, CD₃CN) δ 149.2. MS (ESI+APCI), calculated for C₂₁H₃₀F₃N₂O₂PS₂ [M+H]⁺ exact mass m/z=495.14, found 495.2.

Compound 156 (R=Et): Compound 156 was prepared analogously from compound 116 (2.5 mmol). 35% Yield, ¹H NMR (600 MHz, CD₃CN) δ 4.23-4.15 (m, 1H), 3.91-3.83 (m, 1H), 3.76-3.65 (m, 4H), 2.72-2.64 (m, 2H), 2.16-1.94 (m, 1H), 1.79-1.65 (m, 1H), 1.51 (d, J=9.9 Hz, 3H), 1.46 (d, J=16.0 Hz, 3H), 1.27-1.17 (m, 12H), 1.01 (dt, J=10.7, 7.4 Hz, 3H). ¹³C NMR (151 MHz, CD₃CN) δ 119.2, 87.1, 86.3, 62.1, 61.3, 60.6, 60.1, 58.4, 43.7, 30.5, 28.7, 28.3, 24.0, 23.7, 23.5, 20.5, 13.4. ³¹P NMR (243 MHz, CD₃CN) S 150.9, 149.6. MS (ESI+APCI), calculated for C₁₆H₃N₂O₂PS₂ [M] exact mass m/z=378.16.

Compound 157 (R=i-Pr): Compound 157 was prepared analogously from compound 117 (0.75 g, 3.9 mmol). 90% Yield, ¹H NMR (600 MHz, CD₃CN) δ 4.30 (dd, J=10.8, 3.6 Hz, 0.4H), 4.27 (dd, J=10.2, 3.0 Hz, 0.6H), 3.92-3.84 (m, 1H), 3.76-3.66 (m, 3.4H), 3.64 (ddd, J=9.6, 3.0, 1.8 Hz, 0.6H), 2.73-2.63 (m, 2H), 2.42-2.34 (m, 0.4H), 2.23-2.12 (m, 0.6H), 1.57 (s, 2H), 1.55 (d, J=1.2 Hz, 1H), 1.51-1.44 (m, 3H), 1.25 (s, 3H), 1.24 (s, 3H), 1.23-1.19 (m, 6H), 1.14 (d, J=6.6 Hz, 1.8H), 1.11 (d, J=6.0 Hz, 1.2H), 1.07 (d, J=7.2 Hz, 1.8H), 1.05 (d, J=7.2 Hz, 1.2H). ¹³C NMR (151 MHz, CD₃CN) δ 119.3, 86.0, 85.1, 69.0, 68.0, 64.7, 58.3, 43.7, 29.6, 29.3, 29.1, 28.8, 25.2, 24.5, 24.4, 23.2, 23.1, 23.0, 22.6, 20.6, 20.5, 20.4. ³¹P NMR (243 MHz, CD₃CN) δ 150.6, 149.6. MS (ESI+APCI), calculated for C₁₇H₃₃N₂O₂PS₂ [M] exact mass m/z=392.17.

Compound 150 (R=Me): Compound 150 was prepared analogously from compound 110 (R=Me) (0.20 g, 1.2 mmol). Yield: 54% (0.24 g). ¹H NMR (500 MHz, CD₃CN) δ 4.19-4.10 (m, 1H), 4.01-3.64 (m, 5H), 2.75-2.63 (m, 2H), 1.52 (s, 3H), 1.50-1.37 (m, 6H), 1.27-1.20 (m, 12H). ³¹P NMR (202 MHz, CD₃CN) δ 152.39, 150.75. MS (ESI+APCI), calculated for C₁₅H₂₉N₂O₂PS₂ [M] exact mass m/z=364.14.

Compound 170 (R=Me): Compound 170 was prepared analogously to compound 142 from carbinol 130 (R=Me) (0.49 g, 3 mmol). Yield: 0.99 g, 94%. ¹H NMR (600 MHz, CD₃CN) δ 3.90-3.83 (m, 2H), 3.74-3.63 (m, 3H), 3.57-3.48 (m, 1H), 2.73-2.63 (m, 2H), 1.54-1.51 (m, 2H); 1.51 (s, 3H), 1.483 (s, 3H), 1.479 (s, 1H), 1.23 (dd, J=1.2 Hz, J=7.2, 6H), 1.20 (d, J=6.6 Hz, 6H); ¹³C NMR (151 MHz, CD₃CN) δ 119.14, 92.35, 92.26, 91.95; 59.59; 59.22; 58.55, 58.51, 58.42, 58.38, 51.64, 51.61; 51.42, 43.62; 43.60; 43.54, 43.52, 26.37; 26.14; 24.57; 24.52, 24.44, 24.42, 24.38, 22.88, 22.38, 20.51, 20.46, 19.42, 19.29, 19.25; ³¹P NMR (243 MHz, CD₃CN) δ 149.39, 149.21. MS (ESI+APCI), calculated for C₁₅H₂₉N₂O₂PS₂ [M] exact mass m/z=364.14.

### Example 12. Synthesis of Cyclic Disulfide Modified Chiral Phosphate Prodrug Derivatives

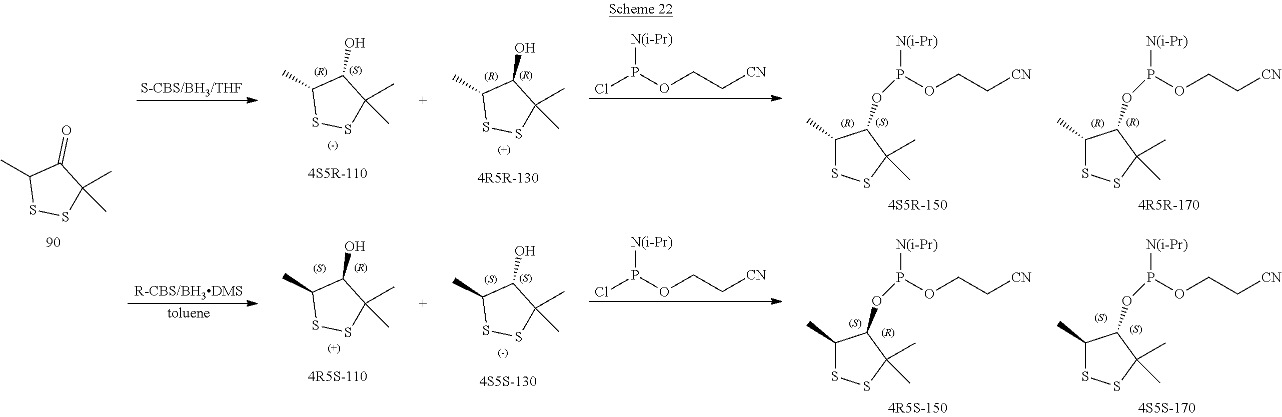

Compounds 4S5R-110 and 4R5R-130. To the solution of S-CBS (1.88 g, 6.8 mmol, 0.2 eq) in THF (55 mL) was added BH₃-THF (1 M, 33.9 mL, 1 eq) at −30° C. under N₂, and the mixture was stirred at −30° C. for 30 mins. The solution of compound 90 (5.5 g, 33.9 mmol, 1 eq) in THF (110 mL) was added to the above mixture dropwise at −30° C., and the reaction mixture was warmed to 20° C. slowly and stirred for an additional 12 hrs. The reaction was quenched by slow addition of aqueous NH₄Cl (sat., 200 mL) at 0° C. The resulting mixture was extracted with ethyl acetate (3×200 mL), the organic layer was dried over Na₂SO₄ and concentrated under reduced pressure at 20-25° C. to give the crude. The residue was purified three times by column chromatography on silica gel (gradient elution, petroleum ether/ethyl acetate) to give 4S5R-110, (3.85 g, yield: 51%) as a yellow solid and 4R5R-130 (1.30 g, yield 17%) as a light-yellow oil. 4S5R-110: 1H NMR (400 MHz, DMSO-d6) δ ppm 1.29 (d, J=6.75 Hz, 3H), 1.38 (d, J=5.25 Hz, 6H), 3.77 (dd, J=6.88, 4.50 Hz, 1H), 3.86 (qd, J=6.71, 4.50 Hz, 1H), 5.16 (d, J=7.00 Hz, 1H). MS (ESI+APCI), calculated for C₆H₁₂OS₂ [M] exact mass m/z=164.03. ee=40% By chiral HPLC on Chiralpak IG-3. [α]_{D}²⁰=−194 (enantiomer purified by chiral HPLC). Absolute configuration of purified enantiomer as established by VCD: 4S-5R. 4R5R-130: 1H NMR (400 MHz, DMSO-d6) δ ppm 1.32 (d, J=3.88 Hz, 6H), 1.38 (d, J=6.38 Hz, 3H), 3.20-3.28 (m, 1H), 3.37 (dd, J=8.38, 6.25 Hz, 1H), 5.68 (d, J=6.25 Hz, 1H). MS (ESI+APCI), calculated for C₆H₁₂OS₂ [M] exact mass m/z=164.03. ee=96% By chiral HPLC on Chiralpak IG-3. [α]_{D}²⁰=+224. Absolute configuration as established by VCD: 4R-5R.

Compounds 4R5S-110 and 4S5S-130. To the solution of R-CBS (1 M, 6.8 mmol, 0.2 eq) in THF (55 mL) was added BH₃-DMS (10 M, 3.39 mL, 1 eq) at −30° C. under N2, and the mixture was stirred at −30° C. for 30 mins. The solution of compound 90 (5.5 g, 33.9 mmol, 1 eq) in THF (110 mL) was added to the above mixture dropwise at −30° C., and the reaction mixture was warmed to 20° C. slowly and stirred for an additional 12 hrs. The reaction was quenched by slow addition of aqueous NH₄Cl (sat., 200 mL) at 0° C. The resulting mixture was extracted with ethyl acetate (3×200 mL), the organic layer was dried over Na₂SO₄ and concentrated under reduced pressure at 20-25° C. to give the crude. The residue was purified three times by column chromatography on silica gel (gradient elution, petroleum ether/ethyl acetate) to give 4R5S-110 (4.6 g, yield 61%) as a yellow solid and 4S5S-130 (1.15 g, yield 15%) as a yellow oil. 4R5S-110: 1H NMR (400 MHz, DMSO-d6) δ ppm 1.29 (d, J=6.75 Hz, 3H), 1.38 (d, J=5.25 Hz, 6H), 3.77 (dd, J=6.88, 4.50 Hz, 1H), 3.86 (qd, J=6.69, 4.57 Hz, 1H), 5.16 (d, J=6.88 Hz, 1H). MS (ESI+APCI), calculated for C₆H₁₂OS₂ [M] exact mass m/z=164.03. ee=40% by chiral HPLC on Chiralpak IG-3. [α]_{D}²⁰=+192 (enantiomer purified by chiral HPLC). Absolute configuration of purified enantiomer as established by VCD: 4R-5S. 4S5S-130: 1H NMR (400 MHz, DMSO-d6) δ ppm 1.32 (d, J=3.88 Hz, 6H), 1.38 (d, J=6.50 Hz, 3H), 3.20-3.29 (m, 1H), 3.37 (dd, J=8.25, 6.25 Hz, 1H), 5.68 (d, J=6.25 Hz, 1H). MS (ESI+APCI), calculated for C₆H₁₂OS₂ [M] exact mass m/z=164.03. ee=98% by chiral HPLC on Chiralpak IG-3. [α]_{D}²⁰=−231. Absolute configuration as established by VCD: 4S-5S.

Compound 4S5R-150: Compound 4S5R-150 was prepared analogously to procedure for compound 142 from carbinol 4S5R-110 (0.82 g, 5 mmol). Yield: 49% (0.90 g). ¹H NMR (600 MHz, CD₃CN) δ 4.18-4.11 (m, 1H), 3.95-3.83 (m, 2H), 3.76-3.65 (m, 3H), 2.73-2.64 (m, 2H), 1.52 (s, 3H), 1.47 (d, J=4.8 Hz, 3H), 1.44-1.40 (m, 3H), 1.27-1.23 (m, 6H), 1.21 (d, J=6.6 Hz, 6H). ³¹P NMR (243 MHz, CD₃CN) δ 150.98, 149.34. ¹³C NMR (151 MHz, CD₃CN) δ 119.19, 87.07, 86.99, 86.68, 86.60, 62.06, 60.84, 58.59, 58.45, 58.34, 52.03, 50.73, 43.72, 43.66, 43.57, 28.44, 27.96, 24.60, 24.53, 24.44, 24.40, 24.58, 23.63, 20.58, 20.53, 16.52, 16.25, 16.22. MS (ESI+APCI), calculated for C₁₅H₂₉N₂O₂PS₂ [M] exact mass m/z=364.14.

Compound 4R5S-150: Compound 4R5S-150 was prepared analogously to procedure for compound 142 from carbinol 4R5S-110 (0.82 g, 5 mmol). Yield: 39% (0.71 g). ¹H NMR (600 MHz, CD₃CN) δ 4.18-4.11 (m, 1H), 3.95-3.83 (m, 2H), 3.76-3.65 (m, 3H), 2.73-2.64 (m, 2H), 1.52 (s, 3H), 1.47 (d, J=4.8 Hz, 3H), 1.44-1.40 (m, 3H), 1.27-1.23 (m, 6H), 1.21 (d, J=6.6 Hz, 6H). ³¹P NMR (243 MHz, CD₃CN) δ 150.98, 149.34. ¹³C NMR (151 MHz, CD₃CN) δ 119.19, 87.07, 86.99, 86.68, 86.60, 62.06, 60.84, 58.59, 58.45, 58.34, 52.03, 50.73, 43.72, 43.66, 43.57, 28.44, 27.96, 24.60, 24.53, 24.44, 24.40, 24.58, 23.63, 20.58, 20.53, 16.52, 16.25, 16.22. MS (ESI+APCI), calculated for C₁₅H₂₉N₂O₂PS₂ [M] exact mass m/z=364.14.

Compound 4R5R-170: Compound 4R5R-170 was prepared analogously to procedure for compound 142 from carbinol 4R5R-130 (0.49 g, 3 mmol). Yield: 0.98 g, 91%. ¹H NMR (600 MHz, CD₃CN) δ 3.90-3.83 (m, 2H), 3.74-3.63 (m, 3H), 3.57-3.48 (m, 1H), 2.73-2.63 (m, 2H), 1.54-1.51 (m, 2H); 1.51 (s, 3H), 1.483 (s, 3H), 1.479 (s, 1H), 1.23 (dd, J=1.2 Hz, J=7.2, 6H), 1.20 (d, J=6.6 Hz, 6H); ³¹P NMR (243 MHz, CD₃CN) δ 149.39, 149.21. ¹³C NMR (151 MHz, CD₃CN) δ 119.14, 92.35, 92.26, 91.95; 59.59; 59.22; 58.55, 58.51, 58.42, 58.38, 51.64, 51.61; 51.42, 43.62; 43.60; 43.54, 43.52, 26.37; 26.14; 24.57; 24.52, 24.44, 24.42, 24.38, 22.88, 22.38, 20.51, 20.46, 19.42, 19.29, 19.25. MS (ESI+APCI), calculated for C₁₅H₂₉N₂O₂PS₂ [M] exact mass m/z=364.14.

Compound 4S5S-170: Compound 4S5S-170 was prepared analogously to procedure for compound 142 from carbinol 4S5S-130 (0.49 g, 3 mmol). Yield: 1.00 g, 92%. ¹H NMR (600 MHz, CD₃CN) δ 3.90-3.83 (m, 2H), 3.74-3.63 (m, 3H), 3.57-3.48 (m, 1H), 2.73-2.63 (m, 2H), 1.54-1.51 (m, 2H); 1.51 (s, 3H), 1.483 (s, 3H), 1.479 (s, 1H), 1.23 (dd, J=1.2 Hz, J=7.2, 6H), 1.20 (d, J=6.6 Hz, 6H); ³¹P NMR (243 MHz, CD₃CN) δ 149.39, 149.21. ¹³C NMR (151 MHz, CD₃CN) δ 119.14, 92.35, 92.26, 91.95; 59.59; 59.22; 58.55, 58.51, 58.42, 58.38, 51.64, 51.61; 51.42, 43.62; 43.60; 43.54, 43.52, 26.37; 26.14; 24.57; 24.52, 24.44, 24.42, 24.38, 22.88, 22.38, 20.51, 20.46, 19.42, 19.29, 19.24. MS (ESI+APCI), calculated for C₁₅H₂₉N₂O₂PS₂ [M] exact mass m/z=364.14.

### Example 13. Synthesis of Spirocyclic Phosphate Prodrug Derivatives

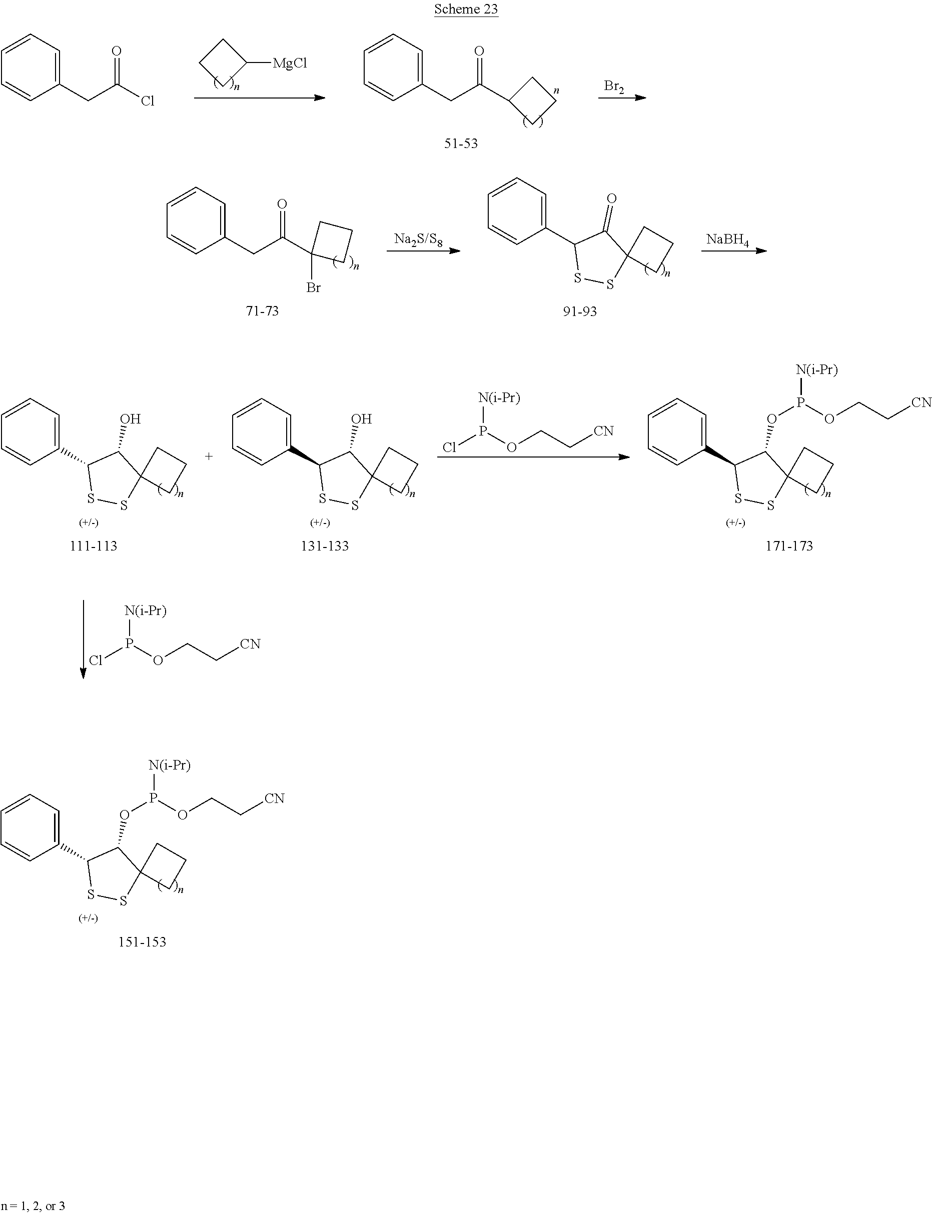

General procedure—Compound 51 (n=1): An oven-dried flask was flashed by argon and magnesium (835 mg, 34.4 mmol), followed by 30 mL of anhydrous THF and cyclobutyl chloride (2.86 g, 31.6 mmol) were added. The reaction mixture was stirred under reflux at 75° C. bath temperature for 5 hours to form the Grignard reagent. To the other oven-dried flask was added phenylacetyl chloride (4.25 g, 27.5 mmol) and anhydrous THF (60 mL) under Ar. CuBr (3.94 g, 27.5 mmol) was added, the reaction mixture was stirred for 5 min and cooled to 0-5° C. The Grignard reagent was then slowly added into the stirring reaction mixture for 20 min, the mixture was allowed to warm up to room temperature and stirred overnight. The reaction was quenched with saturated NH₄Cl (150 mL) at 0° C. and partitioned between EtOAc (300 mL) and water (200 mL). The organic layer was washed with 5% NaCl (1×150 mL), saturated NaCl (1×150 mL), dried over Na₂SO₄, filtered, and concentrated under vacuum to give a green crude oil (5.65 g). The crude was purified by silica gel chromatography (120 g standard flash column) with gradient of 0 to 20% EtOAc in hexanes to afford 51 as a slightly yellow oil. (2.8 g, yield 45%). 1H NMR (600 MHz, DMSO-d6) δ 7.33-7.28 (m, 2H), 7.24 (m, 1H), 7.20-7.16 (m, 2H), 3.69 (s, 2H), 3.40-3.33 (m, 1H), 2.20-1.97 (m, 4H), 1.93-1.82 (m, 1H), 1.74-1.60 (m, 1H). MS (ESI+APCI), calculated for C₁₂H₁₄O [M] exact mass m/z=174.10.

Compound 52 (n=2): Compound 52 was prepared analogously from phenylacetyl chloride (2.5 g) and commercial cyclopentyl magnesium chloride (2M in diethyl ether, 9 mL, 17.8 mmol) with CuBr (2.32 g, 16.2 mmol). Obtained: 2.67 g, yield 87%. ¹H NMR (600 MHz, DMSO-d6) δ 7.32-7.18 (m, 5H), 3.81 (s, 2H), 3.04-2.98 (m, 1H), 1.75 (m, 2H), 1.66 (m, 2H), 1.53 (m, 4H). MS (ESI+APCI), calculated for C₁₃H₆O [M] exact mass m/z=188.12.

Compound 53 (n=3): Compound 53 was prepared analogously from phenylacetyl chloride (10.0 g) and cyclohexyl magnesium chloride (1.0 M in 2-methyltetrafuran, 75 mL, 74.4 mmol) with CuBr (9.28 g, 64.7 mmol) to give compound 53 (8.8 g, yield 67%). 1H NMR (600 MHz, DMSO-d6) δ 7.38-7.31 (m, 2H), 7.31-7.24 (m, 1H), 7.24-7.17 (m, 2H), 3.75 (s, 2H), 2.48 (tt, J=11.5, 3.4 Hz, 1H), 1.88-1.82 (m, 2H), 1.82-1.76 (m, 2H), 1.67 (m, 1H), 1.43-1.33 (m, 2H), 1.32-1.16 (m, 3H). ¹³C NMR (151 MHz, CDCl₃) δ 211.3, 134.5, 129.5, 128.6, 126.8, 50.1, 47.9, 28.6, 25.8, 25.6. MS (ESI+APCI), calculated for C₁₄H₁₈O [M] exact mass m/z=202.14.

Compound 71 (n=1): Compound 51 (n=1), (2.50 g, 14.4 mmol) was dissolved in diethyl ether (20 mL) under inert atmosphere. A small portion (12 drops) of separately prepared solution of bromine (4.93 g, 30.9 mmol) in dichloromethane (10 mL) was added to the ketone solution and stirred for several minutes to initiate the reaction. The rest of the bromine solution was added dropwise to the stirred ketone solution over 0.5 hour and the mixture was stirred at room temperature for 2 hours. The mixture was diluted with diethyl ether (100 mL) and added portion-wise to a stirred aqueous solution of 5% NaCl followed by stirring for 10 minutes. Organic layer was washed with 5% NaCl (2×), 5% Na₂S₂O₅ (1×), saturated NaCl (1×), dried over Na₂SO₄, filtered, and concentrated to afford 61 as a greyish solid, (3.65 g) 76%. ¹H NMR (600 MHz, DMSO) δ 7.70-7.62 (m, 2H), 7.47-7.34 (m, 3H), 6.38 (s, 1H), 2.97 (m, 1H), 2.73-2.57 (m, 2H), 2.50-2.44 (m, 1H), 2.17 (m, 1H), 1.66 (m, 1H). ¹³C NMR (151 MHz, DMSO) δ 197.0, 136.6, 129.7, 129.5, 129.4, 128.7, 128.0, 61.9, 47.6, 36.3, 35.2, 16.6, 16.6. MS (ESI+APCI), calculated for C₁₂H₁₂Br₂O [M] exact mass m/z=329.93.

Compound 72 (n=2): Compound 72 was prepared analogously from compound 52 (n=2), (2.00 g, 11.35 mmol). 79% yield, ¹H NMR (600 MHz, CDCl₃) δ 7.73-7.58 (m, 2H), 7.48-7.32 (m, 3H), 6.15 (s, 1H), 2.52 (m, 1H), 2.41 (m, 1H), 2.21 (m, 1H), 2.17-2.11 (m, 1H), 2.10-1.94 (m, 3H), 1.90-1.82 (m, 1H), 1.76 (m, 1H). ¹³C NMR (151 MHz, CDCl₃) δ 196.1, 135.8, 129.2, 128.9, 74.5, 47.9, 40.0, 39.0, 23.1, 22.9. MS (ESI+APCI), calculated for C₁₃H₁₄Br₂O [M] exact mass m/z=343.94.

Compound 73 (n=3): Compound 73 was prepared analogously from compound 53 (n=3), (5.40 g, 32 mmol). 68% yield, ¹H NMR (600 MHz, CDCl₃) δ 7.77-7.60 (m, 2H), 7.49-7.30 (m, 3H), 6.16 (s, 1H), 2.45-2.22 (m, 1H), 2.14-2.06 (m, 1H), 1.92-1.51 (m, 6H), 1.49-1.13 (m, 2H). MS (ESI+APCI), calculated for C₁₄H₁₆Br₂O [M] exact mass m/z=357.96.

Compound 91(n=1): N-Methylacetamide (15 mL) was heated at 33° C. under inert atmosphere. Disodiumsulfide (1.20 g, 15.4 mmol) and sulfur (985 mg, 30.7 mmol) were added, and the suspension was stirred at 35° C. for 24 hours to dissolve solids. Reaction was cooled to 30° C. and compound 71 (3.4 g, 10.2 mmol) was added. The mixture was stirred at 30° C. for 22 hours and quenched by slowly pouring to a stirred solution of 5% NaCl (250 mL). The mixture was diluted with ethyl acetate (200 mL), washed with 5% NaCl (3×) and saturated NaCl (1×). Organic layer was dried over Na₂SO₄, filtered, and concentrated. Oily residue was diluted in hexanes and stirred for 18 hours. Solids were removed by decanting and the supernatant was concentrated to an oil. The product was purified by silica gel flash chromatography. Compound 91 was isolated as a yellow oil (780 mg, yield 32%). ¹H NMR (600 MHz, CDCl₃) δ 7.48-7.22 (m, 6H), 4.74 (s, 1H), 2.86-2.68 (m, 2H), 2.56-2.43 (m, 1H), 2.36-2.17 (m, 2H), 2.17-2.03 (m, 1H). ¹³C NMR (151 MHz, CDCl₃) δ 208.3, 135.6, 129.1, 128.9, 128.6, 59.8, 59.5, 35.5, 30.3, 15.8. MS (ESI+APCI), calculated for C₁₂H₂OS₂ [M+H]⁺ exact mass m/z=237.03, found 237.0.

Compound 92 (n=2): Compound 92 was prepared analogously from compound 72 (2.1 g, 6.07 mmol). 58% yield, ¹H NMR (600 MHz, DMSO) δ 7.44-7.29 (m, 5H), 5.29 (s, 1H), 2.30-2.14 (m, 2H), 2.04 (m, 1H), 1.97-1.76 (m, 5H). ¹³C NMR (151 MHz, DMSO) δ 210.9, 136.7, 129.6, 129.3, 128.8, 68.1, 59.4, 37.5, 25.9, 25.8. MS (ESI+APCI), calculated for C₁₃H₄OS₂ [M+H]⁺ exact mass m/z=251.05, found 251.0.

Compound 93 (n=3): Compound 93 was prepared analogously from compound 73 (2.1 g, 5.83 mmol). 30%, yield, −80% purity. Major Compound ¹H NMR (600 MHz, DMSO) δ 7.53-7.25 (m, 5H), 5.33 (s, 1H), 2.21-2.07 (m, 1H), 1.99-1.54 (m, 7H), 1.47-1.21 (m, 3H). MS (ESI+APCI), calculated for C₁₄H₆OS₂ [M−H]⁺ exact mass m/z=263.05, found 263.0.

Compound 111 (n=1): Ketone 91(400 mg, 1.7 mmol) was dissolved in ethanol (15 mL) under inert atmosphere and cooled to −78° C. Acetic acid (102 mg, 1.7 mmol) was added, followed by sodium borohydride (64 mg, 1.7 mmol), the mixture was stirred at 0° C. for one hour and stirred at room temperature for 15 hours. A second portion of sodium borohydride (16 mg, 0.44 mmol) was added after the reaction mixture was cooled to 0° C. again. The reaction mixture was stirred for additional 4 hours at room temperature, cooled to 0° C., quenched with saturated NH₄Cl, and allowed to warm up to room temperature. Ethyl acetate (40 mL), saturated NH₄Cl (20 mL), and water (16 mL) were added, and the mixture was stirred at room temperature for 24 hours. The organic layer was separated, washed with 1:1 saturated NH₄Cl: water (Ix), saturated NaHCO₃(Ix), and saturated NaCl (Ix), dried over Na₂SO₄, filtered, and concentrated. The crude product was purified by silica gel flash chromatography to obtain pure compounds 102. (140 mg, 34%). ¹H NMR (600 MHz, DMSO) δ 7.59-7.53 (m, 2H), 7.34-7.24 (m, 3H), 5.38 (d, J=7.0 Hz, 1H), 4.86 (d, J=3.3 Hz, 1H), 4.32 (dd, J=7.0, 3.4 Hz, 1H), 2.54 (m, 1H), 2.31 (m, 2H), 2.17 (m, 1H), 1.96-1.87 (m, 1H), 1.87-1.77 (m, 1H). ¹³C NMR (151 MHz, DMSO) δ 136.6, 130.3, 128.3, 128.1, 81.7, 67.9, 60.1, 37.7, 29.1, 16.1. MS (ESI+APCI), calculated for C₁₂H₁₄OS₂ [M−H]⁻ exact mass m/z=237.05, found 237.0.

Compound 112 (n=2): Compound 112 was prepared analogously from compound 92, (750 mg, 3.0 mmol). 54% yield, ¹H NMR (600 MHz, DMSO) δ 7.58-7.52 (m, 2H), 7.35-7.23 (m, 3H), 5.26 (d, J=7.4 Hz, 1H), 4.91 (d, J=3.7 Hz, 1H), 4.00 (dd, J=7.4, 3.7 Hz, 1H), 2.22 (m, 1H), 2.08-1.99 (m, 1H), 1.85-1.71 (m, 4H), 1.71-1.54 (m, 2H). ¹³C NMR (151 MHz, DMSO) δ 137.1, 130.5, 128.2, 127.9, 82.9, 77.3, 61.8, 33.5, 25.2, 25.1. MS (ESI+APCI), calculated for C₁₃H₁₆OS₂ [M+H]⁺ exact mass m/z=253.05, found 253.0.

Compound 113 (n=3): Compound 113 was prepared analogously from compound 93, (600 mg, 2.27 mmol). 41% yield, ¹H NMR (600 MHz, DMSO) δ 7.57-7.50 (m, 2H), 7.36-7.24 (m, 3H), 5.19 (dd, J=7.6, 1.3 Hz, 1H), 4.96 (d, J=3.4 Hz, 1H), 4.04 (dd, J=7.7, 3.4 Hz, 1H), 2.03 (d, J=14.1 Hz, 1H), 1.98-1.91 (m, 1H), 1.76 (m, 1H), 1.72-1.52 (m, 5H), 1.34-1.17 (m, 2H). ¹³C NMR (151 MHz, DMSO) δ 136.8, 130.4, 128.2, 127.9, 83.5, 72.5, 61.61, 36.9, 33.0, 25.6, 25.0, 23.4. MS (ESI+APCI), calculated for C₁₄H₁₈OS₂ [M+H]⁺ exact mass m/z=267.08, found 267.0.

Compound 151 (n=1): Compound 111 (0.33 g, 1.26 mmol) was dissolved in anhydrous ethyl acetate (14 mL) under inert atmosphere. N,N-diisopropylethylamine (224 mg, 1.89 mmol) and 2-cyanoethyl N,N-diisopropylchlorophosphoramidite (447 mg, 1.89 mmol) were added and the mixture was stirred at room temperature overnight. The mixture was quenched, washed with 5% NaCl (3×), saturated NaCl (1×), dried over Na₂SO₄, filtered, and concentrated. The crude product was purified by silica gel flash chromatography to obtain pure compound 151. (340 mg, 61%), ¹H NMR (CD₃CN) δ 7.50 (m, 2H), 7.36-7.24 (m, 3H), 4.94 (d, J=5.0 Hz, 1H), 4.57-4.46 (m, 1H), 3.79 (m, 1H), 3.65 (m, 1H), 3.59-3.46 (m, 2H), 3.32 (m, 1H), 3.07-2.97 (m, 1H), 2.71-2.61 (m, 1H), 2.59-2.44 (m, 2H), 2.37-2.22 (m, 1H), 2.22-2.04 (m, 2H), 1.09 (d, J=6.8 Hz, 7H), 0.99 (d, J=6.8 Hz, 5H). ³¹P NMR (CD₃CN) δ 151.1, 148.8. MS (ESI+APCI), calculated for C₂₁H₃₁N₂O₂PS₂ [M+H]⁺ exact mass m/z=439.16, found 439.2.

Compound 152 (n=2): Compound 152 was prepared analogously from compound 112, (388 mg, 1.54 mmol). 64% yield, ¹H NMR (CD₃CN) δ 7.59-7.48 (m, 2H), 7.40-7.24 (m, 3H), 5.05-4.94 (m, 1H), 4.43 (dd, J=12.6, 5.2 Hz, 1H), 3.73 (m, 1H), 3.59 (m, 1H), 3.51 (m, 2H), 2.64-2.50 (m, 2H), 2.15-2.07 (m, 1H), 2.07-1.99 (m, 1H), 1.91-1.71 (m, 4H), 1.04 (d, 6H), 0.99 (d, 6H). ³¹P NMR (CD₃CN) δ 151.1, 149.1. MS (ESI+APCI), calculated for C₂₂H₃₃N₂O₂PS₂ [M+H]⁺ exact mass m/z=453.17, found 453.2.

Compound 153 (n=3): Compound 153 was prepared analogously from compound 113, (420 mg, 1.58 mmol). 70% yield, ¹H NMR (CD₃CN) δ 7.57-7.44 (m, 2H), 7.37-7.23 (m, 3H), 4.98 (d, J=5.1 Hz, 1H), 4.37 (m, 1H), 3.74 (m, 1H), 3.64-3.50 (m, 2H), 3.38-3.30 (m, 1H), 2.68-2.60 (m, 2H), 2.48-2.37 (m, 1H), 2.26-2.12 (m, 1H), 2.04-1.98 (m, 2H), 1.85-1.66 (m, 3H), 1.66-1.47 (m, 2H), 1.34-1.18 (m, 1H), 1.06 (d, J=6.5 Hz, 6H), 0.99 (d, J=6.8 Hz, 6H). ³¹P NMR (CD₃CN) δ 151.6, 149.4. MS (ESI+APCI), calculated for C₂₃H₃₅N₂O₂PS₂ [M] exact mass m/z=466.19.

### Example 14. Synthesis of Oligonucleotides Containing Modified Phosphate Prodrug at the 5′ End of the Oligonucleotide

All oligonucleotides were synthesized as described here, or as otherwise described in Table X. Oligonucleotides were synthesized at 1 or IOpmol scale using standard solid phase oligonucleotide protocols, with 500-A controlled pore glass (CPG) solid supports from

Prime Synthesis and commercially available amidites from ChemGenes. The phosphoramidite solutions were 0.15 M in anhydrous acetonitrile with 15% THF as a cosolvent for 2′-O-methyl uridine, 2′-O-methyl cytidine, and the modified phosphate prodrugs. The modified phosphate prodrug monomers were coupled either on synthesizer or manually. For manual coupling, activator (0.25M 5-ethylthio-1H-tetrazole (ETT) in anhydrous ACN) was added followed by equal volume of prodrug solution. Solution was mixed for 20 minutes. Following coupling, the column was put on an ABI for oxidation or sulfurization. Oxidation (0.02M iodine in THF/pyridine/water) or sulfurization solution (0.1M 3-(dimethylaminomethylidene) amino-3H-1,2,4-dithiazole-3-thione (DDTT) in pyridine) was delivered to column for one minute or 30 seconds, respectively, and then held in solution for 10 minutes. This process was repeated for sulfurization. After completion of the solid-phase syntheses (SPS), the CPG solid support was washed with anhydrous acetonitrile and dried with argon. Oligonucleotides were deprotected by incubation with 5% diethanolamine (DEA) in ammonia at room temperature for 2 hours.

Crude oligonucleotides were purified using strong anion exchange through a TSKgel SuperQ-5PW(20) resin with phosphate buffers (pH=8.5) containing sodium bromide at 65° C. The appropriate fractions were pooled and desalted via SEC.

TABLE 20
Single oligonucleotide strands for SAR having modified phosphate prodrug at 5′ end

| | | | | SEQ |
|---|---|---|---|---|
| | | Synthesizer/ | | ID |
| Oligo ID | Sequence | scale | MW | NO: |
| A-1875179 | (Pmmds)usCfsacuUfuAfUfugagUfuU | Precursor (up to prodrug) | 7757.178 | 160 |
| | fcugugscsc | on Akta Oligopilot/12 mL, | | |
| | | Manual coupling of | | |
| | | prodrug | | |
| A-3019037 | (tPmmds)usCfscuUfuAfUfugagUfu | Precursor (up to prodrug) | 7757.178 | 161 |
| | Ufcugugscsc | on Akta Oligopilot/12 mL, | | |
| | | Manual coupling of prodrug | | |
| A-3654050 | (+cPmmds)usCfsacuUfuAfUfugagUf | Precursor (up to prodrug) | 7757.178 | 162 |
| | uUfcugugscsc | on Akta Oligopilot/12 mL, | | |
| | | Manual coupling of prodrug | | |
| A-3654052 | (−cPmmds)usCfsacuUfuAfUfugagUfu | Precursor (up to prodrug) | 7757.178 | 163 |
| | Ufcugugscsc | on Akta Oligopilot/12 mL, | | |
| | | Manual coupling of | | |
| | | prodrug | | |
| A-3654054 | (+tPmmds)usCfsacuUfuAfUfugagUfu | Precursor (up to prodrug) | 7757.178 | 164 |
| | Ufcugugscsc | on Akta Oligopilot/12 mL, | | |
| | | Manual coupling of | | |
| | | prodrug | | |
| A-3654055 | (−tPmmds)usCfsacuUfuAfUfugagUfu | Precursor (up to prodrug) | 7757.178 | 165 |
| | Ufcugugscsc | on Akta Oligopilot/12 mL, | | |
| | | Manual coupling of | | |
| | | prodrug | | |
| A-3019038 | (PdAr1s)usCfsacuUfuAfUfugagUfuU | Precursor (up to prodrug) | 7819.249 | 166 |
| | fcugugscsc | on Akta Oligopilot/12 mL, | | |
| | | Manual coupling of | | |
| | | prodrug | | |
| A-3036616 | (PdAr2s)usCfsacuUfuAfUfugagUfuU | Precursor (up to prodrug) | 7819.249 | 167 |
| | fcugugscsc | on Akta Oligopilot/12 mL, | | |
| | | Manual coupling of | | |
| | | prodrug | | |
| A-3019039 | (PdAr3s)usCfsacuUfuAfUfugagUfuU | Precursor (up to prodrug) | 7833.276 | 168 |
| | fcugugscsc | on Akta Oligopilot/12 mL, | | |
| | | Manual coupling of | | |
| | | prodrug | | |
| A-3036617 | (PdAr4s)usCfsacuUfuAfUfugagUfuU | Precursor (up to prodrug) | 7833.276 | 169 |
| | fcugugscsc | on Akta Oligopilot/12 mL, | | |
| | | Manual coupling of | | |
| | | prodrug | | |
| A-3019040 | (PdAr5s)usCfsacuUfuAfUfugagUfuU | Precursor (up to prodrug) | 7849.275 | 170 |
| | fcugugscsc | on Akta Oligopilot/12 mL, | | |
| | | Manual coupling of | | |
| | | prodrug | | |
| A-3196466 | (PdAr6s)usCfsacuUfuAfUfugagUfuU | Precursor (up to prodrug) | 7849.275 | 171 |
| | fcugugscsc | on Akta Oligopilot/12 mL, | | |
| | | Manual coupling of | | |
| | | prodrug | | |
| A-3768100 | (PdAr7s)usCfsacuUfuAfUfugagUfuU | Precursor (up to prodrug) | 7898.145 | 172 |
| | fcugugscsc | on Akta Oligopilot/12 mL, | | |
| | | Manual coupling of | | |
| | | prodrug | | |
| A-3768101 | (PdAr9s)usCfsacuUfuAfUfugagUfuU | Precursor (up to prodrug) | 7844.259 | 173 |
| | fcugugscsc | on Akta Oligopilot/12 mL, | | |
| | | Manual coupling of | | |
| | | prodrug | | |
| A-3768102 | (PdAr11s)usCfsacuUfuAfUfugagUfu | Precursor (up to prodrug) | 7837.239 | 174 |
| | Ufcugugscsc | on Akta Oligopilot/12 mL, | | |
| | | Manual coupling of | | |
| | | prodrug | | |
| A-3768103 | (PdAr13s)usCfsacuUfuAfUfugagUfu | Precursor (up to prodrug) | 7863.258 | 175 |
| | Ufcugugscsc | on Akta Oligopilot/12 mL, | | |
| | | Manual coupling of | | |
| | | prodrug | | |
| A-3768104 | (PdAr15s)usCfsacuUfuAfUfugagUfu | Precursor (up to prodrug) | 7833.276 | 176 |
| | Ufcugugscsc | on Akta Oligopilot/12 mL, | | |
| | | Manual coupling of | | |
| | | prodrug | | |
| A-3768105 | (PdAr17s)usCfsacuUfuAfUfugagUfu | Precursor (up to prodrug) | 7849.275 | 177 |
| | Ufcugugscsc | on Akta Oligopilot/12 mL, | | |
| | | Manual coupling of | | |
| | | prodrug | | |
| A-3768108 | (Pmmdpn1)usCfsacuUfuAfUfugagUf | Precursor (up to prodrug) | 7837.274 | 178 |
| | uUfcugugscsc | on Akta Oligopilot/12 mL, | | |
| | | Manual coupling of | | |
| | | prodrug | | |
| A-3768109 | (Pmmdpn2)usCfsacuUfuAfUfugagUf | Precursor (up to prodrug) | 7818.218 | 179 |
| | uUfcugugscsc | on Akta Oligopilot/12 mL, | | |
| | | Manual coupling of | | |
| | | prodrug | | |
| A-3768106 | (PdAr1pn1)usCfsacuUfuAfUfugagUf | Precursor (up to prodrug) | 7899.345 | 180 |
| | uUfcugugscsc | on Akta Oligopilot/12 mL, | | |
| | | Manual coupling of | | |
| | | prodrug | | |
| A-3768107 | (PdAr1pn2)usCfsacuUfuAfUfugagUf | Precursor (up to prodrug) | 7880.289 | 181 |
| | uUfcugugscsc | on Akta Oligopilot/12 mL, | | |
| | | Manual coupling of | | |
| | | prodrug | | |
| A-3903489 | (Pmmds)usUfscauUfuCfCfaccuUfuG | Precursor (up to prodrug) | 7698.205 | 182 |
| | fcccaasgsc | on Akta Oligopilot/12 mL, | | |
| | | Manual coupling of | | |
| | | prodrug | | |
| A-3768113 | (PdAr7s)usUfscauUfuCfCfaccuUfuG | Precursor (up to prodrug) | 7839.168 | 183 |
| | fcccaasgsc | on Akta Oligopilot/12 mL, | | |
| | | Manual coupling of | | |
| | | prodrug | | |
| A-3768114 | (PdAr9s)usUfscauUfuCfCfaccuUfuG | Precursor (up to prodrug) | 7785.282 | 184 |
| | fcccaasgsc | on Akta Oligopilot/12 mL, | | |
| | | Manual coupling of | | |
| | | prodrug | | |
| A-3768115 | (PdAr11s)usUfscauUfuCfCfaccuUfu | Precursor (up to prodrug) | 7778.262 | 185 |
| | Gfcccaasgsc | on Akta Oligopilot/12 mL, | | |
| | | Manual coupling of | | |
| | | prodrug | | |
| A-3768116 | (PdAr13s)usUfscauUfuCfCfaccuUfu | Precursor (up to prodrug) | 7804.281 | 186 |
| | Gfcccaasgsc | on Akta Oligopilot/12 mL, | | |
| | | Manual coupling of | | |
| | | prodrug | | |
| A-3768117 | (PdAr15s)usUfscauUfuCfCfaccuUfu | Precursor (up to prodrug) | 7774.299 | 187 |
| | Gfcccaasgsc | on Akta Oligopilot/12 mL, | | |
| | | Manual coupling of | | |
| | | prodrug | | |
| A-3768118 | (PdAr17s)usUfscauUfuCfCfaccuUfu | Precursor (up to prodrug) | 7790.298 | 188 |
| | Gfcccaasgsc | on Akta Oligopilot/12 mL, | | |
| | | Manual coupling of | | |
| | | prodrug | | |
| A-3768121 | (Pmmdpn1)usUfscauUfuCfCfaccuUf | Precursor (up to prodrug) | 7778.297 | 189 |
| | uGfcccaasgsc | on Akta Oligopilot/12 mL, | | |
| | | Manual coupling of | | |
| | | prodrug | | |
| A-3768122 | (Pmmdpn2)usUfscauUfuCfCfaccuUf | Precursor (up to prodrug) | 7759.241 | 190 |
| | uGfcccaasgsc | on Akta Oligopilot/12 mL, | | |
| | | Manual coupling of | | |
| | | prodrug | | |
| A-3768119 | (PdAr1pn1)usUfscauUfuCfCfaccuUf | Precursor (up to prodrug) | 7840.368 | 191 |
| | uGfcccaasgsc | on Akta Oligopilot/12 mL, | | |
| | | Manual coupling of | | |
| | | prodrug | | |
| A-3768120 | (PdAr1pn2)usUfscauUfuCfCfaccuUf | Precursor (up to prodrug) | 7821.312 | 192 |
| | uGfcccaasgsc | on Akta Oligopilot/12 mL, | | |
| | | Manual coupling of | | |
| | | prodrug | | |
| A-4213285 | (PdAr19s)usCfsacuUfuAfUfugagUfu | Precursor (up to prodrug) | 7855.230 | 193 |
| | Ufcugugscsc | on Akta Oligopilot/12 mL, | | |
| | | Manual coupling of | | |
| | | prodrug | | |
| A-4213286 | (PdAr21s)usCfsacuUfuAfUfugagUfu | Precursor (up to prodrug) | 7887.247 | 194 |
| | Ufcugugscsc | on Akta Oligopilot/12 mL, | | |
| | | Manual coupling of | | |
| | | prodrug | | |
| A-4213287 | (PdAr23s)usCfsacuUfuAfUfugagUfu | Precursor (up to prodrug) | 7887.247 | 195 |
| | Ufcugugscsc | on Akta Oligopilot/12 mL, | | |
| | | Manual coupling of | | |
| | | prodrug | | |
| A-4213288 | (Pmeds)usCfsacuUfuAfUfugagUfuUf | Precursor (up to prodrug) | 7849.275 | 196 |
| | cugugscsc | on Akta Oligopilot/12 mL, | | |
| | | Manual coupling of | | |
| | | prodrug | | |
| A-4213289 | (Pmids)usCfsacuUfuAfUfugagUfuUf | Precursor (up to prodrug) | 7785.232 | 197 |
| | cugugscsc | on Akta Oligopilot/12 mL, | | |
| | | Manual coupling of | | |
| | | prodrug | | |
| A-4213290 | (PdAr1cbs)usCfsacuUfuAfUfugagUf | Precursor (up to prodrug) | 7831.260 | 198 |
| | uUfcugugscsc | on Akta Oligopilot/12 mL, | | |
| | | Manual coupling of | | |
| | | prodrug | | |
| A-4213291 | (PdAr1cps)usCfsacuUfuAfUfugagUf | Precursor (up to prodrug) | 7845.287 | 199 |
| | uUfcugugscsc | on Akta Oligopilot/12 mL, | | |
| | | Manual coupling of | | |
| | | prodrug | | |
| A-4213292 | (PdAr1chs)usCfsacuUfuAfUfugagUf | Precursor (up to prodrug) | 7859.314 | 200 |
| | uUfcugugscsc | on Akta Oligopilot/12 mL, | | |
| | | Manual coupling of | | |
| | | prodrug | | |
| A-2717577 | (Pmmds)usUfsuagAfgUfGfaggaUfuA | Precursor (up to prodrug) | 8017.465 | 201 |
| | faaaugsasg | on Akta Oligopilot/12 mL, | | |
| | | Manual coupling of | | |
| | | prodrug | | |
| A-4213293 | (PdAr19s)usUfsuagAfgUfGfaggaUfu | Precursor (up to prodrug) | 8115.513 | 202 |
| | Afaaaugsasg | on Akta Oligopilot/12 mL, | | |
| | | Manual coupling of | | |
| | | prodrug | | |
| A-4213294 | (PdAr12s)usUfsuagAfgUfGfaggaUfu | Precursor (up to prodrug) | 8147.50 | 203 |
| | Afaaaugsasg | on Akta Oligopilot/12 mL, | | |
| | | Manual coupling of | | |
| | | prodrug | | |
| A-4213295 | (PdAr23s)usUfsuagAfgUfGfaggaUfu | Precursor (up to prodrug) | 8109.558 | 204 |
| | Afaaaugsasg | on Akta Oligopilot/12 mL, | | |
| | | Manual coupling of | | |
| | | prodrug | | |
| A-4213296 | (Pmeds)usUfsuagAfgUfGfaggaUfuAf | Precursor (up to prodrug) | 8031.488 | 205 |
| | aaaugsasg | on Akta Oligopilot/12 mL, | | |
| | | Manual coupling of | | |
| | | prodrug | | |
| A-4213297 | (Pmids)usUfsuagAfgUfGfaggaUfuAf | Precursor (up to prodrug) | 8045.515 | 206 |
| | aaaugsasg | on Akta Oligopilot/12 mL, | | |
| | | Manual coupling of | | |
| | | prodrug | | |
| A-4213298 | (PdAr1cbs)usUfsuagAfgUfGfaggaUf | Precursor (up to prodrug) | 8091.543 | 207 |
| | uAfaaaugsasg | on Akta Oligopilot/12 mL, | | |
| | | Manual coupling of | | |
| | | prodrug | | |
| A-4213299 | (PdAr1cps)usUfsuagAfgUfGfaggaUf | Precursor (up to prodrug) | 8105.570 | 208 |
| | uAfaaaugsasg | on Akta Oligopilot/12 mL, | | |
| | | Manual coupling of | | |
| | | prodrug | | |
| A-4213300 | (PdAr1chs)usUfsuagAfgUfGfaggaUf | Precursor (up to prodrug) | 8119.597 | 209 |
| | uAfaaaugsasg | on Akta Oligopilot/12 mL, | | |
| | | Manual coupling of | | |
| | | prodrug | | |

Pmmd (racemic cis−) 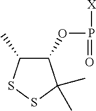
tPmmd (racemic trans−) 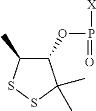
+cPmmd 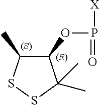
+tPmmd 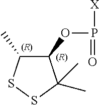
−cPmmd 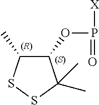
−tPmmd 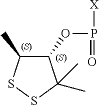
PdAr7 (+/−) cis 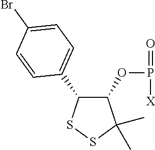
PdAr9 (+/−) cis 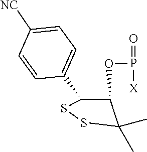
PdAr11 (+/−) cis 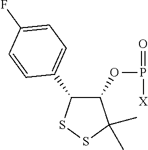
PdAr13 (+/−) cis 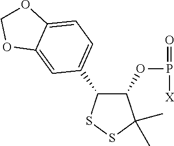
PdAr15 (+/−) cis 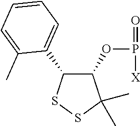
PdAr17 (+/−) cis 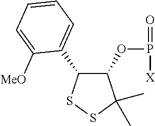
Pmmdpn1 (+/−) cis 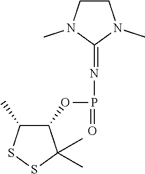
PdAr1pn1 (+/−) cis 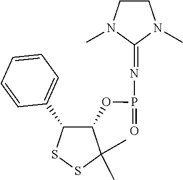
Pmmdpn2 (+/−) cis 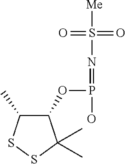
PdAr1 (cis−) 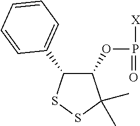
PdAr2 (trans−) 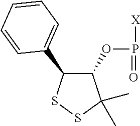
PdAr3 (cis−) 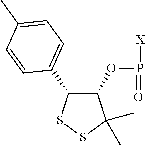
PdAr4 (trans−) 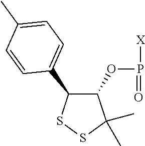
PdAr5 (cis−) 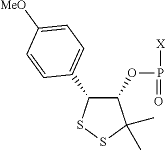
PdAr6 (trans−) 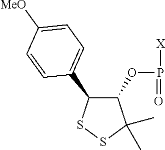
PdAr19 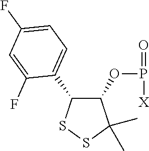
PdAr21 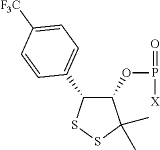
PdAr23 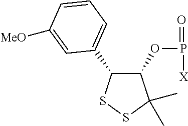
Pmed 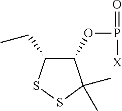
Pmid 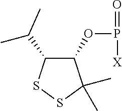
PdAr1cb 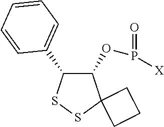
PdAr1cp 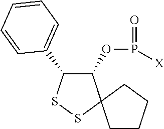
PdAr1ch 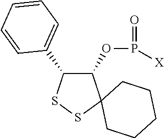
PdAr1pn2 (+/−) cis 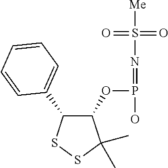
Upper case etter followed with f- 2,-deoxy-2′-fluoro (2′-F) sugar modi:!cation; lower case letter- 2′-O-methyl
(2′-OMe) sugar modification; s- phosphorothioate (PS) linkage wherein X is O or S.

TABLE 21
siRNAs having modified phosphate prodrug at the 5′-end of the antisense strand used for in vitro and in vivo studies

| Duplex Id | Oligo Id | Strand | Target | Oligo Seq | SEQ ID NO: | Calculated MW | Found MW |
|---|---|---|---|---|---|---|---|
| AD-73603 | A-147464 | sense | F12 | csascagaAfaCfUfCfaauaaagugaL96 | 210 | 8801.656 | 8797.128 |
| | A-147465 | antis | F12 | usCfsacuUfuAfUfugagUfuUfcugugscsc | 211 | 7514.875 | 7511.025 |
| AD-291896 | A-147464 | sense | F12 | csascagaAfaCfUfCfaauuaaagugaL96 | 212 | 8801.656 | 8797.128 |
| | A-447595 | antis | F12 | VPuCfacuUfuAfUfugagUfuUfcugugscsc | 213 | 7558.743 | 7555.05 |
| AD-1023152 | A-147464 | sense | F12 | csascagaAfaCfUfCfaauaaagugaL96 | 214 | 8801.656 | 8797.128 |
| | A-1875179 | antis | F12 | (Pmmds)usCfsacuUfuAfUfugagUfuUfcugugscsc | 215 | 7757.184 | 7752.991 |
| AD-1684744 | A-147464 | sense | F12 | csascagaAfaCfUfCfaauuaaagugaL96 | 216 | 8801.656 | 8797.128 |
| | A-3019037 | antis | F12 | (tPmmds)usCfsacuUfuAfUfugagUfuUfcugugscsc | 217 | 7757.178 | 7752.991 |
| AD-2012286 | A-147464 | sense | F12 | csascagaAfaCfUfCfaauaaagugaL96 | 218 | 8801.656 | 8797.128 |
| | A-3654050 | antis | F12 | (+cPmmds)usCfsacuUfuAfUfugagUfuUfcugugscsc | 219 | 7757.178 | 7752.991 |
| AD-2012287 | A-147464 | sense | F12 | csascagaAfaCfUfCfaauaaagugaL96 | 220 | 8801.656 | 8797.128 |
| | A-3654052 | antis | F12 | (−cPmmds)usCfsacuUfuAfUfugagUfuUfcugugscsc | 221 | 7757.178 | 7752.991 |
| AD-2012288 | A-147464 | sense | F12 | csascagaAfaCfUfCfaauaaagugaL96 | 222 | 8801.656 | 8797.128 |
| | A-3654054 | antis | F12 | (+tPmmds)usCfsacuUfuAfUfugagUfuUfcugugscsc | 223 | 7757.178 | 7752.991 |
| AD-2012289 | A-147464 | sense | F12 | csascagaAfaCfUfCfaauaaagugaL96 | 224 | 8801.656 | 8797.128 |
| | A-3654055 | antis | F12 | (−tPmmds)usCfsacuUfuAfUfugagUfuUfcugugscsc | 225 | 7757.178 | 7752.991 |
| AD-1684745 | A-147464 | sense | F12 | csascagaAfaCfUfCfaauaaagugaL96 | 226 | 8801.656 | 8797.128 |
| | A-3019038 | antis | F12 | (PdAr1s)usCfsacuUfuAfUfugagUfuUfcugugscsc | 227 | 7819.249 | 7815.007 |
| AD-1684746 | A-147464 | sense | F12 | csascagaAfaCfUfCfaauaaagugaL96 | 228 | 8801.656 | 8797.128 |
| | A-3036616 | antis | F12 | (PdAr2s)usCfsacuUfuAfUfugagUfuUfcugugscsc | 229 | 7819.249 | 7815.007 |
| AD-1684747 | A-147464 | sense | F12 | csascagaAfaCfUfCfaauaaagugaL96 | 230 | 8801.656 | 8797.128 |
| | A-3019039 | antis | F12 | (PdAr3s)usCfsacuUfuAfUfugagUfuUfcugugscsc | 231 | 7833.276 | 7829.023 |
| AD-1684748 | A-147464 | sense | F12 | csascagaAfaCfUfCfaauaaagugaL96 | 232 | 8801.656 | 8797.128 |
| | A-3036617 | antis | F12 | (PdAr4s)usCfsacuUfuAfUfugagUfuUfcugugscsc | 233 | 7833.276 | 7829.023 |
| AD-1684749 | A-147464 | sense | F12 | csascagaAfaCfUfCfaauaaagugaL96 | 234 | 8801.656 | 8797.128 |
| | A-3019040 | antis | F12 | (PdAr5s)usCfsacuUfuAfUfugagUfuUfcugugscsc | 235 | 7849.275 | 7845.017 |
| AD-1784131 | A-147464 | sense | F12 | csascagaAfaCfUfCfaauaaagugaL96 | 236 | 8801.656 | 8797.128 |
| | A-3196466 | antis | F12 | (PdAr6s)usCfsacuUfuAfUugagUfuUfcugugscscf | 237 | 7850.283 | 7846.025 |
| AD-2097008 | A-147464 | sense | F12 | csascagaAfaCfUfCfaauaaagugaL96 | 238 | 8801.656 | 8797.128 |
| | A-3768100 | antis | F12 | (PdAr7s)usCfsacuUfuAfUfugagUfuUfcugugscsc | 239 | 7898.145 | 7892.917 |
| AD-2097009 | A-147464 | sense | F12 | csascagaAfaCfUfCfaauaaagugaL96 | 240 | 8801.656 | 8797.128 |
| | A-3768101 | antis | F12 | (PdAr9s)usCfsacuUfuAfUfugagUfuUfcugugscsc | 241 | 7844.259 | 7840.002 |
| AD-2097010 | A-147464 | sense | F12 | csascagaAfaCfUfCfaauaaagugaL96 | 242 | 8801.656 | 8797.128 |
| | A-3768102 | antis | F12 | (PdAr11s)usCfsacuUfuAfUfugagUfuUfcugugscsc | 243 | 7837.239 | 7832.997 |
| AD-2097011 | A-147464 | sense | F12 | csascagaAfaCfUfCfaauaaagugaL96 | 244 | 8801.656 | 8797.128 |
| | A-3768103 | antis | F12 | (PdAr13s)usCfsacuUfuAfUfugagUfuUfcugugscsc | 245 | 7863.258 | 7858.997 |
| AD-2097012 | A-147464 | sense | F12 | csascagaAfaCfUfCfaauaaagugaL96 | 246 | 8801.656 | 8797.128 |
| | A-3768104 | antis | F12 | (PdAr15s)usCfsacuUfuAfUfugagUfuUfcugugscsc | 247 | 7833.276 | 7829.023 |
| AD-2097013 | A-147464 | sense | F12 | csascagaAfaCfUfCfaauaaagugaL96 | 248 | 8801.656 | 8797.128 |
| | A-3768105 | antis | F12 | (PdAr17s)usCfsacuUfuAfUfugagUfuUfcugugscsc | 249 | 7849.275 | 7845.017 |
| AD-2097016 | A-147464 | sense | F12 | csascagaAfaCfUfCfaauaaagugaL96 | 250 | 8801.656 | 8797.128 |
| | A-3768108 | antis | F12 | (Pmmdpn1)usCfsacuUfuAfUfugagUfuUfcugugscsc | 251 | 7837.274 | 7833.107 |
| AD-2097017 | A-147464 | sense | F12 | csascagAfaCfUfCfaauaaagugaL96 | 252 | 8801.656 | 8797.128 |
| | A-3768109 | antis | F12 | (Pmmdpn2)usCfsacuUfuAfUfugagUfuUfcugugscsc | 253 | 7818.218 | 7814.008 |
| AD-2097014 | A-147464 | sense | F12 | csascagaAfaCfUfCfaauaaagugaL96 | 254 | 8801.656 | 8797.128 |
| | A-3768106 | antis | F12 | (PdAr1pn1)usCfsacuUfuAfUfugagUfuUfcugugscsc | 255 | 7899.345 | 7895.122 |
| AD-2097015 | A-147464 | sense | F12 | csascagaAfaCfUfCfaauaaagugaL96 | 256 | 8801.656 | 8797.128 |
| | A-3768107 | antis | F12 | (PdAr1pn2)usCfsacuUfuAfUfugagUfuUfcugugscsc | 257 | 7880.289 | 7876.023 |
| AD-2140909 | A-1684938 | sense | SOD1 | ususggg(Chd)AfaAfGfGfuggaaaugsasa | 258 | 7384.248 | 7380.379 |
| | A-1813286 | antis | SOD1 | usUfscauUfuCfCfaccuUfuGfcccaasgsc | 259 | 7455.895 | 7452.072 |
| AD-2140910 | A-1684938 | sense | SOD1 | ususggg(Chd)AfaAfGfGfuggaaaugsasa | 260 | 7384.248 | 7380.379 |
| | A-3823724 | antis | SOD1 | VPusUfscauUfuCfCfaccuUfuGfcccaasgsc | 261 | 7531.894 | 7528.051 |
| AD-2140911 | A-1684938 | sense | SOD1 | ususggg(Chd)AfaAfGfGfuggaaaugsasa | 262 | 7384.248 | 7380.379 |
| | A-3903489 | antis | SOD1 | (Pmmds)usUfscauUfuCfCfccuUfuGfcccaasgsca | 263 | 7698.205 | 7694.038 |
| AD-2140912 | A-1684938 | sense | SOD1 | ususggg(Chd)AfaAfGfGfuggaaaugsasa | 264 | 7384.248 | 7380.379 |
| | A-3768113 | antis | SOD1 | (PdAr7s)usUfscauUfuCfCfaccuUfuGfcccaasgsc | 265 | 7839.168 | 7833.964 |
| AD-2140913 | A-1684938 | sense | SOD1 | ususggg(Chd)AfaAfGfGfuggaaaugsasa | 266 | 7384.248 | 7380.379 |
| | A-3768114 | antis | SOD1 | (PdAr9s)usUfscauUfuCfCfaccuUfuGfcccaasgsc | 267 | 7785.282 | 7781.049 |
| AD-2140914 | A-1684938 | sense | SOD1 | ususggg(Chd)AfaAfGfGfuggaaaugsasa | 268 | 7384.248 | 7380.379 |
| | A-3768115 | antis | SOD1 | (PdAr11s)usUfscauUfuCfCfaccuUfuGfcccaasgsc | 269 | 7778.262 | 7774.044 |
| AD-2140915 | A-1684938 | sense | SOD1 | ususggg(Chd)AfaAfGfGfuggaaaugsasa | 270 | 7384.248 | 7380.379 |
| | A-3768116 | antis | SOD1 | (PdAr13s)usUfscauUfuCfCfaccuUfuGfcccaasgsc | 271 | 7804.281 | 7800.043 |
| AD-2140916 | A-1684938 | sense | SOD1 | ususggg(Chd)AfaAfGfGfuggaaaugsasa | 272 | 7384.248 | 7380.379 |
| | A-3768117 | antis | SOD1 | (PdAr15s)usUfscauUfuCfCfaccuUfuGfcccaasgsc | 273 | 7774.299 | 7770.069 |
| AD-2140917 | A-1684938 | sense | SOD1 | ususggg(Chd)AfaAfGfGfuggaaaugsasa | 274 | 7384.248 | 7380.379 |
| | A-3768118 | antis | SOD1 | (PdAr17s)usUfscauUfuCfCfaccuUfuGfcccaasgsc | 275 | 7790.298 | 7786.064 |
| AD-2140920 | A-1684938 | sense | SOD1 | ususggg(Chd)AfaAfGfGfuggaaaugsasa | 276 | 7384.248 | 7380.379 |
| | A-3768121 | antis | SOD1 | (Pmmdpn1)usUfscauUfuCfCfaccuUfuGfcccaasgsc | 277 | 7778.297 | 7774.153 |
| AD-2140921 | A-1684938 | sense | SOD1 | ususggg(Chd)AfaAfGfGfuggaaaugsasa | 278 | 7384.248 | 7380.379 |
| | A-3768122 | antis | SOD1 | (Pmmdpn2)usUfscauUfuCfCfaccuUfuGfcccaasgsc | 279 | 7759.241 | 7755.054 |
| AD-2140918 | A-1684938 | sense | SOD1 | ususggg(Chd)AfaAfGfGfuggaaaugsasa | 280 | 7384.248 | 7380.379 |
| | A-3768119 | antis | SOD1 | (PdAr1pn1)usUfscauUfuCfCfaccuUfuGfcccaasgsc | 281 | 7840.368 | 7836.169 |
| AD-2140919 | A-1684938 | sense | SOD1 | ususggg(Chd)AfaAfGfGfuggaaaugsasa | 282 | 7384.248 | 7380.379 |
| | A-3768120 | antis | SOD1 | (PdAr1pn2)usUfscauUfuCfCfaccuUfuGfcccaasgsc | 283 | 7821.312 | 7817.07 |
| AD-2143214 | A-3340763 | sense | SOD1 | ususggg(Cda)AfaAfGfGfuggaaaugsasa | 284 | 7468.423 | 7464.473 |
| | A-1813286 | antis | SOD1 | usUfscauUfuCfCfaccuUfuGfcccaasgsc | 285 | 7455.895 | 7452.072 |
| AD-2143215 | A-3340763 | sense | SOD1 | ususggg(Cda)AfaAfGfGfuggaaaugsasa | 286 | 7468.423 | 7464.473 |
| | A-3823724 | antis | SOD1 | VPusUfscauUfuCfCfaccUfuGfcccaasgsc | 287 | 7531.894 | 7528.051 |
| AD-2143216 | A-3340763 | sense | SOD1 | ususggg(Cda)AfaAfGfGfuggaaaugsasa | 288 | 7468.423 | 7464.473 |
| | A-3903489 | antis | SOD1 | (Pmmds)usUfscauUfuCfCfaccuUfuGfcccaasgsc | 289 | 7698.205 | 7694.038 |
| AD-2143217 | A-3340763 | sense | SOD1 | ususggg(Cda)AfaAfGfGfuggaaaugsasa | 290 | 7468.423 | 7464.473 |
| | A-3768113 | antis | SOD1 | (PdAr7s)usUfscauUfuCfCfaccuUfuGfcccaasgsc | 291 | 7839.168 | 7833.964 |
| AD-2143218 | A-3340763 | sense | SOD1 | ususggg(Cda)AfaAfGfGfuggaaaugsasa | 292 | 7468.423 | 7464.473 |
| | A-3768114 | antis | SOD1 | (PdAr9s)usUfscauUfuCfCfaccuUfuGfcccaasgsc | 293 | 7785.282 | 7781.049 |
| AD-2143219 | A-3340763 | sense | SOD1 | ususggg(Cda)AfaAfGfGfuggaaaugsasa | 294 | 7468.423 | 7464.473 |
| | A-3768115 | antis | SOD1 | (PdAr11s)usUfscauUfuCfCfaccuUfuGfcccaasgsc | 295 | 7778.262 | 7774.044 |
| AD-2143220 | A-3340763 | sense | SOD1 | ususggg(Cda)AfaAfGfGfuggaaaugsasa | 296 | 7468.423 | 7464.473 |
| | A-3768116 | antis | SOD1 | (PdAr13s)usUfscauUfuCfCfaccuUfuGfcccaasgsc | 297 | 7804.281 | 7800.043 |
| AD-2143221 | A-3340763 | sense | SOD1 | ususggg(Cda)AfaAfGfGfuggaaaugsasa | 298 | 7468.423 | 7464.473 |
| | A-3768117 | antis | SOD1 | (PdAr15s)usUfscauUfuCfCfaccuUfuGfcccaasgsc | 299 | 7774.299 | 7770.069 |
| AD-2143222 | A-3340763 | sense | SOD1 | ususggg(Cda)AfaAfGfGfuggaaaugsasa | 300 | 7468.423 | 7464.473 |
| | A-3768118 | antis | SOD1 | (PdAr17s)usUfscauUfuCfCfaccuUfuGfcccaasgsc | 301 | 7790.298 | 7786.064 |
| AD-2143225 | A-3340763 | sense | SOD1 | ususggg(Cda)AfaAfGfGfuggaaaugsasa | 302 | 7468.423 | 7464.473 |
| | A-3768121 | antis | SOD1 | (Pmmdpn1)usUfscauUfuCfCfaccuUfuGfcccaasgsc | 303 | 7778.297 | 7774.153 |
| AD-2143226 | A-3340763 | sense | SOD1 | ususggg(Cda)AfaAfGfGfuggaaaugsasa | 304 | 7468.423 | 7464.473 |
| | A-3768122 | antis | SOD1 | (Pmmdpn2)usUfscauUfuCCfaccuUfuGfcccaasgscf | 305 | 7759.241 | 7755.054 |
| AD-2140923 | A-3963409 | sense | SOD1 | csasuuu(upn9)AfaUfCfCfucacucuasasa | 306 | 7223.291 | 7219.433 |
| | A-444402 | antis | SOD1 | VPusUfsuagAfgUfGfaggaUfuAfaaaugsasg | 307 | 7851.156 | 7847.154 |
| AD-2143224 | A-3340763 | sense | SOD1 | ususggg(Cda)AfaAfGfGfuggaaaugsasa | 308 | 7468.423 | 7464.473 |
| | A-3768120 | antis | SOD1 | (PdAr1pn2)usUfscauUfuCfCfaccuUfuGfcccaasgsc | 309 | 7821.312 | 7817.07 |
| AD-74843 | A-147464 | sense | F12 | csascagaAfaCfUfCfaauaaaugaL96g | 310 | 8801.656 | 8797.128 |
| | A-150219 | antis | F12 | VPusCCfsacuUfuAfUfugagUfuUfcugugscsc | 311 | 7591.874 | 7587.005 |
| AD-2314809 | A-147464 | sense | F12 | csascagaAfaCfUfCfaauaaag | 312 | 8801.656 | 8797.128 |
| | A-4213285 | antis | F12 | (PdAr19s)usCfsacuUfuAfUfugagUfuUfcugugscsc | 313 | 7855.23 | 7850.988 |
| AD-2314810 | A-147464 | sense | F12 | csascagaAfaCfUfCfaauaaagugaL96 | 314 | 8801.656 | 8797.128 |
| | A-4213286 | antis | F12 | (PdAr21s)usCfsacuUfuAfUfugagUfuUfcugugscsc | 315 | 7887.247 | 7882.994 |
| AD-2314811 | A-147464 | sense | F12 | csascagaAfaCfUfCfaauaaag | 316 | 8801.656 | 8797.128 |
| | A-4213287 | antis | F12 | (PdAr23s)usCfsacuUfuAfUfugagUfuUfcugugscsc | 317 | 7849.275 | 7845.017 |
| AD-2314812 | A-147464 | sense | F12 | csascagaAfaCfUfCfaauaaagugaL96 | 318 | 8801.656 | 8797.128 |
| | A-4213288 | antis | F12 | (Pmeds)usCfsacuUfuAfUfugagUfuUfcugugscsc | 319 | 7771.205 | 7767.007 |
| AD-2314813 | A-147464 | sense | F12 | csascagaAfaCfUfCfaauaaagugaL96 | 320 | 8801.656 | 8797.128 |
| | A-4213289 | antis | F12 | (Pmids)usCfsacuUfuAfUfugagUfuUfcugugscsc | 321 | 7785.232 | 7781.023 |
| AD-2314814 | A-147464 | sense | F12 | csascagaAfaCfUfCfaauaaagugaL96 | 322 | 8801.656 | 8797.128 |
| | A-4213290 | antis | F12 | (PdAr1cbs)usCfsacuUfuAfUfugagUfuUfcugugscsc | 323 | 7831.26 | 7827.007 |
| AD-2314815 | A-147464 | sense | F12 | csascagaAfaCfUfCfaauaaagugaL96 | 324 | 8801.656 | 8797.128 |
| | A-4213291 | antis | F12 | (PdAr1cps)usCfsacuUfuAfUfugagUfuUfcugugscsc | 325 | 7845.287 | 7841.023 |
| AD-2314816 | A-147464 | sense | F12 | csascagaAfaCfUfCfaauaaagugaL96 | 326 | 8801.656 | 8797.128 |
| | A-4213292 | antis | F12 | (PdAr1chs)usCfsacuUfuAfUfugagUfuUfcugugscsc | 327 | 7859.314 | 7855.038 |

Pmmd (racemic cis−) 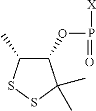
tPmmd (racemic trans−) 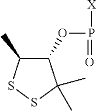
+cPmmd 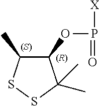
+tPmmd 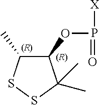
−cPmmd 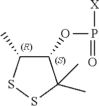
−tPmmd 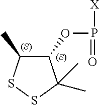
PdAr7 (+/−) cis 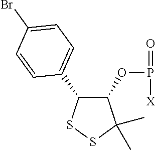
PdAr9 (+/−) cis 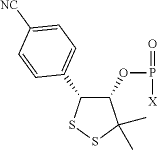
PdAr11 (+/−) cis 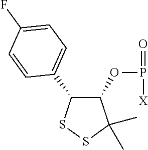
PdAr13 (+/−) cis 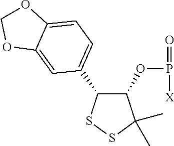
PdAr15 (+/−) cis 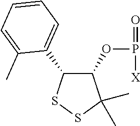
PdAr17 (+/−) cis 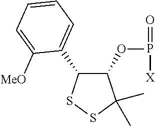
Pmmdpn1 (+/−) cis 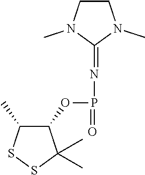
PdAr1pn1 (+/−) cis 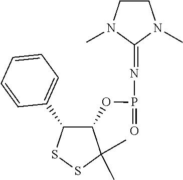
Pmmdpn2 (+/−) cis 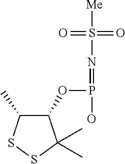
PdAr1 (cis−) 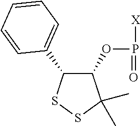
PdAr2 (trans−) 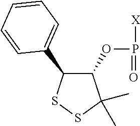
PdAr3 (cis−) 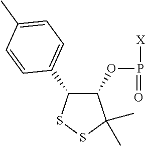
PdAr4 (trans−) 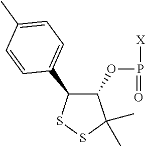
PdAr5 (cis−) 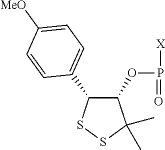
PdAr6 (trans−) 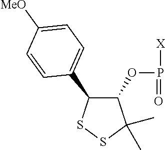
PdAr19 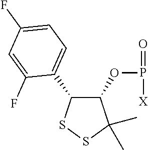
PdAr21 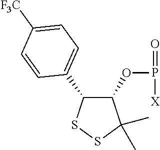
PdAr23 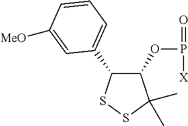
Pmed 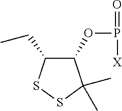
Pmid 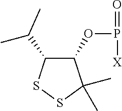
PdAr1cb 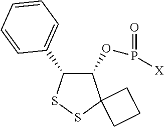
PdAr1cp 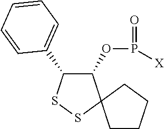
PdAr1ch 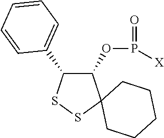
PdAr1pn2 (+/−) cis 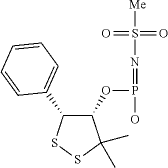
L96 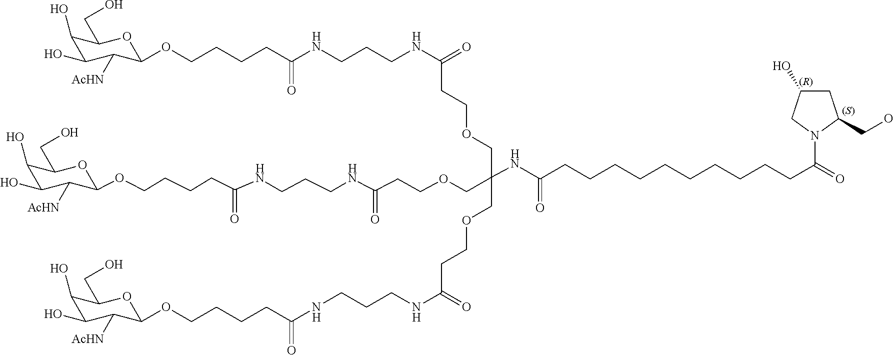
Upper case etter followed with f- 2,-deoxy-2′-fluoro (2′-F) sugar modi:!cation; lower case letter- 2′-O-methyl (2′-OMe) sugar modification;
s- phosphorothioate (PS) linkage wherein X is O or S.

### Example 15. In Vitro Evaluation of siRNA Duplexes Containing Modified Phosphate Prodrugs at the 5′ End

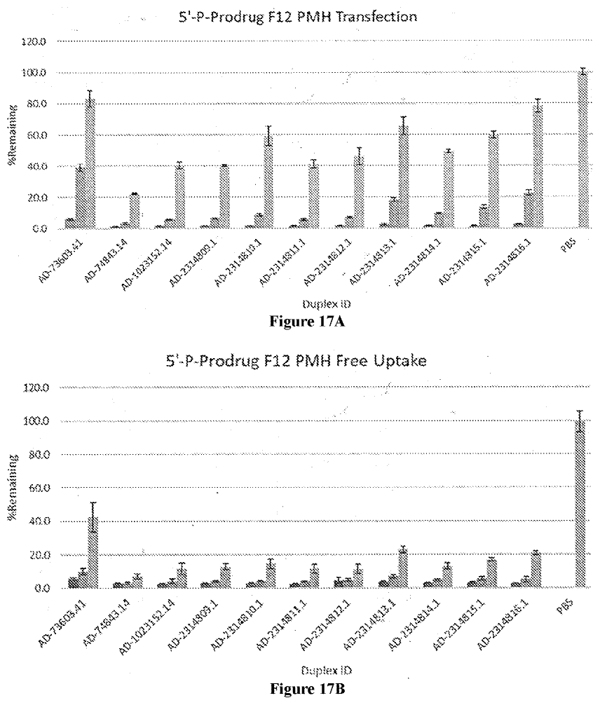

Transfection procedure: siRNA duplexes containing the modified phosphate prodrugs at the 5′ end (see Table 21) was transfected in primary mouse hepatocytes with RNAiMAX at 0.1, 1, 10, and 100 nM concentrations and analyzed 24 hours post-transfection. Percent F12 message remaining was determined by qPCR. The results were plotted against the control, as shown in FIGS. 15A, 16A, and 17A.

Free uptake procedure: siRNA duplexes containing the modified phosphate prodrugs at the 5′ end (see Table 21) was incubated with primary mouse hepatocytes at 0.1, 1, 10, and 100 nM concentrations in cell culture medium and analyzed after 48 hours. Percentage of F12 message remaining was determined by qPCR. The results were plotted against the control, as shown in FIGS. 15B, 16B, and 17B.

### Example 16. DTT Reduction Assay to Check the Cleavability of the Cyclic Phosphate Prodrug Analogues

Reduction of the 5′-cyclic modified phosphate prodrugs (see Table 21) by dithiothreitol (DTT) to 5′ phosphate or 5′ phosphorothioate was explored by incubating 100 μM modified oligonucleotide (23-nt length) with 100 mM DTT in 1×PBS. The amount of full-length (non-reduced) oligonucleotide was observed via LCMS analysis (either Agilent Single-Quad MS or Novatia HTSC) up to 72 hours post incubation.

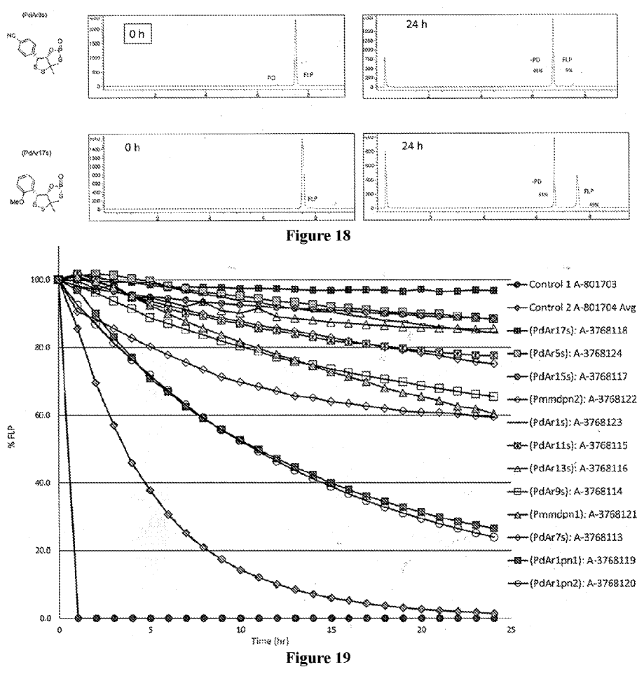

The results of the DTT assay for certain 5′-cyclic modified phosphate prodrugs (see Table 21) are illustrated in FIG. 18, which shows that the sterically unhindered, electron-withdrawing 5′-cyclic modified phosphate prodrugs PdAr9 deprotected much faster than the sterically-hindered, electron-rich prodrug PdAr17. Scheme 24 below shows the rank-order of the rates of unmasking of certain 5′-cyclic modified phosphate prodrugs in both GSH and DTT assays. Both assays show similar trends that the sterically-hindered, electron-rich 5′-cyclic modified phosphate prodrugs had a higher stability and a lower unmasking rate than those sterically unhindered, electron-withdrawing 5′-cyclic modified phosphate prodrugs.

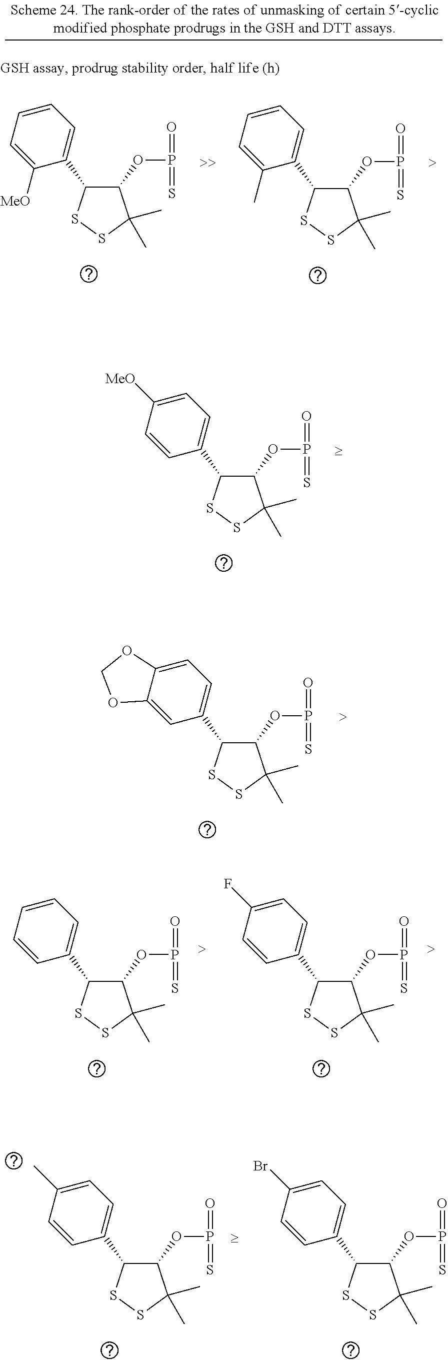

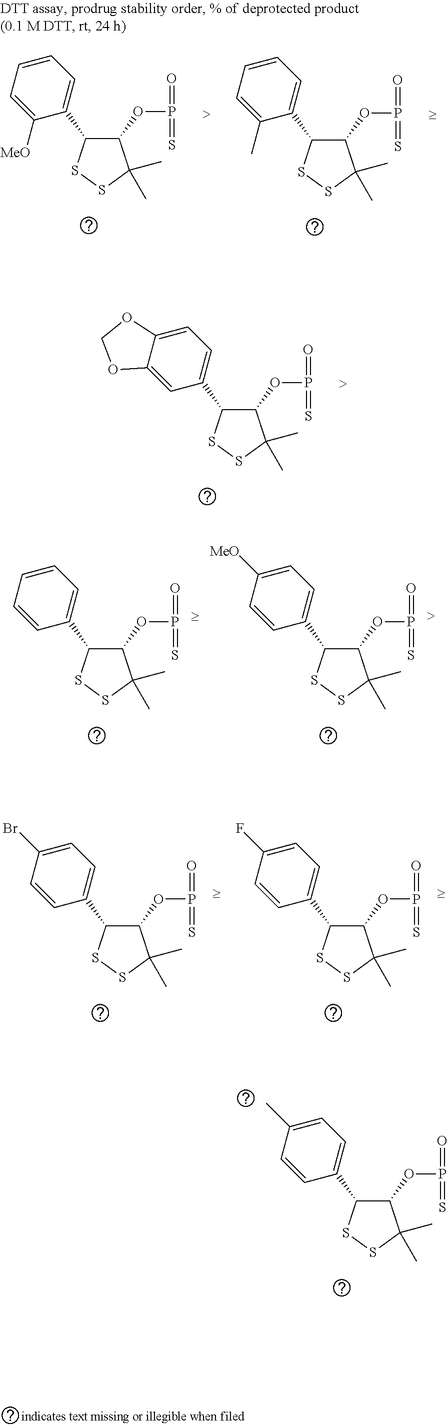

### Example 17. Glutathione Assay to Check the Cleavability of Cyclic Prodrug Analogues

Modified oligonucleotide (11-nt or 23-nt length) was added at 100 μM to a solution of 250 pig (6.25 U/mL) glutathione-S-transferase from equine liver (GST) (Sigma Cat. No. G6511) and 0.1 mg/mL NADPH (Sigma Cat. No. 481973) in 0.1 M Tris pH 7.2. Glutathione (GSH) (MP Biomedicals, Inc. Cat. No. 101814 #) was added to the mixture for a final concentration of 10 mM. Immediately after addition of GSH, sample was injected onto a Dionex DNAPac PA200 column (4×250 mm) at 30° C. and run on an anion exchange gradient of 35-65% (20 mM Sodium Phosphate, 10-15% CH₃CN, 1M Sodium Bromide pH11) at 1 mL/min for 6.5 minutes.

Glutathione-mediated cleavage kinetics were monitored every hour for 24 hours. The area under the main peak for each hour was normalized to the area from the 0 h time point (first injection). First-order decay kinetics were used to calculate half-lives. A control sequence containing modified oligonucleotide (23-nt length) with 5′ Thiol modifier C6 (Glen Research Cat. No. 10-1936-02) between N6 and N7 was run each day of assay run. A second control sequence containing modified oligonucleotide (23-nt length) with the same 5′ thiol modifier C6 at N1 was also run once per set of sequences. Half-lives were reported relative to half-life of control sequence. Glutathione and GST were prepared as stocks of 100 mM and 10 mg/mL in water, respectively, and aliquoted into 1 mL tubes and stored at −80° C. A new aliquot was used for every day the assay was run.

The results of the GTT assays for certain 5′-cyclic modified phosphate prodrugs (see Table 20) are shown in FIG. 19. Table 22 shows the corresponding half-lives. As shown from FIG. 19 and Table 22, high unmasking rates of those 5′-cyclic modified phosphate prodrugs that formed unnatural phosphates (Pmmdpn1, Pmmdpn2, PdAr1pn1, and PdAr1pn2) were due to electron-withdrawing effect of the substituent on the phosphorous atom.

TABLE 22
Half-lives of the 5′-cyclic modified phosphate prodrugs

| Modification | Half-Life (h) |
|---|---|
| Control 1 | <1 |
| Control 2 | 4 |
| Pmmdpn1 | 31 |
| Pmmdpn2 | 57 |
| PdAr1s | 86 |
| PdAr1pn1 | 12 |
| PdAr1pn2 | 12 |
| PdAr5s | 112 |
| PdAr7s | 35 |
| PdAr9s | 39 |
| PdAr11s | 60 |
| PdAr13s | 105 |
| PdAr15s | 151 |
| PdAr17s | 517 |

### Example 18. In Vivo Evaluation of siRNA Duplexes Containing Modified Phosphate Prodrugs at the 5′ End in Liver

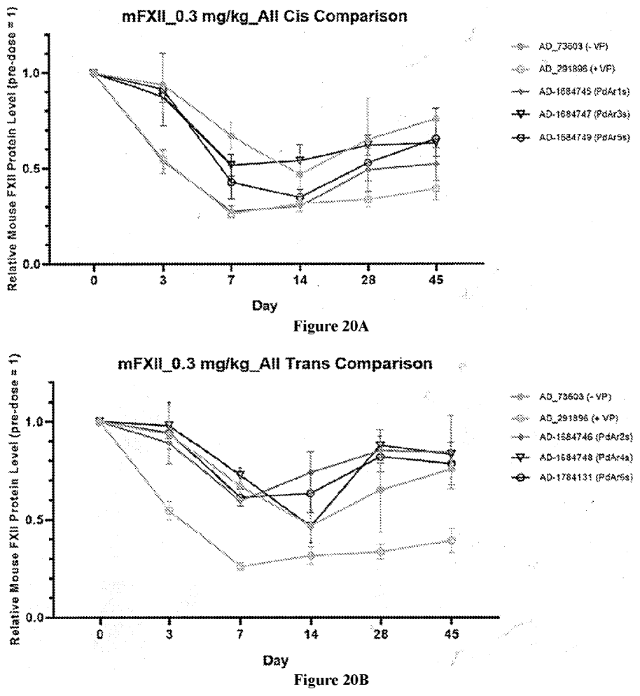

In vivo study procedure: C57bl6 female mice (n=3/group) were dosed with 0.1 mg/kg or 0.3 mg/kg of siRNA duplex containing the modified phosphate prodrugs at the 5′ end (see Table 21). Serum was collected at days 0, 3, 7, 14, 28, and 45 days pos-dose and analyzed via ELISA to determine relative F12 protein levels. The results are shown in FIGS. 20-22.

FIGS. 20A-20B show in vivo activity of F12 siRNAs containing cis- or trans-modified phosphate prodrugs at the 5′-end of antisense strand in mice at 0.3 mg/kg dose. Comparing the results of cis- and trans-isomers, the cis-isomers were found to be generally more active than the corresponding trans-isomers.

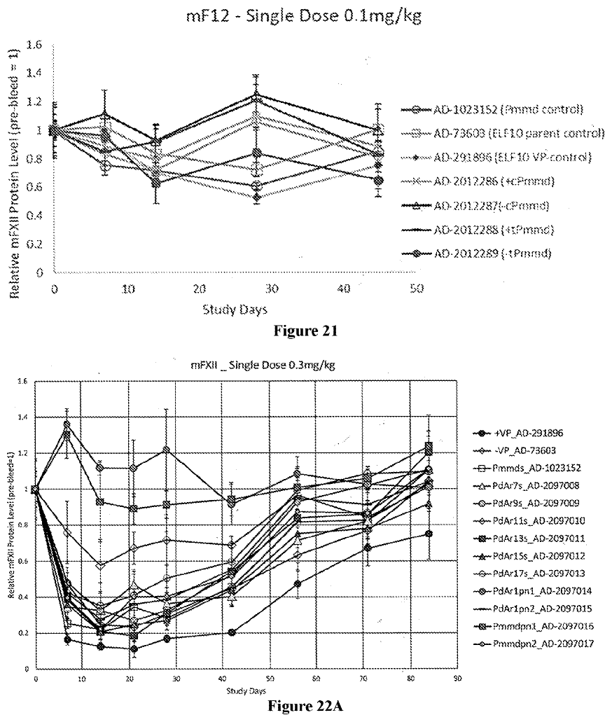

FIG. 21 shows in vivo activity of F12 siRNAs containing chiral or chirally enriched versions of racemic trans-phosphate prodrugs (Pmmd) at the 5′-end of antisense strand in mice at 0.1 mg/kg dose. As shown in the figure, no additive benefit of chirality was observed at this concentration level. The siRNA activated by chirally pure trans-isomer (−tPmmd) preserved activity at day 45.

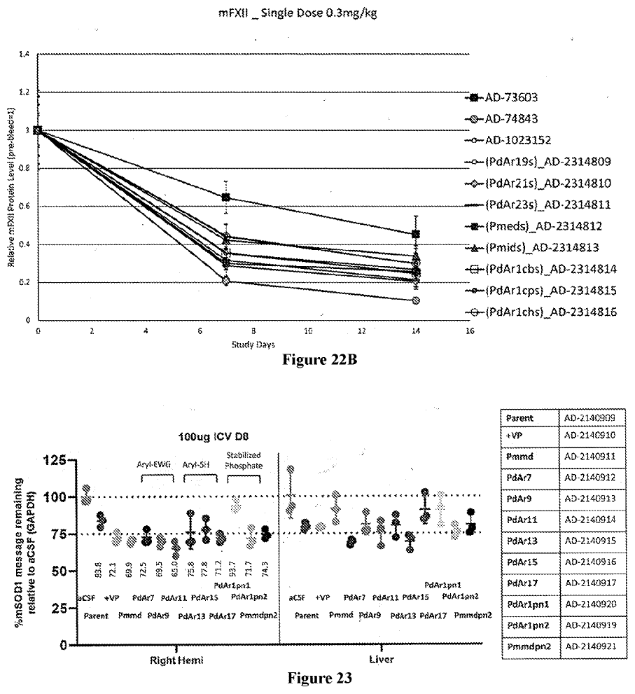

FIGS. 22A-22B shows in vivo hepatic activity of F12 siRNAs containing 5′-phosphate prodrugs at the 5′-end of antisense strand in mice at 0.3 mg/kg dose. As shown in the figures, among the 5′ modified phosphate prodrugs tested, moderately sterically hindered and relatively slowly unmasking PdAr15 appeared to present both superior activity and durability. Quickly unmasking electron-withdrawing-group-containing compounds showed fast onset but not as good durability, while strongly sterically hindered electron-donating-group-containing compounds showed moderate activity likely due to excessive stability. The 5′-phosphate prodrugs that produced singly-charged unnatural phosphate on deprotection (Pmmdpn1 and PdAr1pn1) showed minimum activity. At the same time, the compounds that form doubly-charged unnatural phosphates (mesyl-stabilized) showed good activity at onset but bounced more rapidly.

### Example 19: In Vivo Evaluation of siRNA Duplexes Containing Modified Phosphate Prodrugs at the 5′ End in CNS

In vivo study procedure for CNS (ICV—intracranial ventricular administration): C57bl6 female mice (n=4/group) were dosed with 100 μg of SOD1 siRNA duplex containing the modified phosphate prodrugs at the 5′ end (see Table 21). Brain was collected after 7 days of ICV administration and the right hemisphere was used for qPCR analysis to determine the relative SOD1 mRNA levels. The results are shown in FIG. 23.

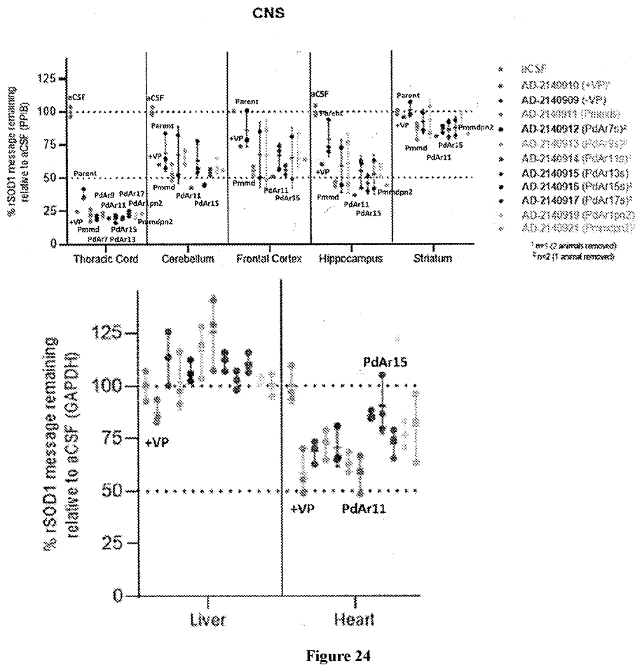

In vivo study procedure for CNS (IT—Intrathecal administration): Female rat (n=3/group) were dosed with 0.1 mg/rat, 0.3 mg/rat, or 0.9 mg/rat of SOD1 siRNA duplex containing the modified phosphate prodrugs at the 5′ end (see Table 21). Brain was collected after 14 or 84 days of IT administration and dissected into different regions for qPCR analysis to determine the relative SOD1 mRNA levels. The results are shown in FIG. 24.

FIG. 23 shows in vivo CNS activity of SOD1 siRNAs containing 5′-phosphate prodrugs at the 5′-end of antisense strand in mice via a single dose of 100 pg via ICV administration. The results show that a number of 5′-cyclic modified phosphate prodrugs showed an activity equal to or higher than that of the VP-control, among which Pmmd and PdAr17 appeared to have certain brain specificity with low/no activity in liver.

FIG. 24 shows in vivo CNS activity of SOD1 siRNAs containing 5′-phosphate prodrugs at the 5′-end of antisense strand in rat vis a single dose of 1.5 mg in 50 μl via IT administration. The results show that a number of 5′-cyclic modified phosphate prodrugs, Pmmd, PdAr11, and PdAr15, showed a consistent activity equal to or higher than that of the VP-control in all brain subsections, among which PdAr15 appeared to have a good brain specificity with low/no activity in heart.

### Example 20: In Vivo Evaluation of siRNA Duplexes Containing Modified Phosphate Prodrugs at the 5′ End in Muscle and Adipose

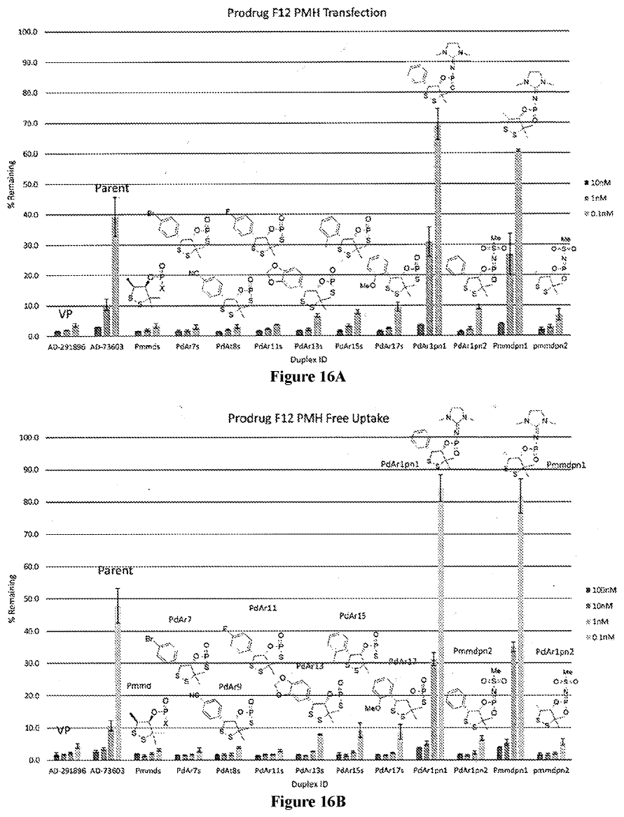

In vivo study procedure: C57bl6 female mice (n=3/group) were dosed with 2 mg/kg of siRNA duplex containing the modified phosphate prodrugs at the 5′ end (see in Table 2. Muscle, heart and adipose tissues were collected at day 14. SOD1 mRNA level was determined by qPCR and data was plotted relative to PBS (FIG. 16-19).

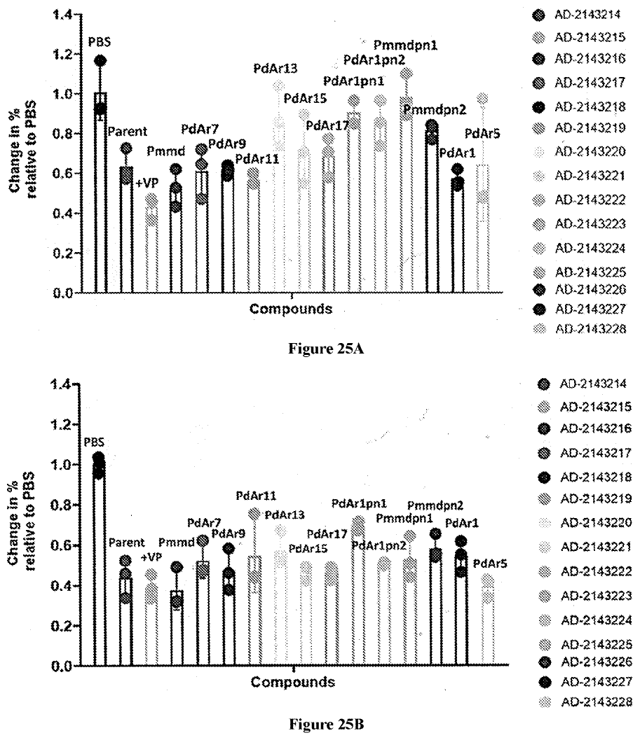

FIGS. 25A-25B show in vivo muscle activity of SOD1 siRNAs containing 5′-phosphate prodrugs at the 5′-end of antisense strand in mice via a single dose of 2 mg/kg. The results show that a number of 5′-cyclic modified phosphate prodrugs, Pmmd, PdAr11, PdAr1, and PdAr5, showed an activity comparable to that of the VP-control in skeletal muscle (FIG. 25A), and Pmmd and PdAr5 showed an activity equal to or exceeding that of the VP-control in gastrocnemius (FIG. 25B).

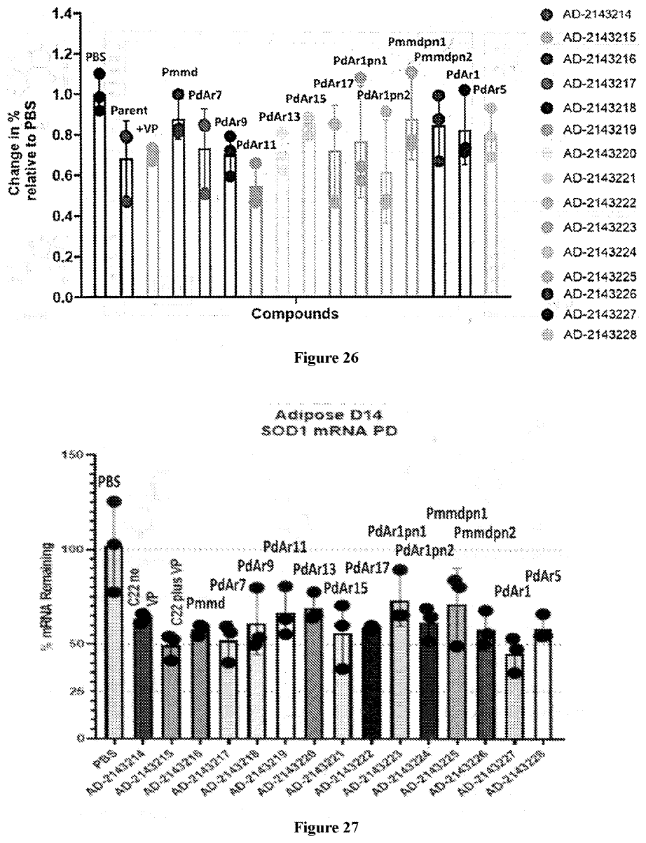

FIG. 26 shows in vivo heart muscle activity of SOD1 siRNAs containing 5′-phosphate prodrugs at the 5′-end of antisense strand in mice via a single dose of 2 mg/kg. The results show that two 5′-cyclic modified phosphate prodrugs, PdAr11 and unnatural phosphate PdAr1pn1, showed an activity greater than that of the VP control.

FIG. 27 shows in vivo adipose tissue activity of SOD1 siRNAs containing 5′-phosphate prodrugs at the 5′-end of antisense strand in mice via a single dose 2 mg/kg. The mRNAs from adipose tissue was measured by qPCR at day 14 and plotted against PBS control. The results show that two 5′-cyclic modified phosphate prodrugs, PdAr7 and PdAr1, showed an activity equal to or greater than that of the VP control.

1. A compound comprising a structure of formula (I), or a salt or stereoisomer thereof: 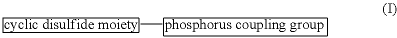
wherein the 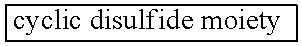
has the structure of: 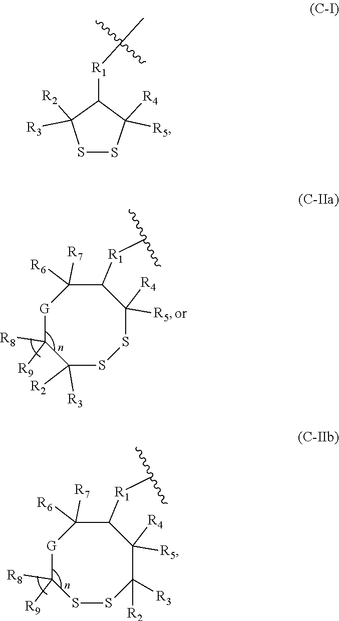
wherein:
R₁ is O or S, and is bonded to the P atom of the 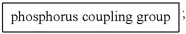
R₂, R₄, R₆, R₇, R₈, and R₉ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups;
R₃ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₃ and R₅, together with the adjacent carbon atoms and the two sulfur atoms, form a second ring;
R₂ and R₃, together with the adjacent carbon atom, can form another ring;
R₄ and R₅, together with the adjacent carbon atom, can form another ring;
R₆ and R₇, together with the adjacent carbon atom, can form another ring;
R₈ and R₉, together with the adjacent carbon atoms, can form another ring;
two or more of R₂, R₃, R₄, R₅, R₆, R₇, R₈, R₉, R¹⁴, and R¹⁵, together with the adjacent carbon atoms can form one or more rings fused with the ring containing the two sulfur atoms;
G is O, N(R′), S, or C(R¹⁴)(R¹⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″, together with the adjacent nitrogen atom, form a ring, and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.

2. The compound of claim 1, wherein in the 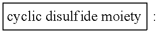
R₁ is O;
G is CH₂;
n is 0 or 1;
R₂, R₄, R₆, R₇, R₈, and R₉ are each independently H, halo, CN or C₁-C₆ alkylene-CN, C(O)OR¹³ or C₁-C₆ alkylene-C(O)OR¹³, S(O)OR¹³ or C₁-C₆ alkylene-S(O)OR¹³, C(O)N(R′)(R″) or C₁-C₆ alkylene-C(O)N(R′)(R″), OR¹³ or C₁-C₆ alkylene-OR¹³, N(R′)(R″) or C₁-C₆ alkylene-N(R′)(R″), C₁-C₆ alkyl, aryl, heteroaryl, each of which can be optionally substituted by one or more R^{sub} groups;
R₃ and R₅ are each independently H, halo, CN or C₁-C₆ alkylene-CN, C(O)OR¹³ or C₁-C₆ alkylene-C(O)OR¹³, S(O)OR¹³ or C₁-C₆ alkylene-S(O)OR¹³, C(O)N(R′)(R″) or C₁-C₆ alkylene-C(O)N(R′)(R″), OR¹³ or C₁-C₆ alkylene-OR¹³, N(R′)(R″) or C₁-C₆ alkylene-N(R′)(R″), C₁-C₆ alkyl, aryl, heteroaryl, each of which can be optionally substituted by one or more R^{sub} groups; or R₃ and R₅, together with the adjacent carbon atoms and the two sulfur atoms, form a second ring of 6-8 atoms;
R₂ and R₃, together with the adjacent carbon atom, can form another ring of 3-7 atoms;
R₄ and R₅, together with the adjacent carbon atom, can form another ring of 3-7 atoms;
R₆ and R₇, together with the adjacent carbon atom, can form another ring of 3-7 atoms;
R₈ and R₉, together with the adjacent carbon atom, can form another ring of 3-7 atoms;
two or more of R₂, R₃, R₄, R₅, R₆, R₇, R₈, R₉, R¹⁴, and R¹⁵, together with the adjacent carbon atoms can form one or more ring of 5-7 atoms fused with the ring containing the two sulfur atoms;
R¹³ is independently for each occurrence H, C₁-C₆ alkyl, aryl, alkylcarbonyl, or arylcarbonyl; and
R′ and R″ are each independently H or C₁-C₆ alkyl.

3-10. (canceled)

11. The compound of claim 1, wherein the 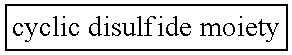
has the structure of: 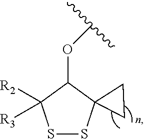
wherein n is 1 to 4.

12. The compound of claim 11, wherein:
R₂ and R₃ are each independently H, C₁₋₆ alkyl, aryl, heteroaryl, CN or CH₂CN, OR¹³ or CH₂OR¹³, C(O)OR¹³ or CH₂C(O)OR¹³, S(O)OR¹³ or CH₂S(O)OR¹³, C(O)N(R′)(R″) or CH₂C(O)N(R′)(R″), or C(R¹⁴)(R¹⁵)(R¹⁶) or CH₂C(R¹⁴)(R¹⁵)(R¹⁶), each of which can be optionally substituted by one or more R^{sub} groups;
R¹³ is independently for each occurrence H, C₁₋₆ alkyl, cycloalkyl, aryl, heteroaryl, or aralkyl;
R¹⁴, R¹⁵, and R¹⁶ are each independently H, halo, C₁₋₆ alkyl, alkaryl, aryl, or heteroaryl; and
R′ and R″ are each independently HK C₁₋₆ alkyl, aryl, or heteroaryl.

13. The compound of claim 11, wherein the 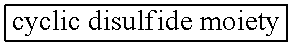
has one of the following structures:

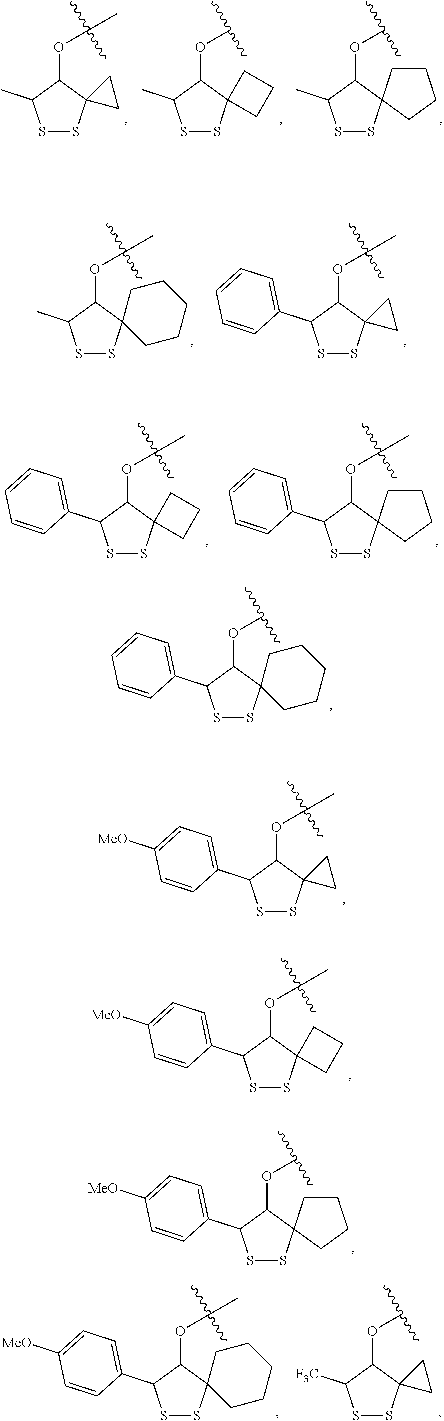

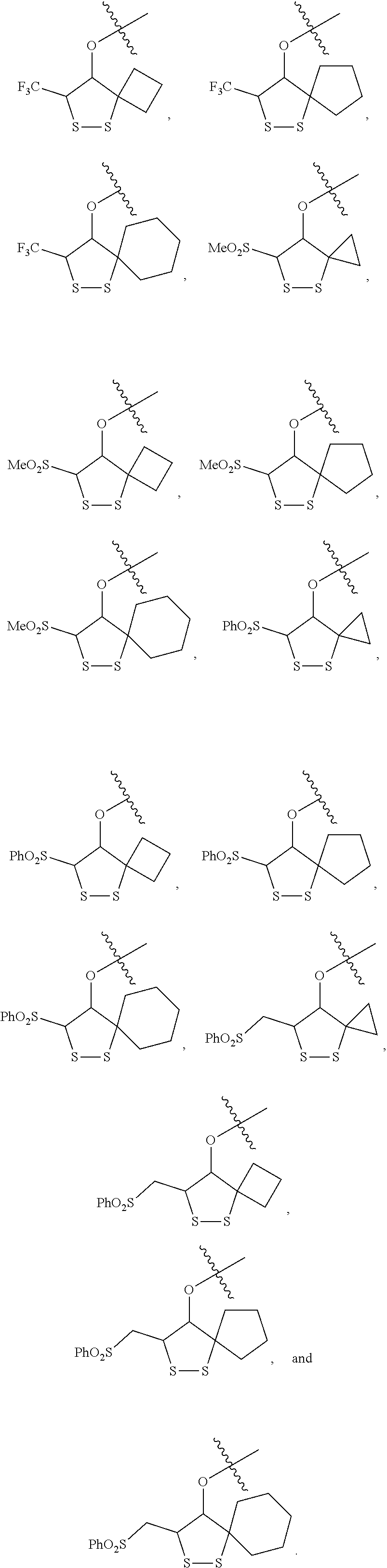

14-17. (canceled)

18. The compound of claim 1, wherein the 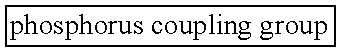
has the structure of: 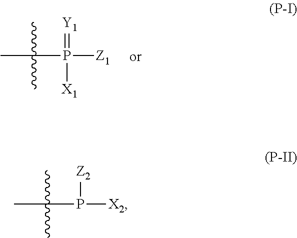
wherein:
X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), NSO₂R′, N═CN(R′)(R″), B(R¹³)₃, BH₃⁻, Se; or D-Q, wherein D is independently for each occurrence absent, O, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide,
Y₁ is S, O, or N(R′);
M is an organic or inorganic cation; and
R¹⁸ is H or alkyl, optionally substituted with one or more R^{sub} groups.

19-20. (canceled)

21. The compound of claim 18, wherein the 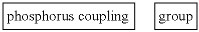
has one of the following structures:

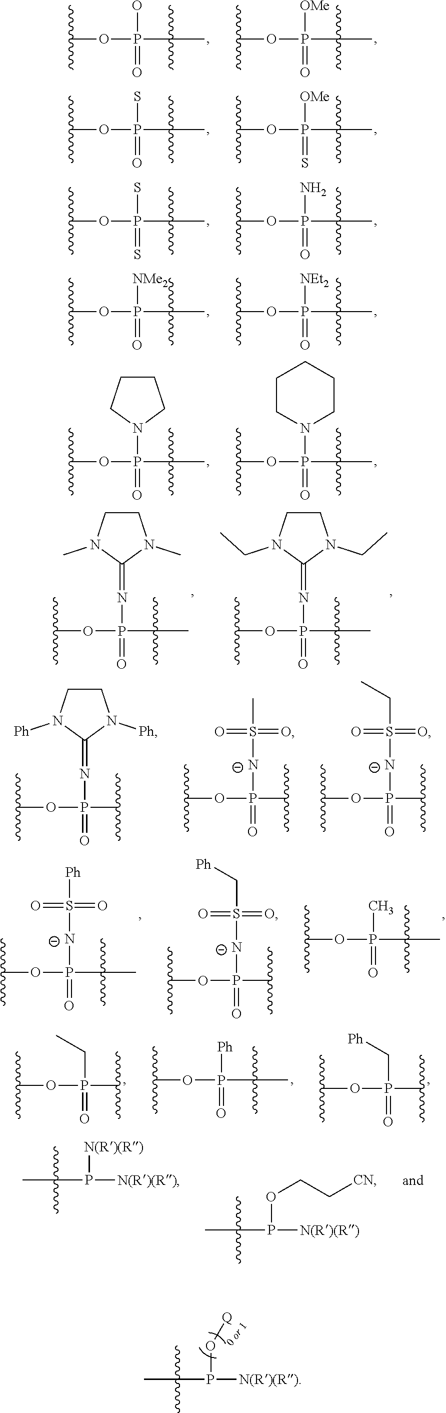

22-25. (canceled)

26. The compound of claim 1, wherein the compound has one of the following structures:

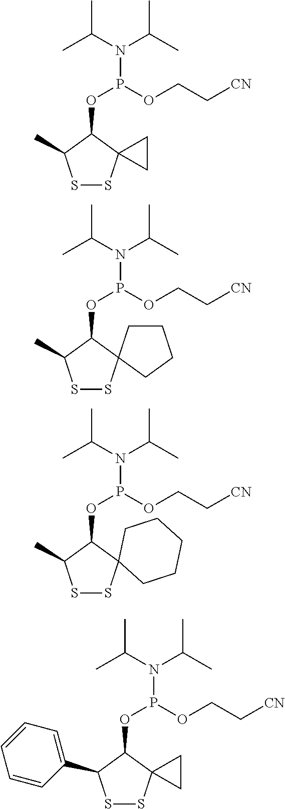

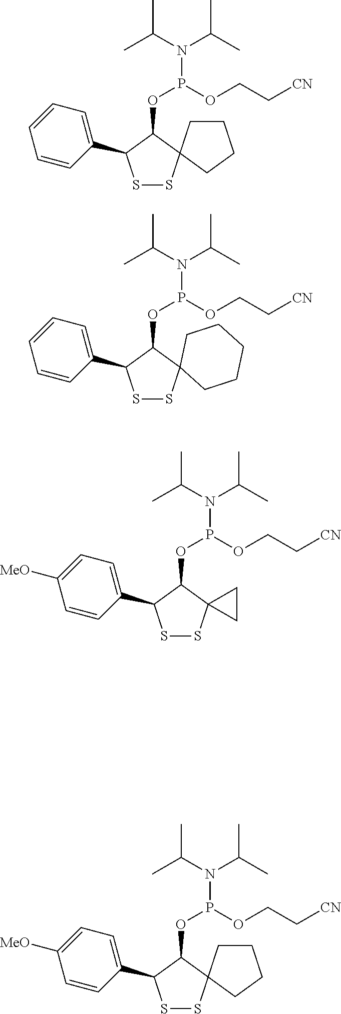

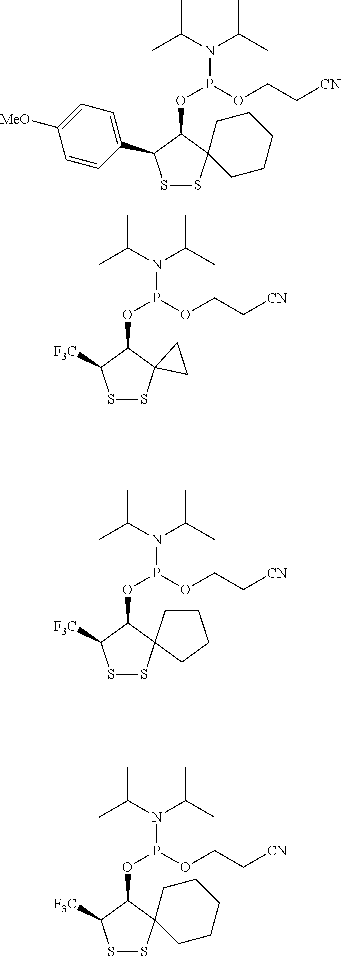

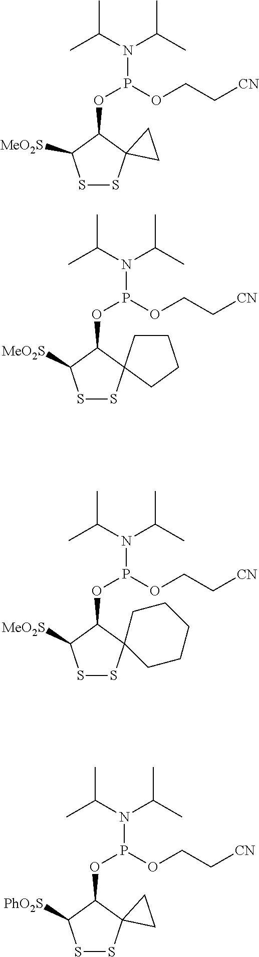

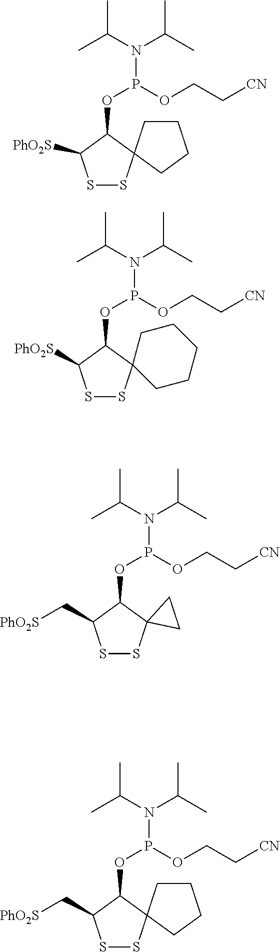

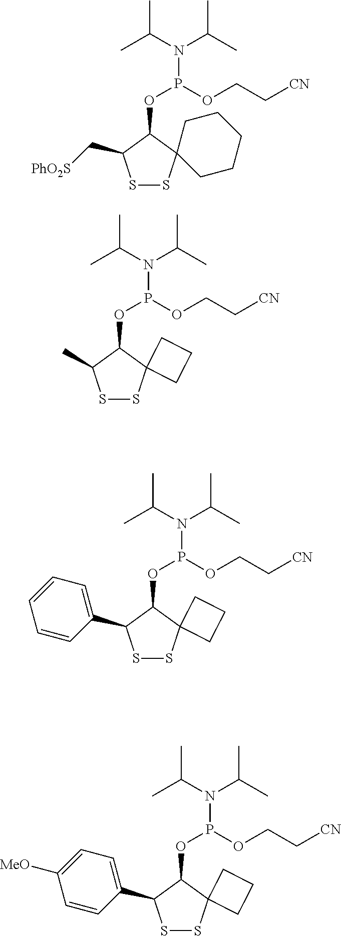

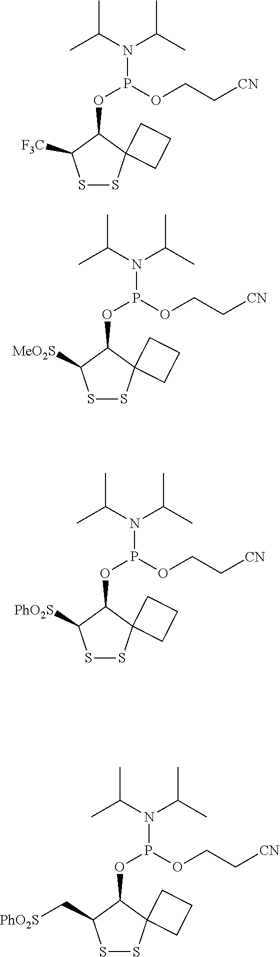

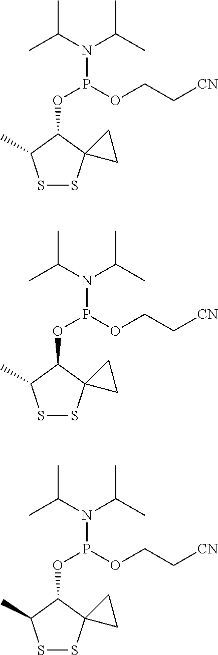 [SMILES: CC(C)N(C(C)C)P(OCCC#N)O[C@H]1[C@@H](C)SSC12CC2 ; CC(C)N(C(C)C)P(OCCC#N)O[C@@H]1[C@@H](C)SSC12CC2 ; CC(C)N(C(C)C)P(OCCC#N)O[C@H]1[C@H](C)SSC12CC2]

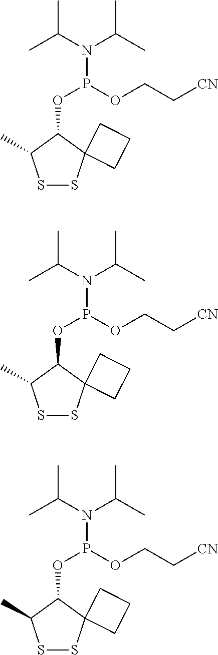 [SMILES: CC(C)N(C(C)C)P(OCCC#N)O[C@H]1[C@@H](C)SSC12CCC2 ; CC(C)N(C(C)C)P(OCCC#N)O[C@@H]1[C@@H](C)SSC12CCC2 ; CC(C)N(C(C)C)P(OCCC#N)O[C@H]1[C@H](C)SSC12CCC2]

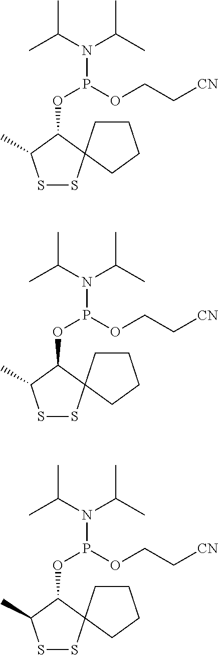 [SMILES: CC(C)N(C(C)C)P(OCCC#N)O[C@H]1[C@@H](C)SSC12CCCC2 ; CC(C)N(C(C)C)P(OCCC#N)O[C@@H]1[C@@H](C)SSC12CCCC2 ; CC(C)N(C(C)C)P(OCCC#N)O[C@H]1[C@H](C)SSC12CCCC2]

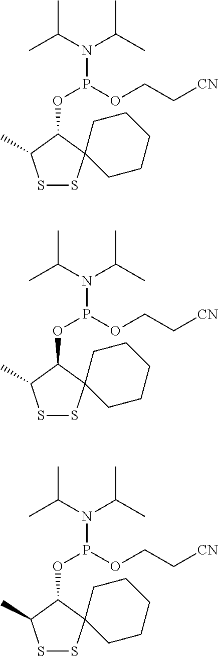 [SMILES: CC(C)N(C(C)C)P(OCCC#N)O[C@H]1[C@@H](C)SSC12CCCCC2 ; CC(C)N(C(C)C)P(OCCC#N)O[C@@H]1[C@@H](C)SSC12CCCCC2 ; CC(C)N(C(C)C)P(OCCC#N)O[C@H]1[C@H](C)SSC12CCCCC2]

27-29. (canceled)

30. The compound of claim 1, wherein one or more ligands are connected to any one of R₂, R₃, R₄, R₅, R₆, R₇, R₈, and R₉ of the 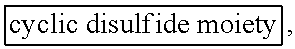
optionally via one or more linkers.

31. (canceled)

32. An oligonucleotide comprising one or more structures of formula (II): 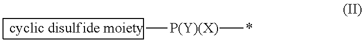
wherein the 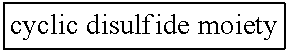
has the structure of: 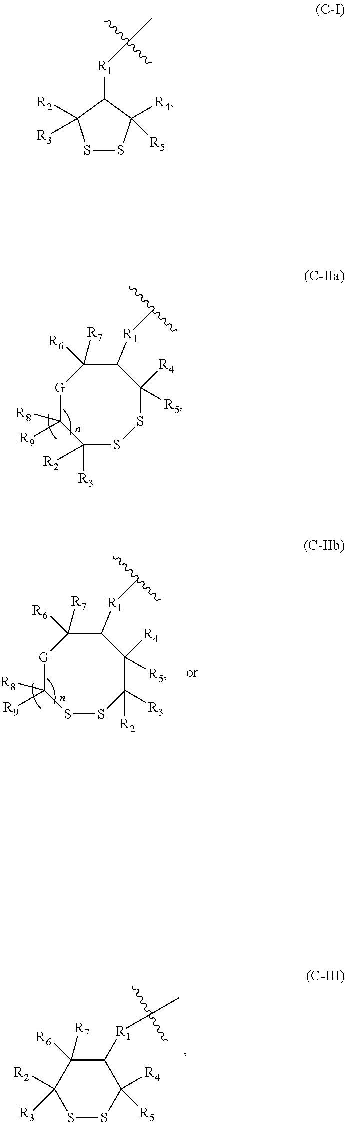
or a salt or stereoisomer thereof,
wherein:
* represents the bond to the oligonucleotide,
Y is absent, N(R′), =O, or =S, X is —OH, —SH, C(O)H, S(O)H, alkyl optionally substituted with one or more R^{sub} groups, N(R′)(R″), NSO₂R′, N═CN(R′)(R″), B(R¹³)₃, BH₃⁻, or X′, wherein X′ is N(R′)(R″), —OR² or —SR¹³;
R₁ is O or S, and is bonded to the P atom of the —P(Y)(X)—* group;
R₂, R₄, R₆, R₇, R₈, and R₉ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups;
R₃ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₃ and R₅, together with the adjacent carbon atoms and the two sulfur atoms, form a second ring;
R₂ and R₃, together with the adjacent carbon atom, can form another ring;
R₄ and R₅, together with the adjacent carbon atom, can form another ring;
R₆ and R₇, together with the adjacent carbon atom, can form another ring;
R₈ and R₉, together with the adjacent carbon atoms, can form another ring;
two or more of R₂, R₃, R₄, R₅, R₆, R₇, R₈, R₉, R¹⁴, and R¹⁵, together with the adjacent carbon atoms can form one or more rings fused with the ring containing the two sulfur atoms;
G is O, N(R′), S, or C(R¹⁴)(R¹⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″, together with the adjacent nitrogen atom, form a ring, and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido;
wherein, when the 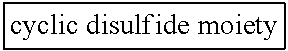
has the structure of formula (C-III), at least on 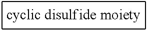
is connected at the 5′ end of the nucleoside or oligonucleotide.

33-37. (canceled)

38. The oligonucleotide of claim 32, comprising a structure having the formula: 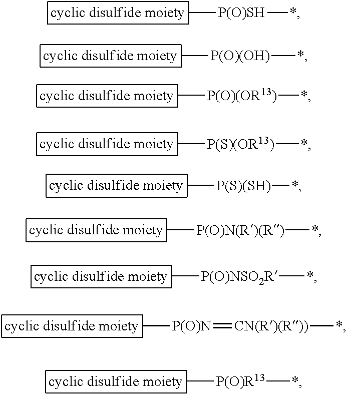
or a salt thereof.

39. The oligonucleotide of claim 38, comprising a structure having one of the following formulas:

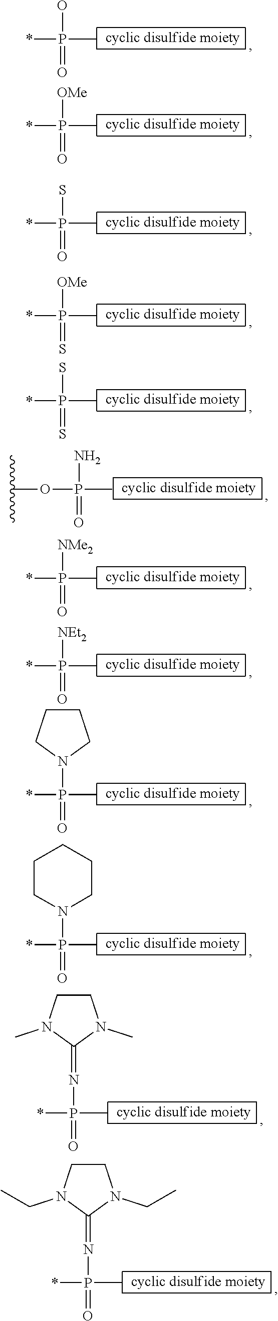

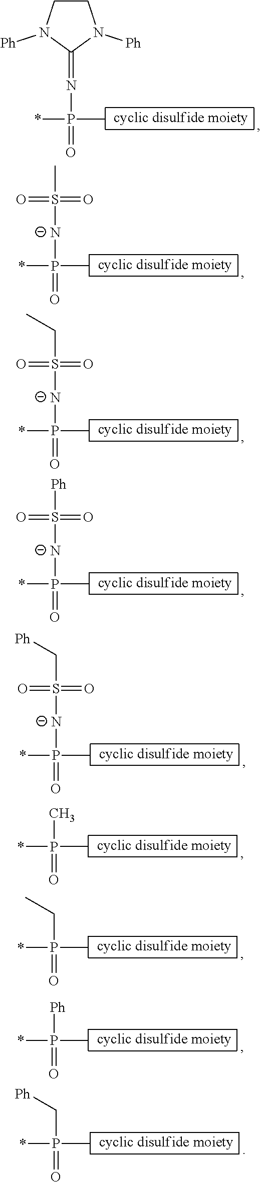

40-42. (canceled)

43. The oligonucleotide of claim 32, wherein the oligonucleotide contains at least one 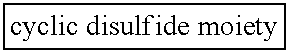
at the 5′-end of the oligonucleotide, and wherein the first nucleotide at the 5′-end of the oligonucleotide has the structure of 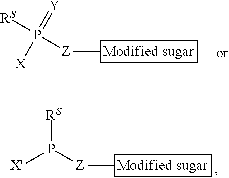
or a salt or a stereoisomer thereof, wherein:
R^{S} is the 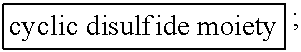
X is —OH, —SH, C(O)H, S(O)H, alkyl optionally substituted with one or more R^{sub} groups, N(R′)(R″), NSO₂R′, N═CN(R′)(R″), B(R¹³)₃, BH₃⁻, or X′, wherein X′ is N(R′)(R″), —OR¹³ or —SR¹³
Y is S, O, or N(R′);
Z is O, S, N(R′), or CH₂; and
Modified sugar is a sugar moiety containing one or more sugar modifications selected from the group consisting of 2′-modification, LNA, isomeric modification, 5′-modification, unnatural cyclic modification, acyclic modification, and abasic modification.

44. The oligonucleotide of claim 43, wherein the sugar modification is 2′-modification, LNA, isomeric modification, 5′-modification, or abasic modification, and wherein the first nucleotide at the 5′-end of the oligonucleotide has the structure of 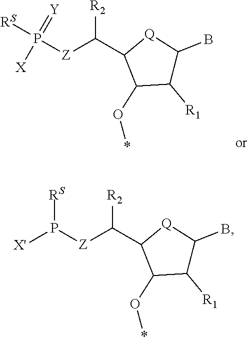
or a salt or a stereoisomer thereof, wherein:
* represents a bond to the subsequent optionally modified internucleotide linkage;
B is an optionally modified nucleobase, or H;
R^{S} is the 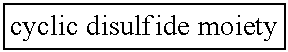
and
R₁ is H, OH, O-methoxyalkyl, O-methyl, O-allyl, CH₂-allyl, fluoro, O-N-methylacetamido (O-NMA), O-N-alkylacetamido, O-dimethoxypropyl, O-dimethylaminoethoxyethyl (O-DMAEOE), O-aminopropyl (O-AP), or ara-F; or R₁ forms a bridge with the 4′ carbon of the ribose sugar;
R₂ is H, alkyl, or aryl;
X is —OH, —SH, C(O)H, S(O)H, alkyl optionally substituted with one or more R^{sub} groups, N(R′)(R″), NSO₂R′, N═CN(R′)(R″), B(R¹³)₃, BH₃⁻, or X′, wherein X′ is N(R′)(R″), —OR¹³ or —SR¹³
Y is S, O, or N(R′);
Z is O, S, N(R′), or CH₂; and
Q is O, S, CH₂, or N(R′).

45. The oligonucleotide of claim 44, wherein the first nucleotide at the 5′-end of the oligonucleotide has one of the following structures selected from groups (i)-(v):

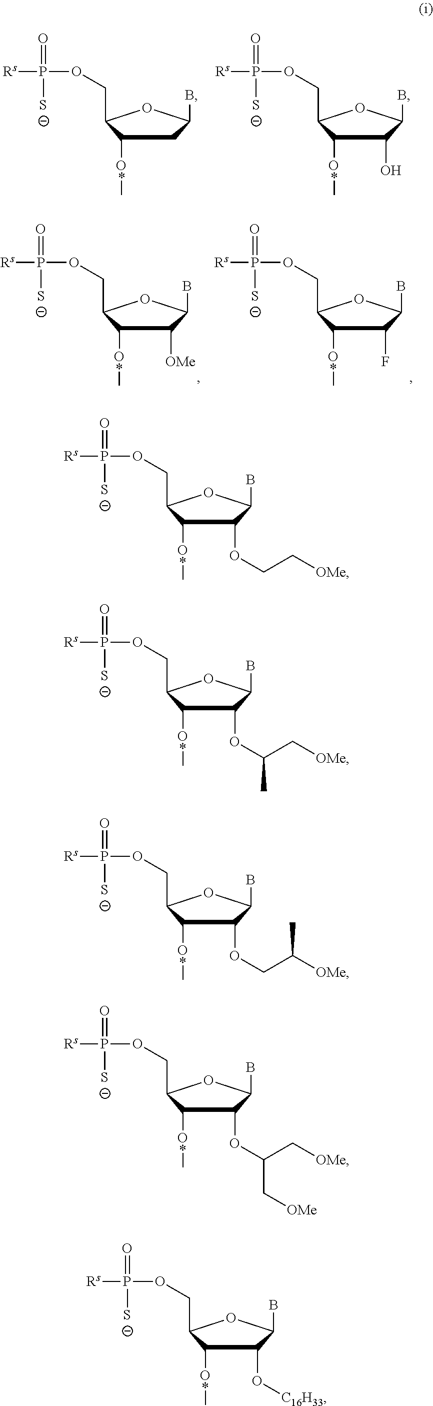

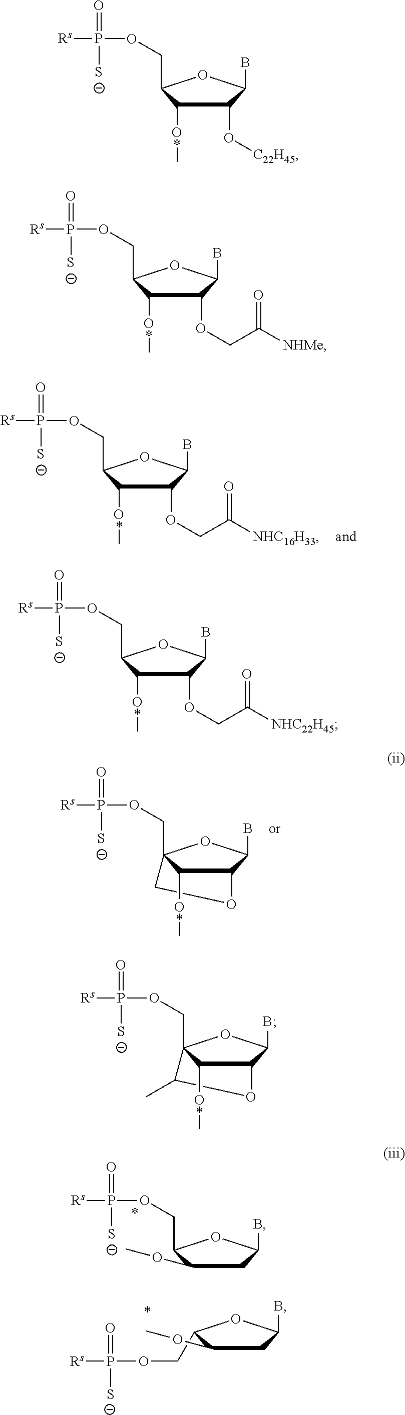

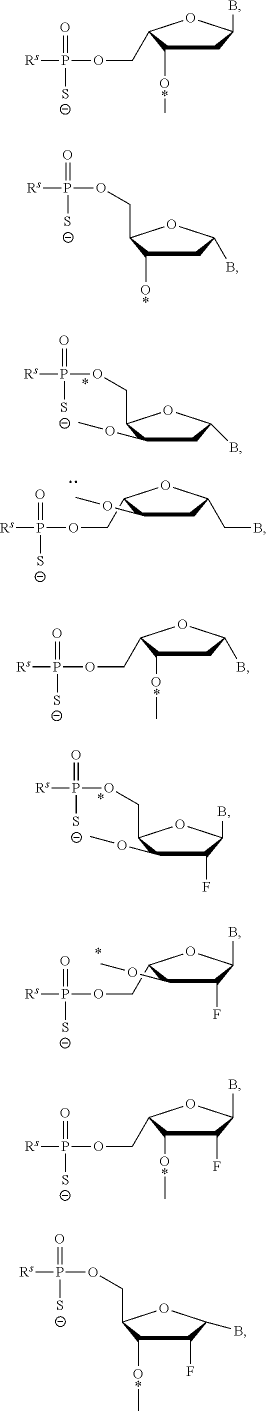

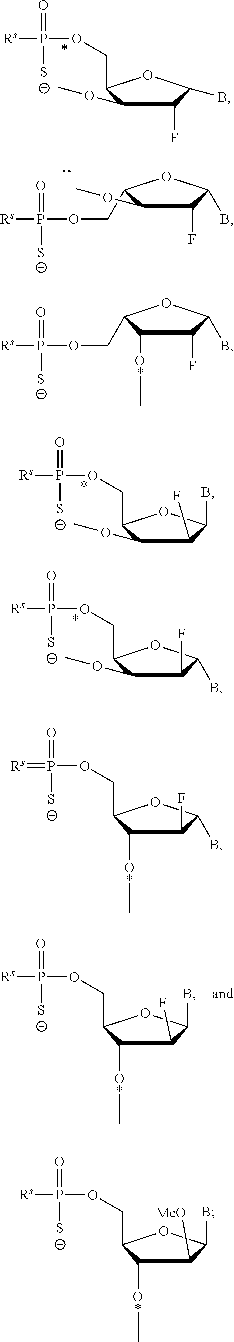

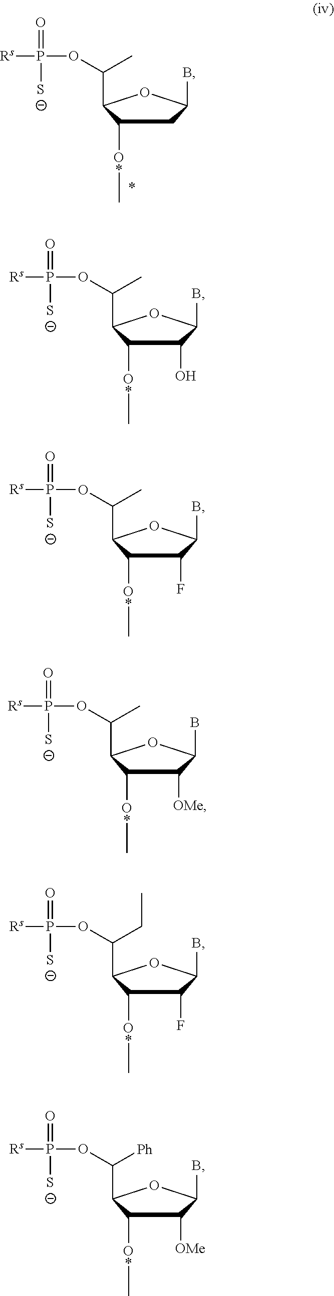

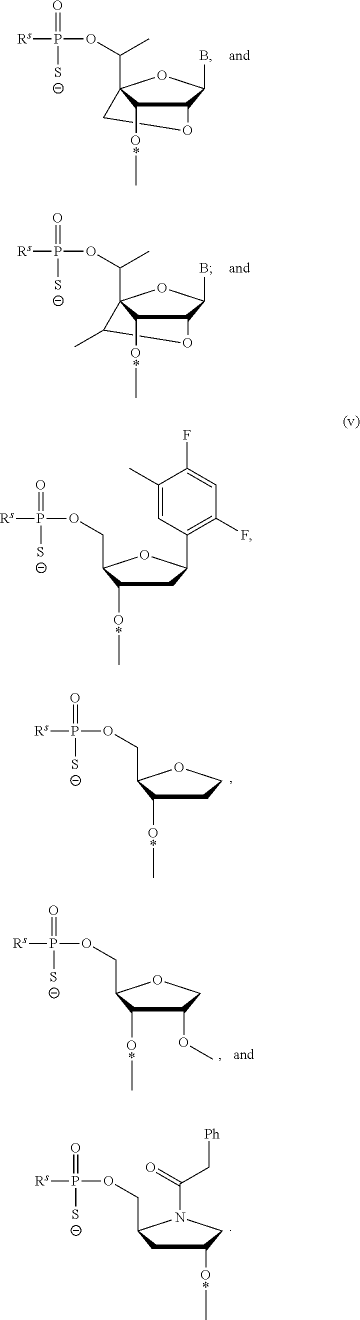

46-49. (canceled)

50. The oligonucleotide of claim 43, wherein the 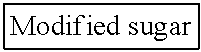
contains (a) unnatural cyclic modification having one of the following structures: 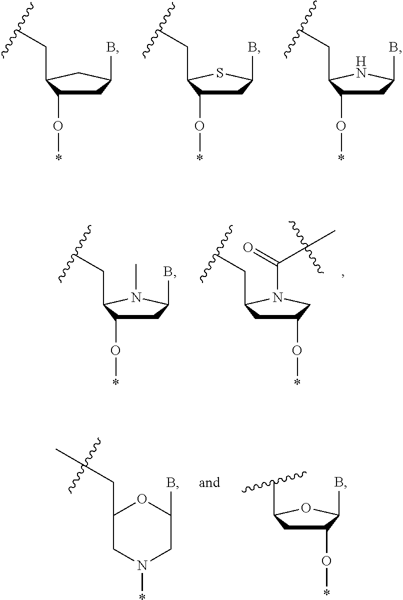
wherein:
* represents a bond to the subsequent optionally modified internucleotide linkage; and
B is an optionally modified nucleobase, or H; and/or
(b) acyclic modification having one of the following structures: 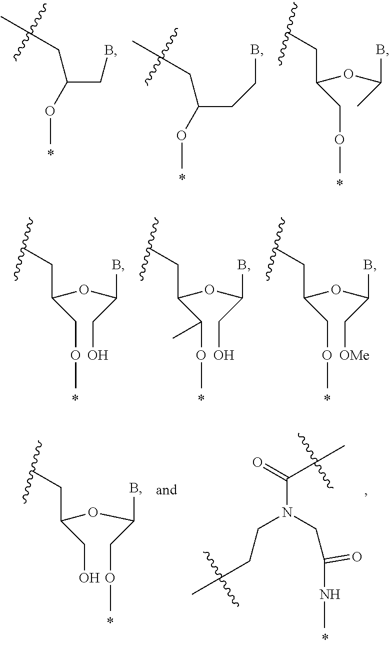
wherein:
* represents a bond to the subsequent optionally modified internucleotide linkage; and
B is an optionally modified nucleobase, or H.

51-54. (canceled)

55. The oligonucleotide of claim 32, wherein the oligonucleotide contains at least one 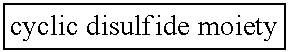
at the 5′-end of the oligonucleotide, and wherein the first nucleotide at the 5′-end of the oligonucleotide has the structure: 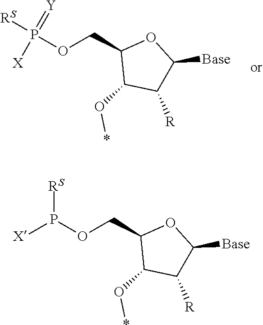
or a salt or a stereoisomer thereof, wherein
* represents a bond to the subsequent optionally modified internucleotide linkage;
Base is an optionally modified nucleobase;
R^{S} is the 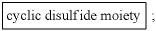
and
R is H, OH, O-methoxyalkyl, O-methyl, O-allyl, CH₂-allyl, fluoro, O-N-methylacetamido (O-NMA), O-dimethylaminoethoxyethyl (O-DMAEOE), O-aminopropyl (O-AP), or ara-F.

56-70. (canceled)

71. A pharmaceutical composition comprising the oligonucleotide of claim 32, and a pharmaceutically acceptable excipient.

72. A method for reducing or inhibiting the expression of a target gene in a subject, comprising:
administering to the subject the oligonucleotide of claim 32, in an amount sufficient to inhibit expression of the target gene.

73-78. (canceled)

79. A precursor compound comprising a structure of formula (I), or a salt or stereoisomer thereof: 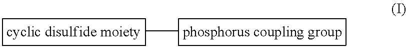
wherein the 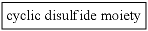
has the structure of: 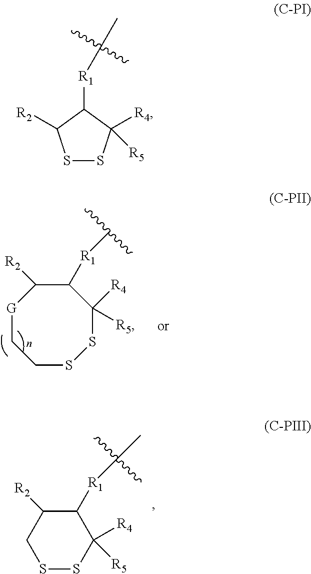
wherein:
R₁ is O or S, and is bonded to the P atom of the 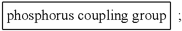
R₂ is (CH₂)ₛ—W, (CH₂)ₛ—O—(CH₂)ₛ—W, (CH₂)ₛ—(CH₂CH₂O)ₜ—(CH₂)ₛ—W, or (CH₂)ₛO(CH₂CH₂O)ₜ—(CH₂)ₛ—W;
s is an integer of 0-22,
t is an integer of 1-20;
W is a reactive group;
R₄ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₄ and R₅ form a second ring;
G is O, N(R′), S, or C(R⁴)(R¹)
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″, together with the adjacent nitrogen atom, form a ring; and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido.

80. The precursor compound of claim 79, wherein the 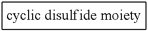
has the structure of: 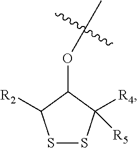
wherein R₄ and R₅ are each independently H, C₁₋₆ alkyl, or phenyl; or R₄ and R₅, together with the adjacent carbon atom, form a second ring of 3-7 atoms.

81-83. (canceled)

84. The precursor compound of claim 79, wherein W in R₂ is NHTFA, N₃, C≡CH, C(O)OR¹³, or OC(O)R¹³, wherein R¹³ is C₁-C₃ alkyl.

85. (canceled)

86. The precursor compound of claim 79, wherein the 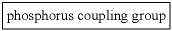
has the structure of: 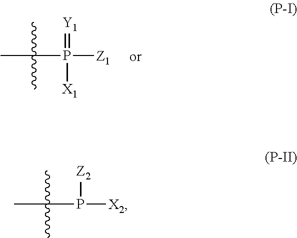
wherein:
X₁ and Z₁ are each independently H, OH, OM, OR¹³, SH, SM, SR¹³, C(O)H, S(O)H, or alkyl, each of which can be optionally substituted with one or more R^{sub} groups, N(R′)(R″), NSO₂R′, B(R¹³)₃, BH₃⁻, Se; or D-Q, wherein D is independently for each occurrence absent, 0, S, N(R′), alkylene, each of which can be optionally substituted with one or more R^{sub} groups, and Q is independently for each occurrence a nucleoside or oligonucleotide;
X₂ and Z₂ are each independently N(R′)(R″), OR¹⁸, or D-Q, wherein D is independently for each occurrence absent, O, S, N, N(R′), alkylene, each of which can be optionally substituted with one or more R^{sub} groups, and Q is independently for each occurrence a nucleoside or oligonucleotide,
Y₁ is S, O, or N(R′);
M is an organic or inorganic cation; and
R¹⁸ is H or alkyl, optionally substituted with one or more R^{sub} groups.

87-90. (canceled)

91. An oligonucleotide comprising one or more structures of formula (II): 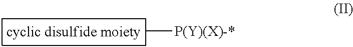
wherein the 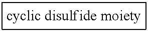
has the structure of: 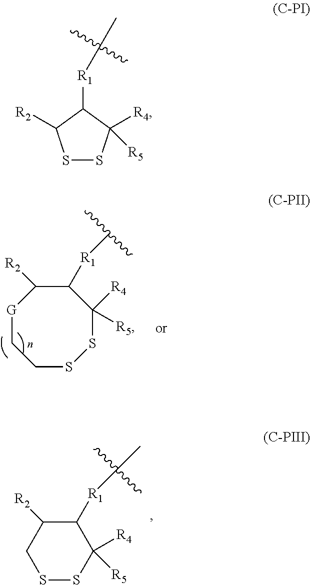
or a salt or stereoisomer thereof,
wherein:
* represents the bond to the oligonucleotide,
Y is absent, N(R′), =O, or =S, X is —OH, —SH, C(O)H, S(O)H, alkyl optionally substituted with one or more R^{sub} groups, N(R′)(R″), NSO₂R′, N═CN(R′)(R″), B(R¹³)₃, BH₃⁻, or X′, wherein X′ is N(R′)(R″), —OR¹³ or —SR³;
R₁ is O or S, and is bonded to the P atom of the —P(Y)(X)—* group;
R₂ is (CH₂)ₛ—W, (CH₂)ₛ—O—(CH₂)ₛ—W, (CH₂)ₛ—(CH₂CH₂O)ₜ—(CH₂)ₛ—W, or (CH₂)ₛO(CH₂CH₂O)ₜ—(CH₂)ₛ—W;
s is an integer of 0-22,
t is an integer of 1-20;
W is a reactive group;
R₄ and R₅ are each independently H, halo, CN or alkylene-CN, C(O)OR¹³ or alkylene-C(O)OR¹³, S(O)OR¹³ or alkylene-S(O)OR¹³, C(O)N(R′)(R″) or alkylene-C(O)N(R′)(R″), OR¹³ or alkylene-OR¹³, N(R′)(R″) or alkylene-N(R′)(R″), alkyl, C(R¹⁴)(R¹⁵)(R¹⁶) or alkylene-C(R¹⁴)(R¹⁵)(R¹⁶), alkenyl, alkynyl, cycloalkyl, heterocyclyl, aryl, heteroaryl, each of which can be optionally substituted by one or more R^{sub} groups; or R₄ and R₅ form a second ring;
G is O, N(R′), S, or C(R¹⁴)(R¹⁵);
n is an integer of 0-6;
R¹³ is independently for each occurrence H, alkyl, alkenyl, alkynyl, cycloalkyl, aryl, heteroaryl, aralkyl, ω-amino alkyl, ω-hydroxy alkyl, ω-hydroxy alkenyl, alkylcarbonyl, or arylcarbonyl, each of which can be optionally substituted with one or more R^{sub} groups;
R¹⁴, R¹⁵, and R¹⁶ are each independently H, halo, haloalkyl, alkyl, alkaryl, aryl, heteroaryl, aralkyl, hydroxy, alkyloxy, aryloxy, N(R′)(R″);
R′ and R″ are each independently H, alkyl, alkenyl, alkynyl, aryl, heteroaryl, hydroxy, alkyloxy, ω-amino alkyl, ω-hydroxy alkyl, ω-hydroxy alkenyl, or ω-hydroxy alkynyl, each of which can be optionally substituted with one or more R^{sub} groups; or R′ and R″ together with the adjacent nitrogen atom form a ring, and
R^{sub} is independently for each occurrence halo, haloalkyl, alkyl, alkaryl, aryl, aralkyl, hydroxy, alkyloxy, aryloxy, oxo, nitro, amino, acylamino, alkylcarbamoyl, arylcarbamoyl, alkylamino, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, arylcarbonyl, acyloxy, cyano, or ureido;
wherein at least one 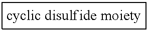
is connected at the 5′ end of the nucleoside or oligonucleotide.

92. The oligonucleotide of claim 91, wherein the 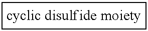
has the structure of: 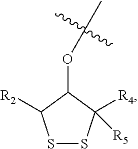
wherein R₄ and R₅ are each independently H, C₁₋₆ alkyl, or phenyl; or R₄ and R₅, together with the adjacent carbon atom, form a second ring of 3-7 atoms.

93-95. (canceled)

96. The oligonucleotide of claim 91, wherein W in R₂ is NHTFA, N₃, C≡CH, C(O)OR¹³, OC(O)R¹³, wherein R¹³ is C₁-C₃ alkyl.

97-98. (canceled)
